SEQUENCE LISTING (166 sequences)

SEQ ID NO: 1 - DNA, 383 bp, Artificial
  humanized antibody variable region
gaagttcaattgttagagtctggtggcggtcttgttcagcctggtggttctttacgtctttcttgcgctgcttccggattcactttctctcattacatgatggcttgggttcgccaagctcctggtaaaggtttggagtgggtttctcgtatcggtccttctggtggccctactcattatgctgactccgttaaaggtcgcttcactatctcagagacaactctaagaatactctctacttgcagatgaacagcttaagggctgaggacacggccgtgtattactgtgcgggatacgatagtggctacgattacgttgcagtggctgggcccgctgaatacttccagcactggggccagggcaccctggtcaccgtctcaagc

SEQ ID NO: 2 - DNA, 321 bp, Artificial
  humanized antibody variable region
gacatccagatgacccagtctccatcctccctgtctgcatctgtaggagacagagtcaccatcacttgccgggccagtcagagtattagtacttggttggcctggtatcagcagaaaccagggaaagcccctaaactcctgatctataaggcatctaatttacatacgggggtcccatctaggttcagcggcagtggatctggaacagaattcagtctcaccatcagcggcctgcagcctgatgattttgcaacctattattgccaacaatataatagttattctcggacgttcggccaagggaccaaggtggaaatcaaa

SEQ ID NO: 3 - protein, 128 aa, Artificial
  humanized antibody variable region
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSS

SEQ ID NO: 4 - protein, 107 aa, Artificial
  humanized antibody VL region
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 5 - protein, 5 aa, Artificial
  Antibody CDR
HYMMA

SEQ ID NO: 6 - protein, 17 aa, Artificial
  Antibody CDR
RIGPSGGPTHYADSVKG

SEQ ID NO: 7 - protein, 19 aa, Artificial
  Antibody CDR
YDSGYDYVAVAGPAEYFQH

SEQ ID NO: 8 - protein, 11 aa, Artificial
  Antibody CDR
RASQSISTWLA

SEQ ID NO: 9 - protein, 7 aa, Artificial
  Antibody CDR
KASNLHT

SEQ ID NO: 10 - protein, 9 aa, Artificial
  Antibody CDR
QQYNSYSRT

SEQ ID NO: 11 - DNA, 387 bp, Artificial
  synthetic construct
gaagttcaattgttagagtctggtggcggtcttgttcagcctggtggttctttacgtctttcttgcgctgcttccggattcactttctctcgttaccagatgatgtgggttcgccaagctcctggtaaaggtttggagtgggtttcttctatctctccttctggtggcgttactctttatgctgactccgttaaaggtcgcttcactatctctagagacaactctaagaatactctctacttgcagatgaacagcttaagggctgaggacacagccgtgtattactgtacgagagaacttttgggtactgtagtagtaccagttgcatggaaaatgcgtggctactttgactactggggccagctcaccctggtcaccgtctcaagc

SEQ ID NO: 12 - DNA, 324 bp, Artificial
  Synthetic construct
gacatccagatgacccagtctccaggcaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcatgcagtctgaagattttgcagtttattactgtcagcagtataataactggcccccgctcactttcggcggagggaccaaggtggagatcaaa

SEQ ID NO: 13 - protein, 129 aa, Artificial
  Synthetic construct
EVQLLESGGGLVQPGGSLRLSCAASGFTFSRYQMMWVRQAPGKGLEWVSSISPSGGVTLYADSVKGRFTISRDNSKNTLYLQMNSLRAEDTAVYYCTRELLGTVVVPVAWKMRGYFDYWGQLTLVTVSS

SEQ ID NO: 14 - protein, 108 aa, Artificial
  Synthetic construct
DIQMTQSPGTLSVSPGERATLSCRASQSVSSNLAWYQQKPGQAPRLLIYGASTRATGIPARFSGSGSGTEFTLTISSMQSEDFAVYYCQQYNNWPPLTFGGGTKVEIK

SEQ ID NO: 15 - protein, 5 aa, Artificial
  Antibody CDR
RYQMM

SEQ ID NO: 16 - protein, 17 aa, Artificial
  Antibody CDR
SISPSGGVTLYADSVKG

SEQ ID NO: 17 - protein, 20 aa, Artificial
  Antibody CDR
ELLGTVVVPVAWKMRGYFDY

SEQ ID NO: 18 - protein, 11 aa, Artificial
  Antibody CDR
RASQSVSSNLA

SEQ ID NO: 19 - protein, 8 aa, Artificial
  Antibody CDR
GASTRAST

SEQ ID NO: 20 - protein, 10 aa, Artificial
  Antibody CDR
QQYNNWPPLT

SEQ ID NO: 21 - DNA, 354 bp, Artificial
  Synthetic construct
gaagttcaattgttagagtctggtggcggtcttgttcagcctggtggttctttacgtctttcttgcgctgcttccggattcactttctctatgtacgctatgcgttgggttcgccaagctcctggtaaaggtttggagtgggtttctgttatcggtccttctggtggctggactccttatgctgactccgttaaaggtcgcttcactatctctagagacaactctaagaatactctctacttgcagatgaacagcttaagggctgaggacacggccgtgtattactgtgcgagagatcggggcatttacggtatggacgtctggggccaagggaccacggtcaccgtctcaagc

SEQ ID NO: 22 - DNA, 321 bp, Artificial
  Synthetic construct
gacatccagatgacccagtctccatccttcctgtctgcatctgtgggagacagagtcaccatcacttgccgggccagtcagggcattagtagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtctagaacttaataattaccctttcactttcggccttgggaccaaagtgcatatcaaa

SEQ ID NO: 23 - protein, 118 aa, Artificial
  Synthetic construct
EVQLLESGGGLVQPGGSLRLSCAASGFTFSMYAMRWVRQAPGKGLEWVSVIGPSGGWTPYADSVKGRFTISRDNSKNTLYLQMNSLRAEDTAVYYCARDRGIYGMDVWGQGTTVTVSS

SEQ ID NO: 24 - protein, 107 aa, Artificial
  Synthetic construct
DIQMTQSPSFLSASVGDRVTITCRASQGISSYLAWYQQKPGKAPKLLIYAASTLQSGVPSRFSGSGSGTEFTLTISSLQPEDFATYYCLELNNYPFTFGLGTKVHIK

SEQ ID NO: 25 - protein, 5 aa, Artificial
  Antibody CDR
MYAMR

SEQ ID NO: 26 - protein, 17 aa, Artificial
  Antibody CDR
VIGPSGGWTPYADSVKG

SEQ ID NO: 27 - protein, 9 aa, Artificial
  Antibody CDR
DRGIYGMDV

SEQ ID NO: 28 - protein, 11 aa, Artificial
  Antibody CDR
RASQGISSYLA

SEQ ID NO: 29 - protein, 7 aa, Artificial
  Antibody CDR
AASTLQS

SEQ ID NO: 30 - protein, 9 aa, Artificial
  Antibody CDR
LELNNYPFT

SEQ ID NO: 31 - DNA, 366 bp, Artificial
  Synthetic construct
gaagttcaattgttagagtctggtggcggtcttgttcagcctggtggttctttacgtctttcttgcgctgcttccggattcactttctctccttacgatatgctttgggttcgccaagctcctggtaaaggtttggagtgggtttctcgtatcggttcttctggtggctatactaagtatgctgactccgttaaaggtcgcttcactatctctagagacaactctaagaatactctctacttgcagatgaacagcttaagggctgaggacacggccgtgtattactgtgcgagagcccgcagcgtagtggttagctctgatgcttttgatatctggggccaagggacaatggtcaccgtctcaagc

SEQ ID NO: 32 - DNA, 321 bp, Artificial
  Synthetic construct
gacatccagatgacccagtctccatcttctgtgtctgcatctgtaggagacagagtcaccatcacttgtcgggcgagtcagggtattagtaagtggttagcctggtatcagcagaaaccagggaaagcccctaagctcctgatctttggtgcatccactttgcaaagtggggtcccatcaaagttcagcggcagtaaatctgggacagatttcactctcaccatcagcagcctgcagcctgaagattctgcaacttattactgccaacaatataatgattacccgctcactttcggcggagggaccaaggtggagattaaa

SEQ ID NO: 33 - protein, 122 aa, Artificial
  Synthetic construct
EVQLLESGGGLVQPGGSLRLSCAASGFTFSPYDMLWVRQAPGKGLEWVSRIGSSGGYTKYADSVKGRFTISRDNSKNTLYLQMNSLRAEDTAVYYCARARSVVVSSDAFDIWGQGTMVTVSS

SEQ ID NO: 34 - protein, 107 aa, Artificial
  Synthetic construct
DIQMTQSPSSVSASVGDRVTITCRASQGISKWLAWYQQKPGKAPKLLIFGASTLQSGVPSKFSGSKSGTDFTLTISSLQPEDSATYYCQQYNDYPLTFGGGTKVEIK

SEQ ID NO: 35 - protein, 5 aa, Artificial
  Antibody CDR
PYDML

SEQ ID NO: 36 - protein, 17 aa, Artificial
  Antibody CDR
RIGSSGGYTKYADSVKG

SEQ ID NO: 37 - protein, 13 aa, Artificial
  Antibody CDR
ARSVVVSSDAFDI

SEQ ID NO: 38 - protein, 11 aa, Artificial
  Antibody CDR
RASQGISKWLA

SEQ ID NO: 39 - protein, 7 aa, Artificial
  Antibody CDR
GASTLQS

SEQ ID NO: 40 - protein, 9 aa, Artificial
  Antibody CDR
QQYNDYPLT

SEQ ID NO: 41 - DNA, 351 bp, Artificial
  Synthetic construct
gaagttcaattgttagagtctggtggcggtcttgttcagcctggtggttctttacgtctttcttgcgctgcttccggattcactttctctaattacaatatgtattgggttcgccaagctcctggtaaaggtttggagtgggtttctgttatcgttccttctggtggcaagacttcttatgctgactccgttaaaggtcgcttcactatctctagagacaactctaagaatactctctacttgcagatgaacagcttaagggctgaggacacggccgtgtattactgtgcgagatcgtacggagggggatttgactactggggccagggcaccctggtcaccgtctcaagc

SEQ ID NO: 42 - DNA, 321 bp, Artificial
  Synthetic construct
gacatccagatgacccagtctccatcttccgtgtctgcatctgttggagacaaagtcaccatcacttgtcgggcgagtcaggatattctcacctggttagcctggtatcagtggaaaccagggaaagcccctaagctcctgatctatgctgcatccagtttgcaaagtggggtcccatcaaggttcagcggcagtggatctgggacagatttcactctcatcatcgacaccctgcagcctgaggattttgcaacttactactgtcaacaggctatccgtttcccgctcactttcggcggagggaccaaggtggagatcaag

SEQ ID NO: 43 - protein, 117 aa, Artificial
  Synthetic construct
EVQLLESGGGLVQPGGSLRLSCAASGFTFSNYNMYWVRQAPGKGLEWVSVIVPSGGKTSYADSVKGRFTISRDNSKNTLYLQMNSLRAEDTAVYYCARSYGGGFDYWGQGTLVTVSS

SEQ ID NO: 44 - protein, 107 aa, Artificial
  Synthetic construct
DIQMTQSPSSVSASVGDKVTITCRASQDILTWLAWYQWKPGKAPKLLIYAASSLQSGVPSRFSGSGSGTDFTLIIDTLQPEDFATYYCQQAIRFPLTFGGGTKVEIK

SEQ ID NO: 45 - protein, 5 aa, Artificial
  Antibody CDR
NYNMY

SEQ ID NO: 46 - protein, 16 aa, Artificial
  Antibody CDR
VIVPSGKTSYADSVKG

SEQ ID NO: 47 - protein, 8 aa, Artificial
  Antibody CDR
SYGGGFDY

SEQ ID NO: 48 - protein, 11 aa, Artificial
  Antibody CDR
RASQDILTWLA

SEQ ID NO: 49 - protein, 7 aa, Artificial
  Antibody CDR
AASSLQS

SEQ ID NO: 50 - protein, 9 aa, Artificial
  Antibody CDR
QQAIRFPLT

SEQ ID NO: 51 - DNA, 375 bp, Artificial
  Synthetic construct
gaagttcaattgttagagtctggtggcggtcttgttcagcctggtggttctttacgtctttcttgcgctgcttccggattcactttctcttattaccgtatgtattgggttcgccaagctcctggtaaaggtttggagtgggtttcttctatctattcttctggtggccctacttattatgctgactccgttaaaggtcgcttcactatctctagagacaactctaagaatactctctacttgcagatgaacagcttaagggctgaggacacggccgtgtattactgtgcgaaagatatgggtaccggtttttggagtggttggggcctaggctctgactactggggccagggaaccctggtcaccgtctcaagc

SEQ ID NO: 52 - DNA, 321 bp, Artificial
  Synthetic construct
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctctcactttcggcggagggaccaaggtggagatcaaa

SEQ ID NO: 53 - protein, 125 aa, Artificial
  Antibody variable region
EVQLLESGGGLVQPGGSLRLSCAASGFTFSYYRMYWVRQAPGKGLEWVSSIYSSGGPTYYADSVKGRFTISRDNSKNTLYLQMNSLRAEDTAVYYCAKDMGTGFWSGWGLGSDYWGQGTLVTVSS

SEQ ID NO: 54 - protein, 107 aa, Artificial
  Antibody variable region
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 55 - protein, 5 aa, Artificial
  Antibody CDR
YYRMY

SEQ ID NO: 56 - protein, 17 aa, Artificial
  Antibody CDR
SIYSSGGPTYYADSVKG

SEQ ID NO: 57 - protein, 16 aa, Artificial
  Antibody CDR
DMGTGFWSGWGLGSDY

SEQ ID NO: 58 - protein, 11 aa, Artificial
  Antibody CDR
RASQGISSWLA

SEQ ID NO: 59 - protein, 7 aa, Artificial
  Antibody CDR
AASSLQS

SEQ ID NO: 60 - protein, 9 aa, Artificial
  Antibody CDR
QQANSFPLT

SEQ ID NO: 61 - DNA, 361 bp, Artificial
  Synthetic construct
gaggtgcagctggtggagtctgggggaggtgtggtacggcctggggggtccctgagactctcctgtgcagcctctgggttcaccgtcagtgattactccatgaactgggtccgccaggctccagggaagggcctggagtggattgggtttattagaaacaaagctaatgcctacacaacagagtacagtgcatctgtgaagggtagattcaccatctcaagagatgattcaaaaaacacgctgtatctgcaaatgaacagcctgaaaaccgaggacacagccgtgtattactgtaccacataccctaggtatcatgctatggactcctggggccagggcaccatggtcaccgtctcctcag

SEQ ID NO: 62 - DNA, 321 bp, Artificial
  synthetic construct
gccatccagttgactcagtctccatcctccctgtctgcatctgtaggagacagagtcaccatcacttgcagggccagccaaagtattagcaacaacctacactggtacctgcagaagccagggcagtctccacagctcctgatctattatggcttccagtccatctctggggtcccatcaaggttcagtggcagtggatctgggacagatttcactctcaccatcagcagtctgcaacctgaagattttgcaacttactactgtcaacaggccaacagctggccgctcacgttcggcggagggaccaagctggagatcaaa

SEQ ID NO: 63 - protein, 120 aa, Artificial
  Antibody Variable Heavy chain
EVQLVESGGGVVRPGGSLRLSCAASGFTVSDYSMNWVRQAPGKGLEWIGFIRNKANAYTTEYSASVKGRFTISRDDSKNTLYLQMNSLKTEDTAVYYCTTYPRYHAMDSWGQGTMVTVSS

SEQ ID NO: 64 - protein, 107 aa, Artificial
  Antibody Variable region
AIQLTQSPSSLSASVGDRVTITCRASQSISNNLHWYLQKPGQSPQLLIYYGFQSISGVPSRFSGSGSGTDFTLTISSLQPEDFATYYCQQANSWPLTFGGGTKLEIK

SEQ ID NO: 65 - protein, 5 aa, Artificial
  Synthetic construct
DYSMN

SEQ ID NO: 66 - protein, 19 aa, Artificial
  Synthetic construct
FIRNKANAYTTEYSASVKG

SEQ ID NO: 67 - protein, 9 aa, Artificial
  Synthetic construct
YPRYHAMDS

SEQ ID NO: 68 - protein, 11 aa, Artificial
  Antibody CDR
RASQSISNNLH

SEQ ID NO: 69 - protein, 7 aa, Artificial
  Antibody CDR
YGFQSIS

SEQ ID NO: 70 - protein, 9 aa, Artificial
  Antibody CDR
QQANSWPLT

SEQ ID NO: 71 - protein, 115 aa, Artificial
  synthetic construct
DVKLVESGGGLVKPGGSLKLSCAASGFTFSSYTMSWVRQTPEKRLEWVATISSGGTYTYYPDSVKGRFTISRDNAKNTLYLQMSSLKSEDTAMYYCTREAIFTYWGQGTLVTVSA

SEQ ID NO: 72 - protein, 107 aa, Artificial
  Antibody variable region
DIKMTQSPSSMYASLGERVTITCKASQDINNYLSWFQQKPGKSPKTLIYRANRLVDGVPSRFSGSGSGQDYSLTISSLEYEDMGIYYCLKYDEFPYTFGGGTKLEIK

SEQ ID NO: 73 - protein, 5 aa, Artificial
  synthetic construct
SYTMS

SEQ ID NO: 74 - protein, 17 aa, Artificial
  synthetic construct
TISSGGTYTYYPDSVKG

SEQ ID NO: 75 - protein, 6 aa, Artificial
  synthetic construct
EAIFTY

SEQ ID NO: 76 - protein, 11 aa, Artificial
  Antibody CDR
KASQDINNYLS

SEQ ID NO: 77 - protein, 7 aa, Artificial
  Antibody CDR
RANRLVD

SEQ ID NO: 78 - protein, 9 aa, Artificial
  Antibody CDR
LKYDEFPYT

SEQ ID NO: 79 - protein, 115 aa, Artificial
  Synthetic construct
EVQLQQSGPELVKTGASVKISCKASGYSFTGYYMHWVKQSHGKSLEWIGYISCYNGVTSYNQKFKGKATFTVDTSSSTAYMQFNSLTSEDSAVYYCARSHAMDYWGQGTSVTVSS

SEQ ID NO: 80 - protein, 112 aa, Artificial
  Antibody variable region
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGSHFPWTFGGGTKLEIK

SEQ ID NO: 81 - protein, 5 aa, Artificial
  synthetic construct
GYYMH

SEQ ID NO: 82 - protein, 17 aa, Artificial
  synthetic construct
YISCYNGVTSYNQKFKG

SEQ ID NO: 83 - protein, 6 aa, Artificial
  synthetic construct
SHAMDY

SEQ ID NO: 84 - protein, 16 aa, Artificial
  Antibody CDR
KSSQSLLYSNGKTYLN

SEQ ID NO: 85 - protein, 7 aa, Artificial
  Antibody CDR
LVSKLDS

SEQ ID NO: 86 - protein, 9 aa, Artificial
  Antibody CDR
VQGSHFPWT

SEQ ID NO: 87 - protein, 120 aa, Artificial
  Synthetic construct
EVKLVESGGGLVQPGGSLSLSCAASGFTFTDYSMNWVRQPPGKALEWLGFIRNKANDYTTEYSASVKGRFTISRDNSQSILYLQMNALRAEDSATYYCVRYPRYHAMDSWGQGTSVTVSS

SEQ ID NO: 88 - protein, 107 aa, Artificial
  Antibody variable region
DIVLTQSPATLSVTPGDSVNLSCRASQSISNNLHWYQQKSHESPRLLIKYVFQSISGIPSRFSGSGSGTDFTLSINSVETEDFGMYFCQQSNSWPLTFGAGTKLELK

SEQ ID NO: 89 - protein, 5 aa, Artificial
  synthetic construct
DYSMN

SEQ ID NO: 90 - protein, 19 aa, Artificial
  synthetic construct
FIRNKANDYTTEYSASVKG

SEQ ID NO: 91 - protein, 9 aa, Artificial
  synthetic construct
YPRYHAMDS

SEQ ID NO: 92 - protein, 11 aa, Artificial
  Antibody CDR
RASQSISNNLH

SEQ ID NO: 93 - protein, 7 aa, Artificial
  Antibody CDR
YVFQSIS

SEQ ID NO: 94 - protein, 9 aa, Artificial
  Antibody CDR
QQSNSWPLT

SEQ ID NO: 95 - protein, 118 aa, Artificial
  synthetic construct
QVQLQQPGAELVKPGASVKLSCKASGYTFTSYWMHWVKQRPGQGLEWIGMIHPNSGSTNYNEKFKSKATLTVDKSSSTAYMRLSSLTSEDSAVYYCARGGNMVGGGYWGQGTTLTVSS

SEQ ID NO: 96 - protein, 106 aa, Artificial
  Antibody variable region
QIVLTQSPALMSASPGEKVTMTCSASSSVSYMYWYQQKPRSSPKPWIYLTTNLASGVPARFSGSGSGTSYSLTISSMEAEDAATYYCQQWSSNPFTFGSGTKLEIR

SEQ ID NO: 97 - protein, 5 aa, Artificial
  synthetic construct
SYWMH

SEQ ID NO: 98 - protein, 17 aa, Artificial
  synthetic construct
MIHPNSGSTNYNEKFKS

SEQ ID NO: 99 - protein, 9 aa, Artificial
  synthetic construct
GGNMVGGGY

SEQ ID NO: 100 - protein, 10 aa, Artificial
  Antibody CDR
SASSSVSYMY

SEQ ID NO: 101 - protein, 7 aa, Artificial
  Antibody CDR
LTTNLAS

SEQ ID NO: 102 - protein, 9 aa, Artificial
  Antibody CDR
QQWSSNPFT

SEQ ID NO: 103 - protein, 120 aa, Artificial
  Synthetic construct
QMQLVQSGPEVKKPGTSVKVSCKASGFTFTDYSMNWVRQARGQRLEWIGFIRNKANDYTTEYSASVKGRVTITRDMSTSTAYMELSSLRSEDTAVYYCARYPRYHAMDSWGQGTSVTVSS

SEQ ID NO: 104 - protein, 108 aa, Artificial
  Antibody variable region
DIQMTQSPSSLSASVGDRVTITCRASQSISNNLHWYQQKPGKAPKLLIKYVFQSISGVPSRFSGSGSGTDFTFTISSLQPEDFATYYCQQSNSWPLTFGGGTKVEIKG

SEQ ID NO: 105 - protein, 5 aa, Artificial
  synthetic construct
DYSMN

SEQ ID NO: 106 - protein, 19 aa, Artificial
  synthetic construct
FIRNKANDYTTEYSASVKG

SEQ ID NO: 107 - protein, 9 aa, Artificial
  synthetic construct
YPRYHAMDS

SEQ ID NO: 108 - protein, 11 aa, Artificial
  Antibody CDR
RASQSISNNLH

SEQ ID NO: 109 - protein, 7 aa, Artificial
  Antibcdy CDR
YVFQSIS

SEQ ID NO: 110 - protein, 9 aa, Artificial
  Antibody CDR
QQSNSWPLT

SEQ ID NO: 111 - DNA, 990 bp, Artificial
  Antibody constant region heavy chain
gcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 112 - DNA, 321 bp, Artificial
  Antibody constant region light chain
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 113 - protein, 330 aa, Artificial
  Antibody constant heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 107 aa, Artificial [sequence omitted]

SEQ ID NO: 115 - protein, 121 aa, Artificial
  Synthetic construct
QVQLLQSGAEVKKPGASVKVPCKASGYTFTSYAMSWVRQAPGQGLEWMGWINTNTGNPTYAQGFTGRFVFSLDTSVSTAYLQISSLKAEDTAVYYCARVRTTVYGDGMDVWGQGTLVTVSS

SEQ ID NO: 116 - protein, 106 aa, Artificial
  Synthetic construct
EIVLTQSPATLSVSPGERATLSCRASQSVSSNLAWYQQKPGQAPRLLIYGASTRATGIPDRFSASGSGTDFTLTISRVEPEDFAVYYCQQYGSSWTFGQGTKVEIK

SEQ ID NO: 117 - protein, 121 aa, Artificial
  Synthetic construct
QVQLLQSGAEVKKPGASVKVPCKASGYTFTSRAMSWVRQAPGQGLEWMGWINTNTGNPTYAQGSTGRFVFSLDTSVSTAYLQISSLKAEDTAVYYCARVRFTVYGDGMDVWGQGTLVTVSS

SEQ ID NO: 118 - protein, 106 aa, Artificial
  Synthetic construct
EIVLTQSPATLSVSPGERATLSCRASQSVSSNPAWYQQKPGQAPRLLIYGASTRATGIPDRFSASGSGTDFTLTISRVEPEDFAVYYCQQYGPSWTFGQGTKVEIK

SEQ ID NO: 119 - protein, 121 aa, Artificial
  Synthetic construct
QVQLLQSGAEVKKPGASVKVPCKASGYTFTSRAMSWVRQAPGQGLEWMGWINTNTGNPTYAQGFTGRFVFSLDTSVSTAYLQISSLKAEDTAVYYCARVRFTVYGDGMDVWGQGTLVTVSS

SEQ ID NO: 120 - protein, 106 aa, Artificial
  Synthetic construct
EIVLTQSPATLSVSPGERATLSCRASQSVSSNPAWYQQKPGQAPRLLIYGASTWATGIPDRFSASGSGTDFTLTISRVEPEDFAVYYCQQYGPSWTFGQGTKVEIK

SEQ ID NO: 121 - protein, 121 aa, Artificial
  Synthetic construct
QVQLLQSGAEVKKPGASVKVPCKASGYTFTSRAMSWVRQAPGQGLEWMGWINTNTGNPTYAQGFTGRFVFSLDTSVSTAYLQISSLKAEDTAVYYCARVRTTVYGDNMDVWGQGTLVTVSS

SEQ ID NO: 122 - protein, 106 aa, Artificial
  Synthetic construct
EIVLTQSPATLSVSPGERATLSCRASQSVSSNLPWYQQKPGQAPRLLIYGASTRATGIPDRFSASGSGTDFTLTISRVEPEDFAVYYCQQYGPSWTFGQGTKVEIK

SEQ ID NO: 123 - protein, 121 aa, Artificial; identical to SEQ ID NO: 119
  Synthetic construct

SEQ ID NO: 124 - protein, 106 aa, Artificial
  Synthetic construct
EIVLTQSPATLSVSPGERATLSCRASQSVSSNLPWYQQKPGQAPRLLIYGASTRPTGIPDRFSASGSGTDFTLTISRVEPEDFAVYYCQQYGPSWTFGQGTKVEIK

SEQ ID NO: 125 - protein, 121 aa, Artificial; identical to SEQ ID NO: 119
  Synthetic construct

SEQ ID NO: 126 - protein, 106 aa, Artificial
  Synthetic construct
EIVLTQSPATLSVSPGERATLSCRASQSVSSNLAWYQQKPGQAPRLLIYGASTRPTGIPDRFSASGSGTDFTLTISRVEPEDFAVYYCQQYGPSWTFGQGTKVEIK

SEQ ID NO: 127 - protein, 121 aa, Artificial; identical to SEQ ID NO: 119
  Synthetic construct

SEQ ID NO: 128 - protein, 106 aa, Artificial
  Synthetic construct
EIVLTQSPATLSVSPGERATLSCRASQSVSSNLAWYQQKPGQAPRLLIYGASTWATGIPDRFSASGSGTDFTLTISRVEPEDFAVYYCQQYGPSWTFGQGTKVEIK

SEQ ID NO: 129 - protein, 121 aa, Artificial
  Synthetic construct
QVQLLQSGAEVKKPGASVKVPCKASGYTFTSRAMSWVRQAPGQGLEWMGWINTNTGNPTYAQGFTGRFVFSLDTSVSTAYLQISSLKAEDTAVYYCARVRLTVYGDGMDVWGQGTLVTVSS

SEQ ID NO: 130 - protein, 106 aa, Artificial
  Synthetic construct
EIVLTQSPATLSVSPGERATLSCRASQSVSSNPPWYQQKPGQAPRLLIYGASTRATGIPDRFSASGSGTDFTLTISRVEPEDFAVYYCQQYGSSWTFGQGTKVEIK

SEQ ID NO: 131 - protein, 121 aa, Artificial; identical to SEQ ID NO: 119
  Synthetic construct

SEQ ID NO: 132 - protein, 106 aa, Artificial; identical to SEQ ID NO: 118
  Synthetic construct

SEQ ID NO: 133 - protein, 121 aa, Artificial; identical to SEQ ID NO: 129
  Synthetic construct

SEQ ID NO: 134 - protein, 106 aa, Artificial
  Synthetic construct
EIVLTQSPATLSVSPGERATLSCRASQSVSSNPAWYQQKPGQAPRLLIYGLSTRATGIPDRFSASGSGTDFTLTISRVEPEDFAVYYCQQYGPSWTFGQGTKVEIK

SEQ ID NO: 135 - protein, 121 aa, Artificial
  Synthetic construct
QVQLLQSGAEVKKPGASVKVPCKASGYTFTSYAMSWVRQAPGQGLEWMGWINTNTGNPTYAQGFTGRFVFSLDTSVSTAYLQISSLKAEDTAVYYCARVRLTVYGDGMDVWGQGTLVTVSS

SEQ ID NO: 136 - protein, 106 aa, Artificial; identical to SEQ ID NO: 116
  Synthetic construct

SEQ ID NO: 137 - protein, 107 aa, Artificial
  Synthetic construct
DIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIK

SEQ ID NO: 138 - protein, 115 aa, Artificial
  Synthetic construct
QVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTVSS

SEQ ID NO: 139 - DNA, 381 bp, Artificial
  Synthetic construct
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagggggggtatagcagcagctggttactggggcttggggtacaactggttcgacccctggggccagggaaccctggtcaccgtctcctca

SEQ ID NO: 140 - protein, 127 aa, Artificial
  Synthetic construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGGIAAAGYWGLGYNWFDPWGQGTLVTVSS

SEQ ID NO: 141 - DNA, 336 bp, Artificial
  Synthetic construct
cagtctgtgttgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacaacagcctgagtggttcggtggttttcggcggagggaccaagctgaccgtccta

SEQ ID NO: 142 - protein, 112 aa, Artificial
  Synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDNSLSGSVVFGGGTKLTVL

SEQ ID NO: 143 - protein, 107 aa, Artificial
  Synthetic construct
DIQLTQSPSSLSASVGDRVTITCKASQDINNYLSWYQQKPGQAPRLLIYRANRLVDGVPDRFSGSGYGTDFTLTINNIESEDAAYYFCLKYDEFPYTFGQGTKVEIK

SEQ ID NO: 144 - protein, 115 aa, Artificial; identical to SEQ ID NO: 138
  Synthetic construct

SEQ ID NO: 145 - protein, 107 aa, Artificial
  Synthetic construct
DIQLTQSPSSLSLSPGERVTLSCKASQDINNYLSWYQQKPDQAPKLLIKRANRLVDGVPDRFSGSGSGTDFTLKISRVEAEDVGVYYCLKYDEFPYTFGQGTRLEIK

SEQ ID NO: 146 - protein, 115 aa, Artificial; identical to SEQ ID NO: 138
  Synthetic construct

SEQ ID NO: 147 - protein, 107 aa, Artificial
  Synthetic construct
DIQMTQSPSSLSASVGDRVTITCRASQSISNNLHWYQQKPGKAPKLLIKYVFQSISGVPSRFSGSGSGTDFTFTISSLQPEDFATYYCQQSNSWPLTFGGGTKVEIK

SEQ ID NO: 148 - protein, 120 aa, Artificial
  Synthetic construct
QMQLVQSGPEVKKPGTSVKVSCKASGFTFTDYSMNWVRQARGQRLEWIGFIRNKANDYTTEYADSVKGRVTITRDMSTSTAYMELSSLRSEDTAVYYCARYPRYHAMDSWGQGTSVTVSS

SEQ ID NO: 149 - protein, 120 aa, Artificial; identical to SEQ ID NO: 103
  Synthetic construct

SEQ ID NO: 150 - protein, 107 aa, Artificial
  Synthetic construct
DIVMTQSPSSLSASVGDRVTIICRASQSISNNLHWYQQKPGKAPKLLIKYVFQSISGVPSRFSGSGSGTDFTFTISSLQPEDFATYYCQQSNSWPLTFGGGTKVEIK

SEQ ID NO: 151 - protein, 120 aa, Artificial
  Synthetic construct
QMQLVQSGPEVKKPGTSVKVSCKASGFTFTDYSMNWVRQARGQRLEWLGFIRNKANDYTTEYSASVKGRVTITRDMSTSTAYMELSSLRSEDTAVYYCARYPRYHAMDSWGQGTSVTVSS

SEQ ID NO: 152 - protein, 107 aa, Artificial; identical to SEQ ID NO: 150
  Synthetic construct

SEQ ID NO: 153 - protein, 120 aa, Artificial
  Synthetic construct
EVQLVESGGGVVRPGGSLRLSCAASGFTVSDYSMNWVRQAPGKGLEWIGFIRNKANDYTTEYSASVKGRFTISRDDSKNTLYLQMNSLKTEDTAVYYCTTYPRYHAMDSWGQGTMVTVSS

SEQ ID NO: 154 - protein, 107 aa, Artificial
  Synthetic construct
AIQLTQSPSSLSASVGDRVTITCRASQSISNNLHWYLQKPGQSPQLLIYYVFQSISGVPSRFSGSGSGTDFTLTISSLQPEDFATYYCQQSNSWPLTFGGGTKLEIK

SEQ ID NO: 155 - protein, 120 aa, Artificial
  Synthetic construct
EVQLVESGGGLVQPGGSLRLSCAASGFTFSDYSMNWVRQAPGKGLEWVAFIRNKANDYTTEYSASVKGRFTISRDNSKNTLYLQMNSLRAEDTAMYYCARYPRYHAMDSWGQGTLVTVSS

SEQ ID NO: 156 - protein, 107 aa, Artificial
  Synthetic construct
EIVLTQSPATLSVSPGERATLSCRASQSISNNLHWYQQKPGKAPKSLIYYVFQSISGVPSRFSGSGSGTDFTLTINSLEAEDAATYYCQQSNSWPLTFGGGTKVDIK

SEQ ID NO: 157 - protein, 120 aa, Artificial
  Synthetic construct
EVQLVESGGGLVKPGGSLRLSCAASGFTFSDYSMNWVRQASGKGLEWVGFIRNKANDYTTEYSASVKGRFTISRDDSKNTLYLQMNSLKTEDTAVYYCTTYPRYHAMDSWGQGTLVTVSS

SEQ ID NO: 158 - protein, 107 aa, Artificial; identical to SEQ ID NO: 156
  Synthetic construct

SEQ ID NO: 159 - protein, 120 aa, Artificial
  Synthetic construct
QMQLVQSGPEVKKPGTSVKVSCKASGFTFDDYSMTWVRQARGQRLEWLGFIRNKANAYTTEYSASVKGRVTITRDMSTSTAYMELSSLRSEDTAVYYCARYPRHHAMDSWGQGTSVTVSS

SEQ ID NO: 160 - protein, 107 aa, Artificial
  Synthetic construct
DIQMTQSPSSLSASVGDRVTITCRASQSISNNLHWYQQKPGKAPKLLIKYAFQSISGVPSRFSGSGSGTDFTFTISSLQPEDFATYYCQQSNSWPLTFGGGTKVEIK

SEQ ID NO: 161 - protein, 120 aa, Artificial
  Synthetic construct
QMQLVQSGPEVKKPGTSVKVSCKASGFTFDDYSMNWVRQARGQRLEWIGFIRNKANDYTTEYSASVKGRVTITRDMSTSTAYMELSSLRSEDTAVYYCARYPRHHAMDSWGQGTSVTVSS

SEQ ID NO: 162 - protein, 107 aa, Artificial
  Synthetic construct
DIQMTQSPSSLSASVGDRVTITCRASQSISNNLHWYQQKPGKAPKLLIKYAFQSISGVPSRFSGSGSGTDFTFTISSLQPEDFATYYCQQANSWPLTFGGGTKVEIK

SEQ ID NO: 163 - protein, 120 aa, Artificial
  Synthetic construct
QMQLVQSGPEVKKPGTSVKVSCKASGFTFDDYSMTWVRQARGQRLEWLGFIRNKANAYTTEYADSVKGRVTITRDMSTSTAYMELSSLRSEDTAVYYCARYPRHHAMDSWGQGTSVTVSS

SEQ ID NO: 164 - protein, 107 aa, Artificial; identical to SEQ ID NO: 160
  Synthetic construct

SEQ ID NO: 165 - protein, 120 aa, Artificial
  Synthetic construct
QMQLVQSGPEVKKPGTSVKVSCKASGFTFDDYSMNWVRQARGQRLEWIGFIRNKANDYTTEYADSVKGRVTITRDMSTSTAYMELSSLRSEDTAVYYCARYPRHHAMDSWGQGTSVTVSS

SEQ ID NO: 166 - protein, 107 aa, Artificial; identical to SEQ ID NO: 162
  Synthetic construct

(19) United States
(12) Patent Application Publication
(10) Pub. No.: US 2009/0304721 A1
(43) Pub. Date: Dec. 10, 2009

(54) TOXIN CONJUGATED EPH RECEPTOR ANTIBODIES

(75) Inventors: Michael S. Kinch, Laytonsville, MD (US); Herren Wu, Boyds, MD (US); Christine Bachy, Gaithersburg, MD (US); David Tice, Gaithersburg, MD (US); Changsou Gao, Potomac, MD (US)

Correspondence Address: MEDIMMUNE, LLC;Patrick Scott Alban, ONE MEDIMMUNE WAY, GAITHERSBURG, MD, 20878, US

(73) Assignees: Medlmmune, Inc, Gaithersburg, MD (US); Seattle Genetics, Inc, Bothell, WA (US)

(21) Appl. No.: 12/065,832

(22) PCT Filed: Sep. 7, 2006
(86) PCT No.: PCT/US06/34894

§ 371 Date: Aug. 4, 2008

Related U.S. Application Data
(60) Provisional application No. 60/714,362, filed on Sep. 7, 2005.
(60) Provisional application No. 60/735,966, filed on Nov. 14, 2005.

(51) Int. Cl. A61K 39/395; C07K 16/18; C12N 15/00; C12N 5/06

(52) U.S. Cl. 424/183.1; 530/391.7; 435/320.1; 435/330

(57) ABSTRACT

The present invention relates to compositions and methods for inducing cell death or stasis in cancer cells or other hyperproliferative cells using anti-EphA2 or anti-EphA4 antibodies conjugated to toxins.

This application claims priority to U.S. Provisional Patent Application No. 60/714,362, filed Sep. 7, 2005 and U.S. Provisional Patent Application No. 60/735,966, filed Nov. 14, 2005, each of which is hereby incorporated herein by reference.

### FIELD OF THE INVENTION

The present invention provides compositions and methods for inducing cell death or stasis in cancer cells or other hyperproliferative cells using anti-EphA2 or anti-EphA4 antibodies conjugated to toxins.

### BACKGROUND OF THE INVENTION

### Cancer

A neoplasm, or tumor, is a neoplastic mass resulting from abnormal uncontrolled cell growth, which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W.B. Saunders Co., Philadelphia, pp. 68-122). Cancer can arise in many sites of the body and behave differently depending upon its origin. Cancerous cells destroy the part of the body in which they originate and then spread to other part(s) of the body where they start new growth and cause more destruction.

More than 1.2 million Americans develop cancer each year. Cancer is the second leading case of death in the United States and if current trends continue, cancer is expected to be the leading cause of the death by the year 2010. Lung and prostate cancer are the top cancer killers for men in the United States. Lung and breast cancer are the top cancer killers for women in the United States. One in two men in the United States will be diagnosed with cancer at some time during his lifetime. One in three women in the United States will be diagnosed with cancer at some time during her lifetime. Current treatment options, such as surgery, chemotherapy and radiation treatment, are oftentimes either ineffective or present serious side effects.

### Metastasis

The most life-threatening forms of cancer often arise when a population of tumor cells gains the ability to colonize distant and foreign sites in the body. These metastatic cells survive by overriding restrictions that normally constrain cell colonization into dissimilar tissues. For example, typical mammary epithelial cells will generally not grow or survive if transplanted to the lung, yet lung metastases are a major cause of breast cancer morbidity and mortality. Recent evidence suggests that dissemination of metastatic cells through the body can occur long before clinical presentation of the primary tumor. These micrometastatic cells may remain dormant for many months or years following the detection and removal of the primary tumor. Thus, a better understanding of the mechanisms that allow for the growth and survival of metastatic cells in a foreign microenvironment is critical for the improvement of therapeutics designed to fight metastatic cancer and diagnostics for the early detection and localization of metastases.

### Cancer Cell Signaling

Cancer is a disease of aberrant signal transduction. Aberrant cell signaling overrides anchorage-dependent constraints on cell growth and survival (Rhim, et al., Critical Reviews in Oncogenesis 8:305, 1997; Patarca, Critical Reviews in Oncogenesis 7:343, 1996; Malik, et al., Biochimica et Biophysica Acta 1287:73, 1996; Cance, et al., Breast Cancer Res Treat 35:105, 1995). Tyrosine kinase activity is induced by ECM anchorage and indeed, the expression or function of tyrosine kinases is usually increased in malignant cells (Rhim, et al., Critical Reviews in Oncogenesis 8:305, 1997; Cance, et al., Breast Cancer Res Treat 35:105, 1995; Hunter, Cell 88:333, 1997). Based on evidence that tyrosine kinase activity is necessary for malignant cell growth, tyrosine kinases have been targeted with new therapeutics (Levitzki, et al., Science 267:1782, 1995; Kondapaka, et al., Molecular & Cellular Endocrinology 117:53, 1996; Fry, et al., Current Opinion in BioTechnology 6: 662, 1995). Unfortunately, obstacles associated with specific targeting to tumor cells often limit the application of these drugs. In particular, tyrosine kinase activity is often vital for the function and survival of benign tissues (Levitzki, et al., Science 267:1782, 1995). To minimize collateral toxicity, it is critical to identify and then target tyrosine kinases that are selectively overexpressed in tumor cells.

### Eph Family of Receptor Tyrosine Kinases

The Eph family of receptors are the largest family of receptor tyrosine kinases (RTKs) (Gale et al., 1997, Cell Tissue Research 290(2): 227-241 and Dodelet et al., 2000, Oncogene 19(49): 5614-9). The Eph receptors, and their membrane bound ephrin ligands are important mediators of cell-cell communication regulating cell attachment, shape, and mobility. Eph RTK signaling events control multiple aspects of embryonic development, particularly in the nervous system (reviewed in Kullander et al., 2002, Nat. Rev. Mol. Cell Biol. 3:473 and Mamling et al., 2002, Trends Biochem Sci 27:514-520. Receptors in the Eph subfamily typically have a single kinase domain and an extracellular region containing a Cys-rich domain and 2 fibronectin type III repeats (see FIG. 18). The ephrin receptors are divided into 2 groups based on the similarity of their extracellular domain sequences and their affinities for binding ephrin-A and ephrin-B ligands. Many members of the Eph receptors have been identified as important markers and/or regulators of the development and progression of cancer (see for example Thaker et al., 2004, Clin. Cancer Res. 10:5145; Fox B P et al., 2004, Biochem. Biophys. Res. Commun. 318:882; Nakada et al., 2004, Cancer Res. 64:3179; Coffman et al., 2003, Cancer Res. 63:7907; also reviewed in Dodelet et al., 2000, Oncogene 19:5614). Of the Eph receptors known to be involved in cancer the role and expression patterns of EphA2 and EphA4 are among the best characterized.

EphA2 is expressed in adult epithelia, where it is found at low levels and is enriched within sites of cell-cell adhesion (Zantek, et al, 1999, Cell Growth & Diff 10:629; Lindberg, et al., 1990, Mol & Cell Biol 10: 6316). This subcellular localization is important because EphA2 binds EphrinsA1 to A5 that are anchored to the cell membrane (Eph Nomenclature Committee, 1997, Cell 90:403; Gale, et al., 1997, Cell & Tissue Res 290: 227). The primary consequence of ligand binding is EphA2 autophosphorylation (Lindberg, et al., 1990, supra). However, unlike other receptor tyrosine kinases, EphA2 retains enzymatic activity in the absence of ligand binding or phosphotyrosine content (Zantek, et al., 1999, supra). EphA2 and ephrin-A1 are upregulated in the transformed cells of a wide variety of tumors including breast, prostate, colon, lung, kidney, skin, and esophageal cancers (Ogawa, et al., 2000, Oncogene 19:6043; Zelinski, et al., 2001, Cancer Res 61:2301; Walker-Daniels, et al., 1999, Prostate 41:275; Easty, et al., 1995, Int J Cancer 60: 129; Nemoto, et al., 1997, Pathobiology 65:195; Hess et al., 2001, Cancer Res 61(8): 3250-5).

EphA4 is expressed in brain, heart, lung, muscle, kidney, placenta, pancreas (Fox, et al, 1995, Oncogene 10:897) and melanocytes (Easty, et al., 1997, Int. J. Cancer 71:1061). EphA4 binds Ephrins A1, A2, A3, A4, A5, B2, and B3, (Pasquale, 1997, Curr. Opin. In Cell Biology 9:608) also ligands B61, AL1/RAGS, LERK4, Htk-L, and Elk-L3, (Martone, et al., 1997, Brain Research 771:238). Ligand binding leads to EphA4 autophosphorylation on tyrosine residues (Ellis, et al., 1996, Oncogene 12:1727). EphA4 tyrosine phosphorylation creates a binding region for proteins with Src Homology 2/3 (SH2/SH3) domains, such as the cytoplasmic tyrosine kinase p59fyn (Ellis, et al., supra; Cheng, et al., Cytokine and Growth Factor Reviews 13:75, 2002). Activation of EphA4 in Xenopus embryos leads to loss of cadherin-dependent cell adhesion (Winning, et al., Differentiation 70:46, 2002; Cheng, et al., supra), suggesting a role for EphA4 in tumor angiogenesis; however, the role of EphA4 in cancer progression is unclear. EphA4 appears to be upregulated in breast cancer, esophageal cancer, and pancreatic cancer (Kuang, et al., Nucleic Acids Res. 26:1116, 1998; Meric, et al, Clinical Cancer Res. 8:361, 2002; Nemoto, et al., Pathobiology 65:195, 1997; Logsdon, et al., Cancer Res. 63:2649, 2003), yet it is downregulated in melanoma tissue (Easty, et al., supra).

EphB2 and EphB4 receptors are also overexpressed in certain tumor tissues. EphB4 overexpression is mainly found in infiltrating ductal breast carcinomas with high grade malignancy-2 (Berclaz et al., 1996, Biochem Biophys Res Commun 226:869) while EphB2 is overexpressed in a majority of gastric tumors (Kiynokawa et al., 1994, Cancer Res 54:3645). Both receptors are overexpressed in many tumor cell lines as well (Berclaz et al., supra; Kiynokawa et al., supra; Bennett et al., 1995, PNAS USA 92:1866). Both EphB2 and EphB4 are also upregulated in colon carcinoma tissue (Liu et al., 2002, Cancer 94:934; Stephenson et al., 2001, BMC Mol Biol 2:15). In addition, EphB2 and EphB4 are also important for vascular development in the embryo and possibly in tumors (Wang et al., 1998, Cell 93:741; Gerety, S. S. et al. 1999 Mol Cell 4:403).

### Cancer Therapy

One barrier to the development of anti-metastasis agents has been the assay systems that are used to design and evaluate these drugs. Most conventional cancer therapies target rapidly growing cells. However, cancer cells do not necessarily grow more rapidly but instead survive and grow under conditions that are non-permissive to normal cells (Lawrence and Steeg, 1996, World J. Urol. 14:124-130). These fundamental differences between the behaviors of normal and malignant cells provide opportunities for therapeutic targeting. The paradigm that micrometastatic tumors have already disseminated throughout the body emphasizes the need to evaluate potential chemotherapeutic drugs in the context of a foreign and three-dimensional microenvironment. Many standard cancer drug assays measure tumor cell growth or survival under typical cell culture conditions (i.e., monolayer growth). However, cell behavior in two-dimensional assays often does not reliably predict tumor cell behavior in vivo.

Currently, cancer therapy may involve surgery, chemotherapy, hormonal therapy and/or radiation treatment to eradicate neoplastic cells in a patient (see, for example, Stockdale, 1998, “Principles of Cancer Patient Management”, in Scientific American: Medicine, vol. 3, Rubenstein and Federman, eds., Chapter 12, Section IV). All of these approaches pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and although can be effective, is often used to prevent or delay recurrence of cancer after other treatments have removed the majority of the cancer cells.

With respect to chemotherapy, there are a variety of chemotherapeutic agents available for treatment of cancer. A significant majority of cancer chemotherapeutics act by inhibiting DNA synthesis (see, for example, Gilman et al., Goodman and Gilman's: The Pharmacological Basis of Therapeutics, Eighth Ed. (Pergamom Press, New York, 1990)). As such, chemotherapy agents are inherently nonspecific. In addition almost all chemotherapeutic agents are toxic, and chemotherapy causes significant, and often dangerous, side effects, including severe nausea, bone marrow depression, immunosuppression, etc. (see, for example, Stockdale, 1998, “Principles Of Cancer Patient Management” in Scientific American Medicine, vol. 3, Rubenstein and Federman, eds., ch. 12, sect. 10). Furthermore, even with administration of combinations of chemotherapeutic agents, many tumor cells are resistant or develop resistance to the chemotherapeutic agents.

Cancer therapy can now also involve biological therapy or immunotherapy. Biological therapies/immunotherapies are limited in number and although more specific then chemotherapeutic agents many still target both health and cancerous cells. In addition, such therapies may produce side effects such as rashes or swellings, flu-like symptoms, including fever, chills and fatigue, digestive tract problems or allergic reactions.

Thus, there is a significant need for alternative cancer treatments, particularly for treatments that more specifically target cancer cells. The identification of members of the Eph receptor family as markers for tumor cells makes them powerful targets for therapeutics. Accordingly, a cancer treatment that would specifically target and destroy tumor cells aberrantly expressing one or more members of the Eph receptor family would be a powerful tool for the treatment and prevention of cancers.

### Antibodies for the Treatment of Cancer

Antibodies are immunological proteins that bind a specific antigen. In most mammals, including humans and mice, antibodies are constructed from paired heavy and light polypeptide chains. Each chain is made up of two distinct regions, referred to as the variable (Fv) and constant (Fc) regions. The light and heavy chain Fv regions contain the antigen binding determinants of the molecule and are responsible for binding the target antigen. The Fc regions define the class (or isotype) of antibody (IgG for example) and are responsible for binding a number of natural proteins to elicit important biochemical events.

The Fc region of an antibody interacts with a number of ligands including Fc receptors and other ligands, imparting an array of important functional capabilities referred to as effector functions. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CID64), including isoforms FcγRIA, FcγRIB, and FcγRIC; FcγRII (CD32), including isoforms FcγRIIA, FcγRIIB, and FcγRIIC; and FcγRIII (CID16), including isoforms FcγRIIIA and FcγRIIB (Jefferis et al., 2002, Immunol Lett 82:57-65). These receptors typically have an extracellular domain that mediates binding to Fc, a membrane spanning region, and an intracellular domain that may mediate some signaling event within the cell. These different FcγR subtypes are expressed on different cell types (reviewed in Ravetch et al., 1991, Annu Rev Immunol 9:457-492). For example, in humans, FcγRIIIB is found only on neutrophils, whereas FcγRIIIA is found on macrophages, monocytes, natural killer (NK) cells, and a subpopulation of T-cells.

Formation of the Fc/FcγR complex recruits effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). Notably, the primary cells for mediating ADCC, NK cells, express only FcγRIIIA, whereas monocytes express FcγRI, FcγRII and FcγRIII (Ravetch et al., 1991, supra).

Another important Fc ligand is the complement protein C1q. Fc binding to C1q mediates a process called complement dependent cytotoxicity (CDC) (reviewed in Ward et al., 1995, Ther Immunol 2:77-94). C1q is capable of binding six antibodies, although binding to two IgGs is sufficient to activate the complement cascade. C1q forms a complex with the C1r and C1s serine proteases to form the C1 complex of the complement pathway.

Several key features of antibodies including but not limited to, specificity for target, ability to mediate immune effector mechanisms, and long half-life in serum, make antibodies and related immunoglobulin molecules powerful therapeutics. Numerous monoclonal antibodies are currently in development or are being used therapeutically for the treatment of a variety of conditions including cancer. Examples of these include Vitaxin® (MedImmune), a humanized Integrin αvβ3 antibody (e.g., PCT publication WO 2003/075957), Herceptin® (Genentech), a humanized anti-Her2/neu antibody approved to treat breast cancer (e.g., U.S. Pat. No. 5,677,171), CNTO 95 (Centocor), a human Integrin αv antibody (PCT publication WO 02/12501), Rituxan® (IDEC/Genentech/Roche), a chimeric anti-CD20 antibody approved to treat Non-Hodgkin's lymphoma (e.g., U.S. Pat. No. 5,736,137) and Erbitux® (ImClone), a chimeric anti-EGFR antibody (e.g., U.S. Pat. No. 4,943,533).

There are a number of possible mechanisms by which antibodies destroy tumor cells, including anti-proliferation via blockage of needed growth pathways, intracellular signaling leading to apoptosis, enhanced down regulation and/or turnover of receptors, ADCC, CDC, and promotion of an adaptive immune response (Cragg et al., 1999, Curr Opin Immunol 11:541-547; Glennie et al., 2000, Immunol Today 21:403-410). However, despite widespread use, antibodies are not yet optimized for clinical use and many have suboptimal anticancer potency. Thus, there is a significant need to enhance the capacity of antibodies to destroy targeted cancer cells.

### Antibody-Drug Conjugates

One effective approach for enhancing the anti-tumor-potency of antibodies involves linking cytotoxic drugs or toxins to mAbs that are capable of being internalized by a target cell. These agents are termed antibody-drug conjugates (ADCs) and immunotoxins, respectively. Upon administration to a patient, ADCs and immunotoxins bind to target cells via their antibody portions and become internalized, allowing the drugs or toxins to exert their effect. See, for example, U.S. Patent Appl. Publ. Nos. US2005/0180972 A1, US2005/0123536 A1. See also, for example, Hamblett et al., Clin Canc Res, 10:7063-7070, Oct. 15, 1999, Law et al., Clin Canc Res, 10:7842-7851, Dec. 1, 2004, Francisco et al., Neoplasia, 102(4):1458-1465, Aug. 15, 2003, Russell et al., Clin Canc Res, 11:843-852, Jan. 15, 2005, Doronina et al., Nat Biotech, 21(7):778-784, July 2003, all of which are hereby incorporated by reference herein in their entirety.

Citation or discussion of a reference herein shall not be construed as an admission that such is prior art to the present invention.

### SUMMARY OF THE INVENTION

The present invention provides an internalizing antibody drug conjugate (ADC) that specifically binds to EphA2, wherein said ADC is conjugated to a toxin. The present invention further provides an ADC, wherein said ADC comprises a toxin, a self-immolative spacer, and a linker. In one embodiment, the linker is a Val-Cit linker. In another embodiment, the toxin is an anti-tubulin agent. In a further embodiment, the toxin is an auristatin, for example, auristatin E, auristatin F, MMAE or MMAF.

The present invention further provides a method of inhibiting cancer cell growth comprising administering to the subject a pharmaceutically effective amount of a composition comprising (a) an ADC of the present invention; and (b) a pharmaceutically acceptable carrier. In one embodiment, the cancer cell is a melanoma cancer cell, a prostate cancer cell, a lung cancer cell, a breast cancer cell, a colon cancer cell, a kidney cancer cell, an ovarian cancer cell, or a pancreatic cancer cell.

The present invention further provides a method of treating cancer comprising administering to the subject a pharmaceutically effective amount of a composition comprising (a) an ADC of the present invention; and (b) a pharmaceutically acceptable carrier. In yet another embodiment, the cancer is selected from the group consisting of melanoma, prostate cancer, lung cancer, breast cancer, colon cancer, kidney cancer, ovarian cancer and pancreatic cancer.

### DEFINITIONS

As used herein, the term “agonist” refers to any compound including a protein, polypeptide, peptide, antibody, antibody fragment, large molecule, or small molecule (less than 10 kD), that increases the activity, activation or function of another molecule. EphA2 or EphA4 agonists cause increased phosphorylation and degradation of EphA2 or EphA4 protein. EphA2 or EphA4 antibodies that agonize EphA2 or EphA4 may or may not also inhibit cancer cell phenotype (e.g., colony formation in soft agar or tubular network formation in a three-dimensional basement membrane or extracellular matrix preparation) and may or may not preferentially bind an EphA2 or EphA4 epitope that is exposed in a cancer cell relative to a non-cancer cell and may or may not have a low K_{off} rate.

As used herein, the term “immunospecifically binds to an Eph receptor” and analogous terms refer to peptides, polypeptides, proteins, fusion proteins and antibodies or fragments thereof that specifically bind to at least one Eph receptor or a fragment thereof. The term “immunospecifically” may be used interchangeably with the term “specifically.” A peptide, polypeptide, protein, or antibody that immunospecifically binds to at least one Eph receptor or a fragment thereof may bind to other peptides, polypeptides, or proteins with lower affinity as determined by, e.g., immunoassays, BIAcore, or other assays known in the art. Antibodies or fragments that immunospecifically bind to at least one Eph receptor or a fragment thereof may be cross-reactive with related antigens. Preferably, antibodies or fragments that immunospecifically bind to at least one Eph receptor or a fragment thereof preferentially bind to at least one Eph receptor over other antigens. However, the present invention specifically encompasses antibodies with multiple specificities (e.g., an antibody with specificity for two or more discrete antigens (reviewed in Cao et al., 2003, Adv Drug Deliv Rev 55:171; Hudson et al., 2003, Nat Med 1:129)) in the definition of an antibody that “immunospecifically binds to an Eph receptor.” For example, bispecific antibodies contain two different binding specificities fused together. In the simplest case a bispecific antibody would bind to two adjacent epitopes on a single target antigen, such an antibody would not cross-react with other antigens (as described supra). Alternatively, bispecific antibodies can bind to two different antigens. Such an antibody immunospecifically binds to two different molecules, but not to other unrelated molecules. Another class of multispecific antibodies may recognize a shared subunit of multi-subunit complexes in the context of one or more specific complexes. In addition, an antibody that specifically binds an Eph receptor may cross-react with related Eph receptors or RTKs.

Antibodies or fragments that specifically bind to an Eph receptor or a fragment thereof can be identified, for example, by immunoassays, BIAcore, or other techniques known to those of skill in the art. An antibody or fragment thereof binds specifically to an Eph receptor or a fragment thereof when it binds to an Eph receptor or a fragment thereof with higher affinity than to any cross-reactive antigen as determined using experimental techniques, such as (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

The term “antibodies or fragments thereof that specifically bind to EphA2 or EphA4” as used herein refers to antibodies or fragments thereof that specifically bind to an EphA2 or EphA4 polypeptide or a fragment of an EphA2 or EphA4 polypeptide and do not specifically bind to other non-EphA2 or non-EphA4 polypeptides. Preferably, antibodies or fragments that specifically bind to an EphA2 or EphA4 polypeptide or fragment thereof do not non-specifically cross-react with other antigens (e.g., binding cannot be competed away with a non-EphA2 or non-EphA4 protein, e.g., BSA, in an appropriate immunoassay). Antibodies or fragments that specifically bind to an EphA2 or EphA4 polypeptide can be identified, for example, by immunoassays or other techniques known to those of skill in the art. Antibodies of the invention include, but are not limited to, synthetic antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen binding site that specifically binds to an EphA2 or EphA4 antigen (e.g., one or more complementarity determining regions (CDRs) of an anti-EphA2 or anti-EphA4 antibody). Preferably, agonistic antibodies or fragments thereof that specifically bind to an EphA2 or EphA4 polypeptide or fragment thereof preferentially agonize EphA2 or EphA4 and do not significantly agonize other molecules or activities.

As used herein, the term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or weblike matrices in a three-dimensional basement membrane or extracellular matrix preparation, such as MATRIGEL™. Non-cancer cells do not form colonies in soft agar and form distinct sphere-like structures in three-dimensional basement membrane or extracellular matrix preparations. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. The term “cancer cell” is meant to encompass both pre-malignant and malignant cancer cells.

As used herein, the phrase “cancer cell phenotype inhibiting” refers to the ability of a compound to prevent or reduce cancer cell colony formation in soft agar or tubular network formation in a three-dimensional basement membrane or extracellular matrix preparation or any other method that detects a reduction in a cancer cell phenotype, for example, assays that detect an increase in contact inhibition of cell proliferation (e.g., reduction of colony formation in a monolayer cell culture). Cancer cell phenotype inhibiting compounds may also cause a reduction or elimination of colonies when added to established colonies of cancer cells in soft agar or the extent of tubular network formation in a three-dimensional basement membrane or extracellular matrix preparation. EphA2 or EphA4 antibodies that inhibit cancer cell phenotype may or may not also agonize EphA2 or EphA4 and may or may not have a low K_{off} rate.

As used herein, the term “delivery vehicle” refers to a substance that can be used to administer a therapeutic or prophylactic agent to a subject, particular a human. A delivery vehicle may preferentially deliver the therapeutic/prophylactic agent(s) to a particular subset of cells. A delivery vehicle may target certain types of cells, e.g., by virtue of an innate feature of the vehicle or by a moiety conjugated to, contained within (or otherwise associated with such that the moiety and the delivery vehicle stay together sufficiently for the moiety to target the delivery vehicle) the vehicle, which moiety specifically binds a particular subset of cells, e.g., by binding to a cell surface molecule characteristic of the subset of cells to be targeted. A delivery vehicle may also increase the in vivo half-life of the agent to be delivered and/or the bioavailability of the agent to be delivered. Non-limiting examples of a delivery vehicle are a viral vector, a virus-like particle, a polycation vector, a peptide vector, a liposome, and a hybrid vector. In specific embodiments, the delivery vehicle is not directly conjugated to the moiety that binds EphA2 and/or EphA4. In other embodiments, the delivery vehicle is not an antibody that binds EphA2 and/or EphA4.

As used herein, the term “derivative” in the context of a proteinaceous agent (e.g., proteins, polypeptides, peptides, and antibodies) refers to a proteinaceous agent that comprises the amino acid sequence which has been altered by the introduction of amino acid residue substitutions, deletions, and/or additions. The term “derivative” as used herein refers to, for example, but not by way of limitation, a polypeptide that comprises an amino acid sequence of an EphA2 or EphA4 polypeptide, a fragment of an EphA2 or EphA4 polypeptide, an antibody that specifically binds to an EphA2 or EphA4 polypeptide, or an antibody fragment that specifically binds to an EphA2 or EphA4 polypeptide, that has been altered by the introduction of amino acid residue substitutions, deletions or additions (i.e., mutations). In some embodiments, an antibody derivative or fragment thereof comprises amino acid residue substitutions, deletions or additions in one or more CDRs. The antibody derivative may have substantially the same binding, better binding, or worse binding when compared to a non-derivative antibody. In specific embodiments, one, two, three, four, or five amino acid residues of the CDR have been substituted, deleted or added (i.e., mutated). The term “derivative” as used herein also refers to a proteinaceous agent which has been modified, i.e., by the covalent attachment of a type of molecule to the proteinaceous agent. The term “derivative” as used herein also refers to, for example, but not by way of limitation, an EphA2 or EphA4 polypeptide, a fragment of an EphA2 or EphA4 polypeptide, an antibody that specifically binds to an EphA2 or EphA4 polypeptide, or an antibody fragment that specifically binds to an EphA2 or EphA4 polypeptide which has been modified, i.e, by the covalent attachment of any type of molecule to the polypeptide. For example, but not by way of limitation, an EphA2 or EphA4 polypeptide, a fragment of an EphA2 or EphA4 polypeptide, an antibody, or antibody fragment may be modified, e.g., by glycosylation, acetylation, pegylation, phosphorylation, amidation, derivatization by known protecting/blocking groups, proteolytic cleavage, linkage to a cellular ligand or other protein, etc. A derivative of an EphA2 or EphA4 polypeptide, a fragment of an EphA2 or EphA4 polypeptide, an antibody, or antibody fragment may be modified by chemical modifications using techniques known to those of skill in the art, including, but not limited to, specific chemical cleavage, acetylation, formylation, metabolic synthesis of tunicamycin, etc. Further, a derivative of a proteinaceous agent may contain one or more non-classical amino acids. For example, a derivative of an EphA2 or EphA4 polypeptide, a fragment of an EphA2 or EphA4 polypeptide, an antibody, or antibody fragment may contain one or more non-classical amino acids. In one embodiment, a polypeptide derivative possesses a similar or identical function as an EphA2 or EphA4 polypeptide, a fragment of an EphA2 or EphA4 polypeptide, an antibody, or antibody fragment described herein. In another embodiment, a derivative of EphA2 or EphA4 polypeptide, a fragment of an EphA2 or EphA4 polypeptide, an antibody, or antibody fragment has an altered activity when compared to an unaltered polypeptide. For example, a derivative antibody or fragment thereof can bind to its epitope more tightly or be more resistant to proteolysis.

The term “epitope” as used herein refers to a portion of an EphA2 or EphA4 polypeptide having antigenic or immunogenic activity in an animal, preferably in a mammal, and most preferably in a mouse or a human. An epitope having immunogenic activity is a portion of an EphA2 or EphA4 polypeptide that elicits an antibody response in an animal. An epitope having antigenic activity is a portion of an EphA2 or EphA4 polypeptide to which an antibody specifically binds as determined by any method well known in the art, for example, by immunoassays. Antigenic epitopes need not necessarily be immunogenic.

As used herein, the term “EphA2” or “EphA4” refer to any Eph receptor polypeptide that has been identified and recognized by the Eph Nomenclature Committee (Eph Nomenclature Committee, 1997, Cell 90:403-404). In a specific embodiment, an EphA2 or EphA4 receptor polypeptide or fragment thereof is from any species. In one embodiment, an EphA2 or EphA4 receptor polypeptide or fragment thereof is human. The nucleotide and/or amino acid sequences of Eph receptor polypeptides can be found in the literature or public databases (e.g., GenBank), or the nucleotide and/or amino acid sequences can be determined using cloning and sequencing techniques known to one of skill in the art. For example, the GenBank Accession Nos. for the nucleotide and amino acid sequences of the human EphA2 are NM_{—}004431.2 and NP_{—}004422.2, respectively. The GenBank Accession Nos. for the nucleotide and amino acid sequences of the human EphA4 are NM_{—}004438.3 and NP_{—}004429.1, respectively.

As used herein, the term “Ephrin” or “Ephrin ligand” refers to any Ephrin ligand that has or will be identified and recognized by the Eph Nomenclature Committee (Eph Nomenclature Committee, 1997, Cell 90:403-404). Ephrins of the present invention include, but are not limited to, EphrinA1, EphrinA2, EphrinA3, EphrinA4, EphrinA5, EphrinB1, EphrinB2 and EphrinB3. In a specific embodiment, an Ephrin polypeptide, particularly EphrinA1, is from any species. In another embodiment, an Ephrin polypeptide, particularly Ephrin A1, is human. The nucleotide and/or amino acid sequences of Ephrin polypeptides can be found in the literature or public databases (e.g., GenBank), or the nucleotide and/or amino acid sequences can be determined using cloning and sequencing techniques known to one of skill in the art. For example, GenBank Accession Nos. for the nucleotide and amino acid sequences of human Ephrin A1 variant 1 are NM_{—}004428.2 and NP_{—}004419.2, respectively. The GenBank Accession Nos. for the nucleotide and amino acid sequences of human Ephrin A1 variant 2 are NM_{—}182685.1 and NP_{—}872626.1 for variant 2, respectively.

The “fragments” in the context of a polypeptide described herein include a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least 90 contiguous amino acid residues, at least contiguous 100 amino acid residues, at least 125 contiguous amino acid residues, at least 150 contiguous amino acid residues, at least 175 contiguous amino acid residues, at least contiguous 200 amino acid residues, or at least 250 contiguous amino acid residues of the amino acid sequence of an EphA2 or EphA4 polypeptide or an antibody that specifically binds to an EphA2 or EphA4 polypeptide. Preferably, antibody fragments are epitope-binding fragments.

As used herein, the term “humanized antibody” refers to forms of non-human (e.g., murine) antibodies that are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, Framework Region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin that specifically binds to an EphA2 or an EphA4 polypeptide, that has been altered by the introduction of amino acid residue substitutions, deletions or additions (i.e., mutations). In some embodiments, a humanized antibody is a derivative. Such a humanized antibody comprises amino acid residue substitutions, deletions or additions in one or more non-human CDRs. The humanized antibody derivative may have substantially the same binding, better binding, or worse binding when compared to a non-derivative humanized antibody. In specific embodiments, one, two, three, four, or five amino acid residues of the CDR have been substituted, deleted or added (i.e., mutated). For further details in humanizing antibodies, see European Patent Nos. EP 239,400, EP 592,106, and EP 519,596; International Publication Nos. WO 91/09967 and WO 93/17105; U.S. Pat. Nos. 5,225,539, 5,530,101, 5,565,332, 5,585,089, 5,766,886, and 6,407,213; and Padlan, 1991, Molecular Immunology 28(4/5):489-498; Studnicka et al., 1994, Protein Engineering 7(6):805-814; Roguska et al., 1994, PNAS 91:969-973; Tan et al., 2002, J. Immunol. 169:1119-25; Caldas et al., 2000, Protein Eng. 13:353-60; Morea et al., 2000, Methods 20:267-79; Baca et al., 1997, J. Biol. Chem. 272:10678-84; Roguska et al., 1996, Protein Eng. 9:895-904; Couto et al., 1995, Cancer Res. 55 (23 Supp):5973s-5977s; Couto et al., 1995, Cancer Res. 55:1717-22; Sandhu, 1994, Gene 150:409-10; Pedersen et al., 1994, J. Mol. Biol. 235:959-73; Jones et al., 1986, Nature 321:522-525; Reichmann et al., 1988, Nature 332:323-329; and Presta, 1992, Curr Op. Struct. Biol. 2:593-596.

As used herein, the term “hypervariable region” refers to the amino acid residues of an antibody which are responsible for antigen binding. The hypervariable region comprises amino acid residues from a “Complementarity Determining Region” or “CDR” (i.e., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5^{th} Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). CDR residues for Eph099B-208.261 and Eph099B-233.152 are listed in Table 2. “Framework Region” or “FR” residues are those variable domain residues other than the hypervariable region residues as herein defined.

As used herein, the term “in combination” refers to the use of more than one therapy (e.g., prophylactic and/or therapeutic agents). The use of the term “in combination” does not restrict the order in which prophylactic and/or therapeutic agents are administered to a subject with a hyperproliferative cell disorder, especially cancer. A first therapy (e.g., prophylactic or therapeutic agent) can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., prophylactic or therapeutic agent) to a subject which had, has, or is susceptible to a hyperproliferative cell disorder, especially cancer. The therapies (e.g., prophylactic or therapeutic agents) are administered to a subject in a sequence and within a time interval such that the therapy of the invention can act together with the other agent to provide an increased benefit than if they were administered otherwise. Any additional therapy (e.g., prophylactic or therapeutic agent) can be administered in any order with the other additional therapies (e.g., prophylactic or therapeutic agents).

As used herein, the phrase “low tolerance” refers to a state in which the patient suffers from side effects from treatment so that the patient does not benefit from and/or will not continue therapy because of the adverse effects and/or the harm from the side effects outweighs the benefit of the treatment.

As used herein, the terms “manage,” “managing” and “management” refer to the beneficial effects that a subject derives from administration of a therapy (e.g., prophylactic or therapeutic agent), which does not result in a cure of the disease. In certain embodiments, a subject is administered one or more therapies (e.g., prophylactic or therapeutic agents) to “manage” a disease so as to prevent the progression or worsening of the disease.

As used herein, the phrase “non-responsive/refractory” is used to describe patients treated with one or more currently available therapies (e.g., cancer therapies) such as chemotherapy, radiation therapy, surgery, hormonal therapy and/or biological therapy/immunotherapy, particularly a standard therapeutic regimen for the particular cancer, wherein the therapy is not clinically adequate to treat the patients such that these patients need additional effective therapy, e.g., remain unsusceptible to therapy. The phrase can also describe patients who respond to therapy yet suffer from side effects, relapse, develop resistance, etc. In various embodiments, “non-responsive/refractory” means that at least some significant portion of the cancer cells are not killed or their cell division arrested. The determination of whether the cancer cells are “non-responsive/refractory” can be made either in vivo or in vitro by any method known in the art for assaying the effectiveness of treatment on cancer cells, using the art-accepted meanings of “refractory” in such a context. In various embodiments, a cancer is “non-responsive/refractory” where the number of cancer cells has not been significantly reduced, or has increased during the treatment.

As used herein, the term “potentiate” refers to an improvement in the efficacy of a therapeutic agent at its common or approved dose.

As used herein, the terms “prevent,” “preventing” and “prevention” refer to the prevention of the onset, recurrence, or spread of a disease in a subject resulting from the administration of a therapy (e.g., prophylactic or therapeutic agent).

As used herein, the term “prophylactic agent” refers to any agent that can be used in the prevention of the onset, recurrence or spread of a disease or disorder associated with EphA2 or EphA4 overexpression and/or cell hyperproliferative disease, particularly cancer. In a specific embodiment, the term “prophylactic agent” refers to any composition comprising a therapeutically or prophylactically effective amount of (a) a delivery vehicle conjugated to (or otherwise associated with) a moiety that binds EphA2 and/or EphA4; (b) one or more therapeutic or prophylactic agents that treat or prevent said hyperproliferative disease; and (c) a pharmaceutically acceptable carrier. In certain embodiments, the term “prophylactic agent” refers to an EphA2 or EphA4 agonistic antibody, an EphA2 or EphA4 cancer cell phenotype inhibiting antibody, an exposed EphA2 or EphA4 epitope antibody, or an antibody that binds EphA2 or EphA4 with a K_{off} of less than 3×10⁻³ s⁻¹ (e.g., Eph099B-102.147, Eph099B-208.261, Eph099B-210.248, Eph099B-233.152, EA44, or any of the antibodies listed in Tables 2-4 or 6). In a specific embodiment, an EphA4 agonistic antibody for use in the compositions and methods of the invention is EA44, an anti-EphA4 scFV antibody which is disclosed in U.S. Non-Provisional application Ser. No. 10/863,729, filed Jun. 7, 2004 and is incorporated by reference herein in its entirety. Cells that express the anti-EphA4 scFv EA44 have been deposited with the American Type Culture Collection (P.O. Box 1549, Manassas, Va. 20108) on Jun. 4, 2004 under the provisions of the Budapest Treaty on the International Recognition of the Deposit of Microorganisms for the Purposes of Patent Procedures, and assigned accession number PTA-6044. In certain other embodiments, the term “prophylactic agent” refers to cancer chemotherapeutics, radiation therapy, hormonal therapy, biological therapy (e.g., immunotherapy), and/or EphA2 or EphA4 antibodies of the invention. In other embodiments, more than one prophylactic agent may be administered in combination.

As used herein, a “prophylactically effective amount” refers to that amount of a therapy (e.g., a prophylactic agent) sufficient to result in the prevention of the onset, recurrence or spread of cell hyperproliferative disease, preferably, cancer. A prophylactically effective amount may refer to the amount of a therapy (e.g., a prophylactic agent) sufficient to prevent the onset, recurrence or spread of hyperproliferative disease, particularly cancer, including but not limited to those predisposed to hyperproliferative disease, for example, those genetically predisposed to cancer or previously exposed to carcinogens. A prophylactically effective amount may also refer to the amount of the therapy (e.g., a prophylactic agent) that provides a prophylactic benefit in the prevention of hyperproliferative disease. Further, a prophylactically effective amount with respect to a prophylactic agent of the invention means that amount of prophylactic agent alone, or in combination with other agents, that provides a prophylactic benefit in the prevention of hyperproliferative disease. Used in connection with an amount of an EphA2 or EphA4 antibody of the invention, the term can encompass an amount that improves overall prophylaxis or enhances the prophylactic efficacy of or synergies with another therapy (e.g., a prophylactic agent).

A used herein, a “protocol” includes dosing schedules and dosing regimens.

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky. Side effects from chemotherapy include, but are not limited to, gastrointestinal toxicity such as, but not limited to, early and late-forming diarrhea and flatulence, nausea, vomiting, anorexia, □luorourac, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (58^{th} ed., 2004).

As used herein, the terms “single-chain Fv” or “scFv” refer to antibody fragments comprise the VH and VL domains of antibody, wherein these domains are present in a single polypeptide chain. Generally, the Fv polypeptide further comprises a polypeptide linker between the VH and VL domains which enables the scFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994). In specific embodiments, scFvs include bi-specific scFvs and humanized scFvs.

As used herein, the terms “subject” and “patient” are used interchangeably.

As used herein, a subject is preferably a mammal such as a non-primate (e.g., cows, pigs, horses, cats, dogs, rats etc.) and a primate (e.g., monkey and human), most preferably a human.

As used herein, the term “targeting moiety” or “binding moiety” refers to any moiety that, when linked to another agent (such as a delivery vehicle or another compound), enhances the transport of that agent to a target tissue or a subset of cells with a common characteristic, thereby increasing the local concentration of the agent in and around the targeted tissue or subset of cells. For example, a targeting moiety may bind to a molecule on the surface of some or all of the cells in the target tissue or cell subset. In specific embodiments, a targeting moiety binds to EphA2 or EphA4. In another embodiment, a targeting moiety binds to EphA2 or EphA4 on cancer cells (e.g., EphA2 or EphA4 not bound to a ligand) rather than EphA2 or EphA4 on non-cancer cells (e.g., EphA2 or EphA4 bound to a ligand).

As used herein, the terms “treat,” “treating” and “treatment” refer to the eradication, reduction or amelioration of symptoms of a disease or disorder, particularly, the eradication, removal, modification, or control of primary, regional, or metastatic cancer tissue that results from the administration of one or more therapeutic agents. In certain embodiments, such terms refer to the minimizing or delaying the spread of cancer resulting from the administration of one or more therapies (e.g., prophylactic or therapeutic agents) to a subject with such a disease.

As used herein, the term “therapeutic agent” refers to any agent that can be used in the prevention, treatment, or management of a disease or disorder associated with overexpression of EphA2, EphA4 and/or cell hyperproliferative diseases or disorders, particularly, cancer. In a specific embodiment, the term “therapeutic agent” refers to any composition comprising a therapeutically or prophylactically effective amount of (a) a delivery vehicle conjugated to (or otherwise associated with) a moiety that binds EphA2 and/or EphA4; (b) one or more therapeutic or prophylactic agents that treat or prevent said hyperproliferative disease; and (c) a pharmaceutically acceptable carrier. In certain embodiments, the term “therapeutic agent” refers to an EphA2 or EphA4 agonistic antibody, an EphA2 or EphA4 cancer cell phenotype inhibiting antibody, an exposed EphA2 or EphA4 epitope antibody, or an antibody that binds EphA2 or EphA4 with a K_{off} of less than 3×10⁻³ s⁻¹ (e.g., Eph099B-102.147, Eph099B-208.261, Eph099B-210.248, Eph099B-233.152, EA44 or any of the antibodies listed in Tables 2-4 or 6). In certain other embodiments, the term “therapeutic agent” refers to cancer chemotherapeutics, radiation therapy, hormonal therapy, biological therapy/immunotherapy, and/or EphA2 or EphA4 antibody of the invention. In other embodiments, more than one therapeutic agent may be administered in combination.

As used herein, a “therapeutically effective amount” refers to that amount of a therapy (e.g., therapeutic agent) sufficient to treat or manage a disease or disorder associated with EphA2 or EphA4 overexpression and/or cell hyperproliferative disease and, preferably, the amount sufficient to destroy, modify, control or remove primary, regional or metastatic cancer tissue. A therapeutically effective amount may refer to the amount of a therapy (e.g., therapeutic agent) sufficient to delay or minimize the onset of the hyperproliferative disease, e.g., delay or minimize the spread of cancer. A therapeutically effective amount may also refer to the amount of the therapy (e.g., therapeutic agent) that provides a therapeutic benefit in the treatment or management of cancer. Further, a therapeutically effective amount with respect to a therapy (e.g., therapeutic agent) of the invention means that amount of therapeutic agent alone, or in combination with other therapies, that provides a therapeutic benefit in the treatment or management of hyperproliferative disease or cancer. Used in connection with an amount of an EphA2 or EphA4 antibody of the invention, the term can encompass an amount that improves overall therapy, reduces or avoids unwanted effects, or enhances the therapeutic efficacy of or synergies with another therapy (e.g., therapeutic agent).

As used herein, the term “therapy” refers to any protocol, method and/or agent that can be used in the prevention, treatment, management or amelioration of a hyperproliferative disorder. In certain embodiments, the terms “therapies” and “therapy” refer to a biological therapy, supportive therapy, and/or other therapies useful in treatment, management, prevention, or amelioration of a hyperproliferative disorder or one or more symptoms thereof known to one of skill in the art such as medical personnel.

It will be understood that the complementarity determining regions (CDRs) residue numbers referred to herein are those of Kabat et al. (1991, NIH Publication 91-3242, National Technical Information Service, Springfield, Va.). Specifically, residues 24-34 (CDR1), 50-56 (CDR2) and 89-97 (CDR3) in the light chain variable domain and 31-35 (CDR1), 50-65 (CDR2) and 95-102 (CDR3) in the heavy chain variable domain. Note that CDRs vary considerably from antibody to antibody (and by definition will not exhibit homology with the Kabat consensus sequences). Maximal alignment of framework residues frequently requires the insertion of “spacer” residues in the numbering system, to be used for the Fv region. It will be understood that the CDRs referred to herein are those of Kabat et al. supra. In addition, the identity of certain individual residues at any given Kabat site number may vary from antibody chain to antibody chain due to interspecies or allelic divergence.

In the case where there are two or more definitions of a term that are used and/or accepted within the art, the definition of the term as used herein is intended to include all such meanings unless explicitly stated to the contrary. A specific example is the use of the term “CDR” to describe the non-contiguous antigen combining sites found within the variable region of both heavy and light chain polypeptides. This particular region has been described by Kabat et al., 1991, NIH Publication 91-3242, National Technical Information Service, Springfield, Va.) and by Chothia et al. (1987, J. Mol. Biol. 196:901-17) and additionally by MacCallum et al.(1996, J. Mol. Biol. 262:732-45), each of which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table 1 as a comparison. The exact residue numbers which encompass a particular CDR will vary depending on the sequence and size of the CDR.

Those skilled in the art can routinely determine which residues comprise a particular CDR given the variable region amino acid sequence of the antibody.

TABLE 1
CDR Definitions

| | Kabat¹ | Chothia² | MacCallum³ |
|---|---|---|---|
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL, CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

### BRIEF DESCRIPTION OF THE FIGURES

For the purpose of illustrating the invention, there are depicted in the figures certain embodiments on the invention. However, the invention is not limited to the precise arrangements and instrumentalities of the embodiments depicted in the figures.

FIG. 1. The light chain amino acid sequences of various anti-EphA2 and anti-EphA4 antibodies.

FIG. 2. The heavy chain amino acid sequences of various anti-EphA2 and EphA4 antibodies.

FIG. 3. The variable chain amino acid sequences of the anti-EphA2 antibodies G5 and 3 F2.

FIG. 4. The variable chain amino acid sequences of anti-EphA2 antibodies EA2, 4H5, and 10D9.

FIG. 5. Amino acid sequences of the variable heavy (V_{H}) and light (V_{L}) chains of various affinity-matured versions of the anti-Eph antibody GEA44. Amino acid sequences for the heavy chains of the following antibodies are listed in the upper half of the Figure: GEA44, 1A4, 1B10, 1D11, 1G11, 2 C9, 3A12, 3C6, 6B7, 6B4, and 11H1 (SEQ ID Nos. 115, 117, 119, 121, 123, 125, 127, 129, 131, 133, and 135, respectively). Amino acid sequences for the light chains of the following antibodies are listed in the lower half of the Figure: GEA44, 1A4, 1B10, D11, 1G11, 2C9, 3A12, 3C6, 6B7, 6B4, and 11H1 (SEQ ID Nos. 116, 117, 120, 122, 124, 126, 128, 130, 132, 134, and 136, respectively). The boxed portion of the sequences indicates the CDRs (Kabat definition).

FIG. 6. Nucleotide and amino acid sequences of the variable heavy (V_{H}) and variable light (V_{L}) chains of the pan-Eph antibody 10C12 (GEA10C12). The variable region heavy chain nucleotide and amino acid sequences are listed in the upper half of the Figure (SEQ ID's Nos. 141 and 140, respectively); the variable region light chain nucleotide and amino acid sequences are listed in the lower half of the Figure (SEQ ID's Nos. 142 and 141, respectively).

FIGS. 7A-7B. Amino acid sequences and alignments of the variable heavy (V_{H}) and light (V_{L}) chains of various phage derived anti-EphA2 antibodies. Amino acid sequences and alignments for the heavy chains of the following antibodies are listed in FIG. 7A: 5A8, 1C1, 1D3, 1F12, 1H3, 2B12 (SEQ ID Nos. 53, 3, 33, 13, 23, and 43, respectively). Amino acid sequences and alignments for the light chains of the following antibodies are listed in FIG. 7B: 5A8, 1C1, 1D3, 1F12, 1H3, 2B12 (SEQ ID Nos. 54, 4, 34, 14, 24, and 43, respectively). The boxed portion of the sequences indicates the CDRs (Kabat definition).

FIG. 8. Nucleic acid sequences and amino acid variable region sequences of the anti-EphA2 antibody 1C1 (SEQ ID Nos. 1-4).

FIG. 9. Nucleic acid sequences and amino acid variable region sequences of the anti-EphA2 antibody 1F12 (SEQ ID Nos. 11-14).

FIG. 10. Nucleic acid sequences and amino acid variable region sequences of the anti-EphA2 antibody 1H3 (SEQ ID Nos. 21-24).

FIG. 11. Nucleic acid sequences and amino acid variable region sequences of the anti-EphA2 antibody 1D3 (SEQ ID Nos. 31-34).

FIG. 12. Nucleic acid sequences and amino acid variable region sequences of the anti-EphA2 antibody 2B12 (SEQ ID Nos. 41-44).

FIG. 13. Nucleic acid sequences and amino acid variable region sequences of the anti-EphA2 antibody 5A8 (SEQ ID Nos. 51-54).

FIG. 14. Nucleic acid sequences and amino acid sequences for the constant region (heavy chain and kappa light chain) of the anti-EphA2 antibodies 1C1, 1F12, 1H3, 1D3, 2B12, and 5A8 (SEQ ID Nos. 111-114).

FIGS. 15A-15B. Comparison of cell surface binding of various anti-EphA2 antibodies to various cell lines via flow cytometry analysis. FIG. 14A compares antibodies 1C1, 1F12, 1H3, and 3F2 on the following cell lines: A549, Hey-A8, PC3, KC-231, Panc-02.03, SK Mel-28, ACHN, 498, D-145, HT-29, SKOV-3, and SW-480. FIG. 14B compares antibodies 1C1, 1F12, and 3F2 on the following cell lines: Balb/3T3, NIH/3T3, CT26, F98, RG2, YPEN.

FIG. 16. Comparison of internalization of several different anti-EphA2 antibodies. Internalization of the anti-EphA2 antibodies B233, EA5, and B208 is compared to controls in the MCF-10A cell line.

FIG. 17. Comparison of internalization of several different anti-EphA2 antibodies. Internalization of the anti-EphA2 antibodies B233, B208, EA2, G5, 3F2, 1C1, C2, 3B2 is compared to controls in the following cell lines: PC3, SK Mel-28, HuVec, MCF10A, and CT26.

FIG. 18. Internalization of the anti-EphA2 antibody, G5, is demonstrated by immunofluorescence. PC3 cells were labeled with either human α-EphA2 mAb (G5; panels A and B) or R347 isotype control (panel C). Cell surface attached antibodies were then allowed to internalize by incubating the cells under growth conditions for either zero (non-internalized: panels A and C) or 60 minutes (internalized: panel B). All cells were then fixed (4% formaldehyde), permeabilized (0.5% Triton X-100), and stained with AlexaFluor 488-Ab prior to addition of antifade mounting media and fluorescent microscopy examination. Panel B demonstrates internalization of the G5 anti-EphA2 antibody.

FIGS. 19A-19C. Internalization of the anti-EphA2 antibodies 1C1 (FIG. 18A), 1F12 (FIG. 18B), and 3F2 (FIG. 18C) on HuVec cells is demonstrated by immunofluorescence.

FIG. 20. Internalization of the anti-EphA2 antibodies 1C1 and 1F12 on Ct-26 and PC-3 cells is demonstrated by immunofluorescence.

FIG. 21. EphA2 phosphorylation by anti-EphA2 antibodies 1C1 and 1F12 is demonstrated in the following cell lines: CT26, 4T1, F98, YPEN1, PC3, and ES2.

FIG. 22. EphA2 phosphorylation by anti-EphA2 antibodies 1C1, 1F12, and 3F2 is demonstrated in HuVec cells.

FIG. 23. Properties (activation, internalization, and tissue cross reactivity (TCR)) of various anti-EphA2 antibodies (1C1, 1F12, 1H3, 1D3, 2B12, and 5A8) are summarized in this figure.

FIG. 24. Specificity of the anti-EphA2 antibodies 1C1 and 1F12 to different murine members of the Eph family of receptors is summarized in this figure. 1C1 demonstrates specific binding to murine EphA2 and 4. 1F12 demonstrates binding to murine EphA2, 3, 4, 5, 6, 7, and 8, and also to murine EphB1 and 2.

FIG. 25. The chemical structure of monomethyl auristatin E, including a spacer moiety and VC linker is shown.

FIG. 26. The chemical structure of monomethyl auristatin F, including a spacer moiety and VC linker is shown.

FIG. 27. The chemical structures of monomethyl auristatin E and F, including a spacer moiety and two different linkers (valine-citrulline and maleimidocaproyl-citrulline) are shown.

FIG. 28. Conjugation of ADC. Conjugation of a representative anti-EphA2 antibody is represented in this figure. An average of four drug linkers per molecule of antibody are conjugated via a stable peptide linker (Hamblett et al., Clinical Cancer Research 2004).

FIG. 29. Conjugation of anti-EphA2 antibodies with mcMMAF. This figure summarizes the yields (mg) and other properties (% aggregate and endotoxin concentration) of the mcMMAF conjugated 1C1, 1F12, and 1H3 antibodies.

FIG. 30. In vitro growth inhibition comparisons of different linker and drug combinations with anti-EphA2 antibodies of several different cancer cell lines. The anti-EphA2 antibody G5, conjugated to vcMMAF, was compared to the anti-EphA2 antibody 3F2, conjugated to vcMMAE, vcMMAF or mcMMAF, in SKMEL, PC-3, and MDA231 cell lines. Concentrations tested ranged from 0.001 to 100 μg/ml. Results are summarized in three different panels of graphs.

FIG. 31. In vitro growth inhibition by the anti-EphA2 antibodies EA5 linked to MMAF with the vc linker as compared to the control 1A7 antibody linked to MMAF. The following cell lines were tested: A549, MDA231, and A375. Concentrations tested ranged from 0.001 to 100 μg/ml. Results are summarized in four different panels of graphs.

FIGS. 32A-32C. In vitro growth inhibition by the anti-EphA2 antibody EA5 linked to MMAF with the vc linker as compared to the control 1A7 antibody linked to MMAF, EA5 without MMAF, EA5 in competition, and free MMAE. The following cell lines were tested: MDA231 (FIG. 32A), A549 (FIG. 32B), and A375 (FIG. 32C). Concentrations tested ranged from 0.001 to 100 μg/ml.

FIG. 33. In vitro growth inhibition by the anti-EphA2 antibody EA5 linked to MMAF with the vc linker as compared to the control 1A7 antibody linked to MMAF, EA5 in competition, and free MMAE. The following cell lines were tested: HCT-116 and SW620. Concentrations tested ranged from 0.001 to 100 μg/ml. Results are summarized in two different panels of graphs.

FIG. 34. In vitro growth inhibition of MDA-231 cells by the anti-EphA2 antibody EA5 linked to MMAF with the vc linker as compared to the control 1A7 antibody linked to MMAF, EA5 alone, EA5 in competition, and free MMAE. Concentrations tested ranged from 0.001 to 100 μg/ml. Results are summarized in two different panels of graphs.

FIG. 35. In vitro growth inhibition of PC-3 cells and MDA-MB-468 cells by the anti-EphA2 antibody G5 linked to MMAF with the vc linker as compared to the control 1A7 antibody linked to MMAF, G5 in competition, and free MMAE. Concentrations tested ranged from 0.001 to 100 μg/ml. Results are summarized in four different panels of graphs.

FIG. 36. In vitro growth inhibition of A498 cells, PC-3 cells, and MDA-MB-468 cells by the anti-EphA2 antibody G5 and EA5 linked to MMAF with the vc linker. Concentrations tested ranged from 0.001 to 100 μg/ml. Results are summarized in three different panels of graphs.

FIG. 37. In vitro growth inhibition of PC-3 cells, 231KC cells, and T-231 cells by the anti-EphA2 antibody G5 linked to MMAF with the vc linker as compared to the control R3-47 control antibody linked to MMAF, G5 in competition, and G5 alone. Concentrations tested ranged from 0.001 to 100 μg/ml. Results are summarized in four different panels of graphs.

FIG. 38. In vitro growth inhibition of normal HUVEC cells by the anti-EphA2 antibody G5vcMMAF, 3F2vcMMAF, 3F2vcMMAE compared to 3F2 in competition, and free MMAE. Concentrations tested ranged from 0.001 to 100 μg/ml. Results are summarized in two different panels of graphs.

FIG. 39. In vitro growth inhibition of PC-3 cells by the anti-EphA2 antibody G5 linked to MMAF with the vc linker as compared to the control R3-47 antibody linked to MMAF, G5 in competition, and free MMAE. Concentrations tested ranged from 0.001 to 100 μg/ml.

FIG. 40. Summary of cell lines tested in vitro with the anti-EphA2 antibody G5 conjugated to MMAF with the vc linker.

FIG. 41. Average IC50's (μg/ml) of the anti-EphA2 antibody G5 was determined for a panel of different EphA2 positive cell lines. The IC50 value was extrapolated from the in vitro growth inhibition assays performed on the cell lines.

FIG. 42. IC50 values of the anti-EphA2 antibodies 3F2vcMMAE and 3F2mcMMAF were determined for a panel of different EphA2 positive cell lines. The IC50 value was extrapolated from the in vitro growth inhibition assays performed on the cell lines. The cell lines assayed are as follows, with EphA2 expression levels ordered highest to lowest: HEY-A8, PANC.02.03, KC231, PC3, DU-145, ACHN, A498, A549, SKMEL28.

FIGS. 43A-43B. IC50 values of the anti-EphA2 antibodies 3F2mcMMAF, 1C1mcMMAF, and 1F12mcMMAF were determined for a panel of different EphA2 positive human carcinoma cell lines. The IC50 value was extrapolated from the in vitro growth inhibition assays performed on the cell lines. The cell lines assayed are as follows: PC3, KC231, SKOV3, and HEY-A8. FIG. 44B demonstrates an acceptable IC₅₀ concentration for in vivo administration.

FIG. 44. In vitro growth inhibition of MCF10-A and HUVEC cells by the anti-EphA2 antibodies 1C1, 1F12, and 3F2 linked to MMAF with the mf linker as compared to free MMAE. EphA2 surface expression on the HUVEC and MCF10-A cells, and binding to the surface expressed EphA2 by the tested antibodies is also summarized in a separate panel.

FIG. 45. In vitro growth inhibition of PC-3 cells by the anti-EphA2 antibodies 1C1, 1F12, and 3F2 linked to MMAF with the mf linker is compared to the same antibodies with their unlinked corresponding antibodies in competition. Concentrations tested ranged from 0.001 to 10 μg/cc.

FIG. 46. In vitro growth inhibition of KC-231 and PC3 cells by the anti-EphA2 antibodies 1C1 and 1F12 linked to MMAF with the mf linker, and linked to MMAE with the vc linker. Different lots of the conjugated antibodies were compared in this set of experiments. Concentrations tested ranged from 0.001 to 100 μg/cc.

FIG. 47. Cross species activity of the anti-EphA2 ADC's 1C1 and 1F12 in EphA2+ cell lines. The anti-EphA2 antibodies 1C1 and 1F12 linked to MMAF with the mc linker were compared to the control R347 antibody linked to MMAF with the mc linker in the following cells: F98 (rat glioma), PC3 (human prostate cancer), CT26 (mouse colon cancer), and CYNO-MK. Concentrations tested ranged from 0.001 to 10 μg/cc.

FIG. 48. In vivo comparison of the anti-EphA2 G5 antibody conjugated to MMAF with the vc linker as compared to unconjugated G5, control IgG conjugated to MMAF, and control unconjugated IgG in the PC-3 human prostate cancer cell line. Doses of antibodies were 20 μg and 200 μg.

FIG. 49. In vivo comparison of the anti-EphA2 G5 antibody conjugated to MMAF with the vc linker as compared to control IgG conjugated to MMAF in the MDA-MB-231KC human breast cancer cell line. Doses of antibodies were 20 μg, 50 μg, and 100 μg.

FIG. 50. In vivo comparison of the anti-EphA2 G5 antibody conjugated to MMAF with the vc linker, or MMAF with the mc linker as compared to control IgG conjugated to MMAF and control conjugated and unconjugated R347 in the PC3 human prostate cancer cell line. Doses of antibodies were 20 μg and 200 μg.

FIG. 51. In vivo comparison of the anti-EphA2 3F2 antibody conjugated to MMAE with the vc linker and the anti-EphA2 3F2 antibody conjugated to MMAF with the mc linker as compared to control R347 conjugated to MMAE or MMAF, and PBS in the PC-3 human prostate cancer cell line. Doses of antibodies were 3 mg/kg (60 μg) for the MMAE conjugates and 10 mg/kg (200 μg) for the MMAF.

FIG. 52. In vivo comparison of the anti-EphA2 antibodies 1C1 and 1F12 conjugated to MMAF with the mc linker as compared to the control R347 conjugated to MMAF and PBS in the PC-3 human prostate cancer cell line. Doses of antibodies were 3 mg/kg (60 μg) or 1 mg/kg (20 μg).

FIGS. 53A-53C. In vivo comparison of the anti-EphA2 antibodies 1C1 and 1F12 conjugated to MMAE with the vc linker, or conjugated to MMAF with the mc linker as compared to PBS in the PC-3 human prostate cancer cell line. Doses of antibodies were 6.0 mg/kg (FIG. 54A), 3.0 mg/kg (FIG. 54B), and 1.0 mg/kg (FIG. 54C).

FIG. 54. In vivo comparison of the anti-EphA2 antibodies 1C1 and 1F12 conjugated to MMAF with the mc linker as compared to PBS, the control R347 conjugated to MMAF, and the unconjugated anti-EphA2 antibody 3F2-3M in the MDA-231KC human breast cancer cell line. Doses of antibodies were 1 mg/kg (20 μg), 3 mg/kg (60 μg), 6 mg/kg (120 μg), or 10 mg/kg (200 μg).

FIG. 55. In vivo comparison of the anti-EphA2 antibodies 1C1 and 1F12 conjugated to MMAE with the vc linker, or conjugated to MMAF with the mc linker as compared to PBS in the MDA-231KC human breast adenocarcinoma cell line. Doses of antibodies were 1 mg/kg (20 μg) or 6 mg/kg (120 μg).

FIG. 56A-56C. In vivo comparison of the anti-EphA2 antibodies 1C1 and 1F12 conjugated to MMAE with the vc linker, or conjugated to MMAF with the mc linker as compared to PBS in the MDA-231KC human breast adenocarcinoma cell line. Doses of antibodies were 6.0 mg/kg (FIG. 57A), 3.0 mg/kg (FIG. 57B), and 1.0 mg/kg (FIG. 57C).

FIG. 57. MMAE free drug growth inhibition. Several different mouse, rat, human and monkey cell lines was tested for sensitivity to free MMAE in vitro, with the resulting IC50's (μM) summarized.

FIG. 58. Anti-EphA2 ADC toxicity as a measurement of body weight loss in Balb/c mice. The anti-EphA2 antibodies 1C1 and 1F12 conjugated to MMAE with the vc linker, or conjugated to MMAF with the mc linker were tested in vivo to determine the effect of administration on weight of mice as compared to control PBS administration. Doses of the vcMMAE antibodies were 40 mg/kg, 50 mg/kg, and 60 mg/kg. Doses of the 1C1-mcMMAF antibody were 120 mg/kg, 180 mg/kg, and 240 mg/kg. Doses of the 1F12-mcMMAF antibody were 90 mg/kg, 120 mg/kg, 180 mg/kg, 210 mg/kg, and 240 mg/kg.

FIG. 59. Anti-EphA2 ADC's therapeutic windows. Potential therapeutic windows for 1C1-mcMMAF, 1C1-vcMMAE, 1F12-mcMMAF, and 1F12-vcMMAE based on in vitro and in vivo data observations are summarized in this figure.

### DETAILED DESCRIPTION OF THE INVENTION

The receptor tyrosine kinases (RTKs) are transmembrane molecules which relay signals from the extracellular environment into the cytoplasm. The Eph family of RTKs is the largest subfamily of RTKs. This group is distinguished by a cysteine-rich region and two fibronectin type III repeats in the extracellular domain. The Eph receptors are activated by a second family of cell surface-anchored proteins, the ephrins. Members of both the Eph tyrosine kinases and the ephrin ligands mediate signaling after receptor-ligand interaction (Bruckner et al., 1997, Science 275:1640; Holland et al., 1996, Nature 383:722). This bi-directional signaling are known to affect processes involving cellular interaction, like cell adhesion, cell migration and tissue border formation (Boyd et al., 2001 Sci STKE RE20; Schmucher et al., 2001, Cell 105:701-4; Kullander et al., 2002 Nat. Rev. Mol. Cell Biol. 3:475). More recently, the Eph receptors have been linked to the development and progression of cancers.

As cell surface molecules, the Eph receptors are readily accessible target molecules for antibody directed therapies. In tumor cells that overexpress EphA2, for example, the increased presence of surface receptor causes unstable cell-cell contacts, which disrupts cell cycle regulation and leads to tumor cell growth, proliferation and invasiveness. Naked antibodies against different members of the Eph receptor family (e.g. EphA2) have shown agonist activity through phosphorylation, internalization, and degradation of the receptor (see for example U.S. Pat. No. 6,927,203, U.S. Provisional Application No. 60/717,209, U.S. Patent Application Publication No. US2006/0121042-A1, U.S. patent application Ser. Nos. 09/952,560, 10/994,129, 10/436,782, 10/863,729, and 11/203,251, each of which is hereby incorporated by reference herein in its entirety). In one embodiment, the ADCs of the invention are variants of an antibody that specifically binds to at least one Eph receptor. Eph receptors to which the ADCs of the invention specifically binds to include but are not limited to EphA1, EphA2, EphA3a, EphA3b, EphA4, EphA5a, EphA5b, EphA6, EphA7, EphA8, EphB1, EphB2a, EphB2b, EphB3, EphB4 and EphB6.

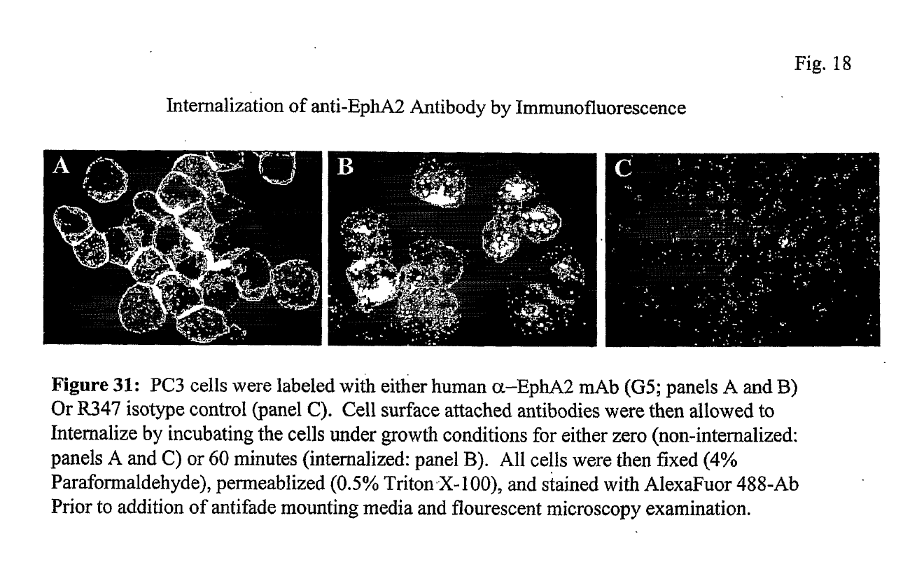

The skilled artisan will appreciate that an Eph receptor of the invention is a molecule that exhibits a substantial degree of homology to known Eph receptors (see, e.g., supra), such that it has been or can be classified as an Eph receptor family molecule based upon, its amino acid sequence. Pairwise comparisons of the known human Eph receptors were performed using the MegaAlign program (DNASTAR) with the Clustal W algorithm (Thompson et al., 1994 Nucleic Acids Res 22:4673-80). The results (FIG. 18) show that there are multiple regions each protein that share a high degree of similarity among the Eph receptor family members. It is specifically contemplated that one skilled in the art could generate antibodies to regions of an Eph receptor that would allow for cross reactivity of said antibody between family members or a more restricted specificity such that said antibody specifically bound only one family member with high affinity. To identify potential immunogenic peptides for use in generating antibodies that could be either protein specific or would bind with one or more Eph receptors, the antigenic index of each protein can be examined using the Protean program (DNASTAR) with the Jameson-Wolf algorithm. The regions with the highest antigenic indices among all members of the Eph receptor family can be identified and those regions which are highly conserved among one or more family members and would be excellent candidates for raising an antibody which recognizes more then one family member. While the use of less conserved regions would likely generate an antibody specific for one Eph receptor family member.

In one embodiment, the ADCs of the invention preferentially bind to an Eph receptor present on a tumor cell and do not bind to an Eph receptor present on a non-tumor cell. In another embodiment, the ADCs of the invention do not stain normal tissues including but not limited to, brain, lung, pancreas, liver, prostate, heart, ovary, skin, kidney, intestine and stomach. Antibody binding and specific staining patterns can be readily determined by immunological labeling methods well known in the art including but not limited to, immunohistochemistry and Fluorescence Activated Cell Scanning/Sorting (FACS). Specific methods and protocols are found in Polak and Van Noorden (1997) Introduction to Immunocytochemistry, second edition, Springer Verlag, N.Y. and in Haugland (2004) Handbook of Fluorescent Probes and Research Chemicals, ninth edition, a combined handbook and catalogue Published by Molecular Probes, Inc., Eugene, Oreg. among others.

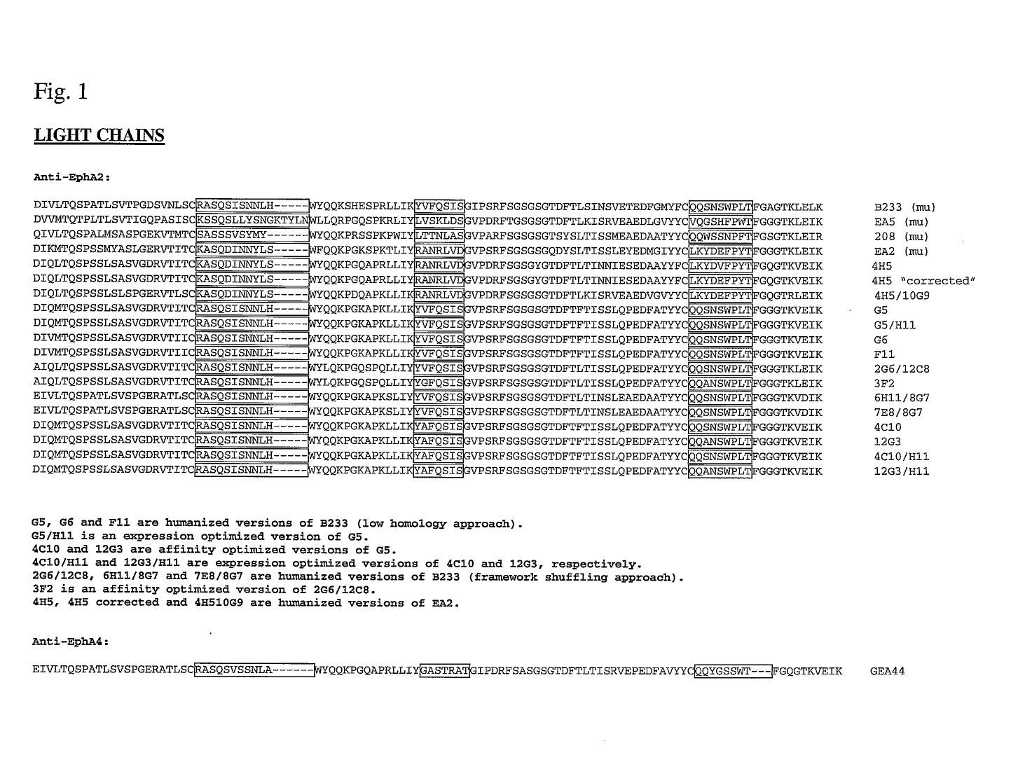

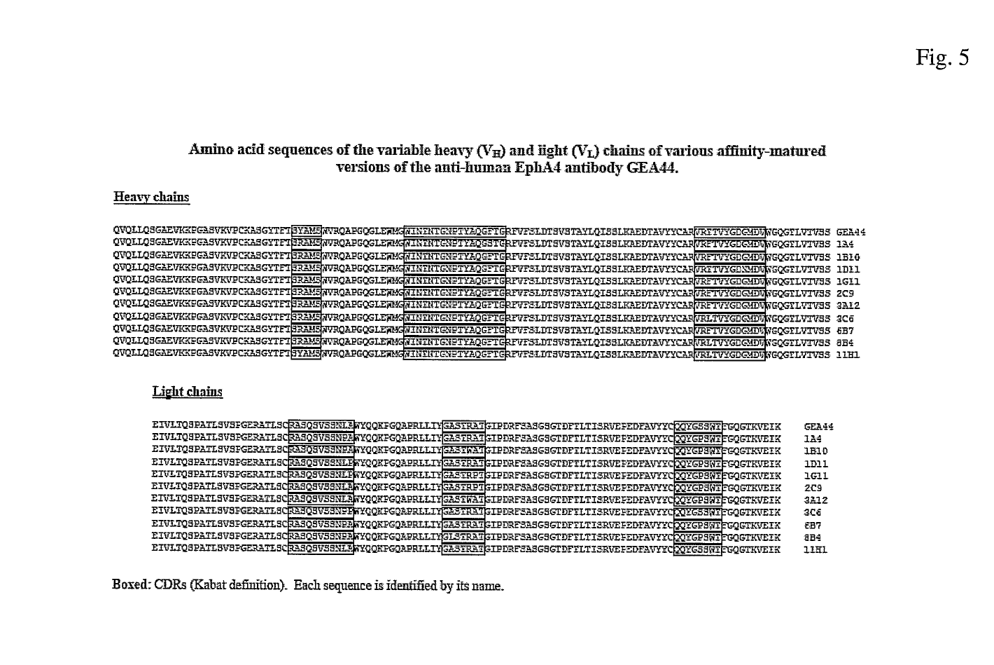

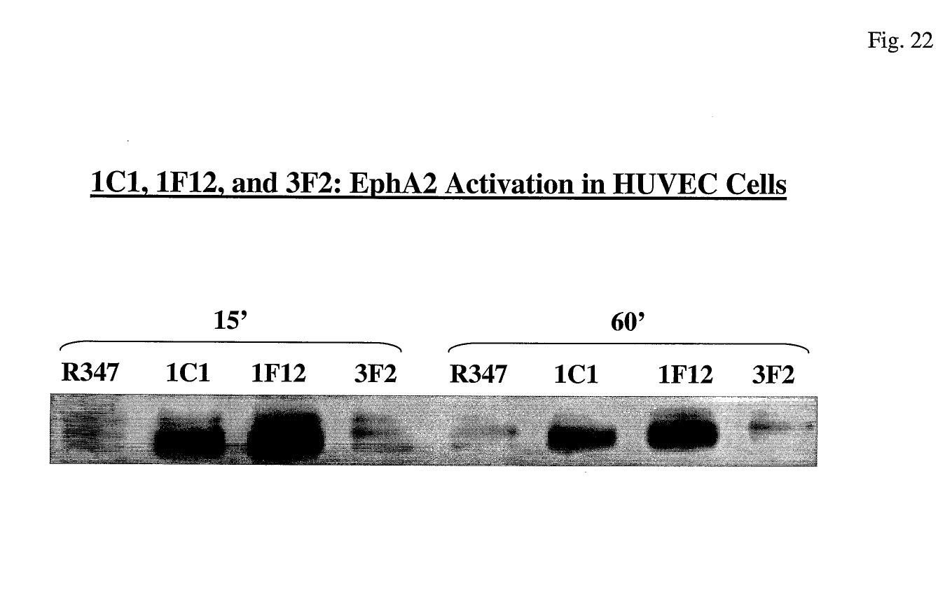

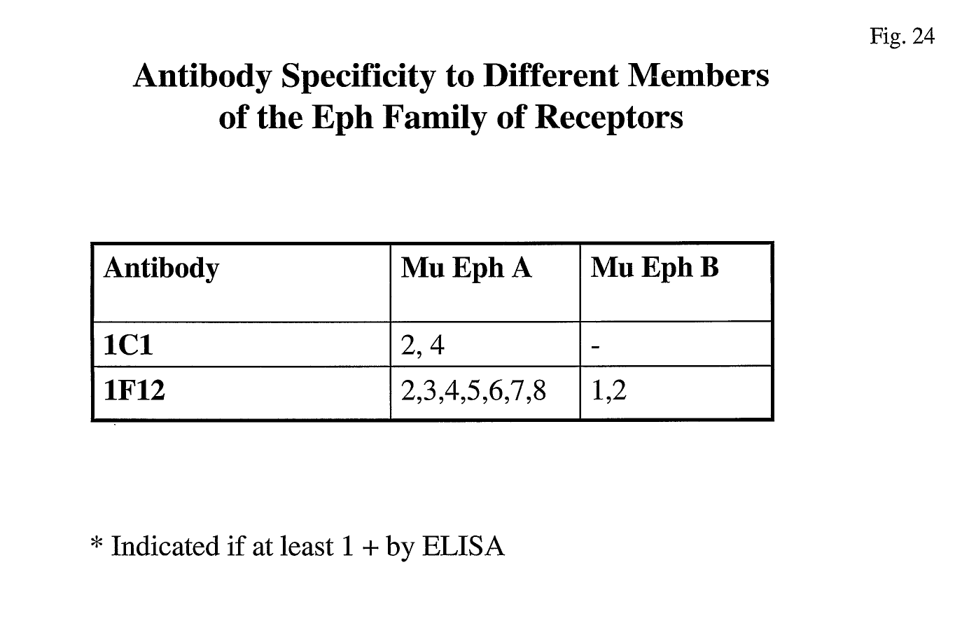

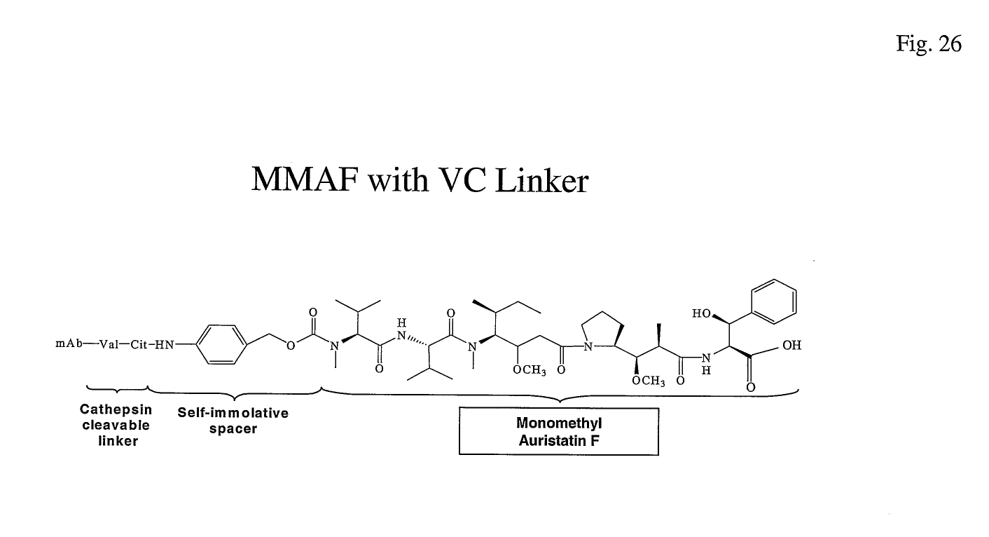

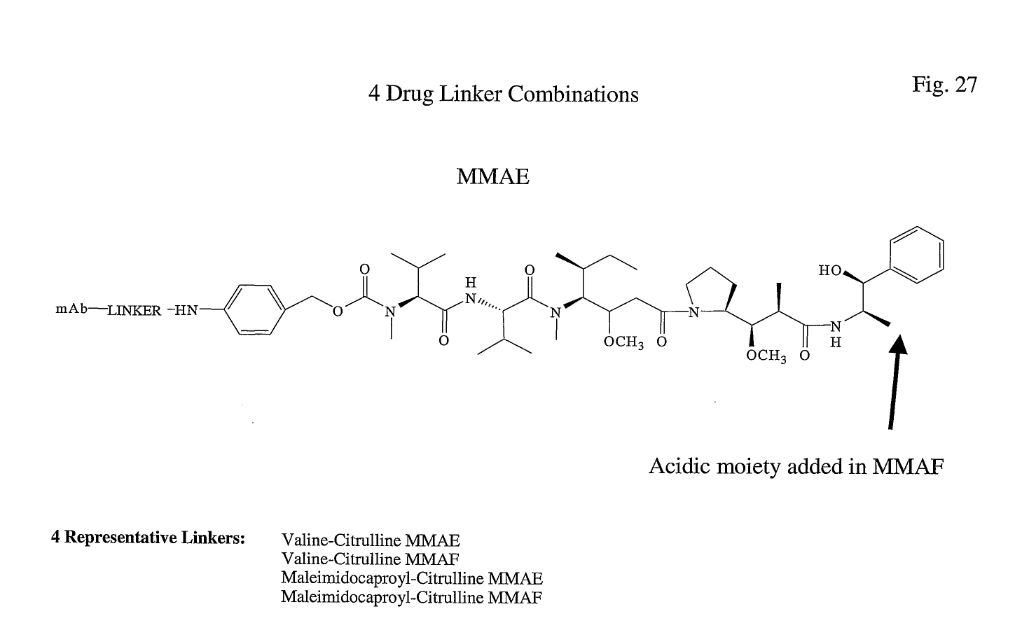

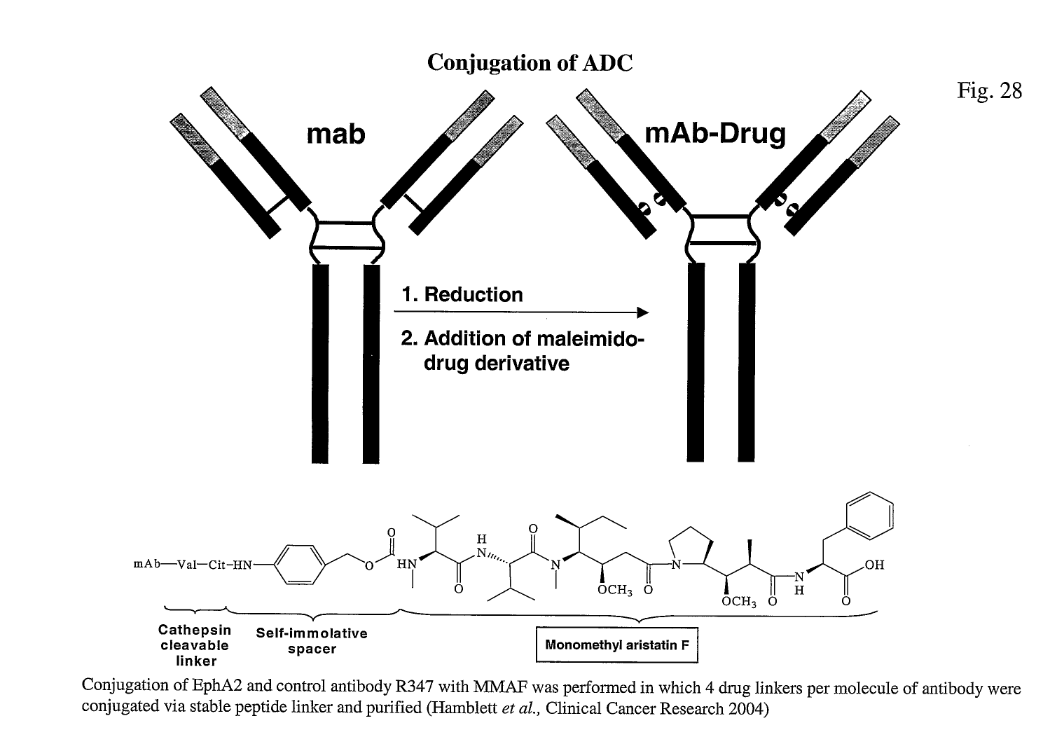

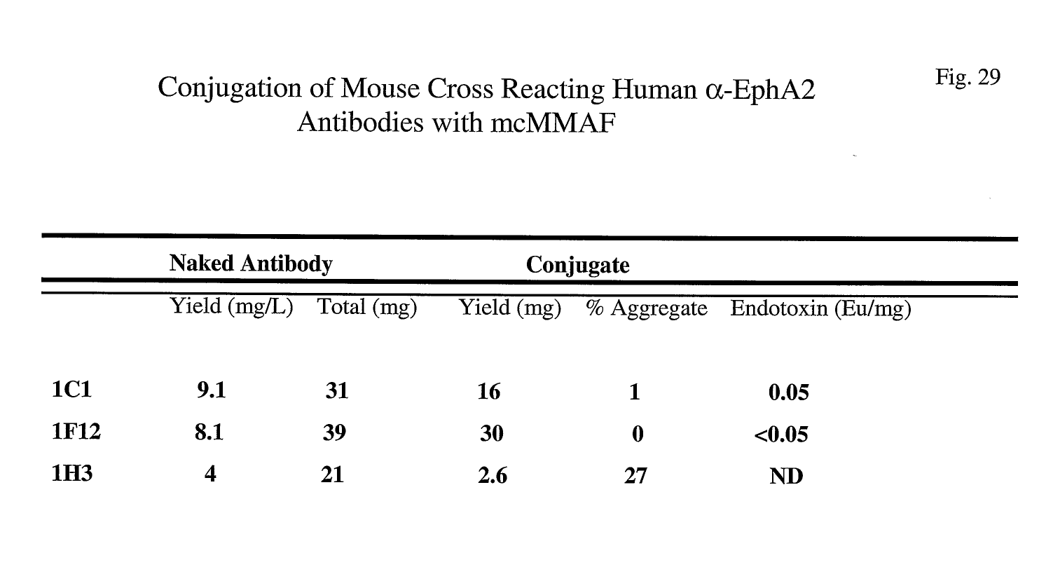

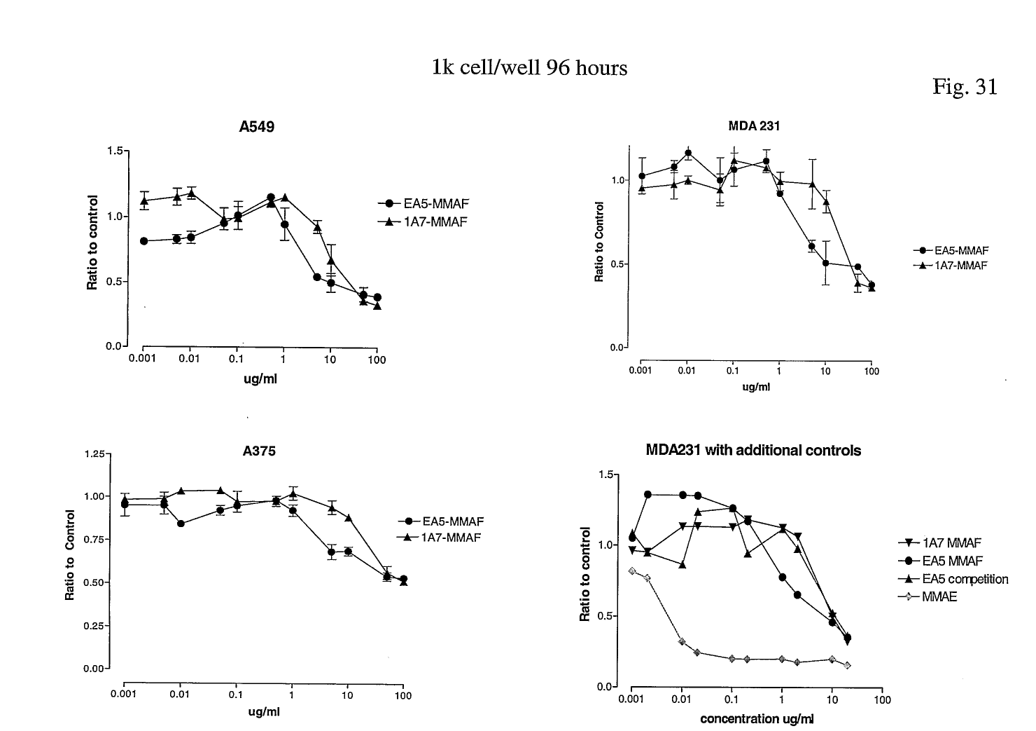

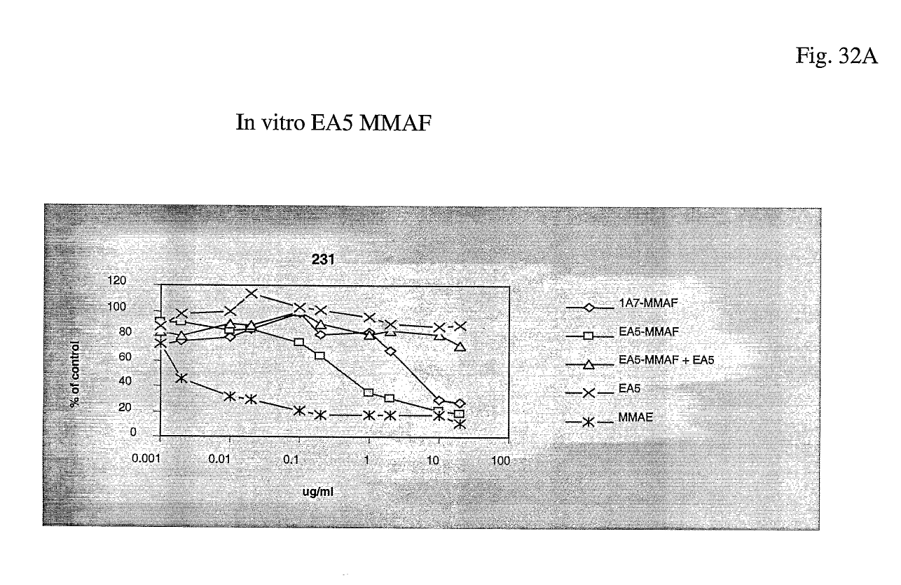

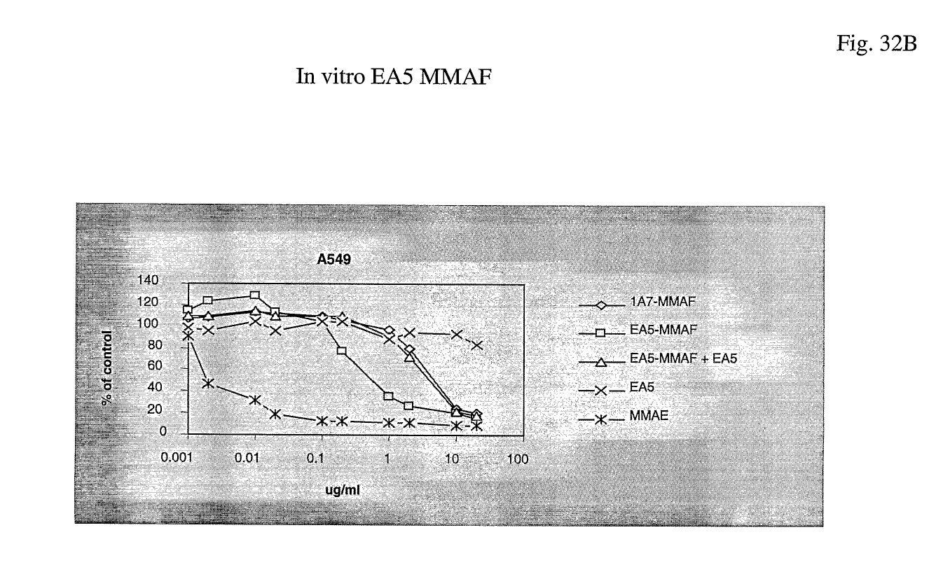

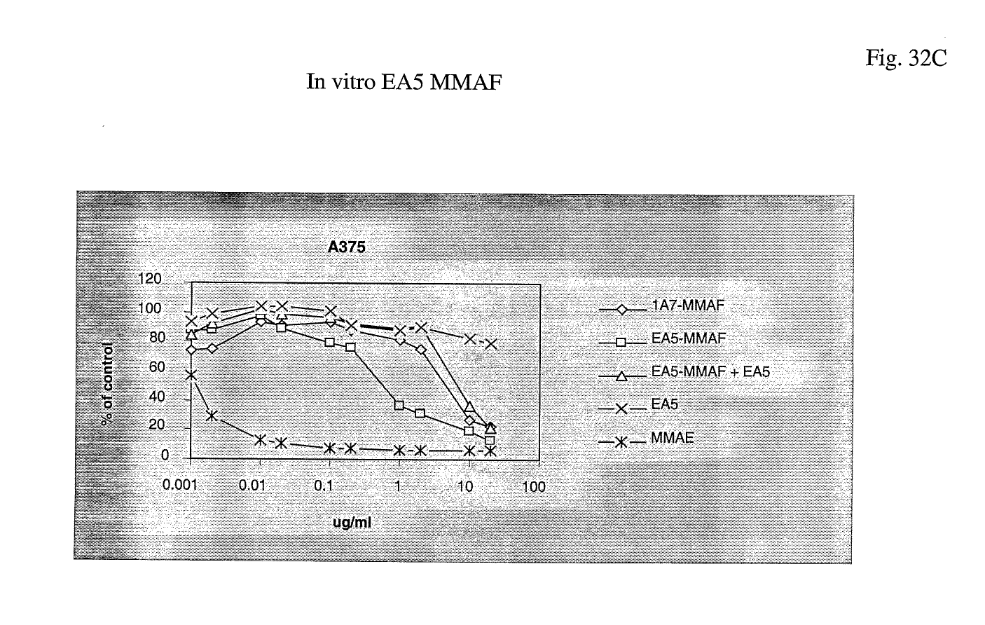

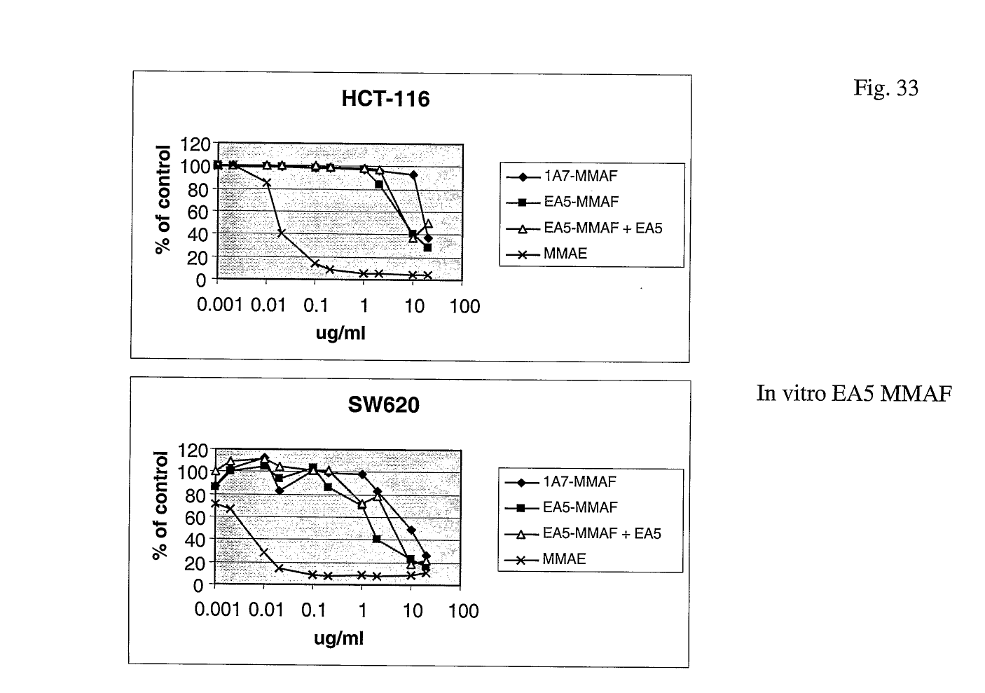

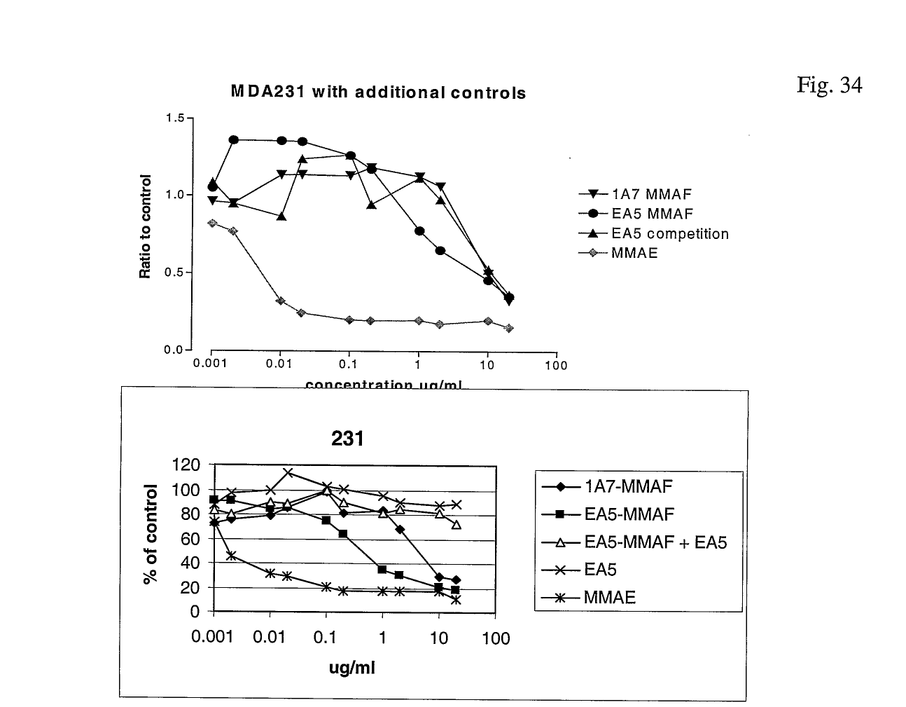

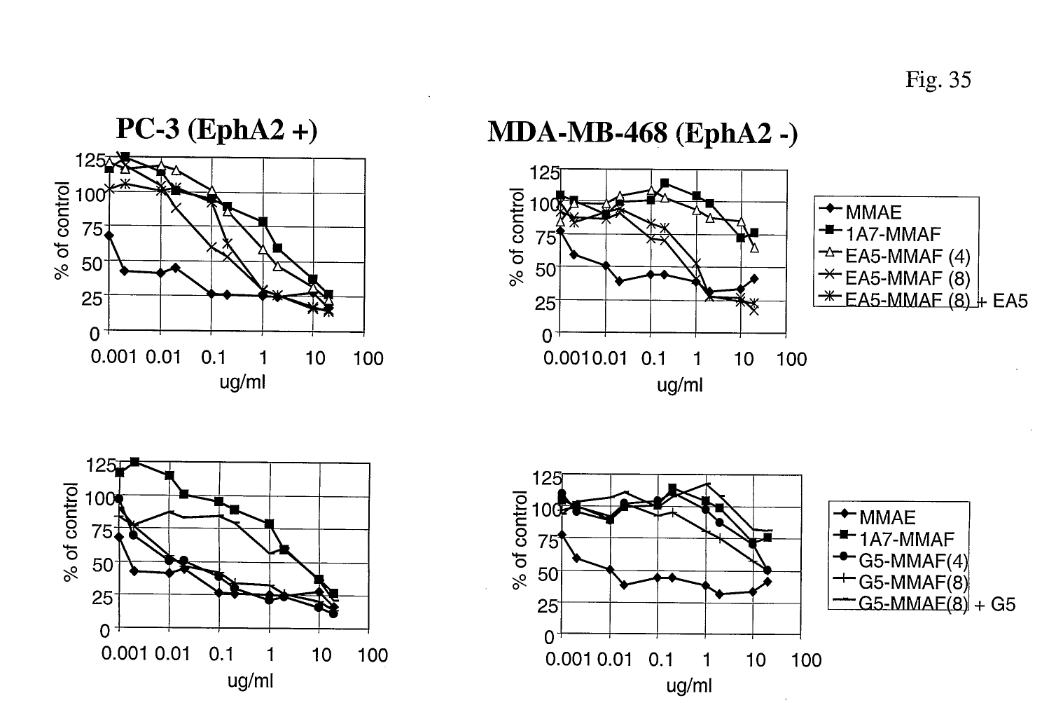

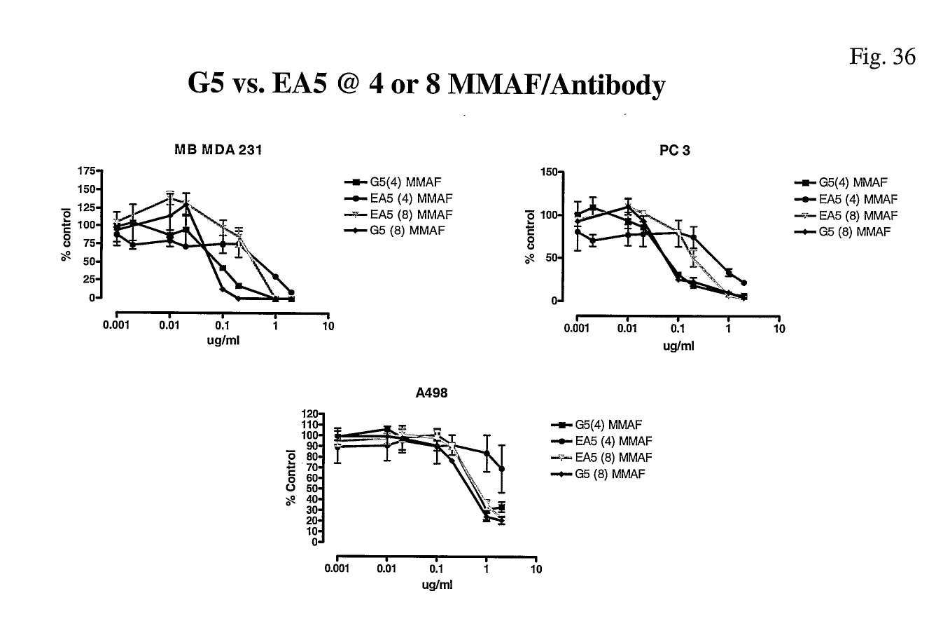

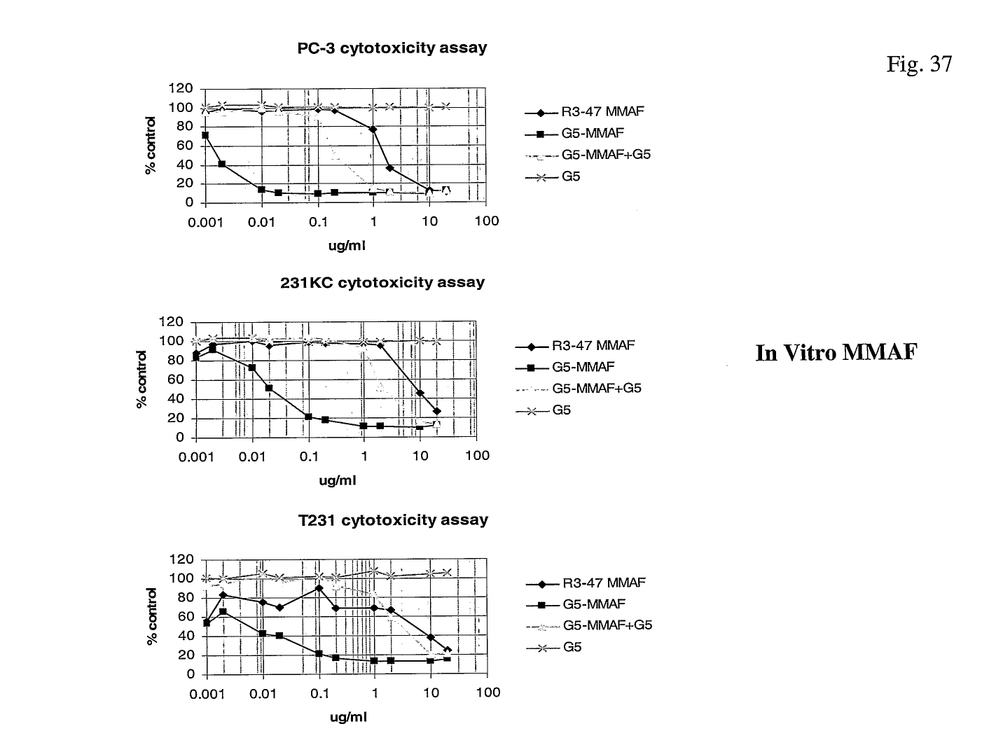

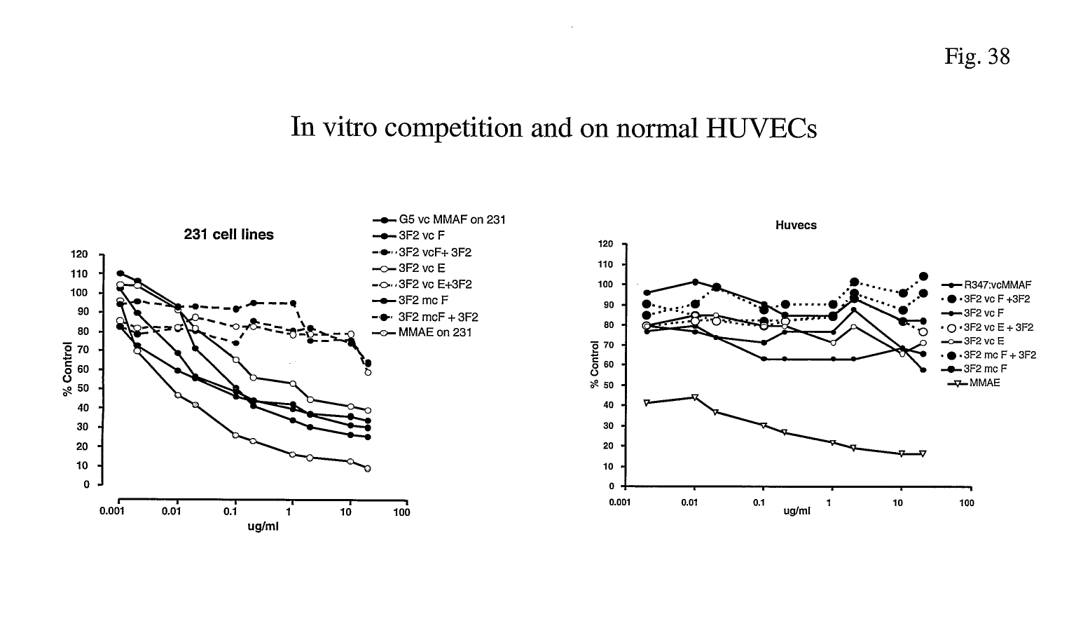

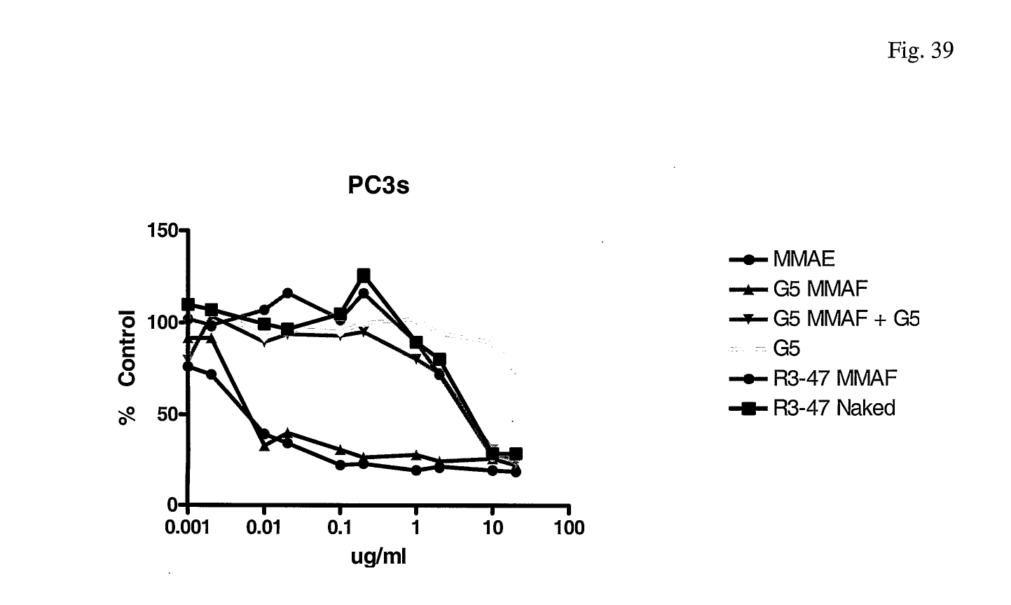

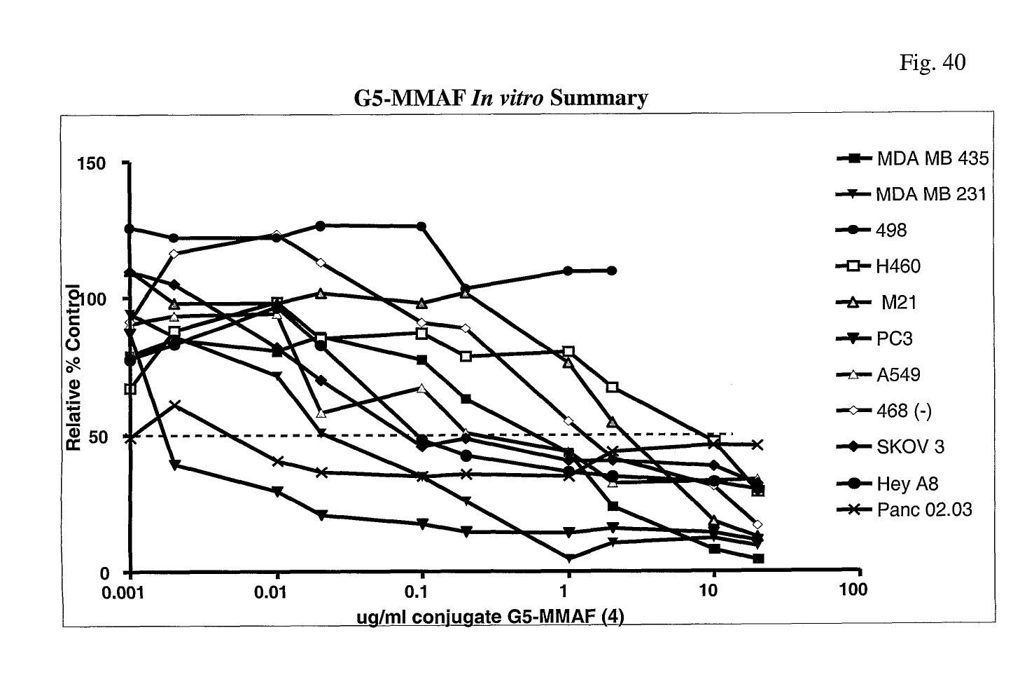

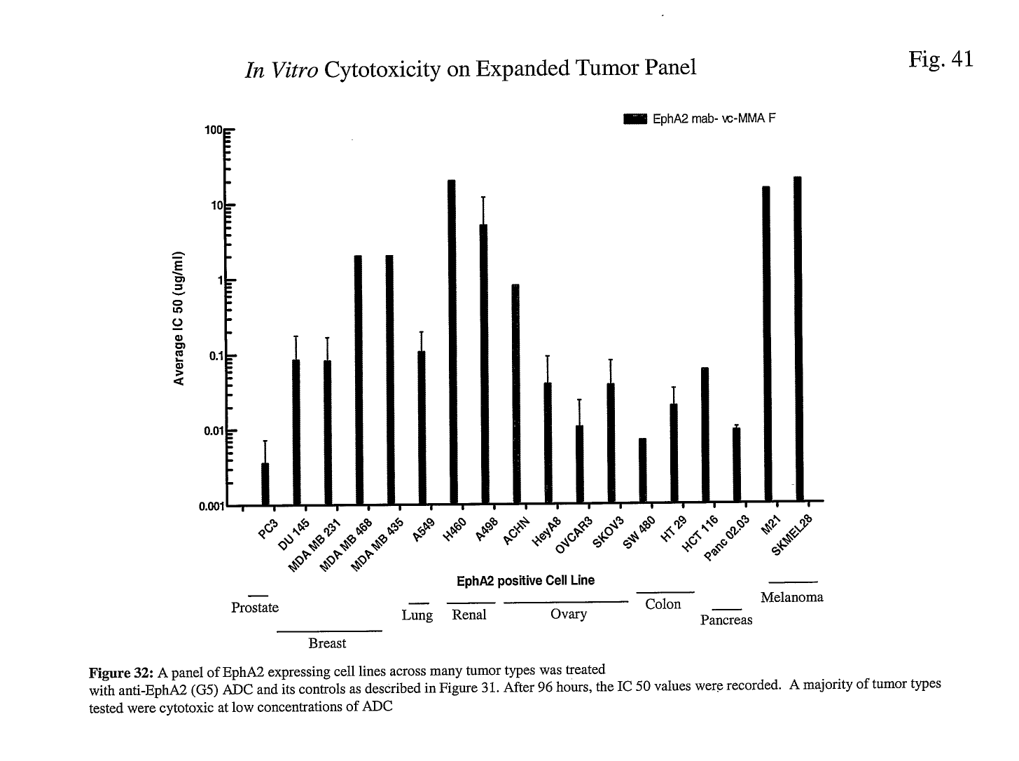

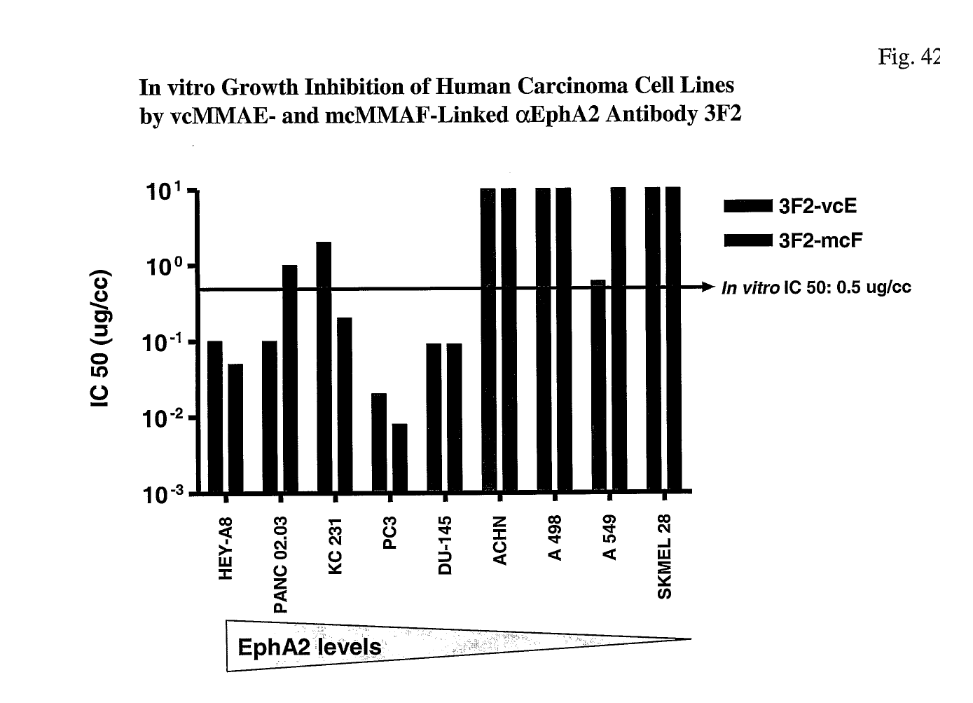

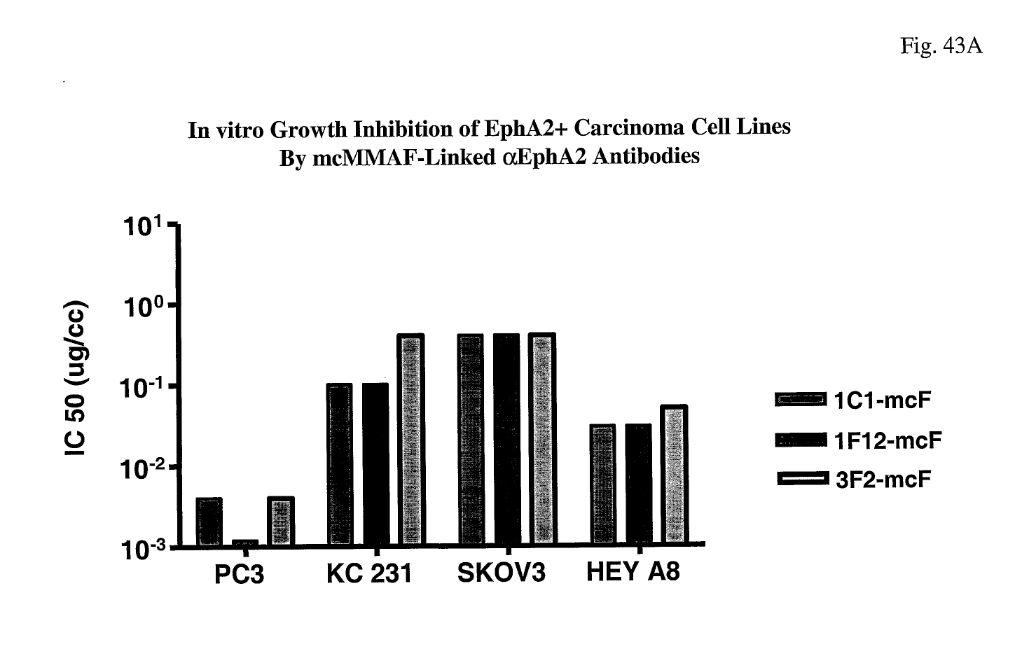

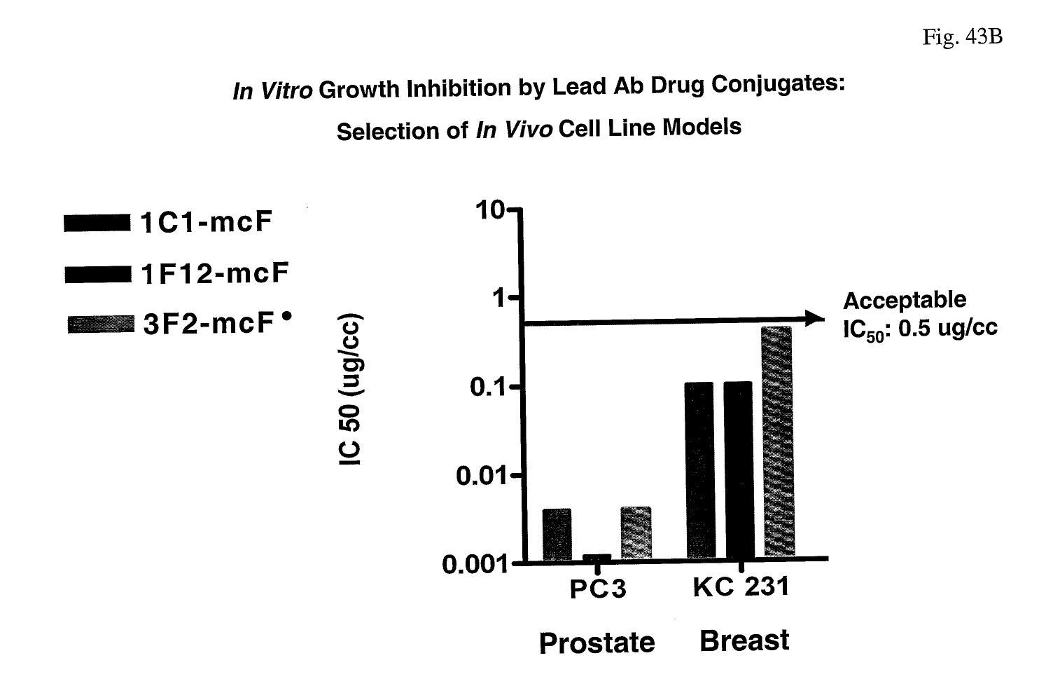

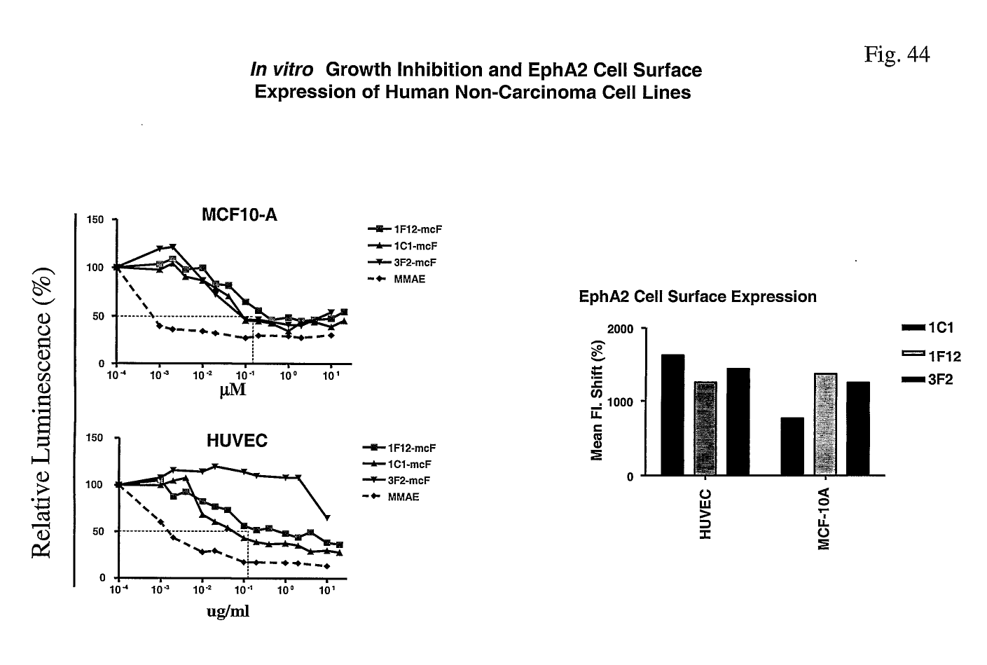

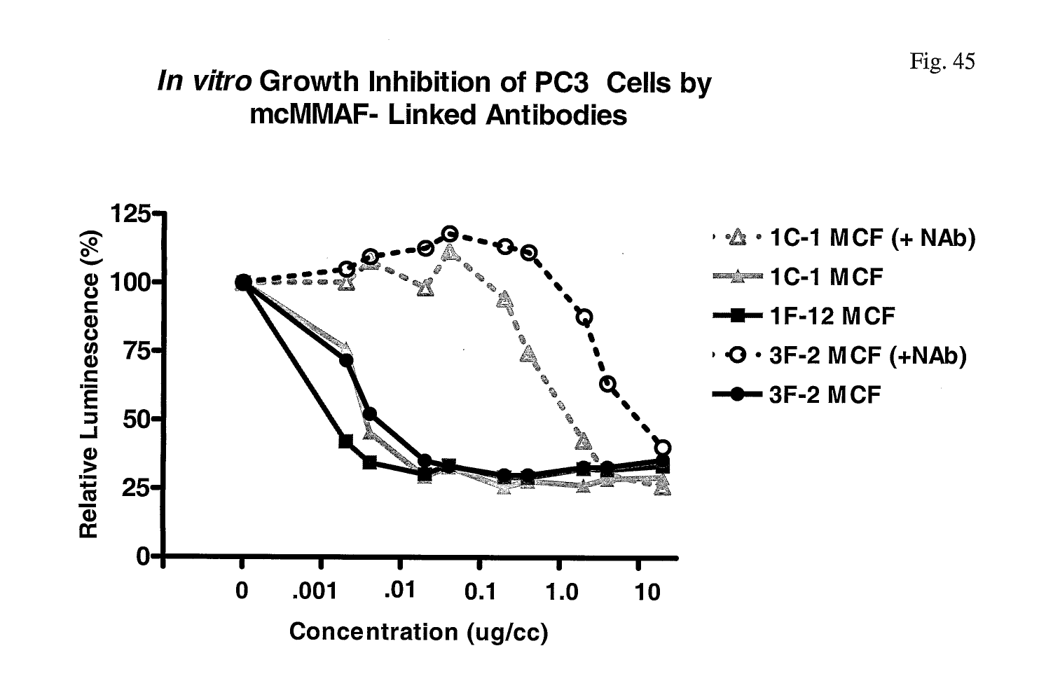

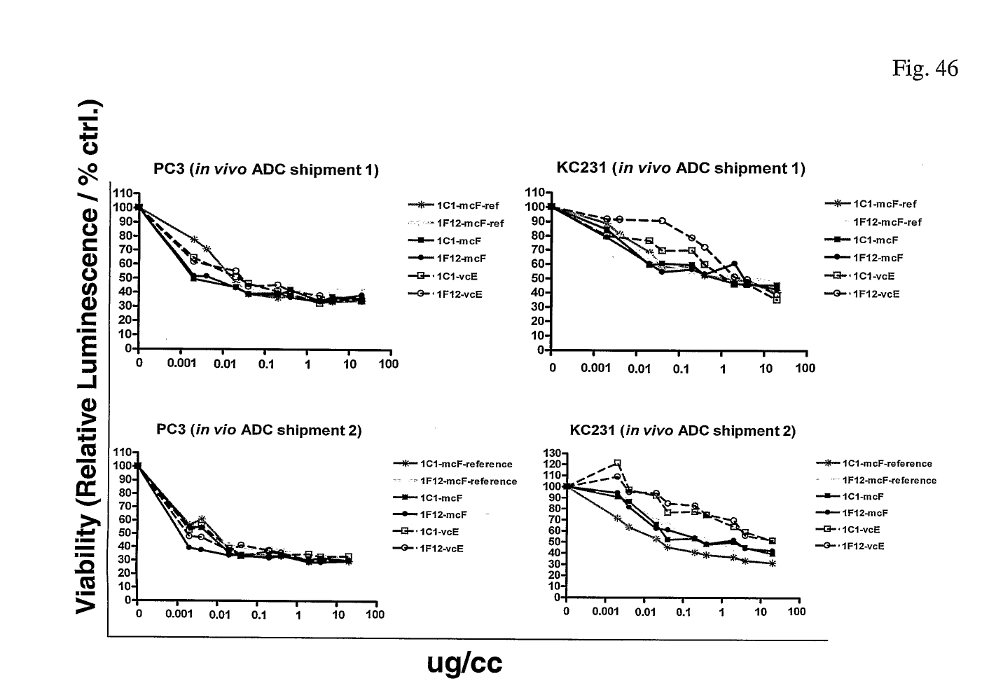

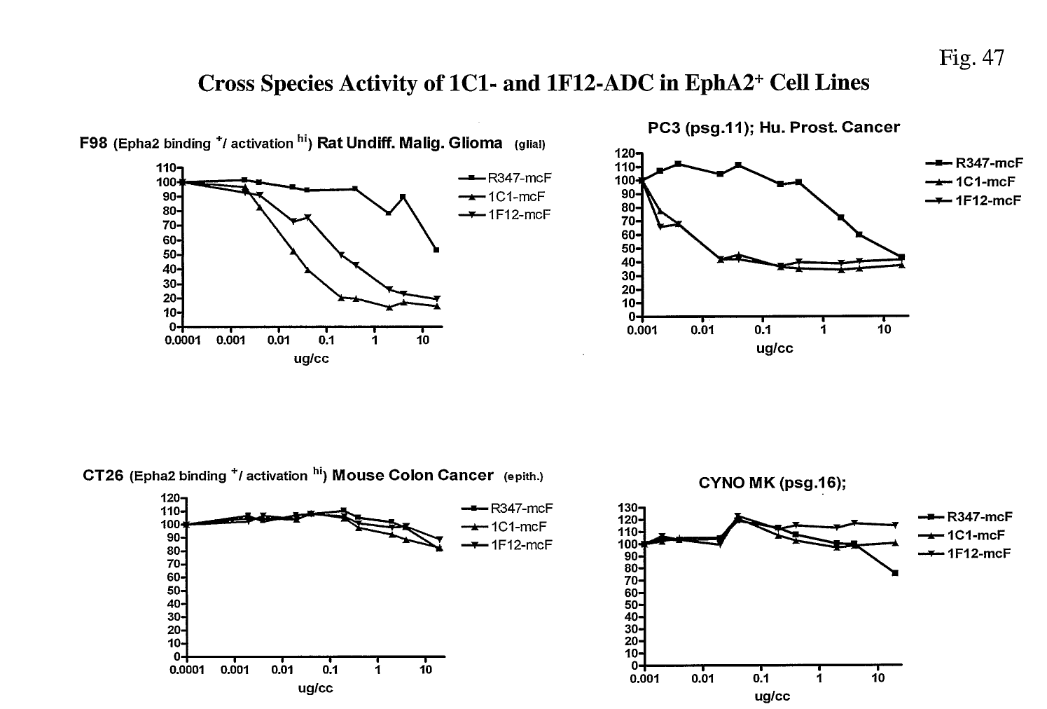

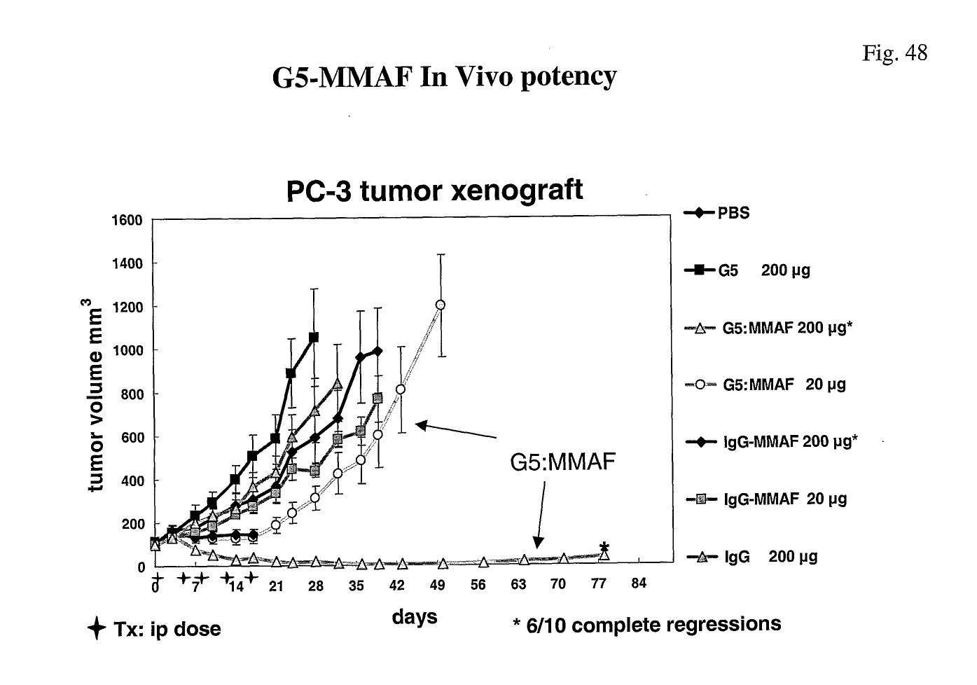

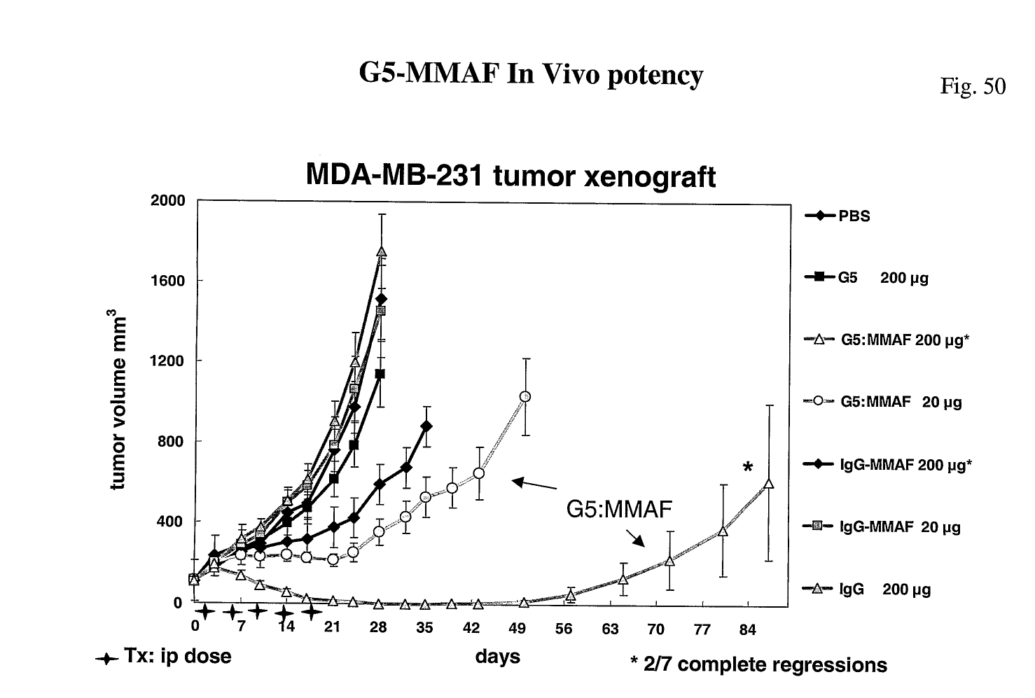

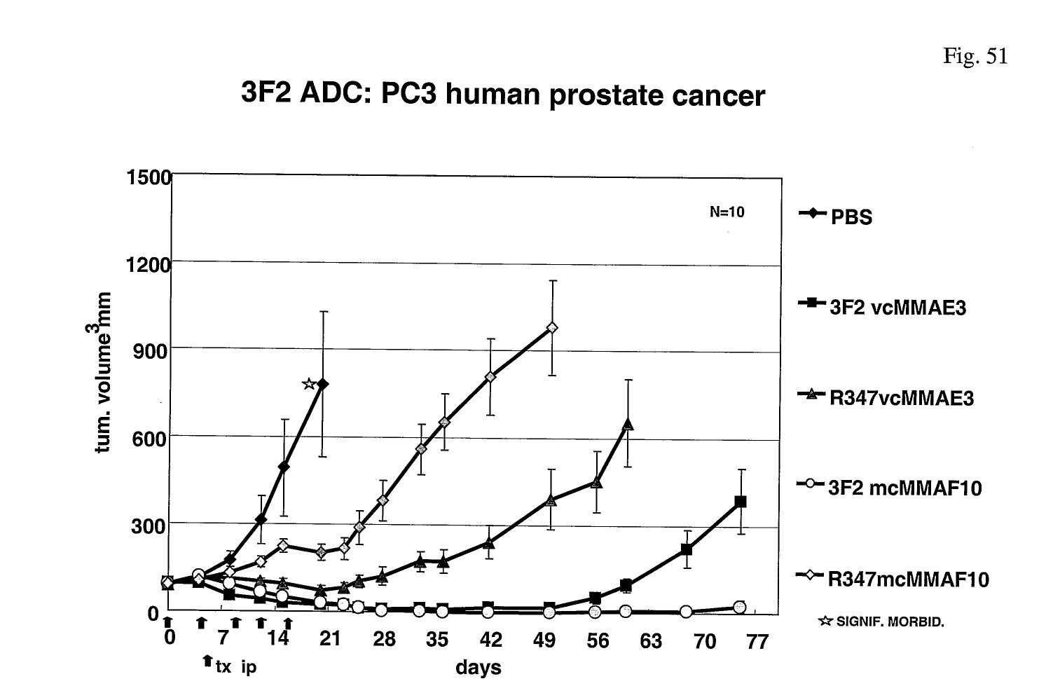

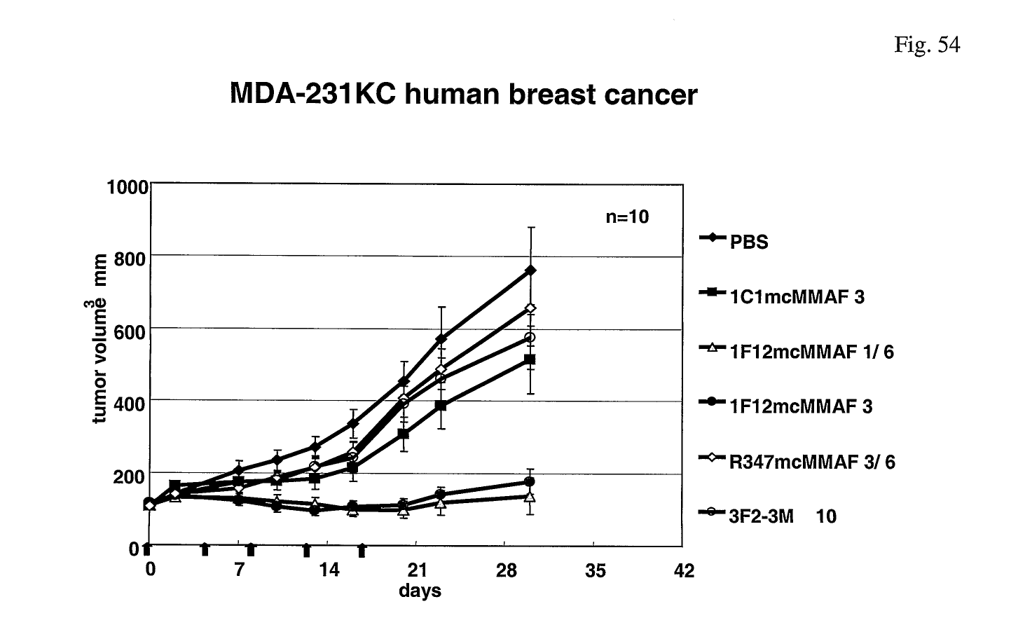

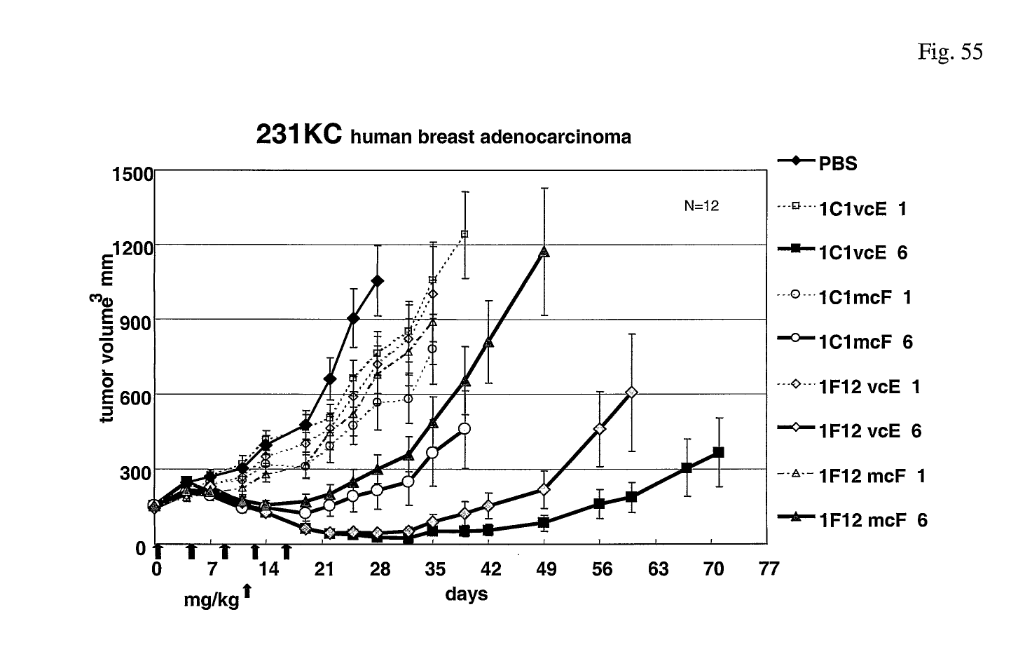

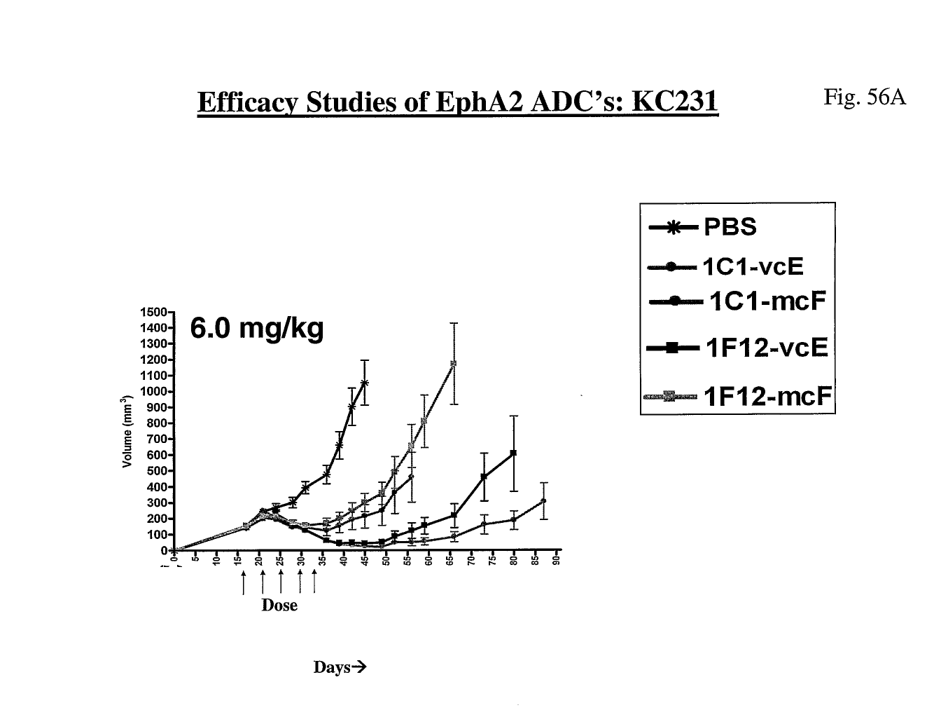

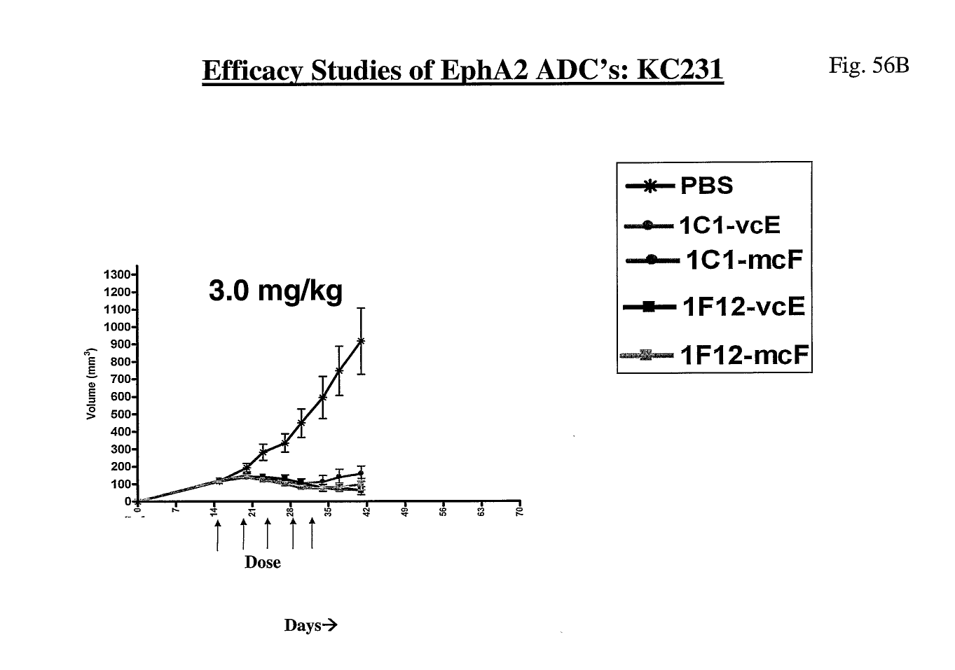

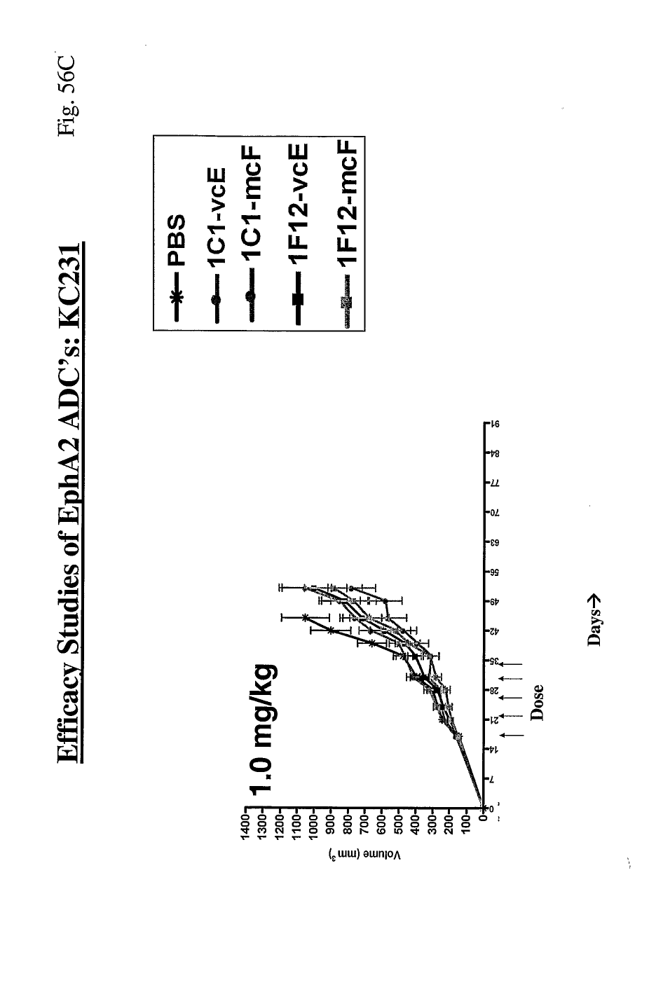

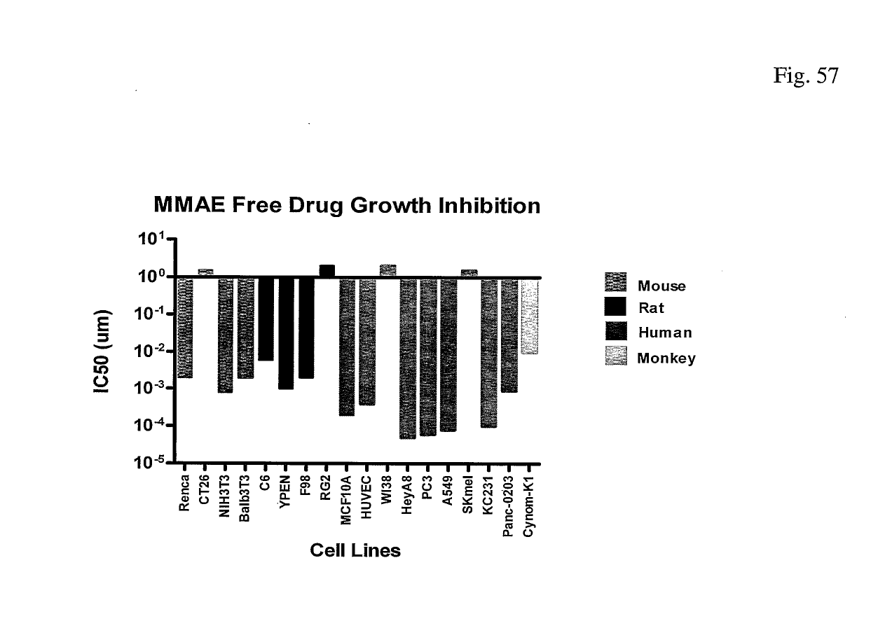

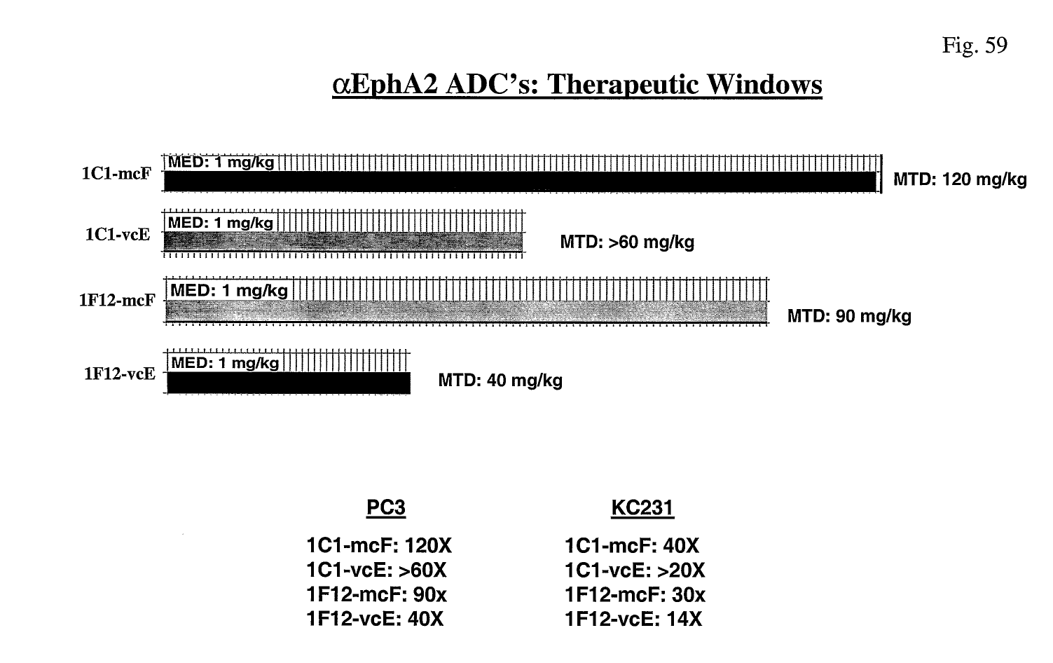

In another embodiment, the ADCs of the invention are variants of antibodies that specifically bind EphA2 and/or EphA4, their derivatives, analogs and epitope-binding fragments thereof, such as but not limited to, those disclosed herein and in PCT Publication Nos. WO 04/014292, WO 03/094859 and U.S. patent application Ser. No. 10/863,729, each of which is incorporated herein by reference in its entirety and any of the antibodies listed in Tables 2-4 or 6, or FIGS. 1-59. In a specific embodiment, the ADCs of the invention are antibodies that specifically bind EphA2 and/or EphA4 which comprise all or a portion of the variable region (e.g., one or more CDR) from 12G3H11, and/or 3F2 and/or 12G3H11, B233, B208, B210, G5, 10C12, 4H5, 10G9, 3F2, 1C1, 1F12, 1H3, 1D3, 2B12, 5A8 and/or any of the antibodies listed in Tables 2-4 or 6, or FIGS. 1-59.

The present invention further encompasses the use of ADCs of the invention that have a high binding affinity for at least on Eph receptor. In a specific embodiment, an ADC of the invention that specifically binds to at least one Eph receptor has an association rate constant or kₒₙ rate ((Ab)+antigen (Ag)^{k}ₒₙ←Ab-Ag) of at least 10⁵M⁻¹s⁻¹, at least 5×10⁵M⁻s⁻¹, at least 10⁶M⁻¹s⁻¹, at least 5×10⁶M⁻¹s⁻¹, at least 10⁷ M⁻¹s⁻¹, at least 5×10⁷M⁻¹s⁻¹, or at least 10⁸M⁻¹s⁻¹. In a further specific embodiment, an ADC of the invention that specifically binds to at least one Eph receptor has an association rate constant or kₒₙ rate ((Ab)+antigen (Ag)^{k}ₒₙ←Ab-Ag) of at least about 10⁵M⁻¹s⁻¹, at least about 5×10⁵M⁻s⁻¹, at least about 10⁶M⁻¹s⁻¹, at least about 5×10⁶M⁻¹s⁻¹, at least about 10⁷M⁻¹s⁻¹, at least about 5×10⁷M⁻¹s⁻¹, or at least about 10⁸M⁻¹s⁻¹. In another embodiment, an ADC that specifically binds to at least one Eph receptor has a kₒₙ of at least 2×10⁵M⁻¹s⁻¹, at least 5×10⁵M⁻¹s⁻¹, at least 10⁶M⁻¹s⁻¹, at least 5×10⁶M⁻¹s⁻¹, at least 10⁷M⁻¹s⁻¹, at least 5×10⁷M⁻¹s⁻¹, or at least 10⁸M⁻¹s⁻¹. In a further embodiment, an ADC that specifically binds to at least one Eph receptor has a kₒₙ, of at least about 2×10⁵M⁻¹s⁻¹, at least about 5×10⁵M⁻¹s⁻¹, at least about 10⁶M⁻¹s⁻¹, at least about 5×10⁶M⁻¹s⁻¹, at least about 10⁷M⁻¹s⁻¹, at least about 5×10⁷M⁻¹s⁻¹, or at least about 10⁸M⁻¹s⁻¹.

In another embodiment, an ADC of the invention that specifically binds to least on Eph receptor has a k_{off} rate ((Ab)+antigen (Ag)^{k}_{off}←Ab-Ag) of less than 10⁻¹s⁻¹, less than 5×10⁻¹s⁻¹, less than 10⁻²s⁻¹, less than 5×10⁻²s⁻¹, less than 10⁻³s⁻¹, less than 5×10⁻³s⁻¹, less than 10⁻⁴s⁻¹, less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰⁻¹s⁻¹. In still another embodiment, an ADC of the invention that specifically binds to least on Eph receptor has a k_{off} rate ((Ab)+antigen (Ag)^{k}_{off}←Ab-Ag) of less than about 10⁻¹s⁻¹, less than about 5×10⁻¹s⁻¹, less than about 10⁻²s⁻¹, less than about 5×10⁻²s⁻¹, less than about 10⁻³s⁻¹, less than about 5×10⁻³s⁻¹, less than about 10⁻⁴s⁻¹, less than about 5×10⁻⁴s⁻¹, less than about 10⁻⁵ s⁻¹, less than about 5×10⁻⁵s⁻¹, less than about 10⁻⁶s⁻¹, less than about 5×10⁻⁶s⁻¹, less than about 10⁻⁷s⁻¹, less than about 5×10⁻⁷s⁻¹, less than about 10⁻⁸s⁻¹, less than about 5×01-s⁻¹, less than about 10⁻⁹s⁻¹, less than about 5×10⁻⁹s⁻¹, or less than about 10⁻¹⁰⁻¹s⁻¹. In a further embodiment, an ADC that specifically binds to least on Eph receptor has a k_{off}, of less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰s⁻¹. In another embodiment, an ADC that specifically binds to least on Eph receptor has a k_{off}, of less than about 5×10⁻⁴s⁻¹, less than about 10⁻⁵s⁻¹, less than about 5×10⁻⁵s⁻¹, less than about 10⁻⁶s⁻¹, less than about 5×10⁻⁶s⁻¹, less than about 10⁻⁷s⁻¹, less than about 5×10⁻⁷s⁻¹, less than about 10⁻⁸s⁻¹, less than about 5×10⁻⁸s⁻¹, less than about 10⁻⁹s⁻¹, less than about 5×10⁻⁹s⁻¹, or less than about 10⁻¹⁰s⁻¹.

In another embodiment, an ADC of the invention that specifically binds to least on Eph receptor has an affinity constant or Kₐ (kₒₙ/k_{off}) of at least 10²M⁻¹, at least 5×10²M⁻¹, at least 10³M⁻¹, at least 5×10³M⁻¹, at least 10⁴M⁻¹, at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹M⁻¹, at least 5×10⁹M⁻¹, at least 10¹⁰M⁻¹, at least 5×10¹M⁻¹, at least 10¹¹M⁻¹, at least 5×10¹¹M⁻¹, at least 10¹²M⁻¹, at least 5×10¹²M, at least 10¹³M⁻¹, at least 5×10¹³M⁻¹, at least 10¹⁴M⁻¹, at least 5×10¹⁴M⁻¹, at least 10¹⁵M⁻¹, or at least 5×10¹⁵M⁻¹. In a further embodiment, an ADC of the invention that specifically binds to least on Eph receptor has an affinity constant or Kₐ (kₒₙ/k_{off}) of at least about 10²M⁻¹, at least about 5×10²M⁻¹, at least about 10³M⁻¹, at least about 5×10³M⁻¹, at least about 10⁴M⁻¹, at least about 5×10⁴M⁻¹, at least about 10⁵M⁻¹, at least about 5×10⁵M⁻¹, at least about 10⁶ M⁻¹, at least about 5×10⁶M⁻¹, at least about 10⁷M⁻¹, at least about 5×10⁷M⁻¹, at least about 10⁸M⁻¹, at least about 5×10⁸M⁻¹, at least about 10⁹M⁻¹, at least about 5×10⁹M⁻¹, at least about 10¹⁰M⁻¹, at least about 5×10¹M⁻¹, at least about 10¹¹M⁻¹, at least about 5×10¹³M⁻¹, at least about 10¹²M⁻¹, at least about 5×10¹²M, at least about 10¹³M⁻¹, at least about 5×10¹³M⁻¹, at least about 10¹⁴M⁻¹, at least about 5×10¹⁴M⁻¹, at least about 10¹⁵M⁻¹, or at least about 5×10⁵M⁻¹.

In yet another embodiment, an ADC that specifically binds to least on Eph receptor has a dissociation constant or K_{d} (k_{off}/kₒₙ) of less than 10⁻²M, less than 5×10⁻²M, less than 10⁻³M, less than 5×10⁻³M, less than 10⁻⁴M, less than 5×10⁻⁴M, less than 10⁻⁵M, less than 5×10⁻⁵M, less than 10⁻⁶M, less than 5×10⁻⁶M, less than 10⁻⁷M, less than 5×10⁻⁷M, less than 10⁻⁸M, less than 5×10⁻⁸M, less than 10⁻⁹M, less than 5×10⁻⁹M, less than 10⁻¹⁰M, less than 5×10⁻¹⁰M, less than 10⁻¹¹M, less than 5×10⁻¹¹M, less than 10⁻¹²M, less than 5×10⁻¹²M, less than 10⁻¹³M, less than 5×10⁻¹³M, less than 10⁻¹⁴M, less than 5×10⁻¹⁴M, less than 10⁻¹⁵M, or less than 5×10⁻¹⁵M. In a further embodiment, an ADC that specifically binds to least on Eph receptor has a dissociation constant or K_{d} (k_{off}/kₒₙ) of less than about 10⁻²M, less than about 5×10⁻²M, less than about 10⁻³M, less than about 5×10⁻³M, less than about 10⁻⁴M, less than about 5×10⁻⁴M, less than about 10⁻⁵M, less than about 5×10⁻⁵M, less than about 10⁻⁶M, less than about 5×10⁻⁶M, less than about 10⁻⁷M, less than about 5×10⁻⁷M, less than about 10⁻⁸M, less than about 5×10⁻⁸M, less than about 10⁻⁹M, less than about 5×10⁻⁹M, less than about 10⁻¹⁰M, less than about 5×10⁻¹⁰M, less than about 10⁻¹¹M, less than about 5×10⁻¹¹M, less than about 10⁻¹²M, less than about 5×10⁻¹²M, less than about 10⁻¹³M, less than about 5×10⁻¹³M, less than about 10⁻¹⁴M, less than about 5×10⁻¹⁴M, less than about 10⁻¹⁵M, or less than about 5×10⁻¹⁵M.

As discussed above, the invention encompasses ADCs wherein the antibody portion of the ADC comprises a variable region that specifically binds to at least one Eph receptor. The invention further encompasses ADCs that specifically bind to at least one Eph receptor, have altered ADCC and/or CDC activity and modified binding affinities for one or more Fc ligand (e.g., FcγRs, C1q) relative to a comparable molecule. See, for example, US Patent Application Publication No. 2006/0039904 A1. The invention specifically encompasses ADCs derived from anti-Eph receptor antibodies or fragments thereof including, but not limited to, Eph099B-102.147 (ATCC access No. PTA-4572), Eph099B-208.261 (ATCC access No. PTA-4573), Eph099B-210.248 (ATCC access No. PTA-4574), Eph099B-233.152 (ATCC access No. PTA-5194), (PCT Publication No. WO 03/094859 which is incorporated herein by reference in its entirety); EA2 (ATCC access No. PTA-4380), EA3, EA4, EA5 (ATCC access No. PTA-4381), (PCT Publication No. WO 04/014292 which is incorporated herein by reference in its entirety); LX-13 and scFv EA44 (ATCC access No. PTA-6044), (U.S. patent application Ser. No. 10/863,729 which is incorporated herein by reference in its entirety), G2, and 12G3H11 and analogs, derivatives, or fragments thereof. It is specifically contemplated that the ADCs of the invention may comprise all or a portion of the variable region (e.g., one or more CDR) from 12G3H11 (see Table 2) and/or any of the antibodies listed in Tables 2-4 or 6, or FIGS. 1-59.

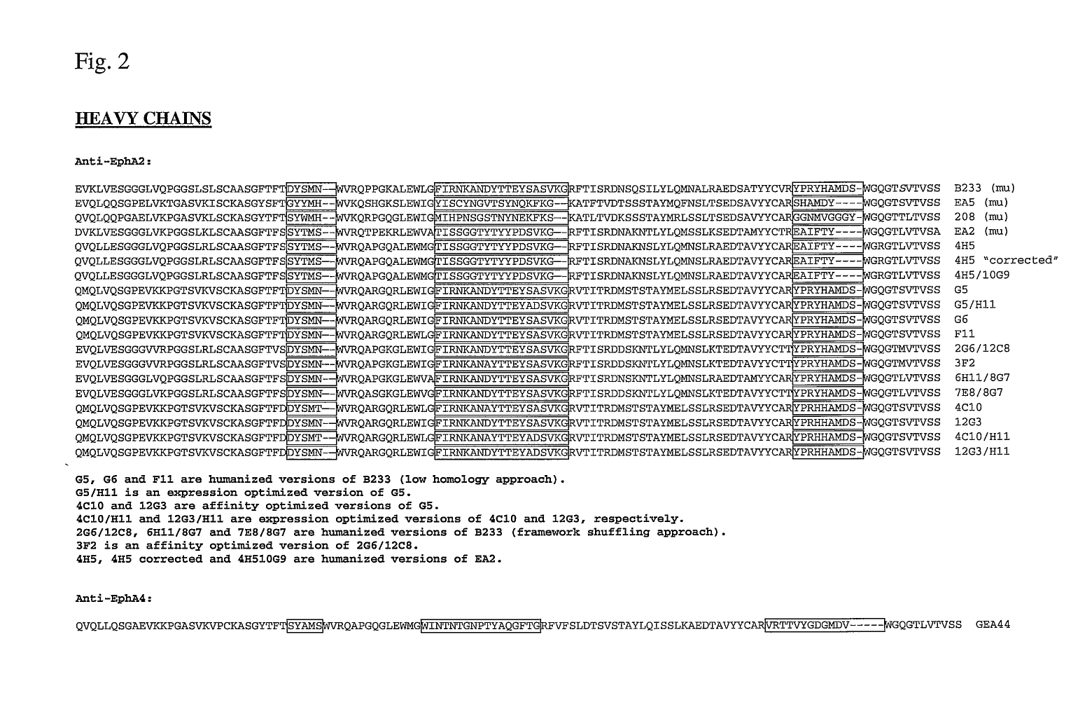

In one embodiment, the ADC is an ADC of 12G3H11, a humanized agonistic monoclonal antibody that binds EphA2. The amino acid sequences for the heavy chain variable region and light chain variable region are provided herein as SEQ ID NO: 165 and SEQ ID NO: 166, respectively (see FIGS. 1 and 2). In another embodiment, the ADC of the present invention binds to the same epitope as 12G3H11 or competes with 12G3H11 for binding to EphA2. In an alternative embodiment, the ADC of the invention that specifically binds to an Eph receptor is not an ADC of 12G3H11.

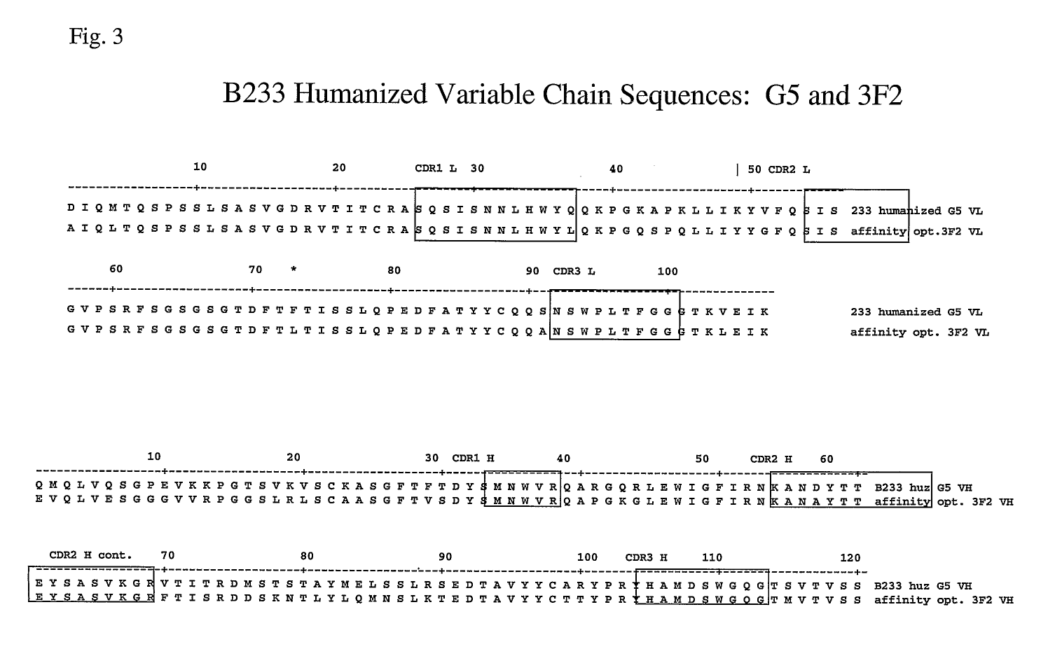

In one embodiment, the ADC is an ADC of 3F2, a humanized agonistic monoclonal antibody that binds EphA2 (see U.S. patent application Ser. No. 11/203,251, which is hereby incorporated by reference herein in its entirety). The amino acid sequences for the heavy chain variable region and light chain variable region are provided herein as SEQ ID NO: 63 and SEQ ID NO: 64, respectively (FIG. 3). In another embodiment, ADC of the present invention binds to the same epitope as 3F2 or competes with 3F2 for binding to EphA2. In an alternative embodiment, the ADC of the invention that immuno-specifically binds to an Eph receptor is not an ADC of 3F2. In another embodiment, the ADC of the invention is not an ADC of 3F2.

In another embodiment, the ADC is an ADC of G5, a humanized agonistic monoclonal antibody that binds EphA2. The amino acid sequence of the variable region of the heavy and light chains of G5 are provided herein as SEQ ID NO. 103 and SEQ ID NO. 104, respectively (FIGS. 1 and 2).

In another embodiment, the ADC is an ADC of the anti-EphA2 antibodies 12G3H11, B233, B208, B210, G5, 10C12, 4H5, 10G9, 3F2, 1C1, 1F12, 1H3, 1D3, 2B12, or 5A8. The amino acid sequences of the variable regions of the heavy and light chains of these antibodies are shown in FIGS. 1-14 (SEQ ID NOS. 165 and 166, 87 and 88, 95 and 96, 103 and 104, 140 and 142, 137 and 138, 63 and 64, 3 and 4, 13 and 14, 23 and 24, 33 and 34, 43 and 44, and 53 and 54).

In one embodiment, the ADC of the invention preferentially binds EphA2 over other Eph receptors. In another embodiment, the ADC of the invention preferentially binds EphA4 over other Eph receptors. In still another embodiment, the ADC of the invention immunoreacts with one or more Eph receptor complex (e.g., an Eph receptor-Ephrin ligand complex). In still another embodiment, an ADC of the invention specifically binds more then one Eph receptor. Combinations of Eph receptors bound by an ADC that specifically binds more then one Eph receptor are represented by the following formulas, EphA(x)+EphB(y); EphA(x)+EphA(x); EphB(y)+EphB(y); wherein (x) is 1, 2, 3, 3a, 3b, 4, 5, 5a, 5b, 6, 7 or 8 and (y) is 1, 2, 2a, 2b, 3, 4, 5 or 6. In a specific embodiment, an ADC that specifically immunoreacts with more then one Eph receptor binds to, e.g., EphA2+EphA4, or EphA2+EphA3, or EphA2+EphB4, or EphA4+EphA3, or EphA4+EphB4. It is specifically contemplated that an ADC that specifically binds more then one Eph receptor is a bispecific antibody. It is further contemplated that an ADC that specifically binds more then one Eph receptor is an antibody that binds a common epitope between two or more Eph receptors. It is further contemplated that an ADC that specifically binds more then one Eph receptor is an antibody that cross-reacts with one or more Eph receptors. In addition, the ADC of the invention may have the same immunoreactivity for more then one Eph receptor (e.g., EphA2 and EphA4) or alternatively, the ADC may immunoreact more strongly with one Eph receptor then with another.

The present invention encompasses ADCs that specifically bind to EphA2, said antibodies comprising a variable heavy (“VH”) domain having an amino acid sequence of the VH domain of 12G3H11, Eph099B-102.147, Eph099B-208.261 (“B208”), Eph099B-210.248 (“B210”), Eph099B-233.152 (“B233”), EA2, EA3, EA4, EA5, 10C12, 4H5, 10G9, 3F2, 1C1, 1F12, 1H3, 1D3, 2B12, or 5A8. The present invention also encompasses ADCs that specifically bind to EphA2, said antibodies comprising a variable light (“VL”) domain having an amino acid sequence of the VL domain of 12G3H11, Eph099B-102.147, B208, B210, B233, EA2, EA3, EA4, EA5, 10C12, 4H5, 10G9, 3F2, 1C1, 1F12, 1H3, 1D3, 2B12, or 5A8. The invention further encompasses ADCs that specifically bind to EphA2, said antibodies comprising a VH domain disclosed herein combined with a VL domain disclosed herein, or other VL domain. The present invention further encompasses ADCs that specifically bind to EphA2, said ADCs comprising a VL domain disclosed herein combined with a VH domain disclosed herein, or other VH domain.

The present invention encompasses ADCs that specifically bind to EphA4, said antibodies comprising a variable heavy (“VH”) domain having an amino acid sequence of the VH domain of LX-13 or scFv EA44. The present invention also encompasses ADCs that specifically bind to EphA4, said antibodies comprising a variable light (“VL”) domain having an amino acid sequence of the VL domain of LX-13 or scFv EA44. The invention further encompasses ADCs that specifically bind to EphA4, said antibodies comprising a VH domain disclosed herein combined with a VL domain disclosed herein, or other VL domain. The present invention further encompasses ADCs that specifically bind to EphA4, said ADCs comprising a VL domain disclosed herein combined with a VH domain disclosed herein, or other VH domain.

The present invention encompasses ADCs that specifically bind to an Eph receptor, said antibodies comprising a VH CDR having an amino acid sequence of any one of the VH CDRs listed in Tables 2 or 3 infra. The present invention also encompasses ADCs that specifically bind to an Eph receptor, said antibodies comprising a VL CDR having an amino acid sequence of any one of the VL CDRs listed in Tables 2 or 3 infra. The present invention also encompasses ADCs that specifically bind to an Eph receptor, said ADCs comprising one or more VH CDRs and one or more VL CDRs listed in Tables 2 or 3. The present invention further encompasses ADCs that specifically binds to an Eph receptor, said ADCs comprising any combination of some or all of the VH CDRs and VL CDRs listed in Tables 2 or 3 infra.

TABLE 2
CDR Sequences Of 12G3H11 and 3F2

| CDR | Sequence | SEQ ID NO: |
|---|---|---|
| 12G3H11 VH1 | DYSMN | *** |
| 12G3H11 VH2 | FIRNKANDYTTEYADSVKG | *** |
| 12G3H11 VH3 | YPRHHAMDS | *** |
| 12G3H11 VL1 | RASQSISNNLH | *** |
| 12G3H11 VL2 | YAFQSIS | *** |
| 12G3H11 VL3 | QQANSWPLT | *** |
| 3F2 VH1 | DYSMN | 65 |
| 3F2 VH2 | FIRNKANAYTTEYSASVKG | 66 |
| 3F2 VH3 | YPRYHAMDS | 67 |
| 3F2 VL1 | RASQSISNNLH | 68 |
| 3F2 VL2 | YGFQSIS | 69 |
| 3F2 VL3 | QQANSWPLT | 70 |

TABLE 3
CDR Sequences of 1C1, 1F12, 1H3,
1D3, 2B12, and 5A8

| | | Seq |
|---|---|---|
| CDR | Sequence | ID No. |
| 1C1VH1 | HYMMA | 5 |
| 1C1VH2 | RIGPSGGPTHYADSVKG | 6 |
| 1C1VH3 | YDSGYDYVAVAGPAEYFQH | 7 |
| 1C1VL1 | RASQSISTWLA | 8 |
| 1C1VL2 | KASNLHT | 9 |
| 1C1VL3 | QQYNSYSRT | 10 |
| 1F12VH1 | RYQMM | 15 |
| 1F12VH2 | SISPSGGVTLYADSVKG | 16 |
| 1F12VH3 | ELLGTVVVPVAWKMRGYFDY | 17 |
| 1F12VL1 | RASQSVSSNLA | 18 |
| 1F12VL2 | GASTRAST | 19 |
| 1F12VL3 | QQYNNWPPLT | 20 |
| 1H3VH1 | MYAMR | 25 |
| 1H3VH2 | VIGPSGGWTPYADSVKG | 26 |
| 1H3VH3 | DRGIYGMDV | 27 |
| 1H3VL1 | RASQGISSYLA | 28 |
| 1H3VL2 | AASTLQS | 29 |
| 1H3VL3 | LELNNYPFT | 30 |
| 1D3VH1 | PYDML | 35 |
| 1D3VH2 | RIGSSGGYTKYADSVKG | 36 |
| 1D3VH3 | ARSVVVSSDAFDI | 37 |
| 1D3VL1 | RASQGISKWLA | 38 |
| 1D3VL2 | GASTLQS | 39 |
| 1D3VL3 | QQYNDYPLT | 40 |
| 2B12VH1 | NYNMY | 45 |
| 2B12VH2 | VIVPSGKTSYADSVKG | 46 |
| 2B12VH3 | SYGGGFDY | 47 |
| 2B12VL1 | RASQDILTWLA | 48 |
| 2B12VL2 | AASSLQS | 49 |
| 2B12VL3 | QQAIRFPLT | 50 |
| 5A8VH1 | YYRMY | 55 |
| 5A8VH2 | SIYSSGGPTYYADSVKG | 56 |
| 5A8VH3 | DMGTGFWSGWGLGSDY | 57 |
| 5A8VL1 | RASQGISSWLA | 58 |
| 5A8VL2 | AASSLQS | 59 |
| 5A8VL3 | QQANSFPLT | 60 |

TABLE 4
Representative anti-Eph receptor Antibodies

| Antibody/Hybridoma | EphR | ATCC No. | Date of deposit | Patent App. No. |
|---|---|---|---|---|
| Eph099B-102.147 | EphA2 | PTA-4572 | Aug. 7, 2002 | WO 03/094859 |
| Eph099B208.261 | EphA2 | PTA-4573 | Aug. 7, 2002 | WO 03/094859 |
| Eph099B-210.248 | EphA2 | PTA-4574 | Aug. 7, 2002 | WO 03/094859 |
| Eph099B-233.152 | EphA2 | PTA-5194 | May 12, 2003 | WO 03/094859 |
| EA2 | EphA2 | PTA-4380 | May 22, 2002 | WO 04/014292 |
| EA5 | EphA2 | PTA-4381 | May 22, 2002 | WO 04/014292 |
| EA44 | EphA4 | PTA-6044 | Jun. 4, 2004 | 10/863,729 |
| 3F2 | EphA2 | | | 11/203,251 |

The present invention also encompasses ADCs that compete with 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44 or an antigen-binding fragment thereof for binding to an Eph receptor. Competition assays, which can be used to identify such antibodies, are well known to one skilled in the art. In a particular embodiment, 1 μg/ml of an antibody of the invention prevents 75%, 80%, 85% or 90% of ORIGEN TAG labeled 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44 from binding to biotin-labeled Eph receptor as measured by well-known ORIGEN analysis.

The present invention also provides ADCs that comprise a framework region known to those of skill in the art. In one embodiment, the fragment region of an antibody of the invention or fragment thereof is human or humanized.

The present invention encompasses ADCs comprising the amino acid sequence of 12G3H11, 3F2, Eph099B-102.147, Eph099B-208.261, Eph099B-210.248, Eph099B-233.152, EA2, EA3, EA4, EA5, 3F2, 1C1, 1F12, 1H3, 1D3, 2B12, 5A8LX-13 or scFv EA44 with mutations (e.g., one or more amino acid substitutions) in the framework or variable regions in addition to any other substitutions or changes (e.g., Fc substitution(s)). In one embodiment, mutations in these antibodies maintain or enhance the avidity and/or affinity of the antibodies for the Eph receptor to which they specifically bind. Standard techniques known to those skilled in the art (e.g., immunoassays) can be used to assay the affinity of an antibody for a particular antigen.

The present invention encompasses the use of a nucleic acid molecule(s), generally isolated, encoding the antibody portion of an ADC that specifically binds to an Eph receptor. In a specific embodiment, an isolated nucleic acid molecule encodes an ADC that specifically binds to an Eph receptor, said ADC having the amino acid sequence of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44 containing one or more Fc substitution. In another embodiment, an isolated nucleic acid molecule encodes an ADC that specifically binds to and Eph receptor, said ADC comprising a VH domain having the amino acid sequence of the VH domain of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44. In another embodiment, an isolated nucleic acid molecule encodes an ADC that specifically binds to an Eph receptor, said antibody comprising a VL domain having the amino acid sequence of the VL domain of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44.

The invention encompasses the use of an isolated nucleic acid molecule encoding an ADC that specifically binds to an Eph receptor, said ADC comprising a VH CDR having the amino acid sequence of any of the VH CDRs listed in Tables 2 or 3 and/or derived from the heavy chain of any of the antibodies listed in Table 4 or 6. In particular, the invention encompasses the use of an isolated nucleic acid molecule encoding an ADC that specifically binds to an Eph receptor, said antibody comprising one, two, or more VH CDRs having the amino acid sequence of any of the VH CDRs listed in Tables 2 or 3 and/or derived from the heavy chain of any of the antibodies listed in Table 4 or 6.

The present invention encompasses the use of an isolated nucleic acid molecule encoding an ADC that specifically binds to an Eph receptor, said ADC comprising a VL CDR having an amino acid sequence of any of the VL CDRs listed in Tables 2 or 3, and/or derived from the light chain of any of the antibodies listed in Table 4 or 6. In particular, the invention encompasses the use of an isolated nucleic acid molecule encoding an ADC that specifically binds to an Eph receptor, said antibody comprising one, two or more VL CDRs having the amino acid sequence of any of the VL CDRs listed in Table 2 or 3 and/or derived from the light chain of any of the antibodies listed in Table 4 or 6.

The present invention encompasses the use of ADCs that specifically bind to an Eph receptor, said ADCs comprising derivatives of the VH domains, VH CDRs, VL domains, or VL CDRs described herein that specifically bind to an Eph receptor. Standard techniques known to those of skill in the art can be used to introduce mutations (e.g., additions, deletions, and/or substitutions) in the nucleotide sequence encoding an antibody of the invention, including, for example, site-directed mutagenesis and PCR-mediated mutagenesis are routinely used to generate amino acid substitutions. In one embodiment, the VH and/or VL CDRs derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions in the relative to the original VH and/or VL CDRs. In another embodiment, the VH and/or VL CDRs derivatives have conservative amino acid substitutions (e.g. supra) are made at one or more predicted non-essential amino acid residues (i.e., amino acid residues which are not critical for the antibody to specifically bind to an Eph receptor). Alternatively, mutations can be introduced randomly along all or part of the VH and/or VL CDR coding sequence, such as by saturation mutagenesis, and the resultant mutants can be screened for biological activity to identify mutants that retain activity. Following mutagenesis, the encoded antibody can be expressed and the activity of the antibody can be determined.

The present invention encompasses ADCs of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44 with one or more additional amino acid residue substitutions in the variable light (VL) domain and/or variable heavy (VH) domain. The present invention also encompasses ADCs of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44 with one or more additional amino acid residue substitutions in one or more VL CDRs and/or one or more VH CDRs. The antibody generated by introducing substitutions in the VH domain, VH CDRs, VL domain and/or VL CDRs of an ADC of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44 can be tested in vitro and in vivo, for example, for its ability to bind to an Eph receptor (by, e.g., immunoassays including, but not limited to ELISAs and BIAcore), or for its ability to mediate, prevent, treat, manage or ameliorate cancer or one or more symptoms thereof.

The present invention also encompasses the use of ADCs that specifically bind to at least one Eph receptor or a fragment thereof, said ADCs comprising an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44. The present invention also encompasses the use of ADCs that specifically bind to at least one Eph receptor or a fragment thereof, said ADCs comprising an amino acid sequence of a variable heavy chain and/or variable light chain that is at least about 45%, at least about 50%, at least about 55%, at least about 60%, at least about 65%, at least about 70%, at least about 75%, at least about 80%, at least about 85%, at least about 90%, at least about 95%, or at least about 99% identical to the amino acid sequence of the variable heavy chain and/or light chain of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44. The present invention further encompasses the use of ADCs that specifically bind to at least one Eph receptor or a fragment thereof, said antibodies or antibody fragments comprising an amino acid sequence of one or more CDRs that is at least 45%, at least 50%, at least 55%, at least 60%, at least 65%, at least 70%, at least 75%, at least 80%, at least 85%, at least 90%, at least 95%, or at least 99% identical to the amino acid sequence of one or more CDRs of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44. The present invention further encompasses the use of ADCs that specifically bind to at least one Eph receptor or a fragment thereof, said antibodies or antibody fragments comprising an amino acid sequence of one or more CDRs that is at least about 45%, at least about 50%, at least about 55%, at least about 60%, at least about 65%, at least about 70%, at least about 75%, at least about 80%, at least about 85%, at least about 90%, at least about 95%, or at least about 99% identical to the amino acid sequence of one or more CDRs of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44. The determination of percent identity of two amino acid sequences can be determined by any method known to one skilled in the art, including BLAST protein searches.

The present invention also encompasses the use of ADCs that specifically bind to at least one Eph receptor or fragments thereof, where said ADCs are encoded by a nucleotide sequence that hybridizes to the nucleotide sequence of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44 under stringent conditions. In another embodiment, the invention encompasses ADCs that specifically bind to an Eph receptor or a fragment thereof, said ADCs comprising one or more CDRs encoded by a nucleotide sequence that hybridizes under stringent conditions to the nucleotide sequence of one or more CDRs of 12G3H11, Eph099B-102.147, B208, B210, B233, EA2, EA3, EA4, EA5, 10C12, 4H5, 10G9, 3F2, 1C1, 1F12, 1H3, 1D3, 2B12, 5A8, 5A8LX-13 or scFv EA44. Stringent hybridization conditions include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The present invention provides antibody drug conjugates that specifically bind to an EphA2 polypeptide. The present invention further provides antibodies that bind a human EphA2 polypeptide, a mouse EphA2 polypeptide and a rat EphA2 polypeptide. In certain embodiments, a single antibody clone can bind the human, mouse and rat forms of the EphA2 polypeptide. In other embodiments, a single antibody clone only binds human EphA2, or only binds mouse EphA2, or only binds rat EphA2. In yet other embodiments, a single antibody clone binds human and mouse EphA2, or binds human and rat EphA2, or binds rat and mouse EphA2.

In particular, the invention provides the following antibodies or ADC's that specifically bind to an EphA2 polypeptide: 12G3H11 or an antigen-binding fragment thereof, Eph099B-102.147 or an antigen binding fragment thereof, B208 or an antigen binding fragment thereof, B210 or an antigen binding fragment thereof, B233 or an antigen binding fragment thereof, EA2 or an antigen binding fragment thereof, EA3 or an antigen binding fragment thereof, EA4 or an antigen binding fragment thereof, EA5 or an antigen binding fragment thereof, 10C12 or an antigen binding fragment thereof, 4H5 or an antigen binding fragment thereof, 10G9 or an antigen binding fragment thereof, 3F2 or an antigen binding fragment thereof, 5A8LX-13 or an antigen binding fragment thereof, scFv EA44 or an antigen binding fragment thereof, 1C1 or an antigen-binding fragment thereof, 1F12 or an antigen-binding fragment thereof, 1H3 or an antigen-binding fragment thereof, 1D3 or an antigen-binding fragment thereof, 2B12 or an antigen-binding fragment thereof, and 5A8 or an antigen-binding fragment thereof. In one embodiment, an antibody that specifically binds to an EphA2 polypeptide is 1C1 or an antigen-binding fragment thereof (e.g., one or more CDRs of 1C1). In another embodiment, an antibody that specifically binds to an EphA2 polypeptide is 1F12 or an antigen-binding fragment thereof (e.g., one or more CDRs of 1F12). In a further embodiment, an antibody that specifically binds to an EphA2 polypeptide is 1H3 or an antigen-binding fragment thereof (e.g., one or more CDRs of 1H3). In another embodiment, an antibody that specifically binds to an EphA2 polypeptide is 1D3 or an antigen-binding fragment thereof (e.g., one or more CDRs of 1D3). In yet another embodiment, an antibody that specifically binds to an EphA2 polypeptide is 2B12 or an antigen-binding fragment thereof (e.g., one or more CDRs of 2B 12). In a further embodiment, an antibody that specifically binds to an EphA2 polypeptide is 5A8 or an antigen-binding fragment thereof (e.g., one or more CDRs of 5A8).

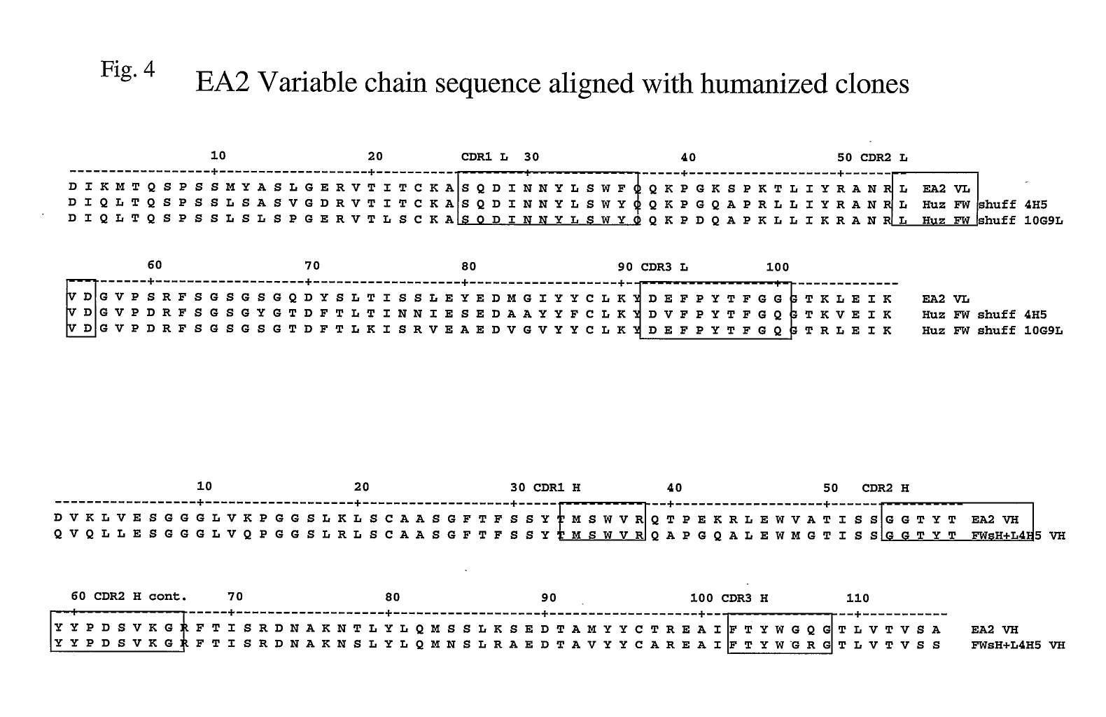

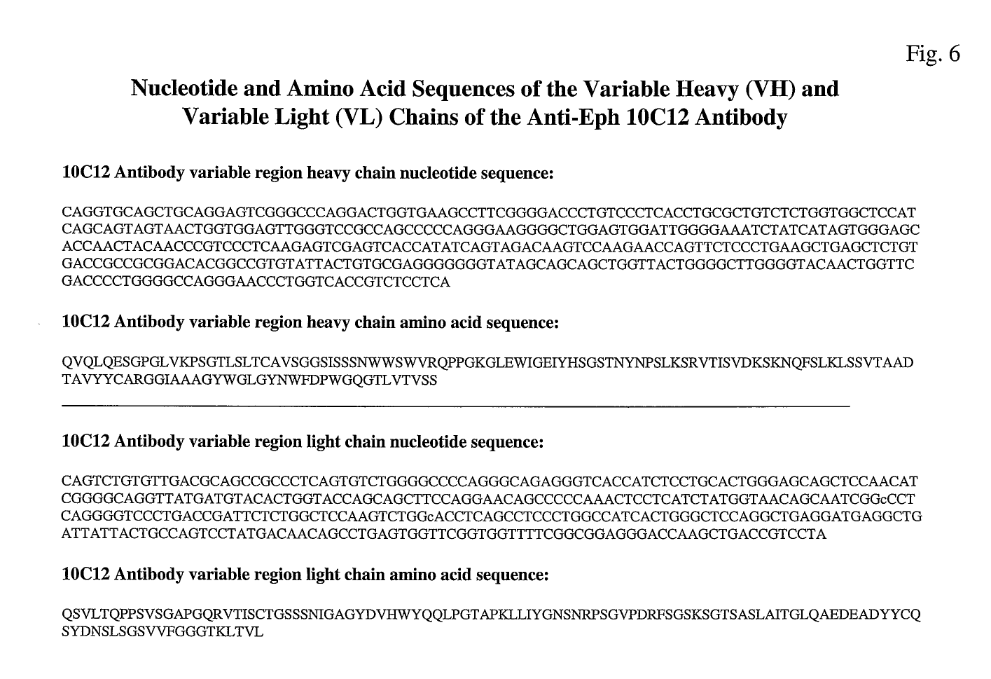

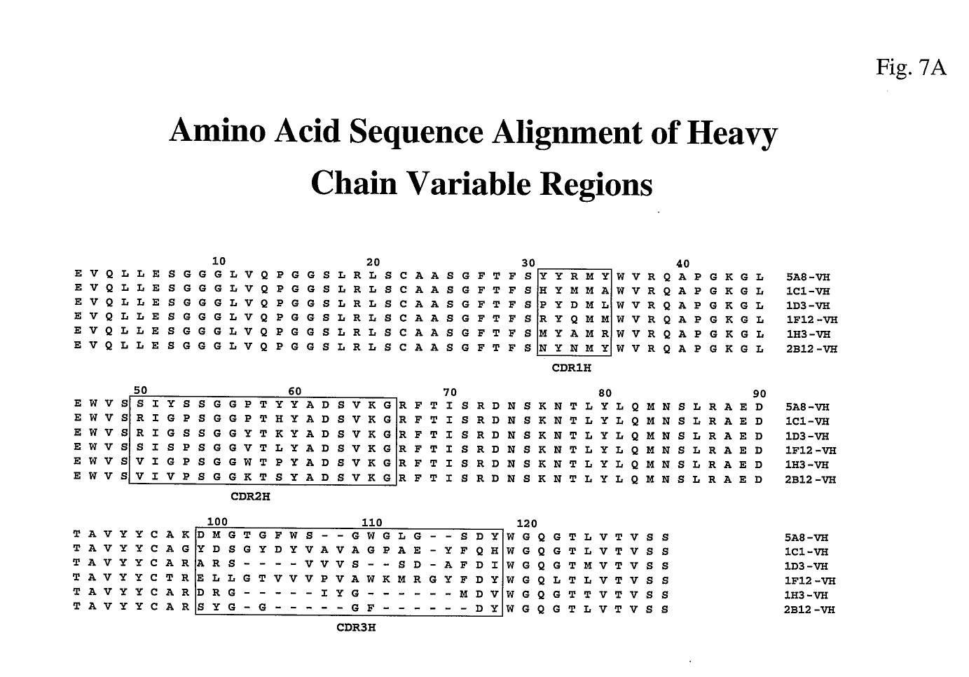

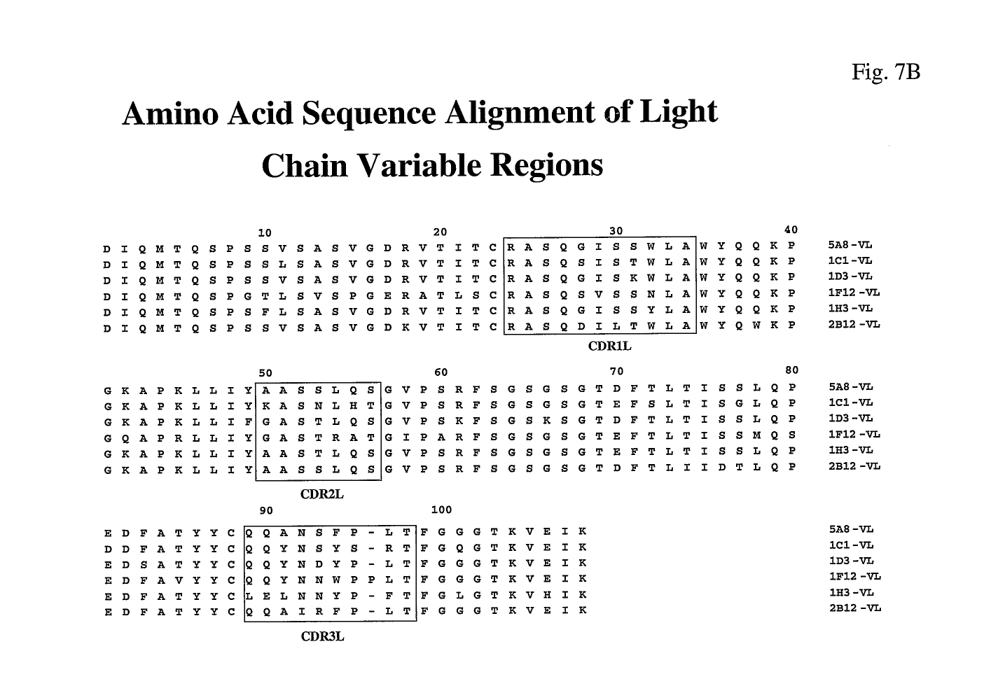

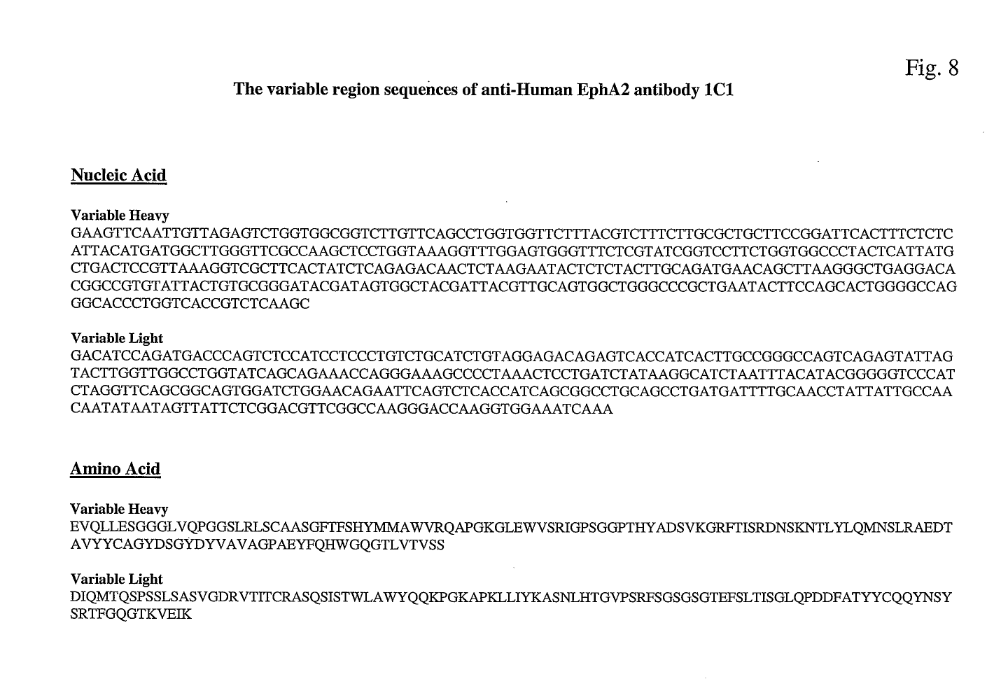

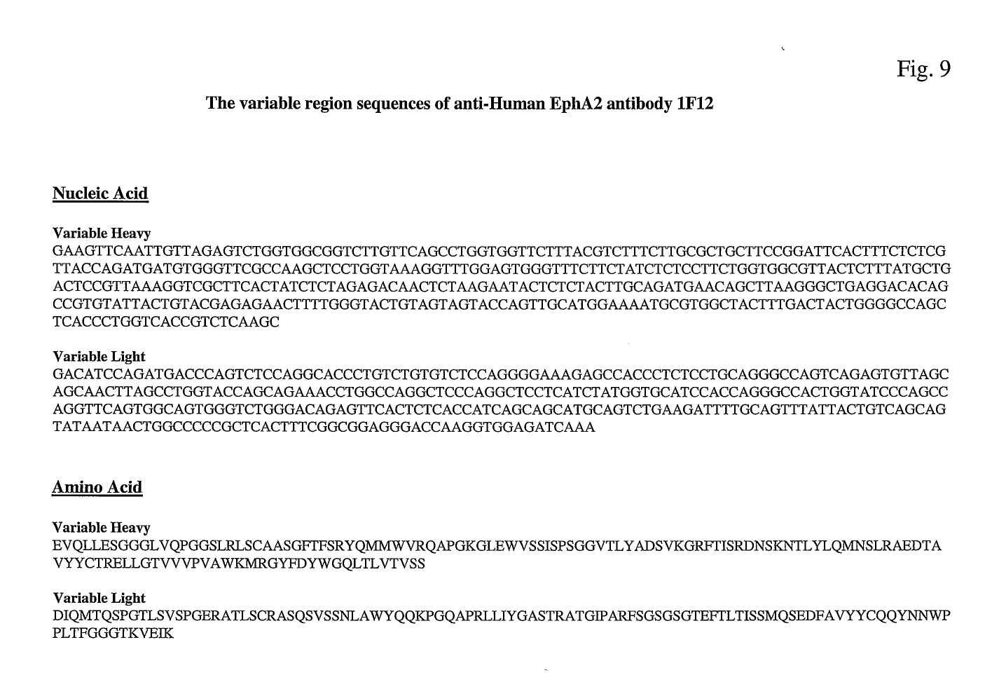

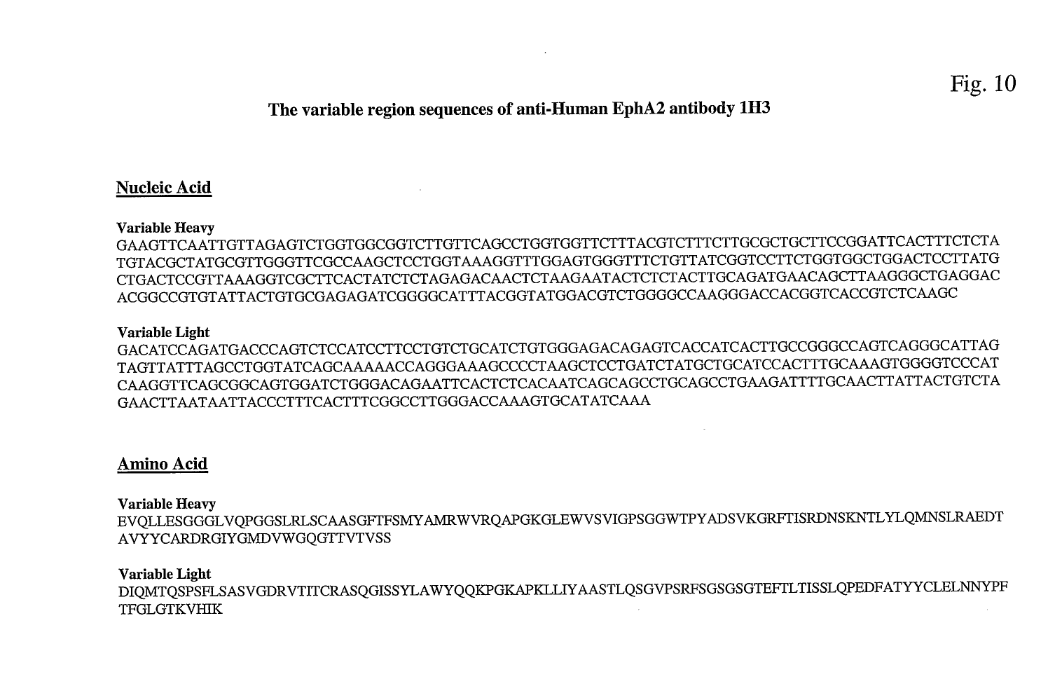

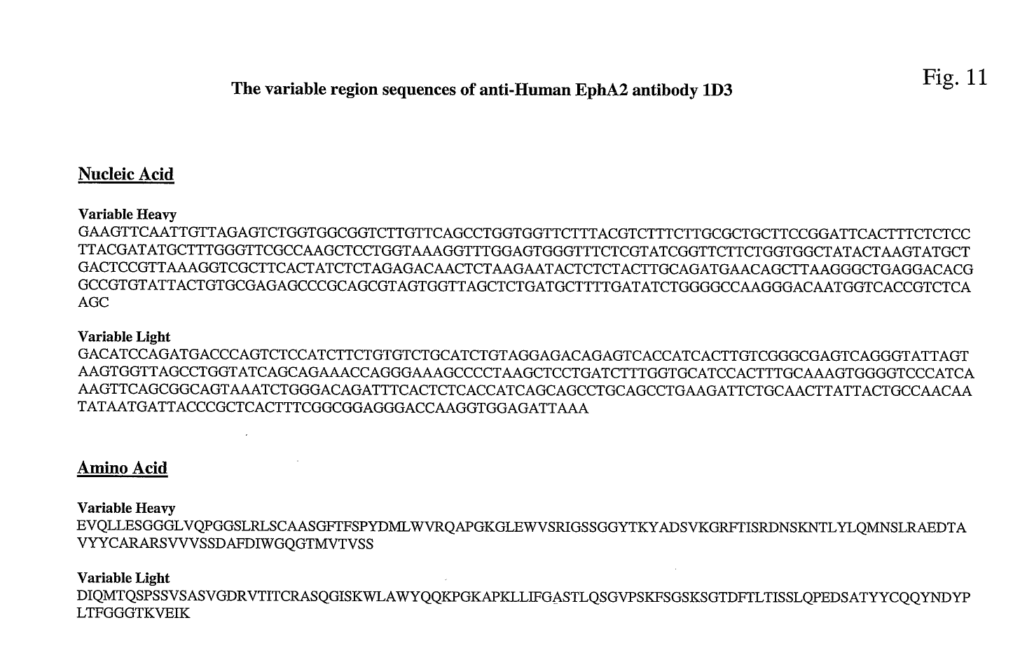

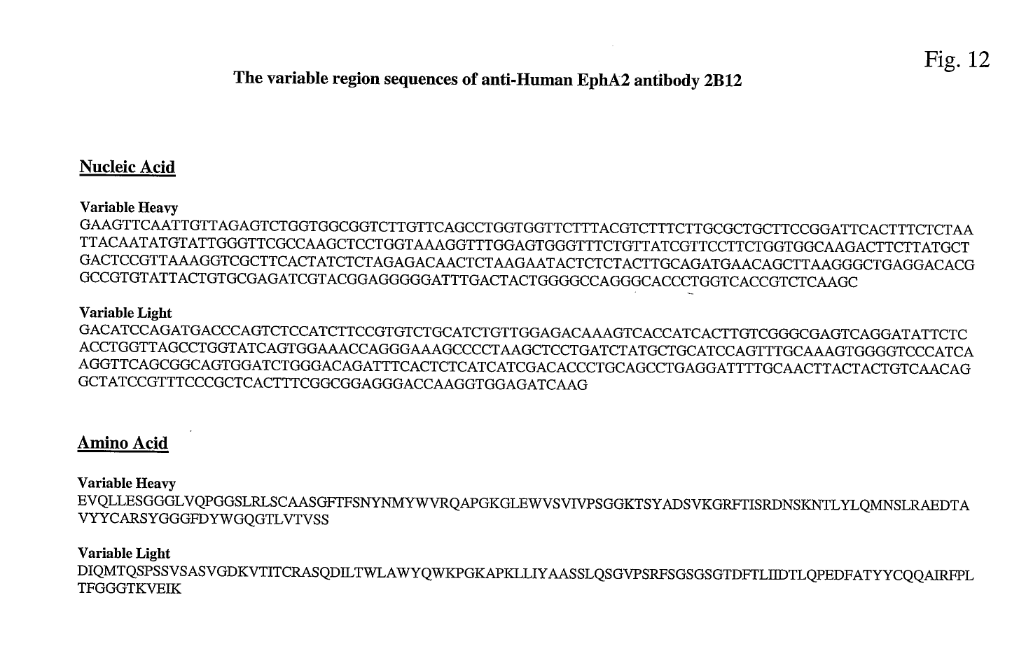

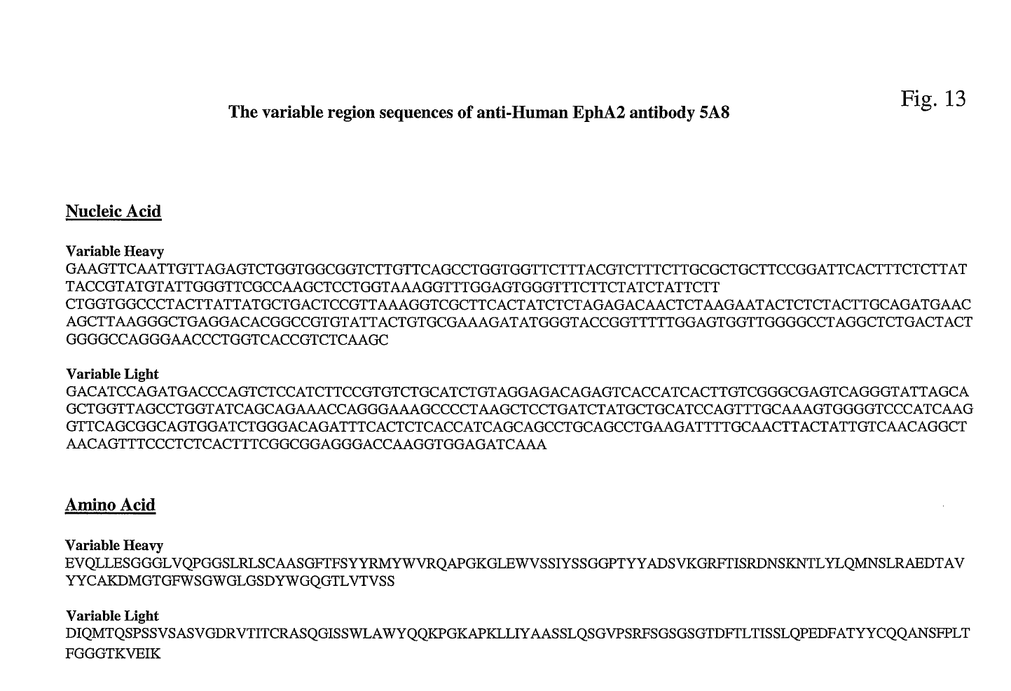

The present invention provides antibodies or ADC's that specifically bind an EphA2 polypeptide, said antibodies comprising a VH domain having an amino acid sequence of the VH domain of 12G3H11 (FIGS. 1,2; SEQ ID NO.: 165), B233 (FIGS. 1, 2; SEQ ID NO.: 87), B208 (FIGS. 1, 2; SEQ ID NO.: 95), B210 (FIGS. 1, 2), G5 (FIGS. 1, 2; SEQ ID NO.: 103), 10C12 (FIG. 6; SEQ ID NO.: 140), 4H5 (FIG. 4; SEQ ID NO.: 138), 10G9 (FIG. 4), 3F2 (FIG. 3; SEQ ID NO.: 63), 1C1 (FIGS. 7A and 8; SEQ ID NO.: 3), 1F12 (FIGS. 7A and 9; SEQ ID NO.: 13), 1H3 (FIGS. 7A and 10; SEQ ID NO.: 23), 1D3 (FIGS. 7A and 11; SEQ ID NO.: 33), 2B12 (FIGS. 7A and 12; SEQ ID NO.: 43), or 5A8 (FIGS. 7A and 13; SEQ ID NO.: 53).

The present invention provides antibodies that specifically bind to an EphA2 polypeptide, said antibodies comprising a VH CDR having an amino acid sequence of any one of the VH CDRs listed in Tables 2 or 3, infra. In particular, the invention provides antibodies that specifically bind to an EphA2 polypeptide, said antibodies comprising (or alternatively, consisting of) one, two, three, four, five or more VH CDRs having an amino acid sequence of any of the VH CDRs listed in Table 2 or 3, infra.

In one embodiment, an antibody that specifically binds to an EphA2 polypeptide comprises a VH CDR1 having the amino acid sequence of SEQ ID NOS.: 5, 15, 25, 35, 45, 55, or 65. In another embodiment, an antibody that specifically binds to an EphA2 polypeptide comprises a VH CDR2 having the amino acid sequence of SEQ ID NOS.: 6, 16, 26, 36, 46, 56, or 66. In another embodiment, an antibody that specifically binds to an EphA2 polypeptide comprises a VH CDR3 having the amino acid sequence of SEQ ID NOS.: 7, 17, 27, 37, 47, 57, or 67.

In another embodiment, an antibody that specifically binds to an EphA2 polypeptide comprises a VH CDR1 having the amino acid sequence of SEQ ID NOS.: 5, 15, 25, 35, 45, 55, or 65, and a VH CDR2 having the amino acid sequence of SEQ ID NOS.: 6, 16, 26, 36, 46, 56, or 66. In another embodiment, an antibody that specifically binds to an EphA2 polypeptide comprises a VH CDR1 having the amino acid sequence of SEQ ID NOS.: 5, 15, 25, 35, 45, 55, or 65, and a VH CDR3 having the amino acid sequence of SEQ ID NOS.: 7, 17, 27, 37, 47, 57, or 67. In another embodiment, an antibody that specifically binds to an EphA2 polypeptide comprises a VH CDR2 having the amino acid sequence of SEQ ID NOS.: 6, 16, 26, 36, 46, 56, or 66, and a VH CDR3 having the amino acid sequence of SEQ ID NOS.: 7, 17, 27, 37, 47, 57, or 67. In another embodiment, an antibody that specifically binds to an EphA2 polypeptide comprises a VH CDR1 having the amino acid sequence of SEQ ID NOS.: 5, 15, 25, 35, 45, 55, or 65, a VH CDR2 having the amino acid sequence of SEQ ID NOS.: 6, 16, 26, 36, 46, 56, or 66, and a VH CDR3 having the amino acid sequence of SEQ ID NOS.: 7, 17, 27, 37, 47, 57, or 67.

The present invention provides antibodies that specifically bind to an EphA2 polypeptide, said antibodies comprising a VL domain having an amino acid sequence of the VL domain for 12G3H11 (FIGS. 1, 2; SEQ ID NO.: 166), B233 (FIGS. 1, 2; SEQ ID NO.: 88), B208 (FIGS. 1, 2; SEQ ID NO.: 96), B210 (FIGS. 1, 2), G5 (FIG. 3; SEQ ID NO.: 104), 10C12 (FIG. 6; SEQ ID NO.: 142), 4H5 (FIG. 4; SEQ ID NO.: 137), 10G9 (FIG. 4), 3F2 (FIG. 3; SEQ ID NO.: 64), 1C1 (FIGS. 7B and 8; SEQ ID NO.: 4), 1F12 (FIG. 7B; SEQ ID NO.: 14), 1H3 (FIGS. 7B and 10; SEQ ID NO.: 24), 1D3 (FIGS. 7B and 11; SEQ ID NO.: 34), 2B12 (FIGS. 7B and 12; SEQ ID NO.: 44), or 5A8 (FIGS. 7B and 13; SEQ ID NO.: 54).

The present invention also provides antibodies that specifically bind to an EphA2 polypeptide, said antibodies comprising a VL CDR having an amino acid sequence of any one of the VL CDRs listed in Table 2 or 3, infra. In particular, the invention provides antibodies that specifically bind to an EphA2 polypeptide, said antibodies comprising (or alternatively, consisting of, or consisting essentially of) one, two, three or more VL CDRs having an amino acid sequence of any of the VL CDRs listed in Table 2 or 3, infra. In one embodiment, an antibody that specifically binds to an EphA2 polypeptide comprises a VL CDR1 having the amino acid sequence of SEQ ID NOS.: 8, 18, 28, 38, 48, 58, or 68. In another embodiment, an antibody that specifically binds to an EphA2 polypeptide comprises a VL CDR2 having the amino acid sequence of SEQ ID NOS.: 9, 19, 29, 39, 49, 59, or 69. In another embodiment, an antibody that specifically binds to an EphA2 polypeptide comprises a VL CDR3 having the amino acid sequence of SEQ ID NOS.: 10, 20, 30, 40, 50, 60, or 70. In another embodiment, an antibody of that specifically binds to an EphA2 polypeptide comprises a VL CDR1 having the amino acid sequence of SEQ ID NOS.: 8, 18, 28, 38, 48, 58, or 68, and a VL CDR2 having the amino acid sequence of SEQ ID NOS.: 9, 19, 29, 39, 49, 59, or 69. In another embodiment of an antibody that specifically binds to an EphA2 polypeptide comprises a VL CDR1 having the amino acid sequence of SEQ ID NOS.: 8, 18, 28, 38, 48, 58, or 68, and a VL CDR3 having the amino acid sequence of SEQ ID NOS.: 10, 20, 30, 40, 50, 60, or 70. In another embodiment, an antibody that specifically binds to an EphA2 polypeptide comprises a VL CDR2 having the amino acid sequence of SEQ ID NOS.: 9, 19, 29, 39, 49, 59, or 69, and a VL CDR3 having the amino acid sequence of SEQ ID NOS.: 10, 20, 30, 40, 50, 60, or 70. In another embodiment, an antibody that specifically binds to an EphA2 polypeptide comprises a VL CDR1 having the amino acid sequence of SEQ ID NOS.: 8, 18, 28, 38, 48, 58, or 68, a VL CDR2 having the amino acid sequence of SEQ ID NOS.: 9, 19, 29, 39, 49, 59, or 69, and a VL CDR3 having the amino acid sequence of SEQ ID NOS.: 10, 20, 30, 40, 50, 60, or 70, being a part of the antibody.

The present invention provides antibodies that specifically bind to an EphA2 polypeptide, said antibodies comprising a VH domain disclosed herein combined with a VL domain disclosed herein, or other known VL domains. The present invention also provides antibodies that specifically bind to an EphA2 polypeptide, said antibodies comprising a VL domain disclosed herein combined with a VH domain disclosed herein, or other known VH domains.

The present invention provides antibodies that specifically bind to an EphA2 polypeptide, said antibodies comprising one or more VH CDRs and one or more VL CDRs listed in Table 2 or 3, supra. In particular, the invention provides an antibody that specifically binds to an EphA2 polypeptide, said antibody comprising (or alternatively, consisting of, or consisting essentially of) a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 2 or 3, supra.

In one embodiment, an antibody that specifically binds to an EphA2 polypeptide comprises a VH CDR1 having the amino acid sequence of SEQ ID NOS.: 5, 15, 25, 35, 45, 55, or 65 and a VL CDR1 having the amino acid sequence of SEQ ID NOS.: 8, 18, 28, 38, 48, 58, or 68. In another embodiment, an antibody that specifically binds to an EphA2 polypeptide comprises a VH CDR1 having the amino acid sequence of SEQ ID NOS.: 5, 15, 25, 35, 45, 55, or 65 and a VL CDR2 having the amino acid sequence of SEQ ID NOS.: 9, 19, 29, 39, 49, 59, or 69. In another embodiment, an antibody that specifically binds to an EphA2 polypeptide comprises a VH CDR1 having the amino acid sequence of SEQ ID NOS.: 5, 15, 25, 35, 45, 55, or 65 and a VL CDR3 having an amino acid sequence of SEQ ID NOS.: 10, 20, 30, 40, 50, 60, or 70.

In one embodiment, an antibody that specifically binds to an EphA2 polypeptide comprises a VH CDR2 having the amino acid sequence of SEQ ID NOS.: 6, 16, 26, 36, 46, 56, or 66 and a VL CDR1 having the amino acid sequence of SEQ ID NOS.: 8, 18, 28, 38, 48, 58, or 68. In another embodiment, an antibody that specifically binds to an EphA2 polypeptide comprises a VH CDR2 having the amino acid sequence of SEQ ID NOS.: 6, 16, 26, 36, 46, 56, or 66 and a VL CDR2 having the amino acid sequence of SEQ ID NO.: 9, 19, 29, 39, 49, 59, or 69. In another embodiment, an antibody that specifically binds to an EphA2 polypeptide comprises a VH CDR2 having the amino acid sequence of SEQ ID NOS.: 6, 16, 26, 36, 46, 56, or 66 and a VL CDR3 having an amino acid sequence of SEQ ID NOS.: 10, 20, 30, 40, 50, 60, or 70.

In one embodiment, an antibody that specifically binds to an EphA2 polypeptide comprises a VH CDR3 having the amino acid sequence of SEQ ID NOS.: 7, 17, 27, 37, 47, 57, or 67 and a VL CDR1 having the amino acid sequence of SEQ ID NOS.: 8, 18, 28, 38, 48, 58, or 68. In another embodiment, an antibody that specifically binds to an EphA2 polypeptide comprises a VH CDR3 having the amino acid sequence of SEQ ID NOS.: 7, 17, 27, 37, 47, 57, or 67 and a VL CDR2 having the amino acid sequence of SEQ ID NOS.: 9, 19, 29, 39, 49, 59, or 69. In another embodiment, an antibody that specifically binds to an EphA2 polypeptide comprises a VH CDR3 having the amino acid sequence of SEQ ID NOS.: 7, 17, 27, 37, 47, 57, or 67 and a VL CDR3 having an amino acid sequence of SEQ ID NOS.: 10, 20, 30, 40, 50, 60, or 70.

The present invention provides antibodies that specifically bind to an EphA2 polypeptide, said antibodies encoded by a nucleic acid sequence comprising the nucleotide sequence of 12G3H11, B233, B208, B210, G5, 10C12, 4H5, 10G9, 3F2, 1C1, 1F12, 1H3, 1D3, 2B12, or 5A8 or an antigen-binding fragment thereof. In a specific embodiment, an antibody that specifically binds to an EphA2 polypeptide comprises a VH domain encoded by a nucleic acid sequence having a nucleotide sequence of the VH domain of 12G3H11, B233, B208, B210, G5, 10C12, 4H5, 10G9, 3F2, 1C1, 1F12, 1H3, 1D3, 2B12, or 5A8. In another embodiment, an antibody that specifically binds to an EphA2 polypeptide comprises a VL domain encoded by a nucleic acid sequence having a nucleotide sequence of the VL domain of 12G3H11, B233, B208, B210, G5, 10C12, 4H5, 10G9, 3F2, 1C1, 1F12, 1H3, 1D3, 2B12, or 5A8. In another embodiment, an antibody that specifically binds to an EphA2 polypeptide comprises a VH domain and a VL domain encoded by a nucleic acid sequence having a nucleotide sequence of the VH domain and VL domain of 12G3H11, B233, B208, B210, G5, 10C12, 4H5, 10G9, 3F2, 1C1, 1F12, 1H3, 1D3, 2B12, or 5A8.

In another embodiment, an antibody that specifically binds to an EphA2 polypeptide comprises a VH CDR encoded by a nucleic acid sequence having a nucleotide sequence of a VH CDR of 12G3H11, B233, B208, B210, G5, 10C12, 4H5, 10G9, 3F2, 1C1, 1F12, 1H3, 1D3, 2B12, or 5A8. In another embodiment, an antibody that specifically binds to an EphA2 polypeptide comprises a VL CDR encoded by a nucleic acid sequence having a nucleotide sequence of a VL CDR of 12G3H11, B233, B208, B210, G5, 10C12, 4H5, 10G9, 3F2, 1C1, 1F12, 1H3, 1D3, 2B12, or 5A8. In another embodiment, an antibody that specifically binds to an EphA2 polypeptide comprises a VH CDR and a VL CDR encoded by a nucleic acid sequence having a nucleotide sequence of a VH CDR and a VL CDR of 12G3H11, B233, B208, B210, G5, 10C12, 4H5, 10G9, 3F2, 1C1, 1F12, 1H3, 1D3, 2B12, or 5A8.

The present invention provides for a nucleic acid molecule, generally isolated, encoding an antibody of the present invention that specifically binds to an EphA2 polypeptide. In particular, the invention provides an isolated nucleic acid molecule encoding an antibody that specifically binds to an EphA2 polypeptide, said antibody having the amino acid sequence of 12G3H11, B233, B208, B210, G5, 10C12, 4H5, 10G9, 3F2, 1C1, 1F12, 1H3, 1D3, 2B12, or 5A8, or an antigen-binding fragment thereof. In a specific embodiment, an isolated nucleic acid molecule encodes an antibody that specifically binds to an EphA2 polypeptide, said antibody having the amino acid sequence of 1C1. In a specific embodiment, an isolated nucleic acid molecule encodes an antibody that specifically binds to an EphA2 polypeptide, said antibody having the amino acid sequence of 1F12. In a specific embodiment, an isolated nucleic acid molecule encodes an antibody that specifically binds to an EphA2 polypeptide, said antibody having the amino acid sequence of 1H3. In a specific embodiment, an isolated nucleic acid molecule encodes an antibody that specifically binds to an EphA2 polypeptide, said antibody having the amino acid sequence of 1D3. In a specific embodiment, an isolated nucleic acid molecule encodes an antibody that specifically binds to an EphA2 polypeptide, said antibody having the amino acid sequence of 2B12. In a specific embodiment, an isolated nucleic acid molecule encodes an antibody that specifically binds to an EphA2 polypeptide, said antibody having the amino acid sequence of 5A8.

The invention provides an isolated nucleic acid molecule encoding an antibody that specifically binds to an EphA2 polypeptide, said antibody comprising (alternatively, consisting of, or consisting essentially of) a VH domain having an amino acid sequence of a VH domain of 12G3H11, B233, B208, B210, G5, 10C12, 4H5, 10G9, 3F2, 1C1, 1F12, 1H3, 1D3, 2B 12, or 5A8. In a specific embodiment, an isolated nucleic acid molecule encodes an antibody that specifically binds to an EphA2 polypeptide, said antibody comprising a VH domain having the amino acid sequence of the VH domain of 1C1. In a specific embodiment, an isolated nucleic acid molecule encodes an antibody that specifically binds to an EphA2 polypeptide, said antibody comprising a VH domain having the amino acid sequence of the VH domain of 1F12. In a specific embodiment, an isolated nucleic acid molecule encodes an antibody that specifically binds to an EphA2 polypeptide, said antibody comprising a VH domain having the amino acid sequence of the VH domain of 1H3. In a specific embodiment, an isolated nucleic acid molecule encodes an antibody that specifically binds to an EphA2 polypeptide, said antibody comprising a VH domain having the amino acid sequence of the VH domain of 1D3. In a specific embodiment, an isolated nucleic acid molecule encodes an antibody that specifically binds to an EphA2 polypeptide, said antibody comprising a VH domain having the amino acid sequence of the VH domain of 2B12. In a specific embodiment, an isolated nucleic acid molecule encodes an antibody that specifically binds to an EphA2 polypeptide, said antibody comprising a VH domain having the amino acid sequence of the VH domain of 5A8.

The invention provides an isolated nucleic acid molecule encoding an antibody that specifically binds to an EphA2 polypeptide, said antibody comprising (alternatively, consisting of, or consisting essentially of) a VH CDR having an amino acid sequence of any of the VH CDRs listed in Table 2 or 3, supra. In particular, the invention provides an isolated nucleic acid molecule encoding an antibody that specifically binds to an EphA2 polypeptide, said antibody comprising one, two, three, four, five or more VH CDRs having an amino acid sequence of any of the VH CDRs listed in Table 2 or 3, supra. In one embodiment, an isolated nucleic acid molecule encodes an antibody that specifically binds to an EphA2 polypeptide, said antibody comprising a VH CDR1 having the amino acid sequence of the VH CDR1 listed in Table 2 or 3, supra. In another embodiment, an isolated nucleic acid molecule encodes an antibody that specifically binds to an EphA2 polypeptide, said antibody comprising a VH CDR2 having the amino acid sequence of the VH CDR2 listed in Table 2 or 3, supra. In another embodiment, an isolated nucleic acid molecule encodes an antibody that specifically binds to an EphA2 polypeptide, said antibody comprising a VH CDR3 having the amino acid sequence of the VH CDR3 listed in Table 2 or 3, supra.

The invention provides an isolated nucleic acid molecule encoding an antibody that specifically binds to an EphA2 polypeptide, said antibody comprising (alternatively, consisting of, or consisting essentially of) a VL domain having an amino acid sequence of a VL domain of 12G3H11, B233, B208, B210, G5, 10C12, 4H5, 10G9, 3F2, 1C1, 1F12, 1H3, 1D3, 2B12, or 5A8. In a specific embodiment, an isolated nucleic acid molecule encodes an antibody that specifically binds to an EphA2 polypeptide, said antibody comprising a VL domain having the amino acid sequence of the VL domain of 1C1. In a specific embodiment, an isolated nucleic acid molecule encodes an antibody that specifically binds to an EphA2 polypeptide, said antibody comprising a VL domain having the amino acid sequence of the VL domain of 1F12. In a specific embodiment, an isolated nucleic acid molecule encodes an antibody that specifically binds to an EphA2 polypeptide, said antibody comprising a VL domain having the amino acid sequence of the VL domain of 1H3. In a specific embodiment, an isolated nucleic acid molecule encodes an antibody that specifically binds to an EphA2 polypeptide, said antibody comprising a VL domain having the amino acid sequence of the VL domain of 1D3. In a specific embodiment, an isolated nucleic acid molecule encodes an antibody that specifically binds to an EphA2 polypeptide, said antibody comprising a VL domain having the amino acid sequence of the VL domain of 2B12. In a specific embodiment, an isolated nucleic acid molecule encodes an antibody that specifically binds to an EphA2 polypeptide, said antibody comprising a VL domain having the amino acid sequence of the VL domain of 5A8.

The invention also provides an isolated nucleic acid molecule encoding an antibody that specifically binds to an EphA2 polypeptide, said antibody comprising (alternatively, consisting of, or consisting essentially of) a VL CDR having an amino acid sequence of any of the VL CDRs listed in Table 2 or 3, supra. In particular, the invention provides an isolated nucleic acid molecule encoding an antibody that specifically binds to an EphA2 polypeptide, said antibody comprising one, two, three or more VL CDRs having an amino acid sequence of any of the VL CDRs listed in Table 2 or 3, supra. In one embodiment, an isolated nucleic acid molecule encodes an antibody that specifically binds to an EphA2 polypeptide, said antibody comprising a VL CDR1 having the amino acid sequence of the VH CDR1 listed in Table 2 or 3, supra. In another embodiment, an isolated nucleic acid molecule encodes an antibody that specifically binds to an EphA2 polypeptide, said antibody comprising a VL CDR2 having the amino acid sequence of the VL CDR2 listed in Table 2 or 3, supra. In another embodiment, an isolated nucleic acid molecule encodes an antibody that specifically binds to an EphA2 polypeptide, said antibody comprising a VL CDR3 having the amino acid sequence of the VL CDR3 listed in Table 2 or 3, supra.

The present invention provides nucleic acid molecules encoding antibodies that specifically bind to an EphA2 polypeptide, said antibodies comprising one or more VH CDRs and one or more VL CDRs listed in Table 2 or 3, supra. In particular, the invention provides an isolated nucleic acid molecule encoding an antibody that specifically binds to an EphA2 polypeptide, said antibody comprising (or alternatively, consisting of, or consisting essentially of) a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 2 or 3, supra.

Further specific embodiments of the invention follow, and are numbered sequentially:

1. An EphA2 antibody or ADC comprising a variable heavy (VH) domain having an amino acid sequence of the VH domain of 12G3H11, B233, B208, B210, G5, 10C12, 4H5, 10G9, 3F2, 1C1, 1F12, 1H3, 1D3, 2B12, or 5A8, wherein the said antibody specifically binds to an EphA2 polypeptide.
2. An EphA2 antibody or ADC comprising a variable light (VL) domain having an amino acid sequence of the VL domain of 12G3H11, B233, B208, B210, G5, 10C12, 4H5, 10G9, 3F2, 1C1, 1F12, 1H3, 1D3, 2B12, or 5A8, wherein the said antibody specifically binds to an EphA2 polypeptide.
3. The antibody or ADC of embodiment 1 further comprising a VL domain having an amino acid sequence of the VL domain of 12G3H11, B233, B208, B210, G5, 10C12, 4H5, 10G9, 3F2, 1C1, 1F12, 1H3, 1D3, 2B 12, or 5A8.
4. An EphA2 antibody or ADC comprising a complementarity determining region (CDR) having an amino acid sequence of a CDR of 12G3H11, B233, B208, B210, G5, 10C12, 4H5, 10G9, 3F2, 1C1, 1F12, 1H3, 1D3, 2B12, or 5A8, wherein the said antibody or ADC specifically binds to an EphA2 polypeptide.
5. The antibody or ADC of embodiment 4, wherein the antibody or ADC comprises a VH CDR having an amino acid sequence of a VH CDR of 12G3H11, B233, B208, B210, G5, 10C12, 4H5, 10G9, 3F2, 1C1, 1F12, 1H3, 1D3, 2B12, or 5A8.
6. The antibody or ADC of embodiment 4, wherein the antibody or ADC comprises a VL CDR having an amino acid sequence of a VL CDR of 12G3H11, B233, B208, B210, G5, 10C12, 4H5, 10G9, 3F2, 1C1, 1F12, 1H3, 1D3, 2B12, or 5A8.
7. The antibody or ADC of embodiment 5 further comprising a VL CDR having the amino acid sequence of a VL CDR of 12G3H11, B233, B208, B210, G5, 10C12, 4H5, 10G9, 3F2, 1C1, 1F12, 1H3, 1D3, 2B12, or 5A8.
8. The antibody or ADC of embodiment 5, wherein the antibody or ADC comprises a VH CDR1 having an amino acid sequence of a VH CDR1 of 12G3H11, B233, B208, B210, G5, 10C12, 4H5, 10G9, 3F2, 1C1, 1F12, 1H3, 1D3, 2B12, or 5A8.
9. The antibody or ADC of embodiment 5, wherein the antibody or ADC comprises a VH CDR2 having an amino acid sequence of a VH CDR2 of 12G3H11, B233, B208, B210, G5, 10C12, 4H5, 10G9, 3F2, 1C1, 1F12, 1H3, 1D3, 2B12, or 5A8.
10. The antibody or ADC of embodiment 5, wherein the antibody or ADC comprises a VH CDR3 having an amino acid sequence of a VH CDR3 of 12G3H11, B233, B208, B210, G5, 10C12, 4H5, 10G9, 3F2, 1C1, 1F12, 1H3, 1D3, 2B12, or 5A8.
11. The antibody or ADC of embodiment 8, wherein the antibody or ADC further comprises a VH CDR2 having an amino acid sequence of a VH CDR2 of 12G3H11, B233, B208, B210, G5, 10C12, 4H5, 10G9, 3F2, 1C1, 1F12, 1H3, 1D3, 2B12, or 5A8.
12. The antibody or ADC of embodiment 8, wherein the antibody or ADC further comprises a VH CDR3 having an amino acid sequence of a VH CDR3 of 12G3H11, B233, B208, B210, G5, 10C12, 4H5, 10G9, 3F2, 1C1, 1F12, 1H3, 1D3, 2B12, or 5A8.
13. The antibody or ADC of embodiment 9, wherein the antibody or ADC further comprises a VH CDR3 having an amino acid sequence of a VH CDR3 of 12G3H11, B233, B208, B210, G5, 10C12, 4H5, 10G9, 3F2, 1C1, 1F12, 1H3, 1D3, 2B12, or 5A8.
14. The antibody or ADC of embodiment 11, wherein the antibody or ADC further comprises a VH CDR3 having an amino acid sequence of a VH CDR3 of 12G3H11, B233, B208, B210, G5, 10C12, 4H5, 10G9, 3F2, 1C1, 1F12, 1H3, 1D3, 2B12, or 5A8.
15. The antibody or ADC of embodiment 7, wherein the antibody or ADC comprises a VH CDR1 having an amino acid sequence of a VH 12G3H11, B233, B208, B210, G5, 10C12, 4H5, 10G9, 3F2, 1C1, 1F12, 1H3, 1D3, 2B12, or 5A8.
16. The antibody or ADC of embodiment 7, wherein the antibody or ADC comprises a VH CDR2 having an amino acid sequence of a VH CDR2 of 12G3H11, B233, B208, B210, G5, 10C12, 4H5, 10G9, 3F2, 1C1, 1F12, 1H3, 1D3, 2B12, or 5A8.
17. The antibody or ADC of embodiment 7, wherein the antibody or ADC comprises a VH CDR3 having an amino acid sequence of a VH CDR3 of 12G3H11, B233, B208, B210, G5, 10C12, 4H5, 10G9, 3F2, 1C1, 1F12, 1H3, 1D3, 2B12, or 5A8.
18. The antibody or ADC of embodiment 15, wherein the antibody or ADC further comprises a VH CDR2 having an amino acid sequence of a VH CDR2 of 12G3H11, B233, B208, B210, G5, 10C12, 4H5, 10G9, 3F2, 1C1, 1F12, 1H3, 1D3, 2B12, or 5A8.
19. The antibody or ADC of embodiment 15, wherein the antibody or ADC further comprises a VH CDR3 having an amino acid sequence of a VH CDR3 of 12G3H11, B233, B208, B210, G5, 10C12, 4H5, 10G9, 3F2, 1C1, 1F12, 1H3, 1D3, 2B12, or 5A8.
20. The antibody or ADC of embodiment 16, wherein the antibody or ADC further comprises a VH CDR3 having an amino acid sequence of a VH CDR3 of 12G3H11, B233, B208, B210, G5, 10C12, 4H5, 10G9, 3F2, 1C1, 1F12, 1H3, 1D3, 2B12, or 5A8.
21. The antibody or ADC of embodiment 18, wherein the antibody or ADC further comprises a VH CDR3 having an amino acid sequence of a VH CDR3 of 12G3H11, B233, B208, B210, G5, 10C12, 4H5, 10G9, 3F2, 1C1, 1F12, 1H3, 1D3, 2B12, or 5A8.
22. The antibody or ADC of embodiment 6, wherein the antibody or ADC comprises a VL CDR1 having an amino acid sequence of a VL CDR1 of 12G3H11, B233, B208, B210, G5, 10C12, 4H5, 10G9, 3F2, 1C1, 1F12, 1H3, 1D3, 2B12, or 5A8.
23. The antibody or ADC of embodiment 6, wherein the antibody or ADC comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
24. The antibody or ADC of embodiment 6, wherein the antibody or ADC comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
25. The antibody or ADC of embodiment 22, wherein the antibody or ADC further comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
26. The antibody or ADC of embodiment 22, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
27. The antibody or ADC of embodiment 23, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
28. The antibody or ADC of embodiment 25, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
29. The antibody or ADC of embodiment 7, wherein the antibody or ADC comprises a VL CDR1 having an amino acid sequence of a VL CDR1 of 12G3H11, B233, B208, B210, G5, 10C12, 4H5, 10G9, 3F2, 1C1, 1F12, 1H3, 1D3, 2B12, or 5A8.
30. The antibody or ADC of embodiment 7, wherein the antibody or ADC comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
31. The antibody or ADC of embodiment 7, wherein the antibody or ADC comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
32. The antibody or ADC of embodiment 29, wherein the antibody or ADC further comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
33. The antibody or ADC of embodiment 29, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
34. The antibody or ADC of embodiment 30, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
35. The antibody or ADC of embodiment 32, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
36. The antibody or ADC of embodiment 15, wherein the antibody or ADC comprises a VL CDR1 having an amino acid sequence of a VL CDR1 of 12G3H11, B233, B208, B210, G5, 10C12, 4H5, 10G9, 3F2, 1C1, 1F12, 1H3, 1D3, 2B12, or 5A8.
37. The antibody or ADC of embodiment 15, wherein the antibody or ADC comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
38. The antibody or ADC of embodiment 15, wherein the antibody or ADC comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
39. The antibody or ADC of embodiment 36, wherein the antibody or ADC further comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
40. The antibody or ADC of embodiment 36, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
41. The antibody or ADC of embodiment 37, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
42. The antibody or ADC of embodiment 39, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
43. The antibody or ADC of embodiment 16, wherein the antibody or ADC comprises a VL CDR1 having an amino acid sequence of a VL CDR1 of 12G3H11, B233, B208, B210, G5, 10C12, 4H5, 10G9, 3F2, 1C1, 1F12, 1H3, 1D3, 2B12, or 5A8.
44. The antibody or ADC of embodiment 16, wherein the antibody or ADC comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
45. The antibody or ADC of embodiment 16, wherein the antibody or ADC comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
46. The antibody or ADC of embodiment 43, wherein the antibody or ADC further comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
47. The antibody or ADC of embodiment 43, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
48. The antibody or ADC of embodiment 44, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
49. The antibody or ADC of embodiment 46, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
50. The antibody or ADC of embodiment 17, wherein the antibody or ADC comprises a VL CDR1 having an amino acid sequence of a VL CDR1 of 12G3H11, B233, B208, B210, G5, 10C12, 4H5, 10G9, 3F2, 1C1, 1F12, 1H3, 1D3, 2B12, or 5A8.
51. The antibody or ADC of embodiment 17, wherein the antibody or ADC comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
52. The antibody or ADC of embodiment 17, wherein the antibody or ADC comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
53. The antibody or ADC of embodiment 50, wherein the antibody or ADC further comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
54. The antibody or ADC of embodiment 50, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
55. The antibody or ADC of embodiment 51, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
56. The antibody or ADC of embodiment 53, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
57. The antibody or ADC of embodiment 18, wherein the antibody or ADC comprises a VL CDR1 having an amino acid sequence of a VL CDR1 of 12G3H11, B233, B208, B210, G5, 10C12, 4H5, 10G9, 3F2, 1C1, 1F12, 1H3, 1D3, 2B12, or 5A8.
58. The antibody or ADC of embodiment 18, wherein the antibody or ADC comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
59. The antibody or ADC of embodiment 18, wherein the antibody or ADC comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
60. The antibody or ADC of embodiment 57, wherein the antibody or ADC further comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
61. The antibody or ADC of embodiment 57, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
62. The antibody or ADC of embodiment 58, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
63. The antibody or ADC of embodiment 60, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
64. The antibody or ADC of embodiment 19, wherein the antibody or ADC comprises a VL CDR1 having an amino acid sequence of a VL CDR1 of 12G3H11, B233, B208, B210, G5, 10C12, 4H5, 10G9, 3F2, 1C1, 1F12, 1H3, 1D3, 2B12, or 5A8.
65. The antibody or ADC of embodiment 19, wherein the antibody or ADC comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
66. The antibody or ADC of embodiment 19, wherein the antibody or ADC comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
67. The antibody or ADC of embodiment 64, wherein the antibody or ADC further comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
68. The antibody or ADC of embodiment 64, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
69. The antibody or ADC of embodiment 65, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
70. The antibody or ADC of embodiment 67, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
71. The antibody or ADC of embodiment 20, wherein the antibody or ADC comprises a VL CDR1 having an amino acid sequence of a VL CDR1 of 12G3H11, B233, B208, B210, G5, 10C12, 4H5, 10G9, 3F2, 1C1, 1F12, 1H3, 1D3, 2B12, or 5A8.
72. The antibody or ADC of embodiment 20, wherein the antibody or ADC comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
73. The antibody or ADC of embodiment 20, wherein the antibody or ADC comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
74. The antibody or ADC of embodiment 71, wherein the antibody or ADC further comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
75. The antibody or ADC of embodiment 71, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
76. The antibody or ADC of embodiment 72, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
77. The antibody or ADC of embodiment 74, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
78. The antibody or ADC of embodiment 21, wherein the antibody or ADC comprises a VL CDR1 having an amino acid sequence of a VL CDR1 of 12G3H11, B233, B208, B210, G5, 10C12, 4H5, 10G9, 3F2, 1C1, 1F12, 1H3, 1D3, 2B12, or 5A8.
79. The antibody or ADC of embodiment 21, wherein the antibody or ADC comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
80. The antibody or ADC of embodiment 21, wherein the antibody or ADC comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
81. The antibody or ADC of embodiment 78, wherein the antibody or ADC further comprises a VL CDR2 having an amino acid sequence of a VL CDR2 of 12G3H11, B233, B208, B210, G5, 10C12, 4H5, 10G9, 3F2, 1C1, 1F12, 1H3, 1D3, 2B12, or 5A8.
82. The antibody or ADC of embodiment 78, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
83. The antibody or ADC of embodiment 79, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.
84. The antibody or ADC of embodiment 81, wherein the antibody or ADC further comprises a VL CDR3 having an amino acid sequence of a VL CDR3 of 12G3H11, B233, B208, B210, G5, 10C12, 4H5, 10G9, 3F2, 1C1, 1F12, 1H3, 1D3, 2B12, or 5A8.

The present invention provides antibodies that specifically bind to an EphA2 polypeptide, said antibodies comprising derivatives of the VH domains, VH CDRs, VL domains, or VL CDRs described herein that specifically bind to an EphA2 polypeptide. Standard techniques known to those of skill in the art can be used to introduce mutations (e.g., deletions, additions, and/or substitutions) in the nucleotide sequence encoding an antibody of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a specific embodiment, the derivatives have conservative amino acid substitutions are made at one or more predicted non-essential amino acid residues (i.e., amino acid residues which are not critical for the antibody to specifically bind to an EphA2 polypeptide). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

The present invention provides for antibodies that specifically bind to an EphA2 polypeptide, said antibodies comprising the amino acid sequence of 12G3H11, B233, B208, B210, G5, 10C12, 4H5, 10G9, 3F2, 1C1, 1F12, 1H3, 1D3, 2B12, or 5A8 with one or more amino acid residue substitutions in the variable light (VL) domain and/or variable heavy (VH) domain. The present invention also provides for antibodies that specifically bind to an EphA2 polypeptide, said antibodies comprising the amino acid sequence of 12G3H11, B233, B208, B210, G5, 10C12, 4H5, 10G9, 3F2, 1C1, 1F12, 1H3, 1D3, 2B12, or 5A8 with one or more amino acid residue substitutions in one or more VL CDRs and/or one or more VH CDRs. The present invention also provides for antibodies that specifically bind to an EphA2 polypeptide, said antibodies comprising the amino acid sequence of 12G3H11, B233, B208, B210, G5, 10C12, 4H5, 10G9, 3F2, 1C1, 1F12, 1H3, 1D3, 2B12, or 5A8, or a VH and/or VL domain thereof with one or more amino acid residue substitutions in one or more VH frameworks and/or one or more VL frameworks. The antibody generated by introducing substitutions in the VH domain, VH CDRs, VL domain, VL CDRs and/or frameworks of 12G3H11, B233, B208, B210, G5, 10C12, 4H5, 10G9, 3F2, 1C1, 1F12, 1H3, 1D3, 2B12, or 5A8 can be tested in vitro and/or in vivo, for example, for its ability to bind to an EphA2 polypeptide, or for its ability to inhibit or reduce EphA2 receptor activation, or for its ability to activate EphA2.

In a specific embodiment, an antibody that specifically binds to an EphA2 polypeptide comprises a nucleotide sequence that hybridizes to the nucleotide sequence encoding 12G3H11, B233, B208, B210, G5, 10C12, 4H5, 10G9, 3F2, 1C1, 1F12, 1H3, 1D3, 2B12, or 5A8, or an antigen-binding fragment thereof under stringent conditions, e.g., hybridization to filter-bound DNA in 6× sodium chloride/sodium citrate (SSC) at about 45 degrees C. followed by one or more washes in 0.2×SSC/0.1% SDS at about 50-65 degrees C., under highly stringent conditions, e.g., hybridization to filter-bound nucleic acid in 6×SSC at about 45 degrees C. followed by one or more washes in 0.1×SSC/0.2% SDS at about 68 degrees C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In another embodiment, an antibody that specifically binds to an EphA2 polypeptide comprises an amino acid sequence of a VH domain or an amino acid sequence a VL domain encoded by a nucleotide sequence that hybridizes to the nucleotide sequence encoding the VH or VL domains of 12G3H11, B233, B208, B210, G5, 10C12, 4H5, 10G9, 3F2, 1C1, 1F12, 1H3, 1D3, 2B12, or 5A8 under stringent conditions described herein or under other stringent hybridization conditions which are known to those of skill in the art. In another embodiment, an antibody that specifically binds to an EphA2 polypeptide comprises an amino acid sequence of a VH domain and an amino acid sequence of a VL domain encoded by a nucleotide sequence that hybridizes to the nucleotide sequence encoding the VH and VL domains of 12G3H11, B233, B208, B210, G5, 10C12, 4H5, 10G9, 3F2, 1C1, 1F12, 1H3, 1D3, 2B12, or 5A8 under stringent conditions described herein or under other stringent hybridization conditions which are known to those of skill in the art. In another embodiment, an antibody that specifically binds to an EphA2 polypeptide comprises an amino acid sequence of a VH CDR or an amino acid sequence of a VL CDR encoded by a nucleotide sequence that hybridizes to the nucleotide sequence encoding any one of the VH CDRs or VL CDRs listed in Table 2 or 3, supra, under stringent conditions described herein or under other stringent hybridization conditions which are known to those of skill in the art. In another embodiment, an antibody that specifically binds to an EphA2 polypeptide comprises an amino acid sequence of a VH CDR and an amino acid sequence of a VL CDR encoded by nucleotide sequences that hybridize to the nucleotide sequences encoding any one of the VH CDRs listed in Table 2 or 3, supra, and any one of the VL CDRs listed Table 2 or 3, supra, under stringent conditions described herein or under other stringent hybridization conditions which are known to those of skill in the art.

In another embodiment, the present invention provides an antibody that specifically binds to an EphA2 polypeptide, said antibody comprising a VH domain and/or VL domain encoded by a nucleotide sequence that hybridizes to the nucleotide sequence of the VH domain and/or VL domain of 1C1 (SEQ ID NOS.: 1 and 2, respectively) under stringent conditions. In another embodiment, the present invention provides an antibody that specifically binds to an EphA2 polypeptide, said antibody comprising a VH domain and/or VL domain encoded by a nucleotide sequence that hybridizes to the nucleotide sequence of the VH domain and/or VL domain of 1F12 (SEQ ID NOS.: 11 and 12, respectively) under stringent conditions. In another embodiment, the present invention provides an antibody that specifically binds to an EphA2 polypeptide, said antibody comprising a VH domain and/or VL domain encoded by a nucleotide sequence that hybridizes to the nucleotide sequence of the VH domain and/or VL domain of 1H3 (SEQ ID NOS.: 21 and 22, respectively) under stringent conditions. In another embodiment, the present invention provides an antibody that specifically binds to an EphA2 polypeptide, said antibody comprising a VH domain and/or VL domain encoded by a nucleotide sequence that hybridizes to the nucleotide sequence of the VH domain and/or VL domain of 1D3 (SEQ ID NOS.: 31 and 32, respectively) under stringent conditions. In another embodiment, the present invention provides an antibody that specifically binds to an EphA2 polypeptide, said antibody comprising a VH domain and/or VL domain encoded by a nucleotide sequence that hybridizes to the nucleotide sequence of the VH domain and/or VL domain of 2B12 (SEQ ID NOS.: 41 and 42, respectively) under stringent conditions. In another embodiment, the present invention provides an antibody that specifically binds to an EphA2 polypeptide, said antibody comprising a VH domain and/or VL domain encoded by a nucleotide sequence that hybridizes to the nucleotide sequence of the VH domain and/or VL domain of 5A8 (SEQ ID NOS.: 51 and 52, respectively) under stringent conditions.

In another embodiment, the present invention provides an antibody that specifically binds to an EphA2 polypeptide, said antibody comprising a VH CDR and/or VL CDR encoded by a nucleotide sequence that hybridizes to the nucleotide sequence of the VH CDR and/or VL CDR of 12G3H11, B233, B208, B210, G5, 10C12, 4H5, 10G9, 3F2, 1C1, 1F12, 1H3, 1D3, 2B12, or 5A8 (FIGS. 1-13) under stringent conditions.

In a specific embodiment, an antibody that specifically binds to an EphA2 polypeptide comprises an amino acid sequence that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of 12G3H11, B233, B208, B210, G5, 10C12, 4H5, 10G9, 3F2, 1C1, 1F12, 1H3, 1D3, 2B12, or 5A8, or an antigen-binding fragment thereof. In another embodiment, an antibody that specifically binds to an EphA2 polypeptide comprises an amino acid sequence of a VH domain that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to the VH domain of 12G3H11, B233, B208, B210, G5, 10C12, 4H5, 10G9, 3F2, 1C1, 1F12, 1H3, 1D3, 2B12, or 5A8. In another embodiment, an antibody that specifically binds to an EphA2 polypeptide comprises an amino acid sequence of a VL domain that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to the VL domain of 12G3H11, B233, B208, B210, G5, 10C12, 4H5, 10G9, 3F2, 1C1, 1F12, 1H3, 1D3, 2B12, or 5A8.

In another embodiment, an antibody that specifically binds to an EphA2 polypeptide comprises an amino acid sequence of one or more VL CDRs that are at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 2 or 3, supra. In another embodiment, an antibody that specifically binds to an EphA2 polypeptide comprises an amino acid sequence of one or more VL CDRs that are at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to any of one of the VL CDRs listed in Table 2 or 3, supra.

In another embodiment, the invention provides an antibody that specifically binds to an EphA2 polypeptide, said antibody encoded by a nucleotide sequence that is at least 65%, preferably at least 70%, at least 75%, at least 80%, at least 85%, at least 90%, at least 95%, or at least 99% identical to the nucleotide sequence encoding 1C1. In another embodiment, the invention provides an antibody that specifically binds to an EphA2 polypeptide, said antibody encoded by a nucleotide sequence that is at least 65%, preferably at least 70%, at least 75%, at least 80%, at least 85%, at least 90%, at least 95%, or at least 99% identical to the nucleotide sequence encoding 1F12. In another embodiment, the invention provides an antibody that specifically binds to an EphA2 polypeptide, said antibody encoded by a nucleotide sequence that is at least 65%, preferably at least 70%, at least 75%, at least 80%, at least 85%, at least 90%, at least 95%, or at least 99% identical to the nucleotide sequence encoding 1H3. In another embodiment, the invention provides an antibody that specifically binds to an EphA2 polypeptide, said antibody encoded by a nucleotide sequence that is at least 65%, preferably at least 70%, at least 75%, at least 80%, at least 85%, at least 90%, at least 95%, or at least 99% identical to the nucleotide sequence encoding 1D3. In another embodiment, the invention provides an antibody that specifically binds to an EphA2 polypeptide, said antibody encoded by a nucleotide sequence that is at least 65%, preferably at least 70%, at least 75%, at least 80%, at least 85%, at least 90%, at least 95%, or at least 99% identical to the nucleotide sequence encoding 2B12. In another embodiment, the invention provides an antibody that specifically binds to an EphA2 polypeptide, said antibody encoded by a nucleotide sequence that is at least 65%, preferably at least 70%, at least 75%, at least 80%, at least 85%, at least 90%, at least 95%, or at least 99% identical to the nucleotide sequence encoding 5A8.

In another embodiment, the invention provides an antibody that specifically binds to an EphA2 polypeptide, said antibody comprising a VH domain and/or VL domain encoded by a nucleotide sequence that is at least 65%, preferably at least 70%, at least 75%, at least 80%, at least 85%, at least 90%, at least 95%, or at least 99% identical to the nucleotide sequence of the VH domain and/or VL domain of 1C1 (SEQ ID NOS.: 1 and 2, respectively). In another embodiment, the invention provides an antibody that specifically binds to an EphA2 polypeptide, said antibody comprising a VH domain and/or VL domain encoded by a nucleotide sequence that is at least 65%, preferably at least 70%, at least 75%, at least 80%, at least 85%, at least 90%, at least 95%, or at least 99% identical to the nucleotide sequence of the VH domain and/or VL domain of 1F12 (SEQ ID NOS.: 11 and 12, respectively). In another embodiment, the invention provides an antibody that specifically binds to an EphA2 polypeptide, said antibody comprising a VH domain and/or VL domain encoded by a nucleotide sequence that is at least 65%, preferably at least 70%, at least 75%, at least 80%, at least 85%, at least 90%, at least 95%, or at least 99% identical to the nucleotide sequence of the VH domain and/or VL domain of 1H3 (SEQ ID NOS.: 21 and 22, respectively). In another embodiment, the invention provides an antibody that specifically binds to an EphA2 polypeptide, said antibody comprising a VH domain and/or VL domain encoded by a nucleotide sequence that is at least 65%, preferably at least 70%, at least 75%, at least 80%, at least 85%, at least 90%, at least 95%, or at least 99% identical to the nucleotide sequence of the VH domain and/or VL domain of 1D3 (SEQ ID NOS.: 31 and 32, respectively). In another embodiment, the invention provides an antibody that specifically binds to an EphA2 polypeptide, said antibody comprising a VH domain and/or VL domain encoded by a nucleotide sequence that is at least 65%, preferably at least 70%, at least 75%, at least 80%, at least 85%, at least 90%, at least 95%, or at least 99% identical to the nucleotide sequence of the VH domain and/or VL domain of 2B12 (SEQ ID NOS.: 41 and 42, respectively). In another embodiment, the invention provides an antibody that specifically binds to an EphA2 polypeptide, said antibody comprising a VH domain and/or VL domain encoded by a nucleotide sequence that is at least 65%, preferably at least 70%, at least 75%, at least 80%, at least 85%, at least 90%, at least 95%, or at least 99% identical to the nucleotide sequence of the VH domain and/or VL domain of 5A8 (SEQ ID NOS.: 51 and 52, respectively).

In another embodiment, the invention provides an antibody that specifically binds to an EphA2 polypeptide, said antibody comprising a VH CDR and/or a VL CDR encoded by a nucleotide sequence that is at last 65%, preferably at least 70%, at least 75%, at least 80%, at least 85%, at least 90%, at least 95%, or at least 99% identical to the nucleotide sequence of the VH CDR and/or VL CDR of 12G3H11, B233, B208, B210, G5, 10C12, 4H5, 10G9, 3F2, 1C1, 1F12, 1H3, 1D3, 2B12, or 5A8 (FIGS. 1-13).

The present invention encompasses antibodies that compete with an antibody described herein for binding to an EphA2 polypeptide. In particular, the present invention encompasses antibodies that compete with 12G3H11, B233, B208, B210, G5, 10C12, 4H5, 10G9, 3F2, 1C1, 1F12, 1H3, 1D3, 2B12, or 5A8 or an antigen-binding fragment thereof for binding to the EphA2 polypeptide. In a specific embodiment, the invention encompasses an antibody that reduces the binding of 12G3H11, B233, B208, B210, G5, 10C12, 4H5, 10G9, 3F2, 1C1, 1F12, 1H3, 1D3, 2B12, or 5A8 to an EphA2 polypeptide by at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more, 25% to 50%, 45 to 75%, or 75 to 99% relative to a control such as PBS in the competition assay described herein or competition assays well known in the art. In another specific embodiment, the invention encompasses an antibody that reduces binding of 12G3H11, B233, B208, B210, G5, 10C12, 4H5, 10G9, 3F2, 1C1, 1F12, 1H3, 1D3, 2B12, or 5A8 to an EphA2 polypeptide by at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more, or 25% to 50%, 45 to 75%, or 75 to 99% relative to a control such as PBS in an ELISA competition assay.

An ELISA competition assay may be performed in the following manner: recombinant EphA2 is prepared in PBS at a concentration of 10 μg/ml. 100 μl of this solution is added to each well of an ELISA 98-well microtiter plate and incubated overnight at 4-8 degrees C. The ELISA plate is washed with PBS supplemented with 0.1% Tween to remove excess recombinant EphA2. Non-specific protein-protein interactions are blocked by adding 100 μl of bovine serum albumin (BSA) prepared in PBS to a final concentration of 1%. After one hour at room temperature, the ELISA plate is washed. Unlabeled competing antibodies are prepared in blocking solution at concentrations ranging from 1 μg/ml to 0.01 μg/ml. Control wells contain either blocking solution only or control antibodies at concentrations ranging from 1 μg/ml to 0.01 μg/ml. Test antibody (e.g., 1C1) labeled with horseradish peroxidase is added to competing antibody dilutions at a fixed final concentration of 1 μg/ml. 100 μl of test and competing antibody mixtures are added to the ELISA wells in triplicate and the plate is incubated for 1 hour at room temperature. Residual unbound antibody is washed away. Bound test antibody is detected by adding 100 μl of horseradish peroxidase substrate to each well. The plate is incubated for 30 min. at room temperature, and absorbance is read using an automated plate reader. The average of triplicate wells is calculated. Antibodies which compete well with the test antibody reduce the measured absorbance compared with control wells.

In another embodiment, the invention encompasses an antibody that reduces the binding of an antibody comprising (alternatively, consisting of) an antigen-binding fragment (for example, but not limited to, a VH domain, a VH CDR, a VL domain or a VL CDR) of 12G3H11, B233, B208, B210, G5, 10C12, 4H5, 10G9, 3F2, 1C1, 1F12, 1H3, 1D3, 2B12, or 5A8 to an EphA2 polypeptide by at least 25%, preferably at least 30%, at least 35%, at least 40%, at least 45%, at least 50%, at least 55%, at least 60%, at least 65%, at least 70%, at least 75%, at least 80%, at least 85%, at least 90%, at least 95% or more, or 25% to 50%, 45 to 75%, or 75 to 99% relative to a control such as PBS in a competition assay described herein or well-known to one of skill in the art.

In another embodiment, the invention encompasses an antibody that reduces the binding of an antibody comprising (alternatively, consisting of, or consisting essentially of) an antigen-binding fragment (e.g., a VH domain, VL domain, a VH CDR, or a VL CDR) of 12G3H11, B233, B208, B210, G5, 10C12, 4H5, 10G9, 3F2, 1C1, 1F12, 1H3, 1D3, 2B12, or 5A8 to an EphA2 polypeptide by at least 25%, preferably at least 30%, at least 35%, at least 40%, at least 45%, at least 50%, at least 55%, at least 60%, at least 65%, at least 70%, at least 75%, at least 80%, at least 85%, at least 90%, at least 95% or more, or 25% to 50%, 45 to 75%, or 75 to 99% relative to a control such as PBS in an ELISA competition assay.

The present invention encompasses polypeptides or proteins comprising (alternatively, consisting of, or consisting essentially of) VH domains that compete with the VH domain of 12G3H11, B233, B208, B210, G5, 10C12, 4H5, 10G9, 3F2, 1C1, 1F12, 1H3, 1D3, 2B 12, or 5A8 for binding to an EphA2 polypeptide. The present invention also encompasses polypeptides or proteins comprising (alternatively, consisting of, or consisting essentially of) VL domains that compete with a VL domain of 12G3H11, B233, B208, B210, G5, 10C12, 4H5, 10G9, 3F2, 1C1, 1F12, 1H3, 1D3, 2B12, or 5A8 for binding to an EphA2 polypeptide.

The present invention encompasses polypeptides or proteins comprising (alternatively, consisting of, or consisting essentially of) VH CDRs that compete with a VH CDR listed in Tables 2 or 3, supra, for binding to an EphA2 polypeptide. The present invention also encompasses polypeptides or proteins comprising (alternatively, consisting of) VL CDRs that compete with a VL CDR listed in Tables 2 or 3, supra for binding to an EphA2 polypeptide.

The antibodies that specifically bind to an EphA2 polypeptide include derivatives that are modified, i.e., by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides antibodies that specifically bind to an EphA2 polypeptide, said antibodies comprising a framework region known to those of skill in the art (e.g., a human or non-human framework). The framework regions may be naturally occurring or consensus framework regions. Preferably, the fragment region of an antibody of the invention is human (see, e.g., Chothia et al., 1998, J. Mol. Biol. 278:457-479 for a listing of human framework regions, which is incorporated herein by reference in its entirety).

The present invention encompasses antibodies that specifically bind to an EphA2 polypeptide, said antibodies comprising the amino acid sequence of 12G3H11, B233, B208, B210, G5, 10C12, 4H5, 10G9, 3F2, 1C1, 1F12, 1H3, 1D3, 2B12, or 5A8 with mutations (e.g., one or more amino acid substitutions) in the framework regions. In certain embodiments, antibodies that specifically bind to an EphA2 polypeptide comprise the amino acid sequence of 12G3H11, B233, B208, B210, G5, 10C12, 4H5, 10G9, 3F2, 1C1, 1F12, 1H3, 1D3, 2B12, or 5A8 with one or more amino acid residue substitutions in the framework regions of the VH and/or VL domains.

The present invention also encompasses antibodies that specifically bind to an EphA2 polypeptide, said antibodies comprising the amino acid sequence of 12G3H11, B233, B208, B210, G5, 10C12, 4H5, 10G9, 3F2, 1C1, 1F12, 1H3, 1D3, 2B12, or 5A8 with mutations (e.g., one or more amino acid residue substitutions) in the variable and framework regions.

In certain embodiments, the antibodies of the invention do not include certain antibodies that specifically bind to an EphA2 polypeptide. In one embodiment, the antibodies of the invention are EphA2 binding antibodies, with the proviso that said EphA2 binding antibody is not 1C1. In one embodiment, the antibodies of the invention are EphA2 binding antibodies, with the proviso that said EphA2 binding antibody is not 1F12. In one embodiment, the antibodies of the invention are EphA2 binding antibodies, with the proviso that said EphA2 binding antibody is not 1H3. In one embodiment, the antibodies of the invention are EphA2 binding antibodies, with the proviso that said EphA2 binding antibody is not 1D3. In one embodiment, the antibodies of the invention are EphA2 binding antibodies, with the proviso that said EphA2 binding antibody is not 2B 12. In one embodiment, the antibodies of the invention are EphA2 binding antibodies, with the proviso that said EphA2 binding antibody is not 5A8. In one embodiment, the antibodies of the invention are EphA2 binding antibodies, with the proviso that said EphA2 binding antibody is not 3F2. In one embodiment, the antibodies of the invention are EphA2 binding antibodies, with the proviso that said EphA2 binding antibody is not EA5. In one embodiment, the antibodies of the invention are EphA2 binding antibodies, with the proviso that said EphA2 binding antibody is not G5. In one embodiment, the antibodies of the invention are EphA2 binding antibodies, with the proviso that said EphA2 binding antibody is, not EA2. In one embodiment, the antibodies of the invention are EphA2 binding antibodies, with the proviso that said EphA2 binding antibody is not B233. In one embodiment, the antibodies of the invention are EphA2 binding antibodies, with the proviso that said EphA2 binding antibody is not B208. In one embodiment, the antibodies of the invention are EphA2 binding antibodies, with the proviso that said EphA2 binding antibody is not 10C12. In one embodiment, the antibodies of the invention are EphA2 binding antibodies, with the proviso that said EphA2 binding antibody is not B210.

In specific embodiments, antibodies of the invention bind antigenic epitope-bearing peptides and polypeptides of EphA2, and said antigenic epitope-bearing peptides and polypeptides comprise or consist of an amino acid sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50 continguous amino acid residues, and, preferably, between about 15 to about 30 continguous amino acids of EphA2 found in any species. Polypeptides comprising immunogenic or antigenic epitopes are at least 8, at least 10, at least 15, at least 20, at least 25, at least at least 30, or at least 35 amino acid residues in length.

EphA2 epitope-bearing peptides, polypeptides, and fragments thereof may be produced by any conventional means. See, e.g., Houghten, R. A. (1985) “General method for the rapid solid-phase synthesis of large numbers of peptides: specificity of antigen-antibody interaction at the level of individual amino acids,” Proc. Natl. Acad. Sci. USA 82:5 13 1-5 135; this “Simultaneous Multiple Peptide Synthesis (SMPS)” process is further described in U.S. Pat. No. 4,631,211 to Houghten et al. (1986).

The present invention provides peptides, polypeptides and/or proteins comprising one or more variable or hypervariable regions of the antibodies described herein. Preferably, peptides, polypeptides or proteins comprising one or more variable or hypervariable regions of antibodies of the invention further comprise a heterologous amino acid sequence. In certain embodiments, such a heterologous amino acid sequence comprises at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 75 contiguous amino acid residues, at least 100 contiguous amino acid residues or more contiguous amino acid residues. Such peptides, polypeptides and/or proteins may be referred to as fusion proteins.

In a specific embodiment, peptides, polypeptides or proteins comprising one or more variable or hypervariable regions of the antibodies of the invention are 10 amino acid residues, 15 amino acid residues, 20 amino acid residues, 25 amino acid residues, 30 amino acid residues, 35 amino acid residues, 40 amino acid residues, 45 amino acid residues, 50 amino acid residues, 75 amino acid residues, 100 amino acid residues, 125 amino acid residues, 150 amino acid residues or more amino acid residues in length. In certain embodiments, peptides, polypeptides, or proteins comprising one or more variable or hypervariable regions of an antibody of the invention specifically bind to an EphA2 polypeptide. In other embodiments, peptides, polypeptides, or proteins comprising one or more variable or hypervariable regions of an antibody of the invention do not specifically bind to an EphA2 polypeptide.

In a specific embodiment, the present invention provides peptides, polypeptides and/or proteins comprising a VH domain and/or VL domain of one of the antibodies described herein (see Table 2 and 3, supra). In a further specific embodiment, the present invention provides peptides, polypeptides and/or proteins comprising one or more CDRs having the amino acid sequence of any of the CDRs listed in Table 2 or 3, supra. In accordance with these embodiments, the peptides, polypeptides or proteins may further comprise a heterologous amino acid sequence.

Peptides, polypeptides or proteins comprising one or more variable or hypervariable regions have utility, e.g., in the production of anti-idiotypic antibodies which in turn may be used to prevent, treat, and/or ameliorate one or more symptoms associated with a disease or disorder (e.g., cancer, a hyper- or hypo-proliferative disorder). The anti-idiotypic antibodies produced can also be utilized in immunoassays, such as, e.g., ELISAs, for the detection of antibodies which comprise a variable or hypervariable region contained in the peptide, polypeptide or protein used in the production of the anti-idiotypic antibodies.

The present invention provides for antibodies that specifically bind to an EphA2 polypeptide which have an extended half-life in vivo. In particular, the present invention provides antibodies that specifically bind to an EphA2 polypeptide which have a half-life in a subject, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies, single chain antibodies and Fab fragments) in vivo, for example, inert polymer molecules such as high molecular weight polyethyleneglycol (PEG) can be attached to the antibodies with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication No. WO 98/23289; International Publication No. WO 97/34631; International Publication No. WO 02/060919; U.S. Patent Application Publication No. 2006/0039904 A1 and U.S. Pat. No. 6,277,375, each of which is incorporated herein by reference in its entirety.

Further, antibodies can be conjugated to albumin in order to make the antibody or antibody fragment more stable in vivo or have a longer half life in vivo. The techniques are well-known in the art, see, e.g., International Publication Nos. WO 93/15199, WO 93/15200, and WO 01/77137; and European Patent No. EP 413,622, all of which are incorporated herein by reference.

In order for the ADCs of the invention to perform as required, a key aspect of the antibodies to be conjugated to the toxin of choice is that the antibody, once bound to the cell surface target (e.g. EphA2 or EphA4), is internalized by the cell. Once internalized, the conjugated toxin can be released, or remain bound, to exert its toxic effect on the cell.

The antibody portion of the ADCs of the invention may include, but are not limited to, synthetic antibodies, monoclonal antibodies, oligoclonal antibodies recombinantly produced antibodies, intrabodies, multispecific antibodies, bispecific antibodies, human antibodies, humanized antibodies, chimeric antibodies, synthetic antibodies, single-chain FvFcs (scFvFc), single-chain Fvs (scFv), and anti-idiotypic (anti-Id) antibodies. In particular, antibodies used in the methods of the present invention include immunoglobulin molecules and immunologically active portions of immunoglobulin molecules. The antibodies of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibody portion of the ADCs of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

Antibodies like all polypeptides have an Isoelectric Point (pI), which is generally defined as the pH at which a polypeptide carries no net charge. It is known in the art that protein solubility is typically lowest when the pH of the solution is equal to the isoelectric point (pI) of the protein. It is possible to optimize solubility by altering the number and location of ionizable residues in the antibody to adjust the pI. For example the pI of a polypeptide can be manipulated by making the appropriate amino acid substitutions (e.g., by substituting a charged amino acid such as a lysine, for an uncharged residue such as alanine). Without wishing to be bound by any particular theory, amino acid substitutions of an antibody that result in changes of the pI of said antibody may improve solubility and/or the stability of the antibody. One skilled in the art would understand which amino acid substitutions would be most appropriate for a particular antibody to achieve a desired pI. The pI of a protein may be determined by a variety of methods including but not limited to, isoelectric focusing and various computer algorithms (see for example Bjellqvist et al., 1993, Electrophoresis 14:1023). In one embodiment, the pI of the ADCs of the invention is between is higher then about 6.5, about 7.0, about 7.5, about 8.0, about 8.5, or about 9.0. In another embodiment, the pI of the ADCs of the invention is between is higher then 6.5, 7.0, 7.5, 8.0, 8.5, or 9.0. In one embodiment, substitutions resulting in alterations in the pI of the ADC of the invention will not significantly diminish its binding affinity for an Eph receptor. As used herein the pI value is defined as the pI of the predominant charge form. The pI of a protein may be determined by a variety of methods including but not limited to, isoelectric focusing and various computer algorithms (see, e.g., Bjellqvist et al., 1993, Electrophoresis 14:1023).

The Tm of the Fab domain of an antibody, can be a good indicator of the thermal stability of an antibody and may further provide an indication of the shelf-life. A lower Tm indicates more aggregation/less stability, whereas a higher Tm indicates less aggregation/more stability. Thus, antibodies having higher Tm are preferable. In one embodiment, the Fab domain of an ADC has a Tm value higher than at least 50° C., 55° C., 60° C., 65° C., 70° C., 75° C., 80° C., 85° C., 90° C., 95° C., 100° C., 105° C., 110° C., 115° C. or 120° C. In another embodiment, the Fab domain of an ADC has a Tm value higher than at least about 50° C., about 55° C., about 60° C., about 65° C., about 70° C., about 75° C., about 80° C., about 85° C., about 90° C., about 95° C., about 100° C., about 105° C., about 110° C., about 115° C. or about 120° C. Thermal melting temperatures I of a protein domain (e.g., a Fab domain) can be measured using any standard method known in the art, for example, by differential scanning calorimetry (see, e.g., Vermeer et al., 2000, Biophys. J. 78:394-404; Vermeer et al., 2000, Biophys. J. 79: 2150-2154).

The antibody portion of the ADCs of the invention may be monospecific, bispecific, trispecific or have greater multispecificity. Multispecific antibodies may specifically bind to different epitopes of desired target molecule or may specifically bind to both the target molecule as well as a heterologous epitope, such as a heterologous polypeptide or solid support material. See, e.g., International Publication Nos. WO 94/04690; WO 93/17715; WO 92/08802; WO 91/00360; and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547; each of which is incorporated herein by reference in their entireties). In one embodiment, one of the binding specificities is for an Eph receptor, the other one is for any other antigen (i.e., another Eph receptor, an Ephrin, a signaling or effector molecule).

Multispecific antibodies have binding specificities for at least two different antigens. While such molecules normally will only bind two antigens (i.e. bispecific antibodies, BsAbs), antibodies with additional specificities such as trispecific antibodies are encompassed by the instant invention. Examples of BsAbs include without limitation those with one arm directed against a Integrin αᵥβ₃ and the other arm directed against any other antigen. Methods for making bispecific antibodies are known in the art. Traditional production of full-length bispecific antibodies is based on the coexpression of two immunoglobulin heavy chain-light chain pairs, where the two chains have different specificities (Millstein et al., 1983, Nature, 305:537-539 which is incorporated herein by reference in its entirety). Because of the random assortment of immunoglobulin heavy and light chains, these hybridomas (quadromas) produce a potential mixture of different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., 1991, EMBO J., 10:3655-3659. A more directed approach is the generation of a Di-diabody a tetravalent bispecific antibody. Methods for producing a Di-diabody are known in the art (see e.g., Lu et al., 2003, J Immunol Methods 279:219-32; Marvin et al., 2005, Acta Pharmacolical Sinica 26:649).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. In one embodiment, the first heavy-chain constant region (CH1) containing the site necessary for light chain binding is present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when, the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm (e.g., an Eph receptor), and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 (incorporated herein by reference in its entirety). For further details of generating bispecific antibodies see, for example, Suresh et al., 1986, Methods in Enzymology, 121:210 (incorporated herein by reference in its entirety). According to another approach described in WO96/27011 (incorporated herein by reference in its entirety), a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

In a specific embodiment, antibodies for use in the methods of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was recently described in WO 99/54440, which is herein incorporated by reference. This publication describes a novel single-chain multifunctional polypeptide that comprises binding sites for the CD19 and CD3 antigens (CD 19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the heavy chain (V_{H}) and light chain (V_{L}) variable domains with a flexible linker to create a single chain, bispecific antibody.

In an embodiment of this invention, an antibody or ligand that specifically binds a polypeptide of interest (e.g., an Eph receptor and/or an Ephrin) will comprise a portion of the BiTE molecule. For example, the V_{H} and/or V_{L} (e.g. a scFV) of an antibody that binds a polypeptide of interest (e.g., an Eph receptor and/or an Ephrin) can be fused to an anti-CD3 binding portion such as that of the molecule described above, thus creating a BiTE molecule that targets the polypeptide of interest (e.g., an Eph receptor and/or an Ephrin). In addition to the heavy and/or light chain variable domains of antibody against a polypeptide of interest (e.g., an Eph receptor and/or an Ephrin), other molecules that bind the polypeptide of interest (e.g., an Eph receptor and/or an Ephrin) can comprise the BiTE molecule, for example receptors (e.g., an Eph receptor and/or an Ephrin). In another embodiment, the BiTE molecule can comprise a molecule that binds to other T cell antigens (other than CD3). For example, ligands and/or antibodies that specifically bind to T-cell antigens like CD2, CD4, CD8, CD11a, TCR, and CD28 are contemplated to be part of this invention. This list is not meant to be exhaustive but only to illustrate that other molecules that can specifically bind to a T cell antigen can be used as part of a BiTE molecule. These molecules can include the VH and/or VL portions of the antibody or natural ligands (for example LFA3 whose natural ligand is CD3).

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). The above references are each incorporated herein by reference in their entireties. Heteroconjugate antibodies may be made using any convenient cross-linking methods. Suitable cross-linking agents are well known in the art, and are disclosed in U.S. Pat. No. 4,676,980, along with a number of cross-linking techniques. Each of the above references is incorporated herein by reference in its entirety.

Antibodies with more than two valencies incorporating at least one hinge modification of the invention are contemplated. For example, trispecific antibodies can be prepared. See, e.g., Tutt et al. J. Immunol. 147: 60 (1991), which is incorporated herein by reference.

The antibody portion of the ADCs of the invention encompass single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079; which are incorporated herein by reference in their entireties).

Other antibodies specifically contemplated are “oligoclonal” antibodies. As used herein, the term “oligoclonal” antibodies” refers to a predetermined mixture of distinct monoclonal antibodies. See, e.g., PCT publication WO 95/20401; U.S. Pat. Nos. 5,789,208 and 6,335,163 which are incorporated by reference herein. Preferably oligoclonal antibodies consist of a predetermined mixture of antibodies against one or more epitopes are generated in a single cell. More preferably oligoclonal antibodies comprise a plurality of heavy chains capable of pairing with a common light chain to generate antibodies with multiple specificities (e.g., PCT publication WO 04/009618 which is incorporated by reference herein). Oligoclonal antibodies are particularly useful when it is desired to target multiple epitopes on a single target molecule (e.g., Integrin αᵥβ₃). Those skilled in the art will know or can determine what type of antibody or mixture of antibodies is applicable for an intended purpose and desired need.

In one embodiment, the ADCs of the invention may be chemically modified (e.g., one or more chemical moieties can be attached to the antibody) or be modified to alter its glycosylation, again to alter one or more functional properties of the antibody.

In still another embodiment, the glycosylation of the ADCs of the invention is modified. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861, each of which is incorporated herein by reference in its entirety.

Additionally or alternatively, an ADC can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342, each of which is incorporated herein by reference in its entirety.

In still another embodiment, the glycosylation of an ADC of the invention is modified. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861, each of which is incorporated herein by reference in its entirety.

Additionally or alternatively, an ADC can be made that has an altered type of glycosylation, such as a hypofucosylated Fc variant having reduced amounts of fucosyl residues or an Fc variant having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342, each of which is incorporated herein by reference in its entirety.

The present invention also encompasses antibodies that are Fc variants with enhanced antibody dependent cell-mediated cytotoxicity activity. Nonlimiting examples of such Fc variant antibodies are disclosed in U.S. patent application Ser. Nos. 11/203,253 (filed Aug. 15, 2005 and published as U.S. Patent Application Publication No. US 2006/0039904 A1) and 11/203,251 (filed Aug. 15, 2005), and U.S. Provisional Patent Applications 60/674,674 (filed Apr. 26, 2005) and 60/713,711 (filed Sep. 6, 2005), each of which is incorporated by reference herein in its entirety.

### Antibody Conjugates

The present invention encompasses the use of antibodies or fragments thereof recombinantly fused or chemically conjugated (including both covalent and non-covalent conjugations) to a heterologous agent to generate a fusion protein as both targeting moieties and anti-EphA2 or anti-EphA4 agents. The heterologous agent may be a polypeptide (or portion thereof, preferably to a polypeptide of at least 10, at least 20, at least 30, at least 40, at least 50, at least 60, at least 70, at least 80, at least 90 or at least 100 amino acids), nucleic acid, small molecule (less than 1000 daltons), or inorganic or organic compound. The fusion does not necessarily need to be direct, but may occur through linker sequences. Antibodies fused or conjugated to heterologous agents may be used in vivo to detect, treat, manage, or monitor the progression of a disorder using methods known in the art. See e.g., International Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties. In some embodiments, the disorder to be detected, treated, managed, or monitored is malignant cancer that overexpresses EphA2 or EphA4. In other embodiments, the disorder to be detected, treated, managed, or monitored is a pre-cancerous condition associated with cells that overexpress EphA2 or EphA4. In a specific embodiment, the pre-cancerous condition is high-grade prostatic intraepithelial neoplasia (PIN), fibroadenoma of the breast, fibrocystic disease, or compound nevi.

The present invention further includes compositions comprising heterologous agents fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341 (said references incorporated by reference in their entireties).

Additional fusion proteins, e.g., of EA2-5, Eph099B-102.147, Eph099B-208.261, Eph099B-210.248, Eph099B-233.152, any of the antibodies listed in Table 2 or 3, or FIGS. 1-59, or EA44 (or any other EphA2/EphA4 agonistic antibody or EphA2/EphA4 cancer cell phenotype inhibiting antibody or exposed EphA2/EphA4 epitope antibody or EphA2/EphA4 antibody that binds EphA2 or EphA4 with a K_{off} of less than 3×10⁻³ s⁻¹), may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions specifically bind to EphA2 or EphA4 may be recombined with one or more components, motifs, sections, parts, domains, fragments, etc. of one or more heterologous agents.

In one embodiment, antibodies of the present invention or fragments or variants thereof are conjugated to a marker sequence, such as a peptide, to facilitate purification. In certain embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies of the present invention or fragments or variants thereof are conjugated to a diagnostic or detectable agent. Such antibodies can be useful for monitoring or prognosing the development or progression of a cancer as part of a clinical testing procedure, such as determining the efficacy of a particular therapy. Additionally, such antibodies can be useful for monitoring or prognosing the development or progression of a pre-cancerous condition associated with cells that overexpress EphA2 or EphA4 (e.g., high-grade prostatic intraepithelial neoplasia (PIN), fibroadenoma of the breast, fibrocystic disease, or compound nevi). In one embodiment, an exposed EphA2 or EphA4 epitope antibody is conjugated to a diagnostic or detectable agent.

Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁷Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴ Mn), molybdenum (⁹⁹Mo), palladium (¹⁰³Pd), phosphorous (³²P), praseodymium (¹⁴²Pr), promethium (¹⁴⁹ Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

In other embodiments, antibodies of the present invention or fragments or variants thereof are conjugated to a therapeutic agent such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

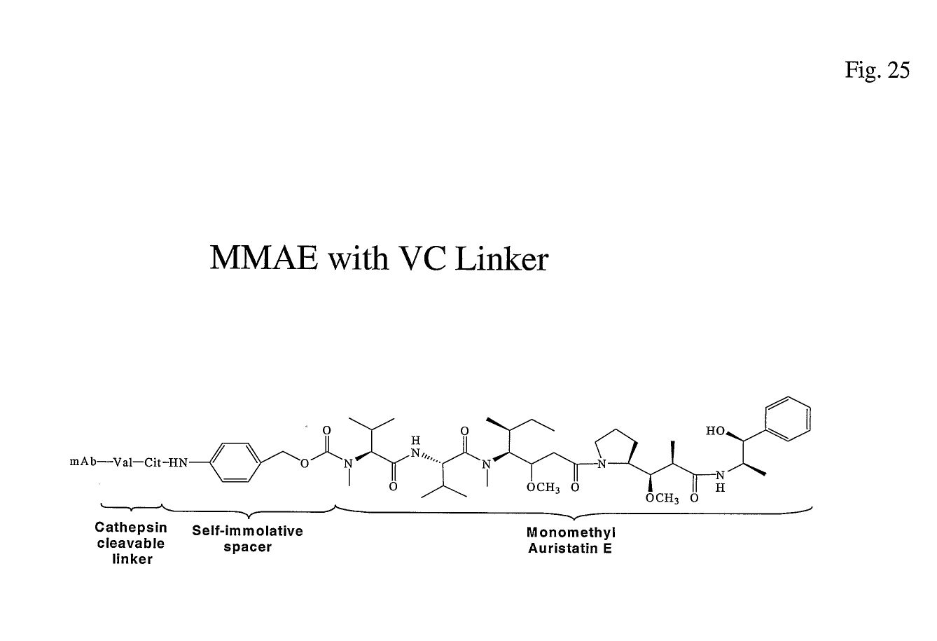

In one embodiment, the cytotoxic agent is selected from the group consisting of an enediyne, a lexitropsin, a duocarmycin, a taxane, a puromycin, a dolastatin, a maytansinoid, and a vinca alkaloid. In other embodiments, the cytotoxic agent is paclitaxel, docetaxel, CC-1065, SN-38, topotecan, morpholino-doxorubicin, rhizoxin, cyanomorpholino-doxorubicin, dolastatin-10, echinomycin, combretastatin, calicheamicin, maytansine, DM-1, an auristatin or other dolastatin derivatives, such as auristatin E or auristatin F, AEB, AEVB, AEFP, MMAE (monomethylauristatin E), MMAF (monomethylauristatin F), eleutherobin or netropsin. The structures of MMAE and MMAF are depicted in FIGS. 25-27.

In yet other embodiments, the cytotoxic agent of an ADC of the invention is an anti-tubulin agent. In more specific embodiments, the cytotoxic agent is selected from the group consisting of a vinca alkaloid, a podophyllotoxin, a taxane, a baccatin derivative, a cryptophysin, a maytansinoid, a combretastatin, and a dolastatin. In more specific embodiments, the cytotoxic agent is vincristine, vinblastine, vindesine, vinorelbine, VP-16, camptothecin, paclitaxel, docetaxel, epithilone A, epithilone B, nocodazole, coichicine, colcimid, estramustine, cemadotin, discodermolide, maytansine, DM-1, an auristatin or other dolastatin derivatives, such as auristatin E or auristatin F, AEB, AEVB, AEFP, MMAE (monomethylauristatin E), MMAF (monomethylauristatin F), eleutherobin or netropsin.

In a specific embodiment, the cytotoxic agent of an ADC of the invention is MMAE. In another specific embodiment, the cytotoxic agent of an ADC of the invention is AEFP. In a further specific embodiment, the cytoxic agent of an ADC of the invention is MMAF.

Further examples of toxins, spacers, linkers, stretchers and the like, and their structures can be found in U.S. Patent Application Publication Nos. 2006/0074008 A1, 2005/0238649 A1, 2005/0123536 A1, 2005/0180972 A1, 2005/0113308 A1, 2004/0157782 A1, U.S. Pat. No. 6,884,869 B2, U.S. Pat. No. 5,635,483, all of which are hereby incorporated herein in their entirety.

As discussed herein, the drugs used for conjugation to the ADCs of the present invention can include conventional chemotherapeutics, such as doxorubicin, paclitaxel, melphalan, vinca alkaloids, methotrexate, mitomycin C, etoposide, and others. In addition, potent agents such CC-1065 analogues, calichiamicin, maytansine, analogues of dolastatin 10, rhizoxin, and palytoxin can be linked to the ADCs using the conditionally stable linkers to form potent immunoconjugates.

In certain embodiments, the ADCs of the invention comprise drugs that are at least 40-fold more potent than doxorubicin on EphA2 or EphA4-expressing cells. Such drugs include, but are not limited to: DNA minor groove binders, including enediynes and lexitropsins, duocarmycins, taxanes (including paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epithilone A and B, estramustine, cryptophysins, cemadotin, maytansinoids, dolastatins, e.g., auristatin E, dolastatin 10, MMAE, MMAF, discodermolide, eleutherobin, and mitoxantrone.

In certain specific embodiments, an ADC of the invention comprises an enediyne moiety. In a specific embodiment, the enediyne moiety is calicheamicin. Enediyne compounds cleave double stranded DNA by generating a diradical via Bergman cyclization.

In other specific embodiments, the cytotoxic or cytostatic agent is auristatin E or an auristatin F, or a derivative thereof. In a further embodiment, the auristatin E derivative is an ester formed between auristatin E and a keto acid. For example, auristatin E can be reacted with paraacetyl benzoic acid or benzoylvaleric acid to produce AEB and AEVB, respectively. Other auristatin derivatives include MMAE, MMAF, and AEFP.

The synthesis and structure of auristatin E, also known in the art as dolastatin-10, and its derivatives are described in U.S. Patent Application Publ. Nos. 2003/0083263 A1 and 2005/0009751 A1; in the International Patent Application No.: PCT/US02/13435, in U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, all of which are incorporated by reference in their entireties herein.

In specific embodiments, the drug is a DNA minor groove binding agent. Examples of such compounds and their syntheses are disclosed in U.S. Pat. No. 6,130,237, which is incorporated by reference in its entirety herein. In certain embodiments, the drug is a CBI compound.

In certain embodiments of the invention, an ADC of the invention comprises an anti-tubulin agent. Anti-tubulin agents are a well established class of cancer therapy compounds. Examples of anti-tubulin agents include, but are not limited to, taxanes (e.g., Taxol® (paclitaxel), docetaxel), T67 (Tularik), vincas, and auristatins (e.g., auristatin E, AEB, AEVB, MMAE, MMAF, AEFP). Antitubulin agents included in this class are also: vinca alkaloids, including vincristine and vinblastine, vindesine and vinorelbine; taxanes such as paclitaxel and docetaxel and baccatin derivatives, epithilone A and B, nocodazole, □luorouraci and colcimid, estramustine, cryptophysins, cemadotin, maytansinoids, combretastatins, dolastatins, discodermolide and eleutherobin.

In a specific embodiment, the drug is a maytansinoid, a group of anti-tubulin agents. In a more specific embodiment, the drug is maytansine. Further, in a specific embodiment, the cytotoxic or cytostatic agent is DM-1 (ImmunoGen, Inc.; see also Chari et al. 1992, Cancer Res 52:127-131). Maytansine, a natural product, inhibits tubulin polymerization resulting in a mitotic block and cell death. Thus, the mechanism of action of maytansine appears to be similar to that of vincristine and vinblastine. Maytansine, however, is about 200 to 1,000-fold more cytotoxic in vitro than these vinca alkaloids. In another specific embodiment, the drug is an AEFP.

In certain specific embodiments of the invention, the drug is not a polypeptide of greater than 50, 100 or 200 amino acids, for example a toxin. In a specific embodiment of the invention, the drug is not ricin.

In other specific embodiments of the invention, an ADC of the invention does not comprise one or more of the cytotoxic or cytostatic agents the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, vinca alkaloids, purine antagonists, and dihydrofolate reductase inhibitors. In more specific embodiments, the high potency drug is not one or more of an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, □luorouraci, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine. VP-16, VM-26, azothioprine, mycophenolate mofetil, methotrexate, acyclovir, gangcyclovir, zidovudine, vidarabine, ribavarin, azidothymidine, cytidine arabinoside, amantadine, dideoxyuridine, iododeoxyuridine, poscarnet, and trifluridine.

In certain embodiments, the cytotoxic or cytostatic agent is a dolastatin. In more specific embodiments, the dolastatin is of the auristatin class. In a specific embodiment of the invention, the cytotoxic or cytostatic agent is MMAE. In another specific embodiment of the invention, the cytotoxic or cytostatic agent is AEFP. In another specific embodiment of the invention, the cytotoxic or cytostatic agent is MMAF.

In other embodiments, antibodies of the present invention or fragments or variants thereof are conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGf (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-4 (“IL-4”), interleukin-6 (“IL-6”), interleukin-7 (“IL-7”), interleukin-9 (“IL-9”), interleukin-15 (“IL-15”), interleukin-12 (“IL-12”), granulocyte macrophage colony stimulating factor (“GM-CSF”), and granulocyte colony stimulating factor (“G-CSF”)), or a growth factor (e.g., growth hormone (“GH”)).

In other embodiments, antibodies of the present invention or fragments or variants thereof are conjugated to a therapeutic agent such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules, further discussed herein below, are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

In a specific embodiment, the conjugated antibody is an EphA2 or EphA4 antibody that preferably binds an EphA2 or EphA4 epitope exposed on cancer cells but not on non-cancer cells (i.e., exposed EphA2 or EphA4 epitope antibody). In another specific embodiment, the conjugated antibody is not EA2 or EA4. In another specific embodiment, the conjugated antibody is not EA44.

Techniques for conjugating therapeutic moieties to antibodies are well known. Moieties can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53:171-216). Additional techniques for conjugating therapeutic moieties to antibodies are well known, see, e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide moieties are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341. The fusion of an antibody to a moiety does not necessarily need to be direct, but may occur through linker sequences. Such linker molecules are commonly known in the art and described in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216, each of which is incorporated herein by reference in its entirety.

Two approaches may be taken to minimize drug activity outside the cells that are targeted by the ADCs of the invention: first, an antibody that binds to cell membrane but not soluble EphA2 or EphA4 may be used, so that the drug, including drug produced by the actions of the prodrug converting enzyme, is concentrated at the cell surface of the activated lymphocyte. A more preferred approach for minimizing the activity of drugs bound to the antibodies of the invention is to conjugate the drugs in a manner that would reduce their activity unless they are hydrolyzed or cleaved off the antibody. Such methods would employ attaching the drug to the antibodies with linkers that are sensitive to the environment at the cell surface of the activated lymphocyte (e.g., the activity of a protease that is present at the cell surface of the activated lymphocyte) or to the environment inside the activated lymphocyte the conjugate encounters when it is taken up by the activated lymphocyte (e.g., in the endosomal or, for example by virtue of pH sensitivity or protease sensitivity, in the lysosomal environment). Examples of linkers that can be used in the present invention are disclosed in U.S. Patent Application Publication Nos. 2005/0123536 A1, 2005/0180972 A1, 2005/0113308 A1, 2004/0157782 A1, and U.S. Pat. No. 6,884,869 B2, all of which are hereby incorporated by reference herein in their entirety.

In one embodiment, the linker is an acid-labile hydrazone or hydrazide group that is hydrolyzed in the lysosome (see, e.g., U.S. Pat. No. 5,622,929). In alternative embodiments, drugs can be appended to antibodies through other acid-labile linkers, such as cis-aconitic amides, orthoesters, acetals and ketals (Dubowchik and Walker, 1999, Pharm. Therapeutics 83:67-123; Neville et al., 1989, Biol. Chem. 264:14653-14661). Such linkers are relatively stable under neutral pH conditions, such as those in the blood, but are unstable at below pH 5, the approximate pH of the lysosome.

In other embodiments, drugs are attached to the antibodies of the invention using peptide spacers that are cleaved by intracellular proteases. Target enzymes include cathepsins B and D and plasmin, all of which are known to hydrolyze dipeptide drug derivatives resulting in the release of active drug inside target cells (Dubowchik and Walker, 1999, Pharm. Therapeutics 83:67-123). The advantage of using intracellular proteolytic drug release is that the drug is highly attenuated when conjugated and the serum stabilities of the conjugates can be extraordinarily high.

In yet other embodiments, the linker is a malonate linker (Johnson et al., 1995. Anticancer Res. 15:1387-93), a maleimidobeiizoyl linker (Lau et al., 1995, Bioorg-Med-Chem. 3(10):1299-1304), or a 3′-N-amide analog (Lau et al, 1995, Bioorg-Med-Chem. 3(103:1305-12).

As discussed above, ADCs are generally made by conjugating a drug to an antibody through a linker. Thus, a majority of the ADCs of the present invention, which comprise an anti-EphA2 or EphA4 antibody and a high potency drug and/or an internalization-promoting drug, further comprise a linker. Any linker that is known in the art may be used in the ADCs of the present invention, e.g., bifunctional agents (such as dialdehydes or imidoesters) or branched hydrazone linkers (see, e.g., U.S. Pat. No. 5,824,805, which is incorporated by reference herein in its entirety).

In certain, non-limiting, embodiments of the invention, the linker region between the drug moiety and the antibody moiety of the ADC is cleavable under certain conditions, wherein cleavage or hydrolysis of the linker releases the drug moiety from the antibody moiety. Preferably, the linker is sensitive to cleavage or hydrolysis under intracellular conditions.

In one embodiment, the linker region between the drug moiety and the antibody moiety of the ADC is cleavable if the pH changes by a certain value or exceeds a certain value. In another embodiment of the invention, the linker is cleavable in the milieu of the lysosome, e.g., under acidic conditions (i.e., a pH of around 5-5.5 or less). In other embodiments, the linker is a peptidyl linker that is cleaved by a peptidase or protease enzyme, including but not limited to a lysosomal protease enzyme, a membrane-associated protease, an intracellular protease, or an endosomal protease. Typically, the linker is at least two amino acids long, more typically at least three amino acids long. Peptidyl linkers that are cleavable by enzymes that are present in EphA2 or EphA4-expressing cancers are preferred. For example, a peptidyl linker that is cleavable by cathepsin-B (e.g., a Gly-Phe-Leu-Gly linker), a thiol-dependent protease that is highly expressed in cancerous tissue, can be used. Other such linkers are described, e.g., in U.S. Pat. No. 6,214,345, which is incorporated by reference in its entirety herein.

In other, non-mutually exclusive embodiments of the invention, the linker by which the anti-EphA2 or EphA4 antibody and the drug of an ADC of the invention are conjugated promotes cellular internalization. In certain embodiments, the linker-drug moiety of the ADC promotes cellular internalization. In certain embodiments, the linker is chosen such that the structure of the entire ADC promotes cellular internalization.

In a specific embodiment of the invention, derivatives of valine-citrulline are used as linker (val-cit linker). The synthesis of doxorubicin with the val-cit linker have been previously described (U.S. Pat. No. 6,214,345 to Dubowchik and Firestone, which is incorporated by reference herein in its entirety).

In a further specific embodiment, the linker is a maleimidocaproyl-citrulline linker or a maleimidocaproyl-valine-citrulline linker.

In another specific embodiment, the linker is a phe-lys linker.

In another specific embodiment, the linker is a thioether linker (see, e.g., U.S. Pat. No. 5,622,929 to Willner et al., which is incorporated by reference herein in its entirety).

In yet another specific embodiment, the linker is a hydrazone linker (see, e.g., U.S. Pat. Nos. 5,122,368 to Greenfield et al. and 5,824,805 to King et al., which are incorporated by reference herein in their entireties).

In yet other specific embodiments, the linker is a disulfide linker. A variety of disulfide linkers are known in the art, including but not limited to those that can be formed using SATA (N-succinimidyl-5-acetylthioacetate), SPDP (N-succinimidyl-3-(2-pyridyldi-thio)propionate), SPDB (N-succinimidyl-3-(2-pyridyldithio)butyrate) and SMPT (N-succinimidyl-oxycarbonyl-alpha-methyl-alpha-(2-pyridyl-dithio)tol-uene). SPDB and SMPT (see, e.g., Thorpe et al., 1987, Cancer Res., 47:5924-5931; Wawrzynczak et al., 1987, In Immunoconjugates: Antibody Conjugates in Radioimagery and Therapy of Cancer, ed. C. W. Vogel, Oxford U. Press, pp. 28-55; see also U.S. Pat. No. 4,880,935 to Thorpe et al., which is incorporated by reference herein in its entirety).

A variety of linkers that can be used with the compositions and methods of the present invention are described in U.S. Patent Application Publication No. US 2004/0018194 A1, which is incorporated by reference in its entirety herein.

In yet other embodiments of the present invention, the linker unit of an anti-EphA2 or EphA4 antibody-linker-drug conjugate (anti-EphA2 or anti-EphA4 ADC) links the cytotoxic or cytostatic agent (drug unit; -D) and the anti-EphA2 or EphA4 antibody unit (-A). As used herein the term anti-EphA2 or anti-EphA4 ADC encompasses anti-EphA2 or anti-EphA4 antibody drug conjugates with and without a linker unit. In certain embodiments, the linker unit has the general formula:

-Tₐ-W_{w}—Y_{y}—

wherein:

-T- is a stretcher unit;

a is 0 or 1;

each —W— is independently an amino acid unit;

w is independently an integer ranging from 2 to 12;

—Y— is a spacer unit; and

y is 0, 1 or 2.

The stretcher unit (-T-), when present, links the anti-EphA2 or anti-EphA4 antibody unit to an amino acid unit (—W—). Useful functional groups that can be present on an anti-EphA2 or anti-EphA4 antibody, either naturally or via chemical manipulation include, but are not limited to, sulfhydryl, amino, hydroxyl, the anomeric hydroxyl group of a carbohydrate, and carboxyl. Preferred functional groups are sulfhydryl and amino. Sulfhydryl groups can be generated by reduction of the intramolecular disulfide bonds of an anti-EphA2 or anti-EphA4 antibody. Alternatively, sulfhydryl groups can be generated by reaction of an amino group of a lysine moiety of an anti-EphA2 or anti-EphA4 antibody with 2-iminothiolane (Traut's reagent) or other sulfhydryl generating reagents. In specific embodiments, the anti-EphA2 or anti-EphA4 antibody is a recombinant antibody and is engineered to carry one or more lysines. In other embodiments, the recombinant anti-EphA2 or anti-EphA4 antibody is engineered to carry additional sulfhydryl groups, e.g., additional cysteines.

In certain specific embodiments, the stretcher unit forms a bond with a sulfur atom of the anti-EphA2 or anti-EphA4 antibody unit. The sulfur atom can be derived from a sulfhydryl (—SH) group of a reduced anti-EphA2 or anti-EphA4 antibody (A). In certain other specific embodiments, the stretcher unit is linked to the anti-CD30 antibody unit (A) via a disulfide bond between a sulfur atom of the anti-CD30 antibody unit and a sulfur atom of the stretcher unit.

In even other specific embodiments, the reactive group of the stretcher contains a reactive site that can be reactive to an amino group of an anti-EphA2 or anti-EphA4 antibody. The amino group can be that of an arginine or a lysine. Suitable amine reactive sites include, but are not limited to, activated esters such as succinimide esters, 4-nitrophenyl esters, pentafluorophenyl esters, anhydrides, acid chlorides, sulfonyl chlorides, isocyanates and isothiocyanates.

In yet another aspect of the invention, the reactive function of the stretcher contains a reactive site that is reactive to a modified carbohydrate group that can be present on an anti-EphA2 or anti-EphA4 antibody. In a specific embodiment, the anti-EphA2 or anti-EphA4 antibody is glycosylated enzymatically to provide a carbohydrate moiety. The carbohydrate may be mildly oxidized with a reagent such as sodium periodate and the resulting carbonyl unit of the oxidized carbohydrate can be condensed with a stretcher that contains a functionality such as a hydrazide, an oxime, a reactive amine, a hydrazine, a thiosemicarbazone, a hydrazine carboxylate, and an arylhydrazide such as those described by Kaneko, T. et al. Bioconjugate Chem 1991, 2, 133-41.

The amino acid unit (—W—) links the stretcher unit (-T-) to the Spacer unit (—Y—) if the Spacer unit is present, and links the stretcher unit to the cytotoxic or cytostatic agent (Drug unit; D) if the spacer unit is absent.

—W_{w}— is a dipeptide, tripeptide, tetrapeptide, pentapeptide, hexapeptide, heptapeptide, octapeptide, nonapeptide, decapeptide, undecapeptide or dodecapeptide unit. The amino acid unit of the linker unit can be enzymatically cleaved by an enzyme including, but not limited to, a tumor-associated protease to liberate the drug unit (-D) which is protonated in vivo upon release to provide a cytotoxic drug (D).

In a one embodiment, the amino acid unit is a phenylalanine-lysine dipeptide (phe-lys or FK linker). In another embodiment, the amino acid unit is a valine-citrulline dipeptide (val-cit or VC linker).

The spacer unit (—Y—), when present, links an amino acid unit to the drug unit. Spacer units are of two general types: self-immolative and non self-immolative. A non self-immolative spacer unit is one in which part or all of the spacer unit remains bound to the drug unit after enzymatic cleavage of an amino acid unit from the anti-EphA2 or anti-EphA4 antibody-linker-drug conjugate or the drug-linker compound. Examples of a non self-immolative spacer unit include, but are not limited to a (glycine-glycine) spacer unit and a glycine spacer unit. When an anti-EphA2 or anti-EphA4 antibody-linker-drug conjugate of the invention containing a glycine-glycine spacer unit or a glycine spacer unit undergoes enzymatic cleavage via a tumor-cell associated-protease, a cancer-cell-associated protease or a lymphocyte-associated protease, a glycine-glycine-drug moiety or a glycine-drug moiety is cleaved from A-T-W.sub.w-. To liberate the drug, an independent hydrolysis reaction should take place within the target cell to cleave the glycine-drug unit bond.

Other examples of self-immolative spacers include, but are not limited to, aromatic compounds that are electronically equivalent to the PAB group such a 2-aminoimidazol-5-methanol derivatives (see Hay et al., Bioorg. Med. Chem. Lett., 1999, 9, 2237 for examples) and ortho or para-aminobenzylacetals. Spacers can be used that undergo facile cyclization upon amide bond hydrolysis, such as substituted and unsubstituted 4-aminobutyric acid amides (Rodrigues et al., Chemistry, Biology, 1995, 2, 223), appropriately substituted ring systems (Storm, et al., J. Amer. Chem. Soc., 1972, 94, 5815) and 2-aminophenylpropionic acid amides (Amsberry, et al., J. Org. Chem., 1990, 55, 5867). Elimination of amine-containing drugs that are substituted at the α-position of glycine (Kingsbury, et al., J. Med. Chem., 1984, 27, 1447) are also examples of self-immolative spacer strategies that can be applied to the antibody-linker-drug conjugates of the invention.

In specific embodiments, the anti-EphA2 or EphA4 antibody of an ADC of the invention is conjugated to the cytotoxic agent via a linker, wherein the linker is peptide linker. In specific embodiments, the anti-EphA2 or EphA4 antibody of an ADC of the invention is conjugated to the cytotoxic agent via a linker, wherein the linker is a val-cit linker, a phe-lys linker, a hydrazone linker, or a disulfide linker. In certain embodiments, the anti-EphA2 or EphA4 antibody of an ADC of the invention is conjugated to the cytotoxic agent via a peptide linker.

In certain embodiments, the conjugate of the invention is anti-EphA2-valine-citrulline-MMAE (anti-EphA2-val-citMMAE or anti-EphA2-vcMMAE), or anti-EphA2-valine-citrulline-MMAF, or anti-EphA2-malaeimidocaproyl-citrulline-MMAE, or anti-EphA2-malaeimidocaproyl-citrulline-MMAF, or anti-EphA2-valine-citrulline-AEFP (anti-EphA2-val-citAEFP or anti-EphA2-vcAEFP). In a specific embodiment, the conjugate of the invention is G5-valine-citrulline-MMAE (G5-val-citMMAE or G5-vcMMAE) or G5-valine-citrulline-AEFP (G5-val-citAEFP or G5-vcAEFP).

In a further specific embodiment, the conjugate of the invention is an antibody selected from those disclosed in FIGS. 1-59 of the present invention linked to -valine-citrulline-MMAE, linked to -valine-citrulline-MMAF, linked to -malaeimidocaproyl-citrulline-MMAE, linked to malaeimidocaproyl-citrulline-MMAF, or to -valine-citrulline-AEFP.

In certain embodiments, the conjugate of the invention is anti-EphA4-valine-citrulline-MMAE (anti-EphA4-val-citMMAE or anti-EphA4-vcMMAE) or anti-EphA4-valine-citrulline-AEFP (anti-EphA4-val-citAEFP or anti-EphA4-vcAEFP).

In other embodiments, the conjugate of the invention is anti-EphA2-phenylalanine-lysine-MMAE (anti-EphA2-phe-lysMMAE or anti-EphA2-fkMMAE) or anti-EphA2-phenylalanine-lysine-AEFP (anti-EphA2-phe-lysAEFP or anti-EphA2-fkAEFP).

In specific embodiments, the conjugate of the invention is G5-phenylalanine-lysine-MMAE (G5-phe-lysMMAE or G5-fkMMAE) or G5-phenylalanine-lysine-AEFP (G5-phe-lysAEFP or G5-fkAEFP).

In specific embodiments, the conjugate of the invention is an antibody selected from those disclosed in FIGS. 1-59 of the present invention linked to -phenylalanine-lysine-MMAE, or to phenylalanine-lysine-MMAF, or to -phenylalanine-lysine-AEFP.

In other embodiments, the conjugate of the invention is anti-EphA4-phenylalanine-lysine-MMAE (anti-EphA4-phe-lysMMAE or anti-EphA4-fkMMAE) or anti-EphA4-phenylalanine-lysine-AEFP (anti-EphA4-phe-lysAEFP or anti-EphA4-fkAEFP).

Thus, in a specific embodiment, the present invention provides methods for the treatment of cancer in a subject, comprising administering to the subject, in an amount effective for said treatment, a pharmaceutical composition comprising (a) G5-val-cit-MMAE; and (b) a pharmaceutically acceptable carrier.

In another specific embodiment, the present invention provides methods for the treatment of cancer in a subject, comprising administering to the subject, in an amount effective for said treatment, a pharmaceutical composition comprising (a) G5-val-cit-AEFP; and (b) a pharmaceutically acceptable carrier.

In another specific embodiment, the present invention provides methods for the treatment of cancer in a subject, comprising administering to the subject, in an amount effective for said treatment, a pharmaceutical composition comprising (a) G5-val-cit-MMAF; and (b) a pharmaceutically acceptable carrier.

In certain embodiments, the anti-EphA2 or EphA4 antibody of an ADC of the invention is conjugated to the cytotoxic agent via a linker, wherein the linker is cleavable at a pH of less than 5.5. In a specific embodiment the linker is cleavable at a pH of less than 5.0.

In certain embodiments, the anti-EphA2 or EphA4 antibody of an ADC of the invention is conjugated to the cytotoxic agent via a linker, wherein the linker is cleavable by a protease. In a specific embodiment, the protease is a lysosomal protease. In other specific embodiments, the protease is, inter alia, a membrane-associated protease, an intracellular protease, or an endosomal protease.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

In one embodiment, the antibodies of the invention once bound, internalize into cells wherein internalization is at least about 10%, at least about 20%, at least about 30%, at least about 40%, at least about 50%, at least about 60%, at least about 70%, at least about 80%, or at least about 90%, at least about 100%, at least about 110%, at least about 130%, at least about 140%, at least about 150%, at least about 160%, or at least about 170% more than control antibodies as described herein.

In another embodiment, the antibodies of the invention once bound, internalize into cells wherein internalization is 1-10%, 10-20%, 20-30%, 30-40%, 40-50%, 50-60%, 60-70%, 70-80%, 80-90%, 90-100%, 100-110%, 110-120%, 120-130%, 130-140%, 140-150%, 150-160%, 160-170% more than control antibodies as described herein.

In another embodiment, the antibodies of the invention once bound, internalize into cells wherein internalization is 1-10%, 10-20%, 20-30%, 30-40%, 40-50%, 50-60%, 60-70%, 70-80%, 80-90%, 90-100%, 100-110%, 110-120%, 120-130%, 130-140%, 140-150%, 150-160%, 160-170% more than control antibodies as determined by the internalization assay using a secondary saponin antibody.

In another embodiment, the antibodies of the invention activate receptors and internalize when bound to cells without exhibiting tissue cross-reactivity with the human heart as described herein.

In another embodiment, the antibodies of the invention activate receptors and internalize when bound to cells without exhibiting tissue cross-reactivity with the human heart when administered at lower doses as described herein.

In another embodiment, the antibodies of the invention do not activate receptors but internalize when bound to cells without exhibiting tissue cross-reactivity with the human heart, as described herein.

In another embodiment, the antibodies of the invention bind to multiple cell types measured by an increase of mean fluorescence of at least about 10%, at least about 100%, at least about 500%, at least about 1000%, at least about 1500%, at least about 2000%, at least about 2500%, at least about 3000%, at least about 3500%, at least about 4000%, at least about 4500%, at least about 5000%, at least about 5500%, at least about 6000%, at least about 6500%, at least about 7000%, at least about 7500%, at least about 8000%, at least about 8500%, at least about 9000%, at least about 9500% or at least about 10000% more than control antibodies as described herein.

In another embodiment, the antibodies of the invention bind to multiple human cell types including but not limited to: A-549, Hey-A8, PC3, KC-231, Panc-02.03, SKMel.28, ACHN, 496, D-145, HT-29, SKOV-3, or SW-480, as described herein.

In another embodiment, the antibodies of the invention specifically bind the mouse cell line CT26 as described herein.

In another embodiment the antibodies of the invention specifically bind rat cell types including, but not limited to: F98, RG2, or YPEN as described herein.

In another embodiment, the antibodies of the invention specifically bind the murine cell line CT26 and the rat cell lines F98 and YPEN as described herein.

In another embodiment, the antibodies of the invention specifically bind the rat cell types F98 and YPEN at least about 2 fold, about 5 fold, about 10 fold, or about 100 fold greater than the rat cell type RG2 as described herein.

In another embodiment, the antibodies of the invention specifically bind the murine cell type CT26 at least about 2 fold, about 5 fold, about 10 fold, or about 100 fold greater than the murine cell types Balb/3T3 or NIH3T3 as described herein.

In another embodiment, the antibodies of the invention stimulate EphA2 phosphorylation when applied to HUVEC cells as described herein.

In another embodiment, the antibodies of the invention stimulate EphA2 phosphorylation in an HUVEC cell assay at least about 10%, at least about 20%, at least about 30%, at least about 40%, at least about 50%, at least about 60%, at least about 70%, at least about 80%, or at least about 90%, at least about 100%, at least about 110%, at least about 130%, at least about 140%, at least about 150%, at least about 160%, or at least about 170% more than control antibodies as described herein.

In another embodiment, the antibodies of the invention stimulate EphA2 phosphorylation in the mouse cell lines including but not limited to CT26 and 4T1 at least about 10%, at least about 20%, at least about 30%, at least about 40%, at least about 50%, at least about 60%, at least about 70%, at least about 80%, or at least about 90%, at least about 100%, at least about 110%, at least about 130%, at least about 140%, at least about 150%, at least about 160%, or at least about 170% more than control antibodies as described herein.

In another embodiment, the antibodies of the invention stimulate EphA2 phosphorylation in the rat cell line F98 at least about 10%, at least about 20%, at least about 30%, at least about 40%, at least about 50%, at least about 60%, at least about 70%, at least about 80%, or at least about 90%, at least about 100%, at least about 110%, at least about 130%, at least about 140%, at least about 150%, at least about 160%, or at least about 170% more than control antibodies as described herein.

In another embodiment, the antibodies of the invention stimulate EphA2 phosphorylation in the human cell lines including but not limited to PC3 and ES2 at least about 10%, at least about 20%, at least about 30%, at least about 40%, at least about 50%, at least about 60%, at least about 70%, at least about 80%, or at least about 90%, at least about 100%, at least about 110%, at least about 130%, at least about 140%, at least about 150%, at least about 160%, or at least about 170% more than control antibodies as described herein.

In another embodiment, the antibodies of the invention specifically bind the murine Eph protein families including but not limited to mEphA and mEphB.

In another embodiment, the antibodies of the invention specifically bind to the murine Eph family members including but not limited to: mEphA2 and mEphB2.

In another embodiment, the antibodies of the invention may exhibit an IC50 dose at least about 1 fold, at least about 5 fold, at least about 10 fold, at least about 25 fold, at least about 100 fold, or at least about 500 fold, less than the in vitro IC50 as described herein.

In another embodiment, the antibodies of the invention may exhibit an IC50 at least about 2 fold, 5 fold, 10 fold, or 100 fold lower for the PC3 cell line as compared to the KC231 cell line as described herein.

In another embodiment, the antibodies of the invention may inhibit tumor growth by at least about 10%, at least about 20%, at least about 30%, at least about 40%, at least about 50%, at least about 60%, at least about 70%, at least about 80%, or at least about 90%, at least about 100%, at least about 110%, at least about 130%, at least about 140%, at least about 150%, at least about 160%, or at least about 170% as compared to control antibodies in a mouse xenograft model described herein.

In another embodiment, the antibodies of the invention may promote tumor regression by at least about 10%, at least about 20%, at least about 30%, at least about 40%, at least about 50%, at least about 60%, at least about 70%, at least about 80%, or at least about 90%, at least about 100%, at least about 110%, at least about 130%, at least about 140%, at least about 150%, at least about 160%, or at least about 170% as compared to control antibodies in a mouse xenograft model described herein.

In another embodiment, the antibodies of the invention may inhibit tumor metastasis by at least about 10%, at least about 20%, at least about 30%, at least about 40%, at least about 50%, at least about 60%, at least about 70%, at least about 80%, or at least about 90%, at least about 100%, at least about 110%, at least about 130%, at least about 140%, at least about 150%, at least about 160%, or at least about 170% as compared to control antibodies in a mouse xenograft model described herein.

In another embodiment, the antibodies of the invention may inhibit tumor angiogenesis by at least about 10%, at least about 20%, at least about 30%, at least about 40%, at least about 50%, at least about 60%, at least about 70%, at least about 80%, or at least about 90%, at least about 100%, at least about 110%, at least about 130%, at least about 140%, at least about 150%, at least about 160%, or at least about 170% as compared to control antibodies in a mouse xenograft model described herein.

In another embodiment, the antibodies of the invention may preferentially bind the human cell lines including but not limited to A-549, Hey-A8, PC3, KC-231, Panc-02.03 by at least about 2 fold, 5 fold, 10 fold, or 100 fold over the human cell lines including but not limited to SKMel.28, ACHN, 496, D-145, HT-29, SKOV-3, or SW-480, as described herein.

### Methods of Producing Antibodies

The antibodies or fragments thereof can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2^{nd} ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with EphA2 or EphA4 (either the full length protein or a domain thereof, e.g., the extracellular domain or the ligand binding domain) and once an immune response is detected, e.g., antibodies specific for EphA2 or EphA4 are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with EphA2 or EphA4 or fragment thereof with myeloma cells and then screening the hybridomas resulting from the fusion for hybridoma clones that secrete an antibody able to bind EphA2 or EphA4.

Antibody fragments which recognize specific EphA2 or EphA4 epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to the EphA2 epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177; Kettleborough et al., 1994, Eur. J. Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

Phage may be screened for EphA2 binding, particularly to the extracellular domain of EphA2 or EphA4. Agonizing EphA2 or EphA4 activity (e.g., increasing EphA2 or EphA4 phosphorylation, reducing EphA2 or EphA4 levels) or cancer cell phenotype inhibiting activity (e.g., reducing colony formation in soft agar or tubular network formation in a three-dimensional basement membrane or extracellular matrix preparation, such as MATRIGEL™) or preferentially binding to an EphA2 or EphA4 epitope exposed on cancer cells but not non-cancer cells (e.g., binding poorly to EphA2 or EphA4 that is bound to ligand in cell-cell contacts while binding well to EphA2 or EphA4 that is not bound to ligand or in cell-cell contacts) may also be screened.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12:864; Sawai et al., 1995, AJRI 34:26; and Better et al., 1988, Science 240:1041 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Fremont, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, PNAS 91:969), and chain shuffling (U.S. Pat. No. 5,565,332). In one embodiment, a chimeric antibody of the invention specifically binds EphA2 and comprises one, two, or three VL CDRs having an amino acid sequence of any of the VL CDRs of EA2-5, Eph099B-102.147, Eph099B-208.261, Eph099B-210.248, Eph099B-233.152 within human framework regions.

In another embodiment, a chimeric antibody of the invention specifically binds EphA4 and comprises one, two, or three VL CDRs having an amino acid sequence of any of the VL CDRs of EA44 (as disclosed in U.S. Non-Provisional application Ser. No. 10/863,729, filed Jun. 7, 2004) within human framework regions. In another embodiment, a chimeric antibody of the invention specifically binds EphA2 and comprises one, two, or three VH CDRs having an amino acid sequence of any of the VH CDRs of EA2, EA5, 12G3H11, B233, B208, B210, G5, 10C12, 4H5, 10G9, 3F2, 1C1, 1F12, 1H3, 1D3, 2B12, or 5A8 within human framework regions.

In another embodiment, a chimeric antibody of the invention specifically binds EphA4 and comprises one, two, or three VH CDRs having an amino acid sequence of any of the VH CDRs of EA44 (as disclosed in U.S. Non-Provisional application Ser. No. 10/863,729, filed Jun. 7, 2004) within human framework regions. In another embodiment, a chimeric antibody of the invention specifically binds EphA2 and comprises one, two, or three VL CDRs having an amino acid sequence of any of the VL CDRs of EA2, EA5, 12G3H11, B233, B208, B210, G5, 10C12, 4H5, 10G9, 3F2, 1C1, 1F12, 1H3, 1D3, 2B12, or 5A8 and further comprises one, two, or three VH CDRs having an amino acid sequence of any of the VH CDRs of EA2, EA5, 12G3H11, B233, B208, B210, G5, 10C12, 4H5, 10G9, 3F2, 1C1, 1F12, 1H3, 1D3, 2B12, or 5A8 within human framework regions. In another embodiment, a chimeric antibody of the invention specifically binds EphA4 and comprises one, two, or three VL CDRs having an amino acid sequence of any of the VL CDRs of EA44 and further comprises one, two, or three VH CDRs having an amino acid sequence of any of the VH CDRs of EA44 within human framework regions.

In a further embodiment, a chimeric antibody of the invention specifically binds EphA2 and comprises three VL CDRs having an amino acid sequence of any of the VL CDRs of EA2, EA5, 12G3H11, B233, B208, B210, G5, 10C12, 4H5, 10G9, 3F2, 1C1, 1F12, 1H3, 1D3, 2B12, or 5A8 and three VH CDRs having an amino acid sequence of any of the VH CDRs of EA2, EA5, 12G3H11, B233, B208, B210, G5, 10C12, 4H5, 10G9, 3F2, 1C1, 1F12, 1H3, 1D3, 2B12, or 5A8 within human framework regions.

Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann et al., 1988, Nature 332:323, which are incorporated herein by reference in their entireties.).

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%.

Humanized antibodies can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55 (23 Supp):5973s-5977s, Couto et al., 1995, Cancer Res. 55:1717-22, Sandhu, 1994, Gene 150:409-10, Pedersen et al., 1994, J. Mol. Biol. 235:959-73, Jones et al., 1986, Nature 321:522-525, Riechmann et al., 1988, Nature 332:323, and Presta, 1992, Curr. Op. Struct. Biol. 2:593-596. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.).

Further, the antibodies of the invention can, in turn, be utilized to generate anti-idiotype antibodies using techniques well known to those skilled in the art. (See, e.g., Greenspan & Bona, 1989, FASEB J. 7:437-444; and Nissinoff, 1991, J. Immunol. 147:2429-2438). The invention provides methods employing the use of polynucleotides comprising a nucleotide sequence encoding an antibody of the invention or a fragment thereof.

### Polynucleotides Encoding an Antibody

The methods of the invention also encompass polynucleotides that hybridize under high stringency, intermediate or lower stringency hybridization conditions, e.g., as defined supra, to polynucleotides that encode an antibody of the invention.

The polynucleotides may be obtained, and the nucleotide sequence of the polynucleotides determined, by any method known in the art. Since the amino acid sequences of the antibodies are known, nucleotide sequences encoding these antibodies can be determined using methods well known in the art, i.e., nucleotide codons known to encode particular amino acids are assembled in such a way to generate a nucleic acid that encodes the antibody or fragment thereof of the invention. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

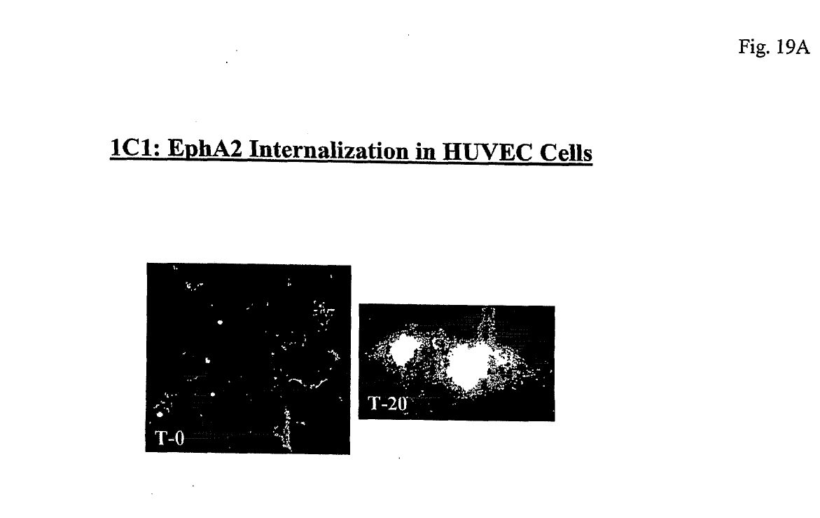

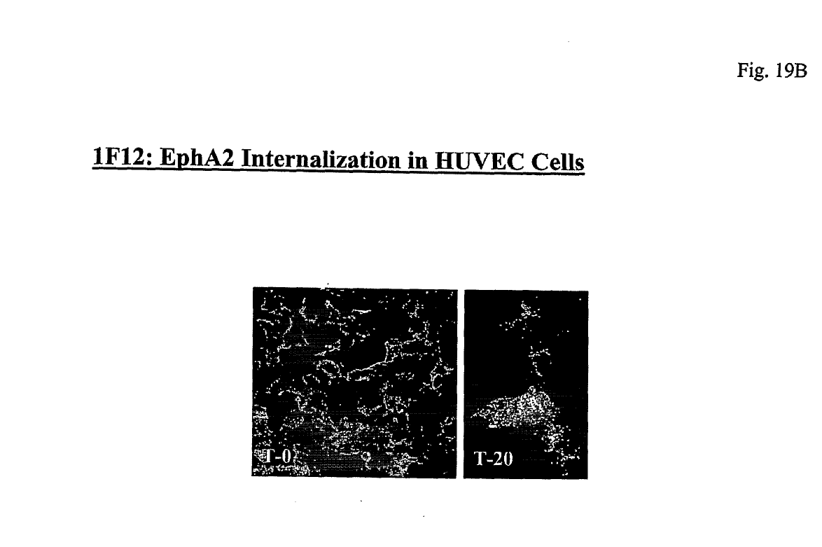

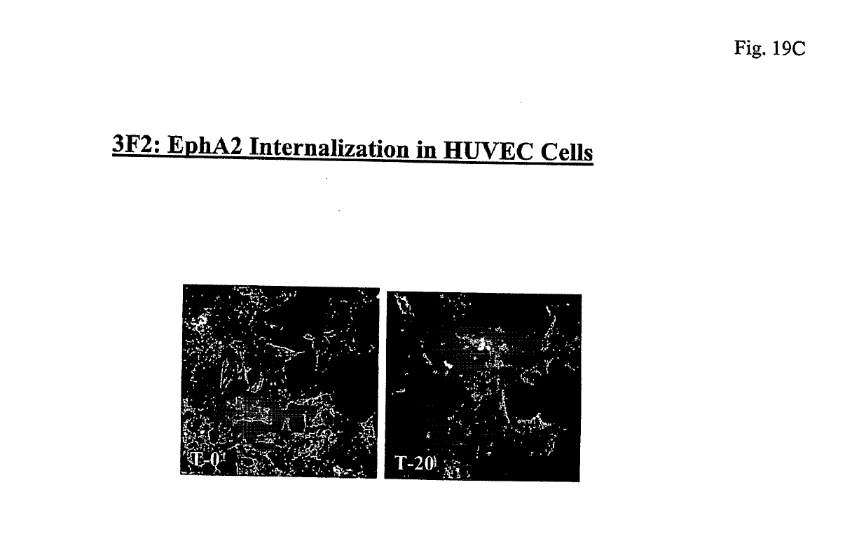

Alternatively, a polynucleotide encoding an antibody may be generated from nucleic acid from a suitable source. If a clone containing a nucleic acid encoding a particular antibody is not available, but the sequence of the antibody is known (e.g., see FIG. 19), a nucleic acid encoding the immunoglobulin may be chemically synthesized or obtained from a suitable source (e.g., an antibody cDNA library, or a cDNA library generated from, or nucleic acid, preferably poly A+RNA, isolated from, any tissue or cells expressing the antibody, such as hybridoma cells selected to express an antibody of the invention, e.g., clones deposited in the ATCC as PTA-4572, PTA-4573, PTA-4574, PTA-4380, PTA-4381) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to EphA2 or EphA4. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibodies lacking one or more intrachain disulfide bonds. Other alterations to the polynucleotide are encompassed by the present invention and within the skill of the art.

### Recombinant Expression of an Antibody

Recombinant expression of an antibody of the invention, derivative, analog or fragment thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody or a heavy or light chain of an antibody, or portion thereof (preferably, but not necessarily, containing the heavy or light chain variable domain), of the invention has been obtained, the vector for the production of the antibody may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination.

The invention, thus, provides replicable vectors comprising a nucleotide sequence encoding an antibody of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In certain embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibodies of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody, are used for the expression of a recombinant antibody.

For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, BioTechnology 8:2). In a specific embodiment, the expression of nucleotide sequences encoding antibodies or fragments thereof which specifically bind to EphA2 or EphA4 and agonize EphA2 or EphA4, inhibit a cancer cell phenotype, preferentially bind epitopes on EphA2 or EphA4 that are selectively exposed or increased on cancer cells but not non-cancer cells and/or have a K_{off} less than 3×10⁻³ s⁻¹ is regulated by a constitutive promoter, inducible promoter or tissue specific promoter.

In bacterial systems, a number of expression vectors may be advantageously selected depending upon the use intended for the antibody being expressed. For example, when a large quantity of such a protein is to be produced, for the generation of pharmaceutical compositions of an antibody, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. PGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the □luorourac gene) of the virus and placed under control of an AcNPV promoter (for example the □luorourac promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody in infected hosts (e.g., see Logan & Shenk, 1984, PNAS 8 1:6355-6359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, BT483, Hs578T, HTB2, BT20, NS1 and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody. Such engineered cell lines may be particularly useful in screening and evaluation of compositions that interact directly or indirectly with the antibody.

A number of selection systems may be used, including but not limited to, the herpes simplex virus thymidine kinase (Wigler et al., 1977, Cell 11:223), glutamine synthetase, hypoxanthine guanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, gs-, hgprt- or aprt-cells, respectively. Also, antimetabolite resistance can be used as the basis of selection for the following genes: dhfr, which confers resistance to methotrexate (Wigler et al., 1980, PNAS 77:357; O'Hare et al., 1981, PNAS 78:1527); gpt, which confers resistance to mycophenolic acid (Mulligan & Berg, 1981, PNAS 78:2072); neo, which confers resistance to the aminoglycoside G-418 (Wu and Wu, 1991, Biotherapy 3:87; Tolstoshev, 1993, Ann. Rev. Pharmacol. Toxicol. 32:573; Mulligan, 1993, Science 260:926; and Morgan and Anderson, 1993, Ann. Rev. Biochem. 62: 191; May, 1993, TIB TECH 11:155-); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, PNAS 77:2197). The coding sequences for the heavy and light chains may comprise cDNA or genomic DNA.

Once an antibody of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

### Prophylactic/Therapeutic Methods

The present invention encompasses methods for treating, preventing, or managing a disease or disorder associated with overexpression of EphA2 or EphA4 and/or a cell hyperproliferative disorder, particularly cancer, in a subject comprising administering an effective amount of a composition that can target cells expressing EphA2 or EphA4, and inhibiting the EphA2 or EphA4 expression or function, and/or having therapeutic or prophylactic effects on the hyperproliferative cell disease. In one embodiment, the method of the invention comprises administering to a subject a composition comprising an EphA2 or EphA4 targeting moiety attached to a therapeutic or prophylactic agent against the hyperproliferative cell disease. In another embodiment, the method of the invention comprises administering to a subject a composition comprising a nucleic acid comprising a nucleotide sequence encoding an EphA2 or EphA4 targeting moiety and a nucleotide sequence encoding a therapeutic or prophylactic agent against the hyperproliferative disease. In another embodiment, the method of the invention comprises administering to a subject a composition comprising an EphA2 or EphA4 targeting moiety and a nucleic acid comprising a nucleotide sequence encoding a therapeutic or prophylactic agent against the hyperproliferative disease, wherein the targeting moiety is associated with the nucleic acid either directly or through a delivery vector for delivery to cells expressing EphA2 or EphA4. In specific embodiments, an EphA2 or EphA4 targeting moiety also inhibits EphA2 or EphA4 expression or activity.

The present invention encompasses methods for treating, preventing, or managing a disease or disorder associated with overexpression of EphA2 or EphA4 and/or a cell hyperproliferative disorder, preferably cancer, in a subject comprising administering one or more ADCs that target EphA2 or EphA4 and/or inhibit EphA2 or EphA4 expression or activity, wherein said ADCs comprise EphA2 or EphA4 agonistic antibodies, EphA2 or EphA4 intrabodies, or EphA2 or EphA4 cancer cell phenotype inhibiting antibodies or exposed EphA2 or EphA4 epitope antibodies or EphA2 or EphA4 antibodies that bind EphA2 or EphA4 with a K_{off} less than 3×10⁻¹s⁻¹, preferably one or more monoclonal EphA2 or EphA4 agonistic antibodies, EphA2 or EphA4 intrabodies, BiTE molecules, or EphA2 or EphA4 cancer cell phenotype inhibiting antibodies or exposed EphA2 or EphA4 epitope antibodies or EphA2 or EphA4 antibodies that bind EphA2 or EphA4 with a K_{off} less than 3×10⁻¹s⁻¹. In a specific embodiment, the disorder to be treated, prevented, or managed is malignant cancer. In another specific embodiment, the disorder to be treated, prevented, or managed is a pre-cancerous condition associated with cells that overexpress EphA2 or EphA4. In more specific embodiments, the pre-cancerous condition is high-grade prostatic intraepithelial neoplasia (PIN), fibroadenoma of the breast, fibrocystic disease, or compound nevi.

In one embodiment, the compositions of the invention can be administered in combination with one or more other therapeutic agents useful in the treatment, prevention or management of diseases or disorders associated with EphA2 or EphA4 overexpression, hyperproliferative disorders, and/or cancer. In certain embodiments, one or more compositions of the invention are administered to a mammal, preferably a human, concurrently with one or more other therapeutic agents useful for the treatment of cancer. The term “concurrently” is not limited to the administration of prophylactic or therapeutic agents at exactly the same time, but rather it is meant that the compositions of the invention and the other agent are administered to a subject in a sequence and within a time interval such that the compositions of the invention can act together with the other agent to provide an increased benefit than if they were administered otherwise. For example, each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route. In other embodiments, the compositions of the invention are administered before, concurrently to, or after surgery. Preferably the surgery completely removes localized tumors or reduces the size of large tumors. Surgery can also be done as a preventive measure or to relieve pain.

In further embodiments, the compositions of the invention comprise one or more EphA2 antibodies consisting of EA2, EA5, 12G3H11, B233, B208, B210, G5, 10C12, 4H5, 10G9, 3F2, 1C1, 1F12, 1H3, 1D3, 2B12, or 5A8, or any of the antibodies listed in Table 2 or 3, or FIGS. 1-59, wherein said antibodies are used as EphA2-targeting moieties or agents against a hyperproliferative cell disease. In one embodiment, the compositions of the invention comprise antibodies consisting of EA2, EA5, B233, B208, B210, G5, 10C12, 4H5, 10G9, 3F2, 1C1, 1F12, 1H3, 1D3, 2B12, 5A8, or any of the antibodies listed in Table 2 or 3, or FIGS. 1-59 that have been humanized. In other embodiments, variants of EA2, EA5, B233, B208, B210, G5, 10C12, 4H5, 10G9, 3F2, 1C1, 1F12, 1H3, 1D3, 2B12, 5A8, or any of the antibodies listed in Tables 1 or 2, e.g., with one or more amino acid substitutions, particularly in the variable domain, are provided that have increased activity, binding ability, etc., as compared to EA2, EA5, B233, B208, B210, G5, 10C12, 4H5, 10G9, 3F2, 1C1, 1F12, 1H3, 1D3, 2B12, 5A8, or any of the antibodies listed in Table 2 or 3, or FIGS. 1-59.

In even further embodiments, the compositions of the invention comprise one or more EphA2 antibodies (as disclosed, for example, in U.S. Non-Provisional application Ser. Nos. 10/994,129, filed Nov. 19, 2004, 10/436,782, filed May 12, 2003, and U.S. Provisional Application Ser. Nos. 60/583,184, filed Jun. 25, 2004, 60/624,153, filed Nov. 2, 2004, 60/601,634, filed Aug. 16, 2004, 60/608,852, filed Sep. 13, 2004, all of which are hereby incorporated by reference herein in their entirety), wherein said antibodies are used as EphA2 targeting moieties or agents against a hyperproliferative cell disease.

In yet further embodiments, the compositions of the invention comprise one or more EphA4 antibodies consisting of EA44 (as disclosed, for example, in U.S. Non-Provisional application Ser. No. 10/863,729, filed Jun. 7, 2004), wherein said antibodies are used as EphA4 targeting moieties or agents against a hyperproliferative cell disease. In a further embodiment, the compositions of the invention comprise antibodies consisting of EA44 that have been humanized. In other embodiments, variants of EA44, e.g., with one or more amino acid substitutions, particularly in the variable domain, are provided that have increased activity, binding ability, etc., as compared to EA44.

### Patient Population

The invention provides methods for treating, preventing, and managing a disease or disorder associated with EphA2 or EphA4 overexpression and/or hyperproliferative cell disease, particularly cancer, by administrating to a subject in need thereof a therapeutically or prophylactically effective amount of one or more compositions of the invention. In another embodiment, the compositions of the invention can be administered in combination with one or more other therapeutic agents. The subject is preferably a mammal such as non-primate (e.g., cows, pigs, horses, cats, dogs, rats, etc.) and a primate (e.g., monkey, such as a cynomolgous monkey and a human). In another embodiment, the subject is a human.

Specific examples of cancers that can be treated by the methods encompassed by the invention include, but are not limited to, cancers that overexpress EphA2 or EphA4. In a further embodiment, the cancer is of an epithelial origin. Examples of such cancers are cancer of the lung, colon, prostate, breast, and skin. Other cancers include cancer of the bladder and pancreas and renal cell carcinoma and melanoma. Additional cancers are listed by example and not by limitation herein below. In particular embodiments, methods of the invention can be used to treat and/or prevent metastasis from primary tumors.

The methods and compositions of the invention comprise the administration of one or more compositions of the invention to subjects/patients suffering from or expected to suffer from cancer, e.g., have a genetic predisposition for a particular type of cancer, have been exposed to a carcinogen, or are in remission from a particular cancer. As used herein, “cancer” refers to primary or metastatic cancers. Such patients may or may not have been previously treated for cancer. The methods and compositions of the invention may be used as a first line or second line cancer treatment. Included in the invention is also the treatment of patients undergoing other cancer therapies and the methods and compositions of the invention can be used before any adverse effects or intolerance of these other cancer therapies occurs. The invention also encompasses methods for administering one or more compositions of the invention to treat or ameliorate symptoms in refractory patients. In a certain embodiment, that a cancer is refractory to a therapy means that at least some significant portion of the cancer cells are not killed or their cell division arrested. The determination of whether the cancer cells are refractory can be made either in vivo or in vitro by any method known in the art for assaying the effectiveness of treatment on cancer cells, using the art-accepted meanings of “refractory” in such a context. In various embodiments, a cancer is refractory where the number of cancer cells has not been significantly reduced, or has increased. The invention also encompasses methods for administering one or more EphA2 or EphA4 ADCs (use as a EphA2 or EphA4-targeting moiety and/or an agent against cancer) to prevent the onset or recurrence of cancer in patients predisposed to having cancer. Preferably, the antibody portion of the ADC is one or more of EA2, EA5, B233, B208, B210, G5, 10C12, 4H5, 10G9, 3F2, 1C1, 1F12, 1H3, 1D3, 2B12, 5A8, or any of the antibodies listed in Table 2 or 3, or FIGS. 1-59. In another embodiment, an EphA4 agonistic antibody for use in the ADC compositions and methods of the invention is EA44.

In particular embodiments, the compositions of the invention are administered to reverse resistance or reduced sensitivity of cancer cells to certain hormonal, radiation and chemotherapeutic agents thereby resensitizing the cancer cells to one or more of these agents, which can then be administered (or continue to be administered) to treat or manage cancer, including to prevent metastasis. In a specific embodiment, compositions of the invention are administered to patients with increased levels of the cytokine IL-6, which has been associated with the development of cancer cell resistance to different treatment regimens, such as chemotherapy and hormonal therapy. In another specific embodiment, compositions of the invention are administered to patients suffering from breast cancer that have a decreased responsiveness or are refractory to tamoxifen treatment. In another specific embodiment, compositions of the invention are administered to patients with increased levels of the cytokine IL-6, which has been associated with the development of cancer cell resistance to different treatment regimens, such as chemotherapy and hormonal therapy.

In alternate embodiments, the invention provides methods for treating patients' cancer by administering one or more compositions of the invention in combination with any other treatment or to patients who have proven refractory to other treatments but are no longer on these treatments. Preferably, one or more of EA2, EA5, B233, B208, B210, G5, 10C12, 4H5, 10G9, 3F2, 1C1, 1F12, 1H3, 1D3, 2B12, 5A8, any of the antibodies listed in Table 2 or 3, or EA44 are used in accordance with the present invention as an anti-EphA2 or anti-EphA4 ADC. In certain embodiments, the patients being treated by the methods of the invention are patients already being treated with chemotherapy, radiation therapy, hormonal therapy, or biological therapy/immunotherapy. Among these patients are refractory patients and those with cancer despite treatment with existing cancer therapies. In other embodiments, the patients have been treated and have no disease activity and one or more compositions of the invention are administered to prevent the recurrence of cancer.

In certain embodiments, the existing treatment is chemotherapy. In particular embodiments, the existing treatment includes administration of chemotherapies including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, etc. Among these patients are patients treated with radiation therapy, hormonal therapy and/or biological therapy/immunotherapy. Also among these patients are those who have undergone surgery for the treatment of cancer.

Alternatively, the invention also encompasses methods for treating patients undergoing or having undergone radiation therapy. Among these are patients being treated or previously treated with chemotherapy, hormonal therapy and/or biological therapy/immunotherapy. Also among these patients are those who have undergone surgery for the treatment of cancer.

In other embodiments, the invention encompasses methods for treating patients undergoing or having undergone hormonal therapy and/or biological therapy/immunotherapy. Among these are patients being treated or having been treated with chemotherapy and/or radiation therapy. Also among these patients are those who have undergone surgery for the treatment of cancer.

Additionally, the invention also provides methods of treatment of cancer as an alternative to chemotherapy, radiation therapy, hormonal therapy, and/or biological therapy/immunotherapy where the therapy has proven or may prove too toxic, i.e., results in unacceptable or unbearable side effects, for the subject being treated. The subject being treated with the methods of the invention may, optionally, be treated with other cancer treatments such as surgery, chemotherapy, radiation therapy, hormonal therapy or biological therapy, depending on which treatment was found to be unacceptable or unbearable.

In other embodiments, the invention provides administration of one or more compositions of the invention without any other cancer therapies for the treatment of cancer, but who have proved refractory to such treatments. In specific embodiments, patients refractory to other cancer therapies are administered one or more compositions of the invention in the absence of cancer therapies.

In other embodiments, patients with a pre-cancerous condition associated with cells that overexpress EphA2 or EphA4 can be administered compositions of the invention to treat the disorder and decrease the likelihood that it will progress to malignant cancer. In a specific embodiments, the pre-cancerous condition is high-grade prostatic intraepithelial neoplasia (PIN), fibroadenoma of the breast, fibrocystic disease, or compound nevi.

In yet other embodiments, the invention provides methods of treating, preventing and managing non-cancer hyperproliferative cell disorders, particularly those associated with overexpression of EphA2 or EphA4, including but not limited to, asthma, chromic obstructive pulmonary disorder (COPD), restenosis (smooth muscle and/or endothelial), psoriasis, etc. These methods include methods analogous to those described above for treating, preventing and managing cancer, for example, by administering the compositions of the invention, as well as combination therapy, administration to patients refractory to particular treatments, etc.

### Cancers

Cancers and related disorders that can be treated, prevented, or managed by methods and compositions of the present invention include but are not limited to cancers of an epithelial cell origin. Examples of such cancers include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to □luoroura, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the skin, lung, colon, breast, prostate, bladder, kidney, pancreas, ovary, or uterus. In other specific embodiments, sarcoma, melanoma, or leukemia is treated or prevented.

In some embodiments, the cancer is malignant and overexpresses EphA2 or EphA4. In other embodiments, the disorder to be treated is a pre-cancerous condition associated with cells that overexpress EphA2 or EphA4. In a specific embodiments, the pre-cancerous condition is high-grade prostatic intraepithelial neoplasia (PIN), fibroadenoma of the breast, fibrocystic disease, or compound nevi.

In other embodiments, the methods and compositions of the invention are used for the treatment and/or prevention of breast, colon, ovarian, lung, and prostate cancers and melanoma and are provided below by example rather than by limitation.

### Treatment of Breast Cancer

In specific embodiments, patients with breast cancer are administered an effective amount of one or more compositions of the invention. In one embodiment, the present invention provides a method of preventing, treating or managing a breast cancer comprising administering to the patient (a) an anti-EphA2 or anti-EphA4 ADC of the present invention, and (b) a pharmaceutical acceptable carrier. In another embodiment, the compositions of the invention can be administered in combination with an effective amount of one or more other agents useful for breast cancer therapy. Agents useful for breast cancer therapy include, but are not limited to: doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), herceptin, tamoxifen, the combination of tamoxifen and cytotoxic chemotherapy, taxanes (such as docetaxel and paclitaxel). In a further embodiment, compositions of the invention may comprise or used in combination with taxanes plus standard doxorubicin and cyclophosphamide for adjuvant treatment of node-positive, localized breast cancer.

In a specific embodiment, patients with pre-cancerous fibroadenoma of the breast or fibrocystic disease are administered a composition of the invention to treat the disorder and decrease the likelihood that it will progress to malignant breast cancer. In another specific embodiment, patients refractory to treatment, particularly hormonal therapy, more particularly tamoxifen therapy, are administered a composition of the invention to treat the cancer and/or render the patient non-refractory or responsive.

### Treatment of Colon Cancer

In specific embodiments, patients with colon cancer are administered an effective amount of one or more compositions of the invention. In another embodiment, the compositions of the invention comprise or used in combination with an effective amount of one or more other agents useful for colon cancer therapy, including but not limited to: the combination of 5-FU and leucovorin, the combination of 5-FU and levamisole, irinotecan (CPT-11) or the combination of irinotecan, 5-FU and leucovorin (IFL).

### Treatment of Prostate Cancer

In specific embodiments, patients with prostate cancer are administered an effective amount of one or more compositions of the invention. In another embodiment, the compositions of the invention comprise or used in combination with an effective amount of one or more other agents useful for prostate cancer therapy, including but not limited to: external-beam radiation therapy, interstitial implantation of radioisotopes (i.e., ¹²⁵I, palladium, iridium), leuprolide or other LHRH agonists, non-steroidal antiandrogens (flutamide, nilutamide, bicalutamide), steroidal antiandrogens (cyproterone acetate), the combination of leuprolide and flutamide, estrogens such as DES, chlorotrianisene, ethinyl estradiol, conjugated estrogens U.S.P., DES-diphosphate, radioisotopes, such as strontium-89, the combination of external-beam radiation therapy and strontium-89, second-line hormonal therapies such as aminoglutethimide, hydrocortisone, flutamide withdrawal, progesterone, and ketoconazole, low-dose prednisone, or other chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel.

In a specific embodiment, patients with pre-cancerous high-grade prostatic intraepithelial neoplasia (PIN) are administered a composition of the invention to treat the disorder and decrease the likelihood that it will progress to malignant prostate cancer.

### Treatment of Melanoma

In specific embodiments, patients with melanoma are administered an effective amount of one or more compositions of the invention. In another embodiment, the compositions of the invention comprise or used in combination with an effective amount of one or more other agents useful for melanoma cancer therapy, including but not limited to: dacarbazine (DTIC), nitrosoureas such as carmustine (BCNU) and lomustine (CCNU), agents with modest single agent activity including vinca alkaloids, platinum compounds, and taxanes, the Dartmouth regimen (cisplatin, BCNU, and DTIC), interferon alpha (IFN-α), and interleukin-2 (IL-2). In a specific embodiment, an effective amount of one or more agonistic monoclonal antibodies of the invention can be administered in combination with isolated hyperthermic limb perfusion (ILP) with melphalan (L-PAM), with or without tumor necrosis factor-alpha (TNF-alpha) to patients with multiple brain metastases, bone metastases, and spinal cord compression to achieve symptom relief and some shrinkage of the tumor with radiation therapy.

In a specific embodiment, patients with pre-cancerous compound nevi are administered a composition of the invention to treat the disorder and decrease the likelihood that it will progress to malignant melanoma.

### Treatment of Ovarian Cancer

In specific embodiments, patients with ovarian cancer are administered an effective amount of one or more compositions of the invention. In another embodiment, the compositions of the invention comprise or used in combination with an effective amount of one or more other agents useful for ovarian cancer therapy including but not limited to: intraperitoneal radiation therapy, such as P³² therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. It is contemplated that an effective amount of one or more compositions of the invention are administered in combination with the administration Taxol for patients with platinum-refractory disease. Included is the treatment of patients with refractory ovarian cancer including administration of: ifosfamide in patients with disease that is platinum-refractory, hexamethylmelamine (HMM) as salvage chemotherapy after failure of cisplatin-based combination regimens, and tamoxifen in patients with detectable levels of cytoplasmic estrogen receptor on their tumors.

### Treatment of Lung Cancers

In specific embodiments, patients with small lung cell cancer are administered an effective amount of one or more compositions of the invention. In another embodiment, the compositions of the invention comprise or used in combination with an effective amount of one or more other agents useful for lung cancer therapy, including but not limited to: thoracic radiation therapy, cisplatin, vincristine, doxorubicin, and etoposide, alone or in combination, the combination of cyclophosphamide, doxorubicin, vincristine/etoposide, and cisplatin (CAV/EP), local palliation with endobronchial laser therapy, endobronchial stents, and/or brachytherapy.

In other specific embodiments, patients with non-small lung cell cancer are administered an effective amount of one or more compositions of the invention in combination with an effective amount of one or more other agents useful for lung cancer therapy including but not limited to: palliative radiation therapy, the combination of cisplatin, vinblastine and mitomycin, the combination of cisplatin and vinorelbine, paclitaxel, docetaxel or gemcitabine, the combination of carboplatin and paclitaxel, interstitial radiation therapy for endobronchial lesions or stereotactic radiosurgery.

### Other Prophylactic/Therapeutic Agents

In some embodiments, the present invention provides a method of preventing, treating or managing a hyperproliferative cell disease comprising administering to the patient (a) an anti-EphA2 or anti-EphA4 ADC of the present invention, and (b) a pharmaceutical acceptable carrier. In some embodiments, the present invention provides a method of preventing, treating or managing a hyperproliferative cell disease comprising administering one or more compositions of the invention in combination with the administration of one or more therapies such as, but not limited to, chemotherapies, radiation therapies, hormonal therapies, biological therapies/immunotherapies and/or surgery.

Prophylactic/therapeutic agents that can be used in accordance with the present invention include, but are not limited to, proteinaceous molecules, including, but not limited to, peptides, polypeptides, proteins, including post-translationally modified proteins, antibodies etc.; or small molecules (less than 1000 daltons), inorganic or organic compounds; or nucleic acid molecules including, but not limited to, double-stranded or single-stranded DNA, or double-stranded or single-stranded RNA, as well as triple helix nucleic acid molecules. Prophylactic/therapeutic agents can be derived from any known organism (including, but not limited to, animals, plants, bacteria, fungi, and protista, or viruses) or from a library of synthetic molecules.

In a specific embodiment, prophylactic/therapeutic agents that can be used in accordance with the present invention are inhibitors of kinases such as, but are not limited to, ABL, ACK, AFK, AKT (e.g., AKT-1, AKT-2, and AKT-3), ALK, AMP-PK, ATM, Aurora1, Aurora2, bARK1, bArk2, BLK, BMX, BTK, CAK, CaM kinase, CDC2, CDK, CK, COT, CTD, DNA-PK, EGF-R, ErbB-1, ErbB-2, ErbB-3, ErbB-4, ERK (e.g., ERK1, ERK2, ERK3, ERK4, ERK5, ERK6, ERK7), ERT-PK, FAK, FGR (e.g., FGF1R, FGF2R), FLT (e.g., FLT-1, FLT-2, FLT-3, FLT-4), FRK, FYN, GSK (e.g., GSK1, GSK2, GSK3-alpha, GSK3-beta, GSK4, GSK5), G-protein coupled receptor kinases (GRKs), HCK, HER2, HKII, JAK (e.g., JAK1, JAK2, JAK3, JAK4), JNK (e.g., JNK1, JNK2, JNK3), KDR, KIT, IGF-1 receptor, IKK-1, IKK-2, INSR (insulin receptor), IRAK1, IRAK2, IRK, ITK, LCK, LOK, LYN, MAPK, MAPKAPK-1, MAPKAPK-2, MEK, MET, MFPK, MHCK, MLCK, MLK3, NEU, NIK, PDGF receptor alpha, PDGF receptor beta, PHK, PI-3 kinase, PKA, PKB, PKC, PKG, PRK1, PYK2, p38 kinases, p135tyk2, p34cdc2, p42cdc2, p42mapk, p44 mpk, RAF, RET, RIP, RIP-2, RK, RON, RS kinase, SRC, SYK, S6K, TAK1, TEC, TIE1, TIE2, TRKA, TXK, TYK2, UL13, VEGFR1, VEGFR2, YES, YRK, ZAP-70, and all subtypes of these kinases (see e.g., Hardie and Hanks (1995) The Protein Kinase Facts Book, I and II, Academic Press, San Diego, Calif.). In further embodiments, one or more prophylactic/therapeutic agents that can be used in accordance with the present invention are inhibitors of Eph receptor kinases (e.g., EphA2, EphA4). In a specific embodiment, one or more prophylactic/therapeutic agents that can be used in accordance with the present invention are inhibitors of EphA2 or EphA4.

In another specific embodiment, one or more prophylactic/therapeutic agents that can be used in accordance with the present invention are angiogenesis inhibitors such as, but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVII fragment); fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-β); Vasculostatin; Vasostatin (calreticulin fragment); ZD6126; ZD6474; farnesyl transferase inhibitors (FTI); and bisphosphonates.

In another specific embodiment, one or more prophylactic/therapeutic agents that can be used in accordance with the present invention are anti-cancer agents such as, but are not limited to: acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decarbazine, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interleukin 2 (including recombinant interleukin 2, or rIL2), interferon alpha-2a, interferon alpha-2b, interferon alpha-n1, interferon alpha-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nitrosoureas, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride. Other anti-cancer drugs include, but are not limited to: 20-epi-1,25 dihydroxyvitamin D3,5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogens, antiestrogens, antineoplaston, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, dihydrotaxol, dioxamycin, diphenyl spiromustine, docetaxel, docosanol, dolasetron, doxifluridine, droloxifene, dronabinol, duocarmycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+ cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, taxol, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thioguanine, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, and zinostatin stimalamer. Preferred additional anti-cancer drugs are 5-fluorouracil and leucovorin.

In more particular embodiments, the present invention also comprises the administration of one or more compositions of the invention comprising or used in combination with one or more therapies such as, but are not limited to, anti-cancer agents such as those disclosed in Table 5, preferably for the treatment of breast, ovary, melanoma, prostate, colon and lung cancers as described above.

TABLE 5

| Therapeutic Agent | Administration | Dose | Intervals |
|---|---|---|---|
| doxorubicin | Intravenous | 60-75 mg/m² on Day 1 | 21 day intervals |
| hydrochloride | | | |
| (Adriamycin RDF ® | | | |
| and Adriamycin | | | |
| PFS ®) | | | |
| epirubicin | Intravenous | 100-120 mg/m² on Day 1 of | 3-4 week cycles |
| hydrochloride | | each cycle or divided equally | |
| (Ellence ™) | | and given on Days 1-8 of the | |
| | | cycle | |
| fluorousacil | Intravenous | How supplied: | |
| | | 5 ml and 10 ml vials | |
| | | (containing 250 and 500 mg | |
| | | fluorouracil respectively) | |
| docetaxel | Intravenous | 60-100 mg/m² over 1 hour | Once every 3 weeks |
| (Taxotere ®) | | | |
| paclitaxel | Intravenous | 175 mg/m² over 3 hours | Every 3 weeks for 4 courses |
| (Taxol ®) | | | (administered sequentially to |
| | | | doxorubicin-containing |
| | | | combination chemotherapy) |
| tamoxifen citrate | Oral | 20-40 mg | Daily |
| (Nolvadex ®) | (tablet) | Dosages greater than 20 mg | |
| | | should be given in divided | |
| | | doses (morning and evening) | |
| leucovorin calcium | Intravenous or | How supplied: | Dosage is unclear from text. |
| for injection | intramuscular | 350 mg vial | PDR 3610 |
| | injection | | |
| luprolide acetate | Single | 1 mg (0.2 ml or 20 unit mark) | Once a day |
| (Lupron ®) | subcutaneous | | |
| | injection | | |
| flutamide | Oral | 250 mg | 3 times a day at 8 hour |
| (Eulexin ®) | (capsule) | (capsules contain 125 mg | intervals (total daily dosage |
| | | flutamide each) | 750 mg) |
| nilutamide | Oral | 300 mg or 150 mg | 300 mg once a day for 30 |
| (Nilandron ®) | (tablet) | (tablets contain 50 or 150 mg | days followed by 150 mg |
| | | nilutamide each) | once a day |
| bicalutamide | Oral | 50 mg | Once a day |
| (Casodex ®) | (tablet) | (tablets contain 50 mg | |
| | | bicalutamide each) | |
| progesterone | Injection | USP in sesame oil 50 mg/ml | |
| ketoconazole | Cream | 2% cream applied once or | |
| (Nizoral ®) | | twice daily depending on | |
| | | symptoms | |
| prednisone | Oral | Initial dosage may vary from | |
| | (tablet) | 5 mg to 60 mg per day | |
| | | depending on the specific | |
| | | disease entity being treated. | |
| Estramustine | Oral | 14 mg/kg of body weight | Daily given in 3 or 4 divided |
| phosphate sodium | (capsule) | (i.e. one 140 mg capsule for | doses |
| (Emcyt ®) | | each 10 kg or 22 lb of body | |
| | | weight) | |
| etoposide or VP-16 | Intravenous | 5 ml of 20 mg/ml solution | |
| | | (100 mg) | |
| dacarbazine | Intravenous | 2-4.5 mg/kg | Once a day for 10 days. |
| (DTIC-Dome ®) | | | May be repeated at 4 week |
| | | | intervals |
| polifeprosan 20 with | wafer placed in | 8 wafers, each containing 7.7 | |
| carmustine implant | resection cavity | mg of carmustine, for a total | |
| (BCNU) (nitrosourea) | | of 61.6 mg, if size and shape | |
| (Gliadel ®) | | of resection cavity allows | |
| cisplatin | Injection | How supplied: | |
| | | solution of 1 mg/ml in multi- | |
| | | dose vials of 50 mL and | |
| | | 100 mL | |
| mitomycin | Injection | supplied in 5 mg and 20 mg | |
| | | vials (containing 5 mg and 20 | |
| | | mg mitomycin) | |
| gemcitabine HCl | Intravenous | For NSCLC-2 schedules | 4 week schedule- |
| (Gemzar ®) | | have been investigated and | Days 1, 8 and 15 of each 28- |
| | | the optimum schedule has not | day cycle. Cisplatin |
| | | been determined | intravenously at 100 mg/m² |
| | | 4 week schedule- | on day 1 after the infusion of |
| | | administration intravenously | Gemzar. |
| | | at 1000 mg/m² over 30 | 3 week schedule- |
| | | minutes on 3 week schedule- | Days 1 and 8 of each 21 day |
| | | Gemzar administered | cycle. Cisplatin at dosage of |
| | | intravenously at 1250 mg/m² | 100 mg/m² administered |
| | | over 30 minutes | intravenously after |
| | | | administration of Gemzar on |
| | | | day 1. |
| Carboplatin | Intravenous | Single agent therapy: | Every 4 weeks |
| (Paraplatin ®) | | 360 mg/m² I.V. on day 1 | |
| | | (infusion lasting 15 minutes | |
| | | or longer) | |
| | | Other dosage calculations: | |
| | | Combination therapy with | |
| | | cyclophosphamide, Dose | |
| | | adjustment recommendations, | |
| | | Formula dosing, etc. | |
| ifosamide | Intravenous | 1.2 g/m² daily | 5 consecutive days |
| (Ifex ®) | | | Repeat every 3 weeks or |
| | | | after recovery from |
| | | | hematologic toxicity |
| topotecan | Intravenous | 1.5 mg/m² by intravenous | 5 consecutive days, starting |
| hydrochloride | | infusion over 30 minutes | on day 1 of 21 day course |
| (Hycamtin ®) | | daily | |

The invention also encompasses administration of the compositions of the invention in combination with radiation therapy comprising the use of x-rays, gamma rays and other sources of radiation to destroy the cancer cells. In certain embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physician's Desk Reference (58^{th} ed., 2004).

### Formulations

Pharmaceutical compositions for use in accordance with the present invention may be formulated in conventional manner using one or more physiologically acceptable carriers or excipients. Thus, the compositions of the invention and their physiologically acceptable salts and solvates may be formulated for administration by inhalation or insufflation (either through the mouth or the nose) or oral, parenteral or mucosal (such as buccal, vaginal, rectal, sublingual) administration. In another embodiment, local or systemic parenteral administration is used.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the prophylactic or therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The prophylactic or therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The prophylactic or therapeutic agents may also be formulated in rectal compositions such as suppositories or retention enemas, e.g., containing conventional suppository bases such as cocoa butter or other glycerides.

In addition to the formulations described previously, the prophylactic or therapeutic agents may also be formulated as a depot preparation. Such long acting formulations may be administered by implantation (for example subcutaneously or intramuscularly) or by intramuscular injection. Thus, for example, the prophylactic or therapeutic agents may be formulated with suitable polymeric or hydrophobic materials (for example as an emulsion in an acceptable oil) or ion exchange resins, or as sparingly soluble derivatives, for example, as a sparingly soluble salt.

The invention also provides that a prophylactic or therapeutic agent is packaged in a hermetically sealed container such as an ampoule or sachette indicating the quantity. In one embodiment, the prophylactic or therapeutic agent is supplied as a dry sterilized lyophilized powder or water free concentrate in a hermetically sealed container and can be reconstituted, e.g., with water or saline to the appropriate concentration for administration to a subject.

In another embodiment of the invention, the formulation and administration of various chemotherapeutic, biological/immunotherapeutic and hormonal therapeutic agents are known in the art and often described in the Physicians Desk Reference, 58^{th} ed. (2004). For instance, in certain specific embodiments of the invention, the therapeutic agents of the invention can be formulated and supplied as provided in Table 5.

In other embodiments of the invention, radiation therapy agents such as radioactive isotopes can be given orally as liquids in capsules or as a drink. Radioactive isotopes can also be formulated for intravenous injections. The skilled oncologist can determine the preferred formulation and route of administration.

In certain embodiments the compositions of the invention, are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### Dosages and Frequency of Administration

The amount of a therapy (e.g., prophylactic or therapeutic agent) or a composition of the invention which will be effective in the prevention, treatment, management, and/or amelioration of a hyperproliferative disease or one or more symptoms thereof can be determined by standard clinical methods. The frequency and dosage will vary also according to factors specific for each patient depending on the specific therapies (e.g., the specific therapeutic or prophylactic agent or agents) administered, the severity of the disorder, disease, or condition, the route of administration, as well as age, body, weight, response, and the past medical history of the patient. For example, the dosage of a prophylactic or therapeutic agent or a composition of the invention which will be effective in the treatment, prevention, management, and/or amelioration of an hyperproliferative disease or one or more symptoms thereof can be determined by administering the composition to an animal model such as, e.g., the animal models disclosed herein or known in to those skilled in the art. In addition, in vitro assays may optionally be employed to help identify optimal dosage ranges. Suitable regimens can be selected by one skilled in the art by considering such factors and by following, for example, dosages are reported in literature and recommended in the Physician's Desk Reference (58^{th} ed., 2004).

In various embodiments, the therapies (e.g., prophylactic or therapeutic agents) are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In certain embodiments, two or more components are administered within the same patient visit.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physician's Desk Reference (58^{th} ed., 2004).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In a specific embodiment, the dosage of ADCs administered to prevent, treat, manage, and/or ameliorate a hyperproliferative disease or one or more symptoms thereof in a patient is 150 μg/kg or less, preferably 125 μg/kg or less, 100 μg/kg or less, 95 μg/kg or less, 90 μg/kg or less, 85 μg/kg or less, 80 g/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of the ADCs of the invention administered to prevent, treat, manage, and/or ameliorate a hyperproliferative disease, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In other embodiments, a subject is administered one or more doses of an effective amount of one or therapies (e.g., therapeutic or prophylactic agents) of the invention, wherein the dose of an effective amount achieves a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the therapies (e.g., therapeutic or prophylactic agents) of the invention. In yet other embodiments, a subject is administered a dose of an effective amount of one or ADCs of the invention to achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the ADCs and a subsequent dose of an effective amount of one or more ADCs of the invention is administered to maintain a serum titer of at least 0.1 μg/ml, 0.5 μg/ml, 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml. In accordance with these embodiments, a subject may be administered 1, 2, 3, 4, 5, 6, 7, 8, 9, 10, 11, 12 or more subsequent doses.

In a specific embodiment, the invention provides methods of preventing, treating, managing, or ameliorating a hyperproliferative disease or one or more symptoms thereof, said method comprising administering to a subject in need thereof a dose of at least 10 μg, preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more therapies (e.g., therapeutic or prophylactic agents), combination therapies, or compositions of the invention. In another embodiment, the invention provides a method of preventing, treating, managing, and/or ameliorating a hyperproliferative disease or one or more symptoms thereof, said methods comprising administering to a subject in need thereof a dose of at least 10 μg, preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more ADCs, combination therapies, or compositions of the invention once every 3 days, preferably, once every 4 days, once every 5 days, once every 6 days, once every 7 days, once every 8 days, once every 10 days, once every two weeks, once every three weeks, or once a month.

The present invention provides methods of preventing, treating, managing, or preventing a hyperproliferative disease or one or more symptoms thereof, said method comprising: (a) administering to a subject in need thereof one or more doses of a prophylactically or therapeutically effective amount of one or more ADCs, combination therapies, or compositions of the invention; and (b) monitoring the plasma level/concentration of the said administered ADCs in said subject after administration of a certain number of doses of the said therapies (e.g., therapeutic or prophylactic agents). Moreover, preferably, said certain number of doses is 1, 2, 3, 4, 5, 6, 7, 8, 9, 10, 11, or 12 doses of a prophylactically or therapeutically effective amount one or more ADCs, compositions, or combination therapies of the invention.

In a specific embodiment, the invention provides a method of preventing, treating, managing, and/or ameliorating a hyperproliferative disease or one or more symptoms thereof, said method comprising: (a) administering to a subject in need thereof a dose of at least 10 μg (preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more therapies (e.g., therapeutic or prophylactic agents) of the invention; and (b) administering one or more subsequent doses to said subject when the plasma level of the ADC administered in said subject is less than 0.1 μg/ml, preferably less than 0.25 μg/ml, less than 0.5 μg/ml, less than 0.75 μg/ml, or less than 1 μg/ml. In another embodiment, the invention provides a method of preventing, treating, managing, and/or ameliorating a hyperproliferative disease or one or more symptoms thereof, said method comprising: (a) administering to a subject in need thereof one or more doses of at least 10 μg (preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more ADCs of the invention; (b) monitoring the plasma level of the administered ADCs in said subject after the administration of a certain number of doses; and (c) administering a subsequent dose of ADCs of the invention when the plasma level of the administered ADC in said subject is less than 0.1 μg/ml, preferably less than 0.25 μg/ml, less than 0.5 μg/ml, less than 0.75 μg/ml, or less than 1 μg/ml. Preferably, said certain number of doses is 1, 2, 3, 4, 5, 6, 7, 8, 9, 10, 11, or 12 doses of an effective amount of one or more ADCs of the invention.

Therapies (e.g., prophylactic or therapeutic agents), other than the ADCs of the invention, which have been or are currently being used to prevent, treat, manage, and/or ameliorate a hyperproliferative disease or one or more symptoms thereof can be administered in combination with one or more ADCs according to the methods of the invention to treat, manage, prevent, and/or ameliorate a hyperproliferative disease or one or more symptoms thereof. Preferably, the dosages of prophylactic or therapeutic agents used in combination therapies of the invention are lower than those which have been or are currently being used to prevent, treat, manage, and/or ameliorate a hyperproliferative disease or one or more symptoms thereof. The recommended dosages of agents currently used for the prevention, treatment, management, or amelioration of a hyperproliferative disease or one or more symptoms thereof can be obtained from any reference in the art including, but not limited to, Hardman et al., eds., 2001, Goodman & Gilman's The Pharmacological Basis Of Basis Of Therapeutics, 10^{th} ed., Mc-Graw-Hill, New York; Physician's Desk Reference (PDR)58^{th} ed., 2004, Medical Economics Co., Inc., Montvale, N.J., which are incorporated herein by reference in its entirety.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In other embodiments, two or more therapies are administered within the same patient visit.

In certain embodiments, one or more ADCs of the invention and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same ADC of the invention may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than an ADC of the invention may be repeated and the administration may be separated by at least at least 1 day, 2 days, 3 days, 5 days, 10 days, 15 days, 30 days, 45 days, 2 months, 75 days, 3 months, or at least 6 months.

### EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### Example 1

### Generation and Expression of the Various Antibody Constructs

Six humanized monoclonal antibodies (G5, 10D3, 12G3, 1E11, 4C10, 4B11) and one human/mouse chimeric antibody (EA5) were generated against a common antigen, EphA2. All of these antibodies were poorly expressed in mammalian cells. One or more heavy chain substitutions at positions 40, 60 and/or 61 were generated in each of these antibodies to determine the effect on producibility by the presence of one or more preferred amino acid residues at these positions. Six of the humanized antibodies contained an Alanine at position H40, these antibodies were substituted with Alanine and Aspartate at positions H60 and H61 respectively. The chimeric antibody, EA5, against the same antigen did not contain any of the preferred amino acids at positions H40, H60 or H61. Two separate heavy chains were generated for EA5, one which contained substitutions at positions 60 and 61 and another which contained substitutions at positions H40, H60 and H61. The specific amino acid residues of the heavy chain that were modified (see FIG. 1B) are described below. In all cases substitutions resulting in one or more preferred heavy chain residues at positions 40, 60 and 61 resulted in improved producibility (see Table 6). Interestingly, in the case of EA5 which contained none of the preferred amino acids, the heavy chain A60/D61 combination by itself significantly increased production yields.

### Materials and Methods

Generation, Characterization and Cloning of Antigen Specific Antibodies: General methods for generating, screening, cloning and expressing antibodies are known to practitioners of the art. See, e.g., Current Protocols in Molecular Biology, F. M. Ausubel et al., ed., John Wiley & Sons (Chichester, England, 1998); Molecular Cloning: A Laboratory Manual, 3nd Edition, J. Sambrook et al., ed., Cold Spring Harbor Laboratory Press (Cold Spring Harbor, N.Y., 2001); Antibodies: A Laboratory Manual, E. Harlow and D. Lane, ed., Cold Spring Harbor Laboratory Press (Cold Spring Harbor, N.Y., 1988); and Using Antibodies: A Laboratory Manual, E. Harlow and D. Lane, ed., Cold Spring Harbor Laboratory (Cold Spring Harbor, N.Y., 1999) which are incorporated by reference herein in their entireties.

Generation Of Heavychain Substitutions: The variable regions of the light chains of antibody clones G5, 10D3, 12G3, 1E11, 4C10, 4B11, and EA5 and the variable regions of the heavy chains of antibody clones G5, 10D3, 12G3, 1E11, 4C10, 4B11, and EA5 were individually cloned into mammalian expression vectors encoding a human cytomegalovirus major immediate early (hCMVie) enhancer, promoter and 5′-untranslated region (Boshart et al., 1985, Cell 41:521-30). In this system, a human γ1 chain is secreted along with a human κ chain (Johnson et al., 1997, J. Infect. Dis. 176:1215-24). All of the heavy chain substitutions were introduced by site-directed mutagenesis using a Quick Change Multi Mutagenesis Kit (Stratagene, Calif.) according to the manufacturer's instructions. Specifically, S60A/A61D were introduced into clones G5, 10D3, 12G3, 1E11, 4C10 and 4B11 using the primer: 5′-ACACAACAGAGTACGCTGACTCTGTGAAGGGTAGAG TCACCATT-3′; this generated heavy chain antibody clones G5/M, 10D3/M, 12G31M, 1E11/M, 4C10/M and 4B 11/M; N60A/Q61D were introduced into EA5 using the primers: 5′-GTTACAATGGTGTTACTAGCTACGCCGACAAGTTCAAGGGCAAGG CCAC-3′ and 5′-GTGGCCTTGCCCTTGAACTTGTCGGCGTAGCT AGTAACACCATTGTAAC-3′ generating EA5/M′; and S40A/N60A/Q61D were introduced into EA5 using the primers: 5′-CTACATGC ACTGGGTCAAGCAGGCCCATGGAAAGAGCCTTGAG-3′,5′-CTCAAGGCTCTTTCCATGGGCCTGCTTGACCCAGTGCATGTAG-3′,5′-GTTACAATGGTGTTACTAGCTACGCCGACAAGTTCAAGGGCAAGGCCAC-3′ and 5′-GTGGCCTTGCCCTTGAACTTGTCGGCGTAGCTAGT AACACCATTGTAAC-3′ generating EA5/M. Note that the light chains remain unaltered (FIG. 1A). The sequences were verified using an ABI 3100 sequencer. Human embryonic kidney (HEK) 293 cells were then transiently transfected with the various antibody constructs in 35 mm, 6-wells dishes using Lipofectamine and standard protocols. Supernatants were harvested twice at 72 and 144 hours post-transfection (referred to as 1^{st} and 2^{nd} harvest, respectively). The secreted, soluble human IgG1s were then assayed in terms of production yields and binding to original antigen (see below).

Measurement Of The Expression Yields: The expression yields of antibody clones G5, G5/M, 10D3, 10D3/M, 12G3, 12G3/M, 1E11, 1E11/M, 4C10, 4C10/M, 4B11 and 4B11/Mut were measured by ELISA. Transfection supernatants collected twice at three days intervals (see above) were assayed for antibody production using an anti-human IgG ELISA. Briefly, individual wells of a 96-well Biocoat plate (BD Biosciences, San Jose, Calif.) coated with a goat anti-human IgG were incubated with samples (supernatants) or standards (human IgG, 0.5-100 ng/ml), then with a horseradish peroxydase conjugate of a goat anti-human IgG antibody. Peroxydase activity was detected with 3,3′,5,5′-tetramethylbenzidine and the reaction was quenched with 0.2 M H₂SO₄. Plates were read at 450 nm. The results are summarized in Table 6.

TABLE 6
Producibility Improvements of Heavy Chain Modified Antibodies^{a}

| | Transfection | Transfection | Transfection | Transfection | Transfection | Fold |
|---|---|---|---|---|---|---|
| | #1 | #2 | #3 | #4 | #5 | increase^{d} |
| Modified | H1^{b} H2^{c} | H1 H2 | H1 H2 | H1 H2 | H1 H2 | H1 H2 |
| Antibody | μg/ml | μg/ml | μg/ml | μg/ml | μg/ml | μg/ml |
| G5 | 0.3-1.2 | 0.5-1.3 | 0.6-1.4 | | | |
| G5/M | 1.6-3.8 | 2.5-6.2 | | | | 4.4-3.8 |
| 1E11 | 0.7-2.0 | 1.2-3.4 | | | | |
| 1E11/M | 1.7-3.3 | 1.3-3.9 | | | | 1.6-1.3 |
| 4C10 | 2.0-3.0 | 2.4-3.2 | 2.1-3.3 | | | |
| 4C10/M | 3.2-5.8 | 3.8-7.3 | 5.0-4.6 | 6.8-7.8 | 5.1-7.7 | 2.2-2.1 |
| 10D3 | 0.7-1.7 | 1.4-3.5 | | | | |
| 10D3/M | 1.2-2.9 | 2.8-5.1 | | | | 2.0-1.5 |
| 12G3 | 0.9-2.3 | 1.8-3.6 | 1.4-2.4 | | | |
| 12G3/M | N.D. | 3.5-8.7 | 3.2-5.4 | 3.3-5.9 | 4.4-8.4 | 2.6-2.6 |
| 4B11 | 0.4-1.5 | 0.7-3.0 | | | | |
| 4B11/M | 1.0-2.3 | 2.4-5.2 | | | | 3.0-1.7 |
| EA5 | 2.7-2.8 | 1.0-1.2 | 4.0-2.9 | | | |
| EA5/M′ | 3.3-3.9 | 1.1-1.9 | 3.6-5.5 | | | 1.1-1.6 |
| EA5/M | 4.6-2.4 | 2.4-2.2 | 4.8-3.9 | | | 1.5-1.2 |

^{a}HEK 293 cells were transiently transfected with the various antibody constructs.
^{b}H1 = First Harvest (72 hours post-transfection).
^{c}H2 = Second Harvest (144 hours post-transfection).
^{d}Fold increase = average yield for each harvest (H1, H2) of the heavy chain modified “Mut” antibody divided by the average yield for each harvest of the unmodified antibody.

### Example 2

### Solid Phage Panning to Identify Clone 1C1

Immunotubes were coated with EphA2-Fc at 20 μg/ml in 0.1 M Carbonate buffer (pH 9.6, Sigma) and incubated at 4° C. overnight. The phage library (Fab310, Dyax) was precipitated with 20% of PEG (Fluka) at ⅕ volume and resuspended in PBS (pH 7.4). The phage library was then blocked with 2% milk and deselected with a non-EphA2 binding monoclonal antibody (to remove Fc binder). After blocking and deselecting, the phage library was transferred to the EphaA2 coated immunotube which was blocked with 2% milk. Two hours later the immunotube was washed with PBST (PBS+0.1% Tween) 10-20 times then with PBS 10-20 times to remove the unbound phage. The bound phage was eluted from the immunotube with 1 ml of 100 mM triethylamine (Sigma) and neutralized by adding 0.5 ml of 1 M Tris-HCl (pH 7.5, Invitrogen). Then, 1 volume of eluted and neutralized phage was mixed with 5 volumes of log phase TG1 cells (Novagen) and 4 volume of 2YT (Teknova). Samples were incubated at 37° C. for 30 min (water bath). Samples were then spun down at 4000 g and the pellet was resuspended in 2YT. Plate the cells on 2YT agar plates (Teknova) with carbenicillin and 2% glucose. The plate was incubated at 30° C. overnight. On the second day, colonies were collected and infected with helper phage (Invitrogen). The infected cells were cultured overnight in 2YT with carbenicillin (Invitrogen) and kanamycin (Sigma) at 30° C. to generate high titer phage. The phage was precipitated from the overnight culture and then the next round of panning occurred following the procedures described above. The anti-EphA2 antibody clone 1C1 was derived from the second round of panning.

### Generation of Anti-EphA2 Antibodies: 1F12, 1H3, 1D3, 2B12 and 5A8

Phage display technology was used to identify Fabs that bind to EphA2. Phage library fab310 from Dyax was used for soluble phase panning. Phage library was blocked with 2% milk and deselected with a non-EphA2 binding monoclonal antibody (to remove Fc binders). Streptavidin coated dynabeads (Dynal Biotech) was blocked in 1% milk. Blocked and deselected phage was exposed to 2.9 μg of biotinylated EphA2 and the EphA2-phage complex was captured by blocked dynabeads (Invitrogen). Bound phage was eluted using 1 mL of 100 mM triethylamine (Sigma) and elute was neutralized by adding 0.5 mL of 1M Tris-HCL. For infection, 1 volume of phage elute was mixed with 5 volumes of TG1 (Novagen) at log phase and four volumes of 2YT (Teknova). This mix was incubated for 30 minutes at 37° C. water bath. After infection, it was spun down at 4000 g for 5 minutes and the pellet was resuspended in 2YT. TG-1 cells were plated on 2YT plates containing 50 ug/ml carbenicillin and 2% glucose (Teknova) and were incubated at 30° C. overnight. On the second day, bacterial colonies were collected and infected with helper phage (Invitrogen). The infected cells were grown overnight in 2YT medium containing carbenicillin (Invitrogen) and kanamycin (Sigma) to generate high titer phage. The phage was concentrated from overnight culture by PEG precipitation. PEG precipitation was done using PEG/NaCl solution at one fifth volume of culture (PEG from Fluka). After precipitation, phage pellet was resuspended in one ML PBS (pH 7.4, Invitrogen) and was used for next round panning. Two more rounds of panning were done, in which biotinylated EphA2 concentration was decreased to 2.0 μg. The anti-EphA2 antibodies 1F12, 1D3, 1H3 and 2B 12 were from second round of panning and the anti-EphA2 antibody 5A8 was from the third round of panning.

### Transient Expression of Anti-EphA2 Antibodies

To express the whole antibody IgG, the variable regions of antibody heavy and light chains were cloned into mammalian cell IgG expression vector pABOE containing antibody constant region of IgG1/□ or IgG1/□ using the standard Molecular Biology techniques. Both the heavy and light chain expression cassettes were under the control of its own CMVie promoter. The antibody genes were transient transfected into HEK 293F by 293fectin transfection reagent following manufacture's protocol (Invitrogen). After three collections within 9 days, the proteins were purified by passing the culture supernatant through Protein A column (GE health care). The bound antibody were eluted with 50 mM citrate buffer (pH 3.2) and then dialyzed in PBS. All proteins were analyzed by SDS-polyacrylamide gel electrophoresis and were applied to quantitative ELISA using BCA kits (PIERCE) to determine antibody concentrations.

### Example 3

### Cell Surface Binding of α-EphA2 Antibodies

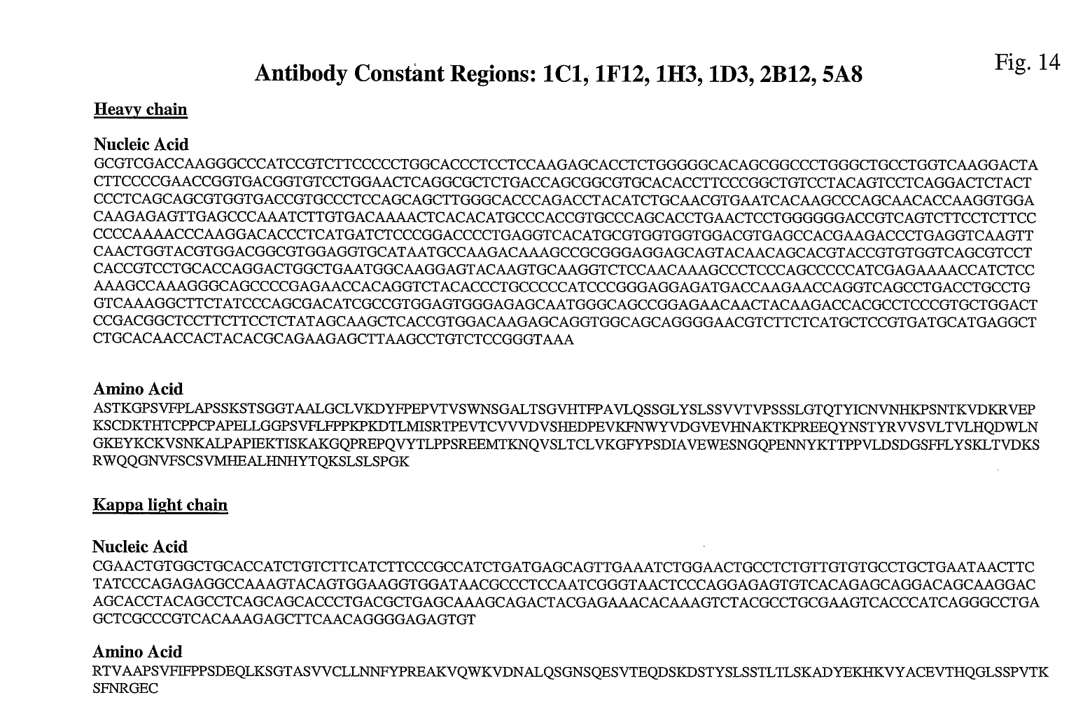

2×10⁵ cells in 150 μl FACS buffer (PBS+2% Fetal Bovine Serum+L-glutamine) were stained with 1 μg primary Abs (1C1, 1F12, 1H3, and 3F2 a-EphA2 Abs and R347 isotype control) for 30 minutes at 4° C. in v-bottom 96-well plates. The cells were then washed 2× with cold PBS and stained for 30 minutes at 4° C. with secondary Abs (Phycoerythin conjugated goat-a-human IgG, Biosource). Fluorescence analysis was performed using a FACS Calibur flow cytometer (BD Biosciences). Results of this experiment are summarized in FIGS. 14A and 14B herein and demonstrate the ability of each of these antibodies to bind to human, mouse and rat EphA2 expressed on tumor cells.

### Example 4

### Internalization of α-EphA2 Antibodies

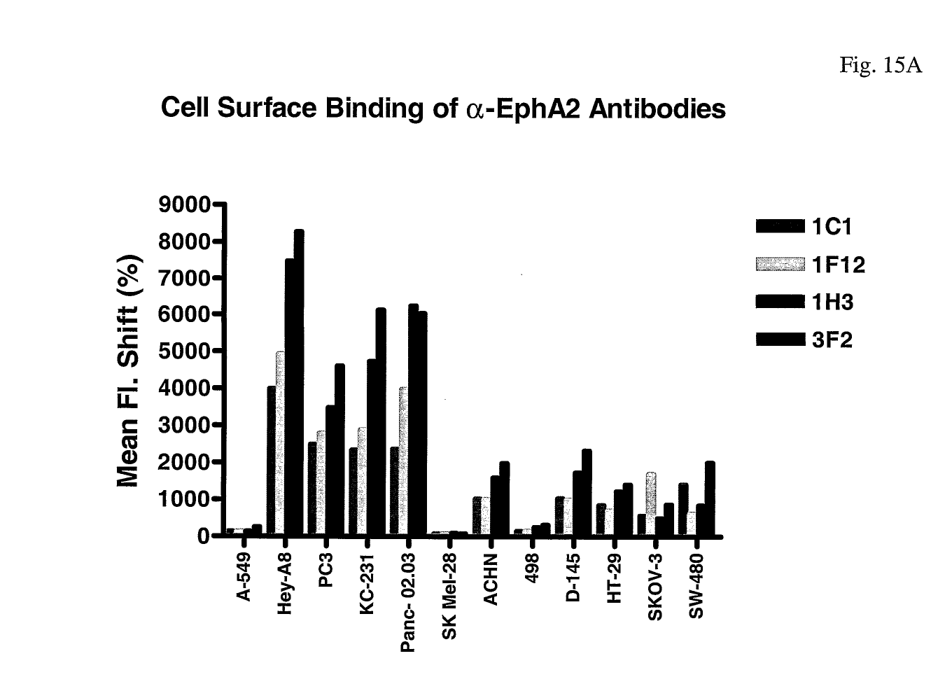

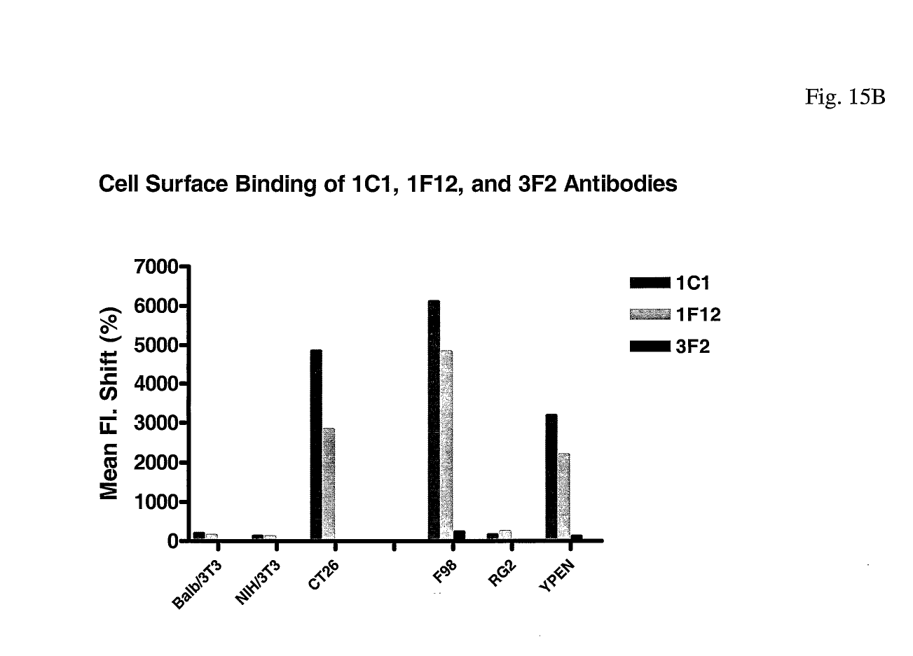

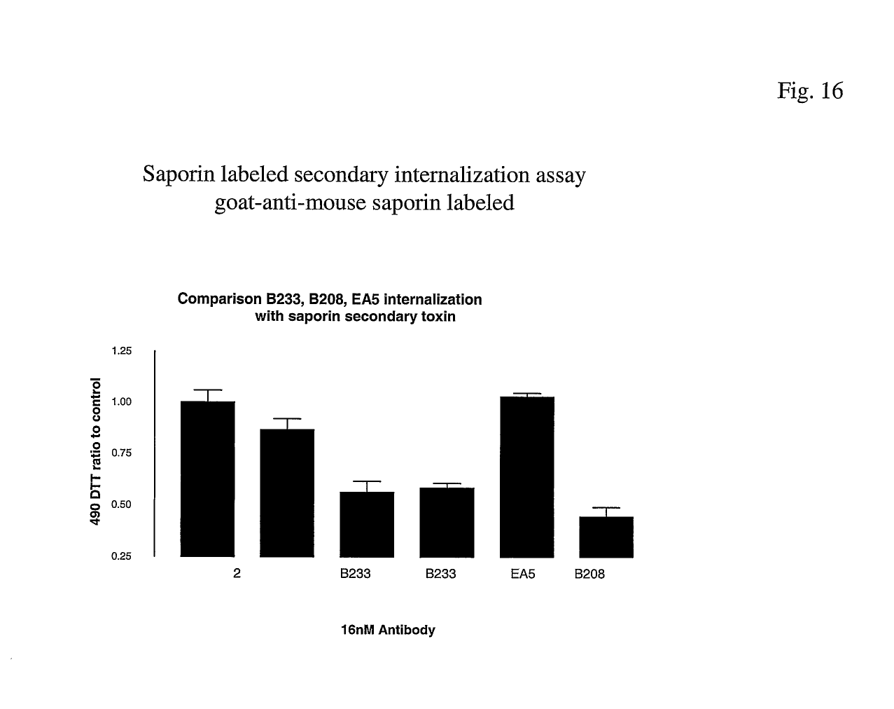

Anti-EphA2 antibodies (B233, B208, and EA5) and a secondary saporin (toxin) labeled monoclonal antibody (mAb) which recognizes the anti-EphA2 antibodies were coincubated and introduced to a tumor cell based monolayer (MCF-10A) and incubated for 72-96 hours. The purpose was to measure cell death, indicating that the mAb complex (anti-EphA2 mAb and the secondary mAb-saporin conjugate) was internalized. This assay was used as a pre-screen to select for an internalizing mAb. See Kohls et al., Biotechniques, 2000 January; 28(1): 162-5. The results of this experiment are summarized in FIGS. 15 and 16 herein.

### Example 5

### Fluorescent Visualization of Internalization of α-EphA2 Antibodies

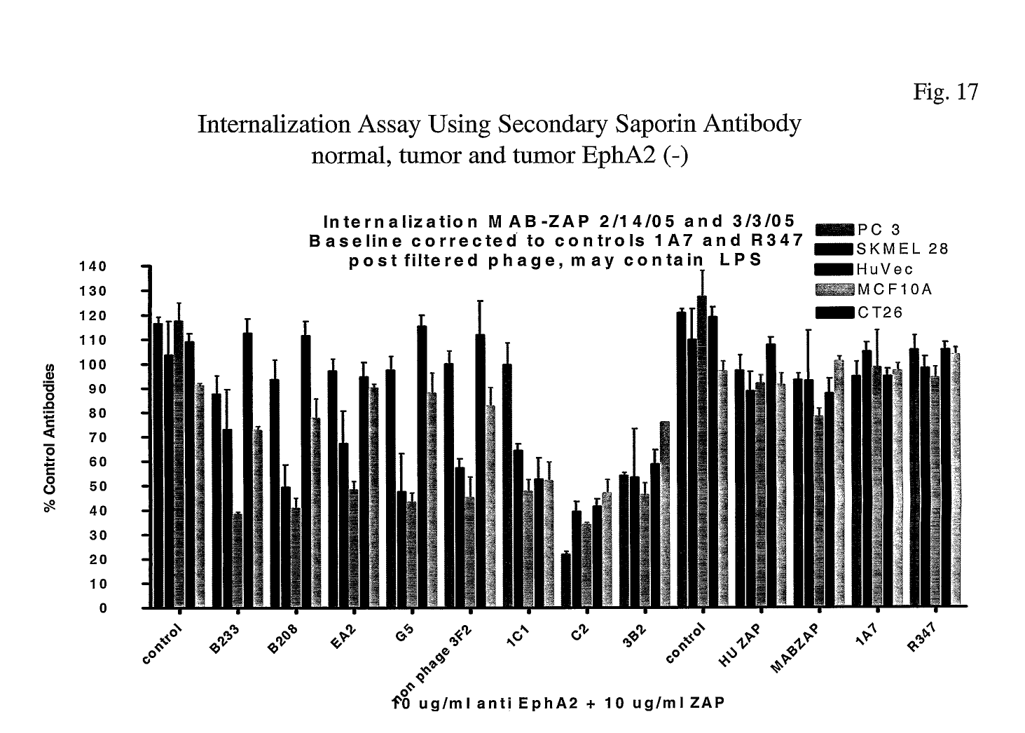

Cells (PC3, HUVEC, or CT26) were grown for 24-48 hours at 37° C./5% CO2 at a concentration of 2.5-5.0×10⁴ cells per 400 μl of appropriate growth media per chamber on Nunc's Tek II 8-chamber slides. Adherent cells were labeled with primary Abs (G5, 1C1, 1F12, or 3F2 anti-EphA2 Abs and R347 isotype control) at a concentration of 50 μg/ml for 30-45 minutes at 4° C. Cells were then washed 2× with PBS and cell-surface-bound primary Abs were allowed to internalize by covering the cells with growth media and incubation at 37° C./5% CO2 for 0 minutes, 20 minutes (FIGS. 18A-C and 19), or 60 minutes (FIG. 17).

Subsequent to internalization, cells were fixed (4% paraformaldehyde), permeabilized (0.5% Triton X-100), and labeled with secondary AlexaFluor 488 goat-α-human IgG Ab (Biosource), with 2× cold PBS washing in between steps.

Finally, the cells were covered with VECTASHIELD Mounting Media with DAPI (Vector Labs) and a coverslip prior to examination and photography with fluorescent confocal microscope. Results of the antibody internalization experiments are shown in the fluorescent microscopy pictures of FIGS. 16, 17, 18A, 18B, 18C, and 19 herein. EphA2 was rapidly internalized in these cell lines upon binding the agonistic EphA2 antibodies.

### Example 6

### EphA2 Receptor Activation

Cells were grown overnight at 37° C./5% CO2 at a concentration of 0.5×10⁶ cells per 3 ml of appropriate growth media per well in 6-well tissue culture plates. The next day old media was removed and replaced with fresh media containing 10 μg of 1C1 or 1F12 α-EphA2 Abs or R347 isotype control. The cells were incubated for 15 minutes at 37° C./5% CO2 to activate EphA2 receptor. Subsequent to activation the cells were washed 1× with cold PBS and lysed on ice with 1% Triton X-100 lysis buffer containing Phosphatase Inhibitor Cocktails 1 and 2 (Sigma) and Complete Protease Inhibitor Cocktail Tablets (Roche), added as per manufacturer's recommendation. D7 α-EphA2 mAb and 50 μl of protein A sepharose beads pre-conjugated to rabbit anti-mouse IgG were mixed with lysate at 4° C. overnight to immunoprecipitate the proteins.

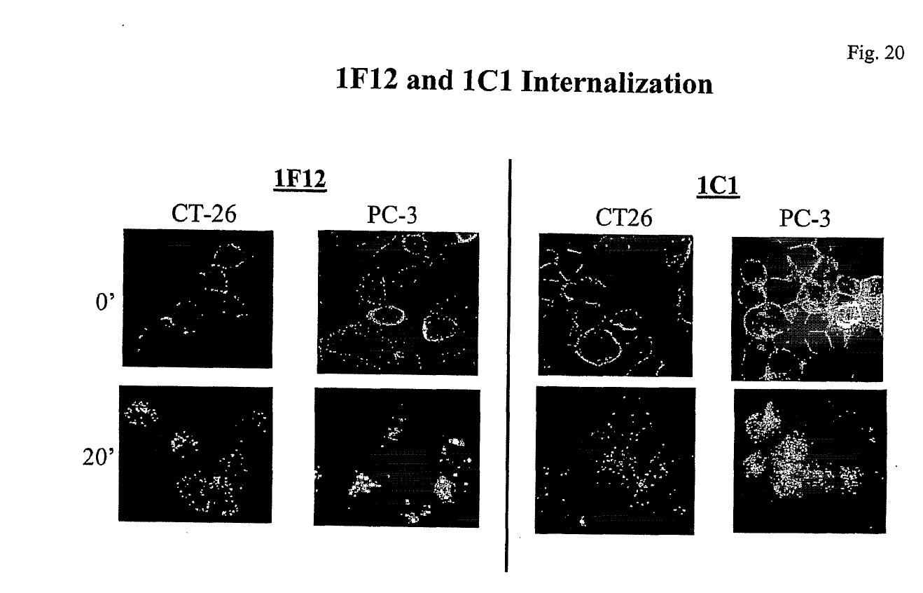

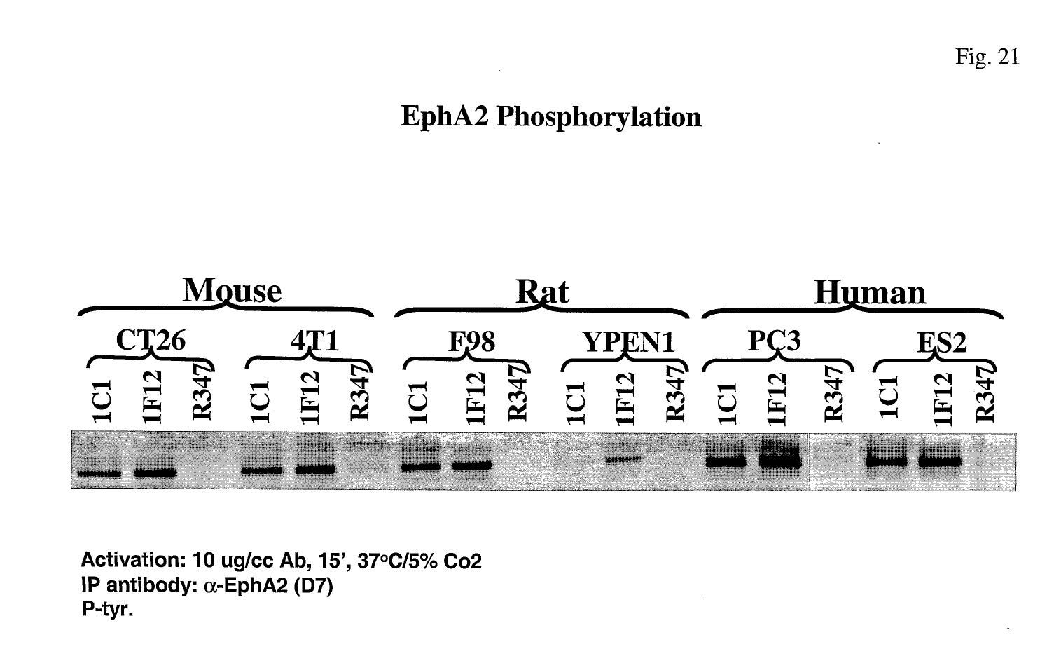

Protein lysates were resolved by 10% Bis-Tris NuPAGE Western gel and transferred electrophoretically to nitrocellulose membranes (Invitrogen) following manufacturer's protocol. The blots were incubated with 1 μg/ml primary (mouse α-phosphotyrosine IgG2bk, clone 4-G10, Upstate) and secondary (peroxidase-conjugated goat-a-mouse IgG, Jackson Immuno Research) Abs to identify activated (phosphorylated) protein bands using the Super Signal ECL kit (Pierce) and developed using Amersham Biosciences Hyperfilm. See also Coffman et al., Cancer Res. 63: 7907-7912, 2003. Results of the EphA2 receptor activation experiments are summarized in FIGS. 20 and 21 herein and demonstrate the ability of each of these antibodies to activate EphA2 on various human, mouse and rat tumor cell lines.

### Example 7

### Eph Receptor Cross Reactivity ELISA Assay

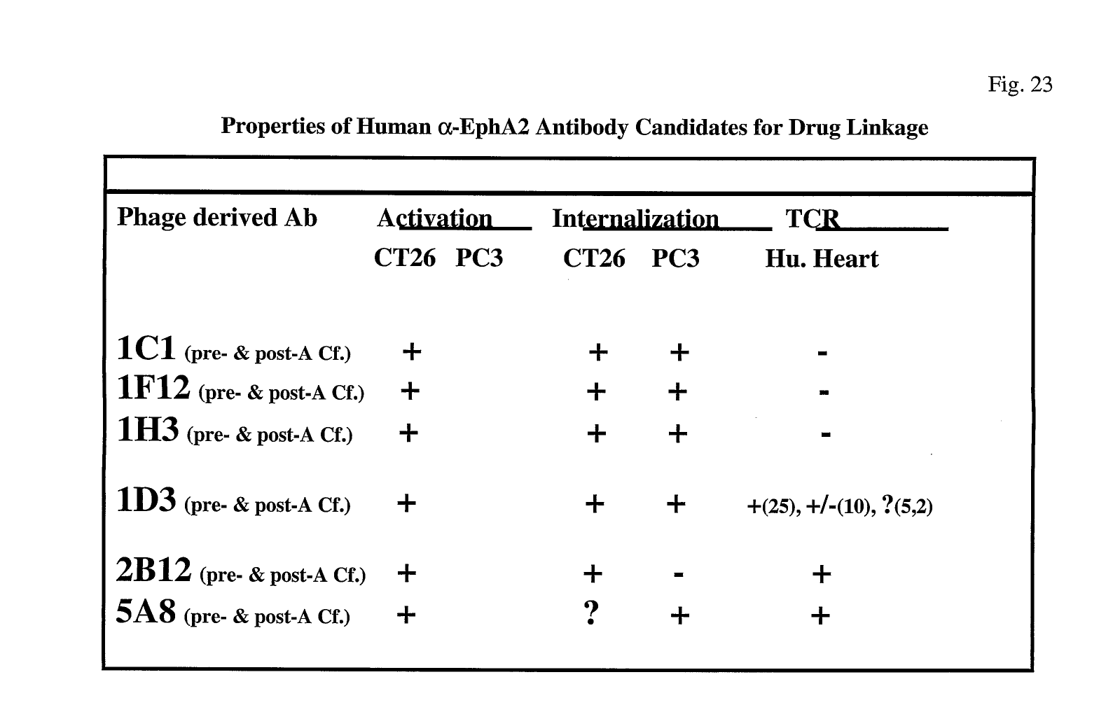

In order to determine if the anti-EphA2 antibodies 1C1 and 1F12 demonstrated any binding to murine members of the Eph family of receptors, the following assay was performed. The anti-EphA antibodies were diluted 1:2 through 8 wells starting at a concentration of 5 μl/ml in PBS (pH 7.2). EIA/RIA ELISA plates (Costar cat. 3690) were coated with 50 μl of the diluted antibodies and incubated at 4° C. overnight. The next day, the plates were washed using an Elₓ405 auto plate washer programmed for five dispense/aspirate wash steps with 1×PBST (1×PSB, 0.1% Tween 20) separated by 3 second shaking intervals. The plates were patted dry on a stack of paper towels and blocked with 240 μl of blocking buffer (2% BSA w/v in 1×PBST) for one hour at room temperature. Eph receptors were biotinylated with EZ-link sulfo-NHS-Biotin Reagent (Pierce cat. 21335) at a challenge ratio of eight biotins/Eph receptor molecule. The biotinylated Eph receptors were quenched with 50 mM Tris-HCl (Invitrogen cat 15506-017) and a dilution to 1 μg/ml was made in blocking buffer. The plates were washed again using the Elₓ405 auto plate washer and patted dry. To each well, 50 μl of the diluted biotinylated Eph receptors were added and incubated at 37° C. for one hour. The plates were washed and dried as before and 50 μl neutravidin-HRP 1:12500 (Pierce cat. 31002) added. After an hour incubation at 37° C., the plates were washed, rotated 180° and washed again. The plates were patted dry and 50 μl of SureBlue TMB peroxidase (KPL cat. 52-00-03) was added to each well and allowed to develop for 5-10 minutes. The reaction was stopped with 50 μl of 0.2M H₂SO₄ and the ELISA signal was read at 450 nM. The results of this experiment are summarized in FIG. 23, with 1C1 demonstrating binding to murine EphA2 and murine EphA4, and 1F12 demonstrating binding to murine EphA2, 3, 4, 5, 6, 7, 8, and murine EphB1 and 2.

### Example 8

### In Vitro Growth Inhibition Assays

Conjugation of Antibodies:

EphA2 was conjugated to either Monomethylyauristatin E (MMAE) or Monomethylauristatin F (MMAF) using a valine-citrulline (vc) or a maleimidocaproyl-citrulline (mc) linker. Antibodies were conjugated at Seattle Genetics, Inc. according to previously described protocols (Doronina et al. BioConjug Chem. 2006; Doronina et al. Nat Biotech 2003).

### In Vitro Growth Inhibition Assays

Cells were grown overnight at 37° C./5% CO2 at a concentration of 2.0−3.0×10³ cells per 150 μl of growth media (RPMI 1640+10% Fetal Bovine Serum) per well in tissue culture treated 96-well plates (Falcon BD). The following day old media was removed and replaced with 120 μl of fresh media per well. Separate drug dilution plates were prepared and 30 μl of each dilution was transferred to the cells.

The plates were incubated at 37° C./5% CO2 for additional 3-4 days and harvested using the CellTiter-GloLuminescent Cell Viability Assay kit (Promega). Cellular viability was determined as measurement of luminescence using a Wallac Victor II plate reader.

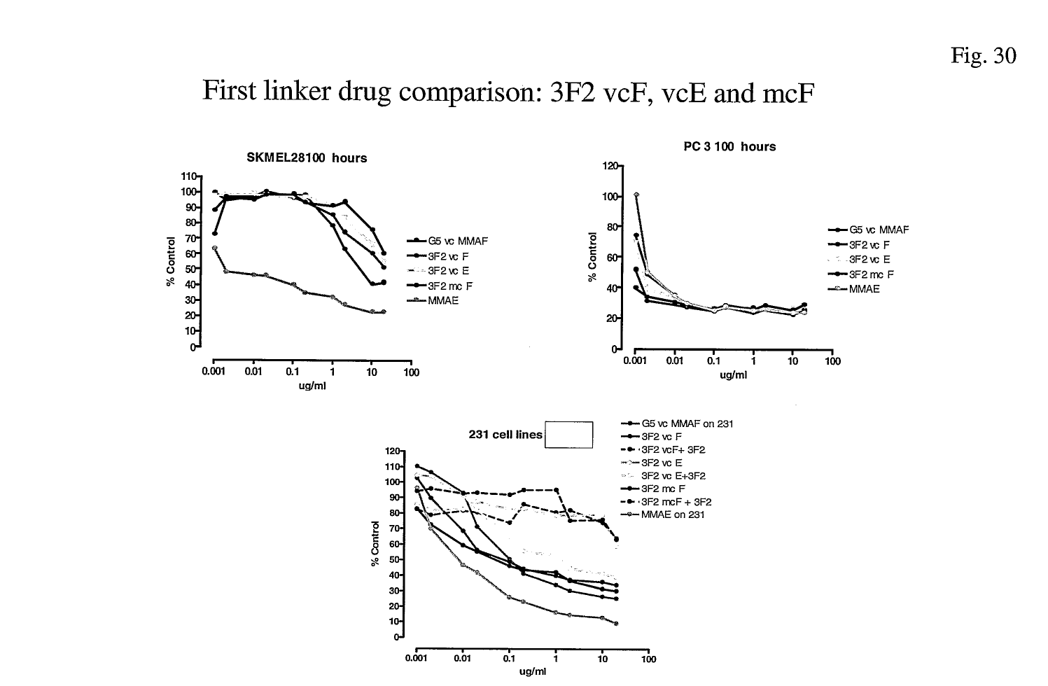

Cells tested in different in vitro growth inhibition assays were the following: PC3, SKMEL-28, A549, MDA-MB-231, 231KC, A375, HCT-116, SW620, MDA-MB-468, MDA-MB-435, T231, HUVEC, H460, M21, SKOV-3, HeyA8, Panc.02.03, DU145, ACHN, OVCAR-3, HT29, MCF10-A, F98, and CYNO-MK. Antibodies tested in different in vitro growth inhibition assays were the following: G5vcMMAF, 3F2vcMMAE, 3F2vcMMAF, 3F2mcMMAF, EA5vcMMAF, 1A7MMAF, R347vcMMAF, R347mcMMAF, 1C1mcMMAF, 1F12mcMMAF, 1C1vcMMAE, and 1F12vcMMAE. Results of the numerous different in vitro growth inhibition assays performed are summarized in FIGS. 30-47 herein and demonstrate the ability of the various EphA2 conjugates to specifically inhibit the growth of EphA2 expressing tumor cell lines.

### Example 9

### In Vivo Efficacy Testing of Anti-EphA2 ADC G5 Against Various Cancer Models

Athymic nu/nu (Harlan, Somerville, N.J.) female mice 4-6 weeks of age were injected subcutaneously with 5×10⁶ tumor cells. Treatments of PBS or antibody drug conjugates were injected every fourth day for a total of 5 doses in the intraperitoneal cavity after the tumors had reached an average size of 100-150 mm³ as indicated in the figure legends. Each treatment group consisted of groups of mice ranging in number from 10-12.

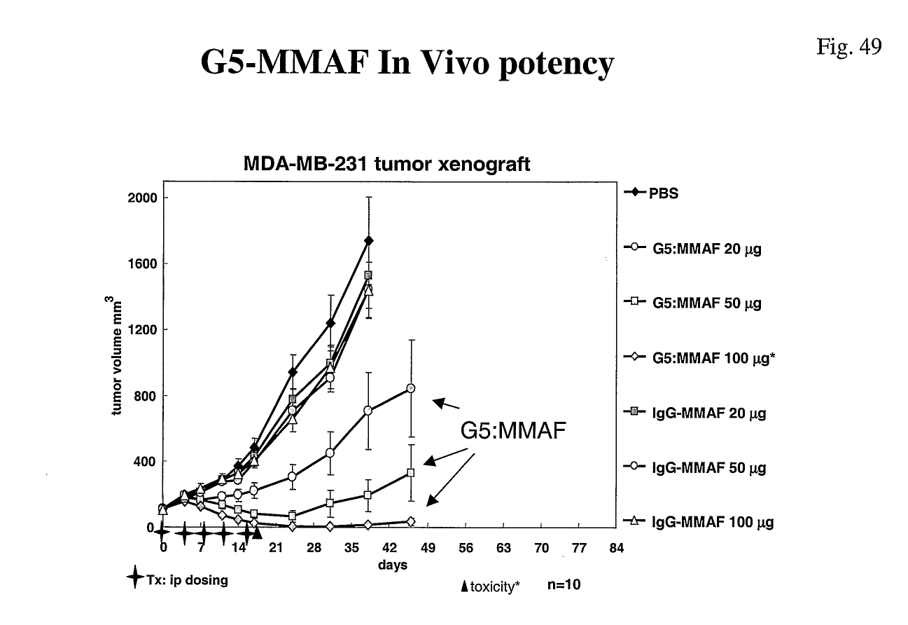

Tumor volume measurements were taken with a caliper routinely (1-2 times/week) starting at the initiation of drug treatment. Results of these studies are summarized in FIGS. 49-51 herein. The results demonstrate that G5 conjugated to MMAF with the vc linker specifically inhibited PC3 and MDA-MB231 tumor growth in vivo in a dose-dependent manner.

### Example 10

### In Vivo Efficacy Testing of Anti-EphA2 ADC 3F2 in a Prostate Cancer Model

Athymic nu/nu (Harlan, Somerville, N.J.) female mice 4-6 weeks of age were injected subcutaneously with 5×10⁶ tumor cells. Treatments of PBS or antibody drug conjugates were injected every fourth day for a total of 5 doses in the intraperitoneal cavity after the tumors had reached an average size of 100-150 mm³ as indicated in the figure legends. Each treatment group consisted of groups of mice ranging in number from 10-12.

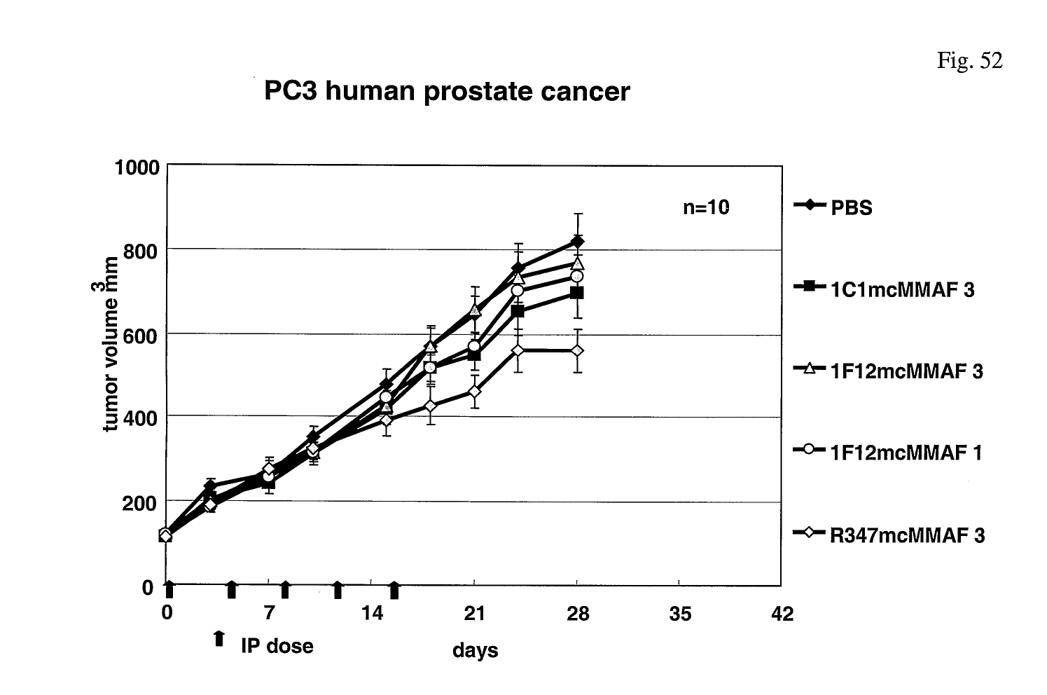

Tumor volume measurements were taken with a caliper routinely (1-2 times/week) starting at the initiation of drug treatment. Results of this study are summarized in FIG. 52 herein. The results demonstrate that 3F2 conjugated to either MMAE with a vc linker or MMAF with a mc linker can specifically inhibit PC3 tumor growth in vivo.

### Example 11

### In Vivo Efficacy Testing of Anti-EphA2 ADC's 1C1 and 1F12 in Various Cancer Models

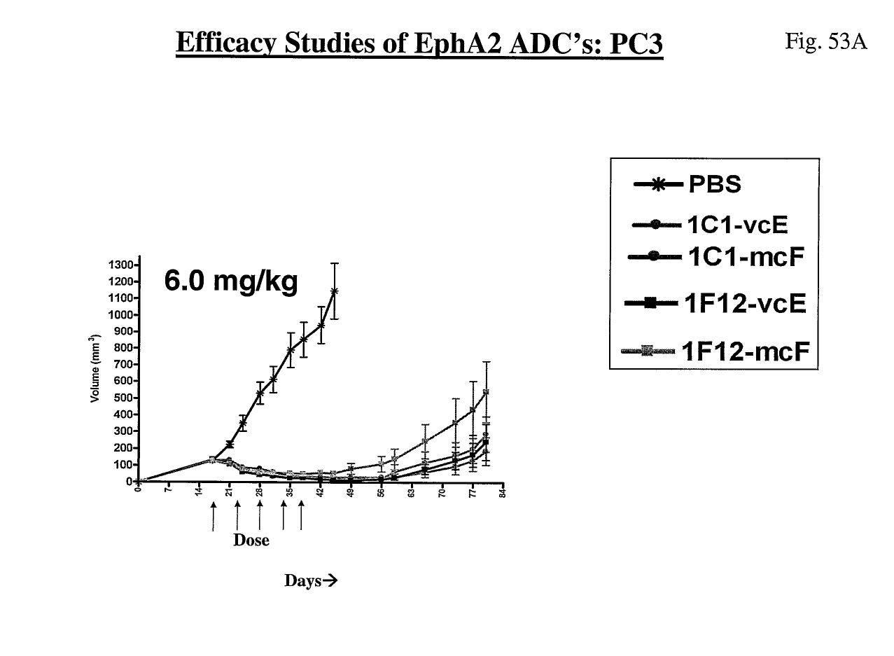

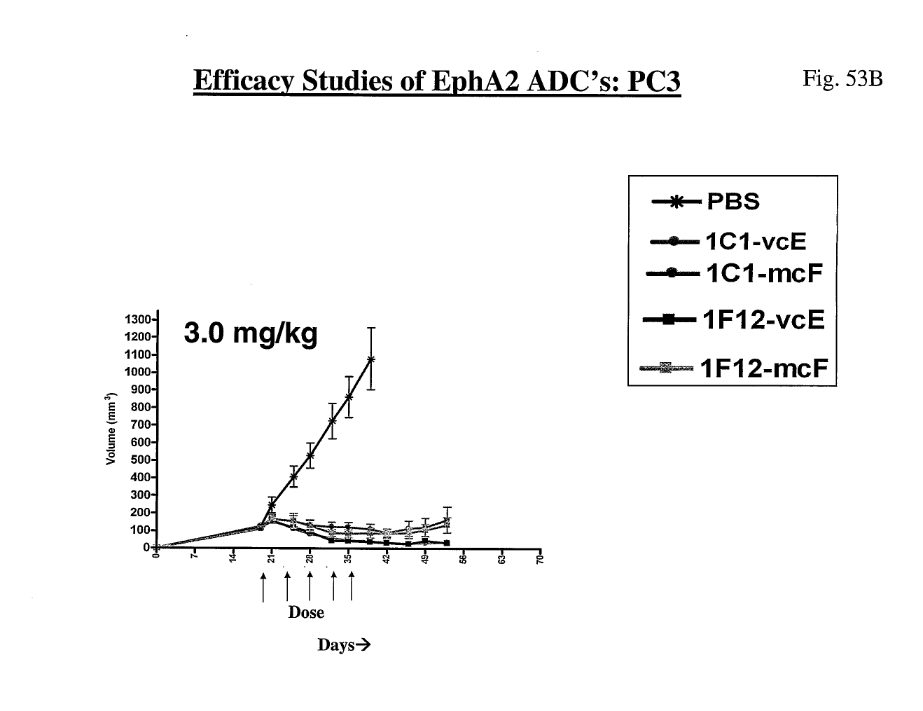

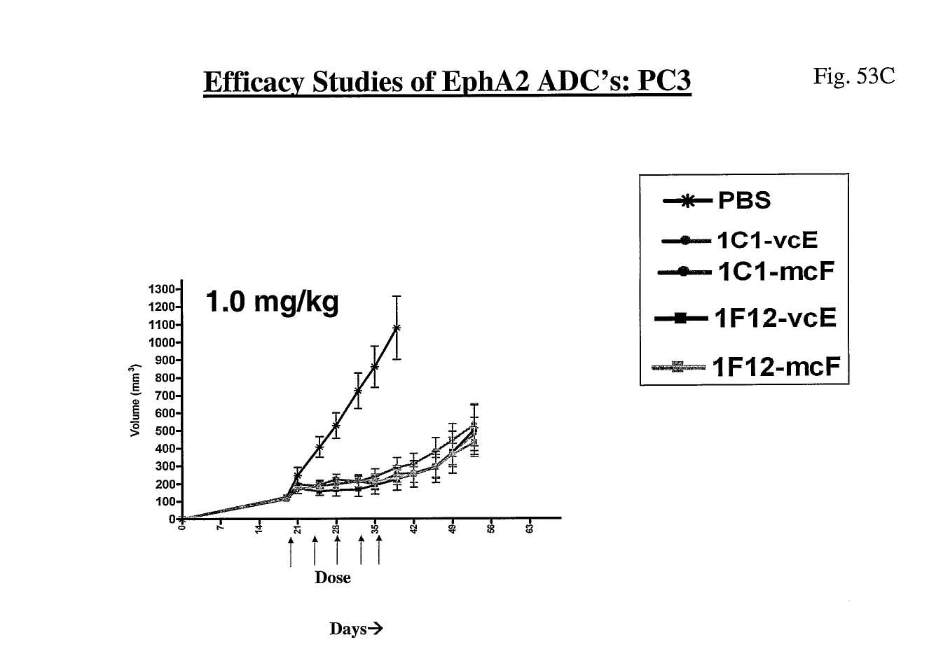

Athymic nu/nu (Harlan, Somerville, N.J.) female mice 4-6 weeks of age were injected subcutaneously with 5×10⁶ tumor cells. Treatments of PBS or antibody drug conjugates were injected every fourth day for a total of 5 doses in the intraperitoneal cavity after the tumors had reached an average size of 100-150 mm³ as indicated in the figure legends. Doses ranged from 1 mg/kg (20 μg) to 10 mg/kg (200 μg) as described herein for each Figures (see FIGS. 53-56C descriptions herein). Each treatment group consisted of groups of mice ranging in number from 10-12.

Tumor volume measurements were taken with a caliper routinely (1-2 times/week) starting at the initiation of drug treatment. Results of these studies are summarized in FIGS. 53-56C herein. The results demonstrate that 1C1 and 1F12 conjugated to MMAF with the mc linker specifically inhibited PC3 and MDA-MB231 tumor growth in vivo in a dose-dependent manner and was well-tolerated.

### Example 12

### In Vivo Toxicity Studies

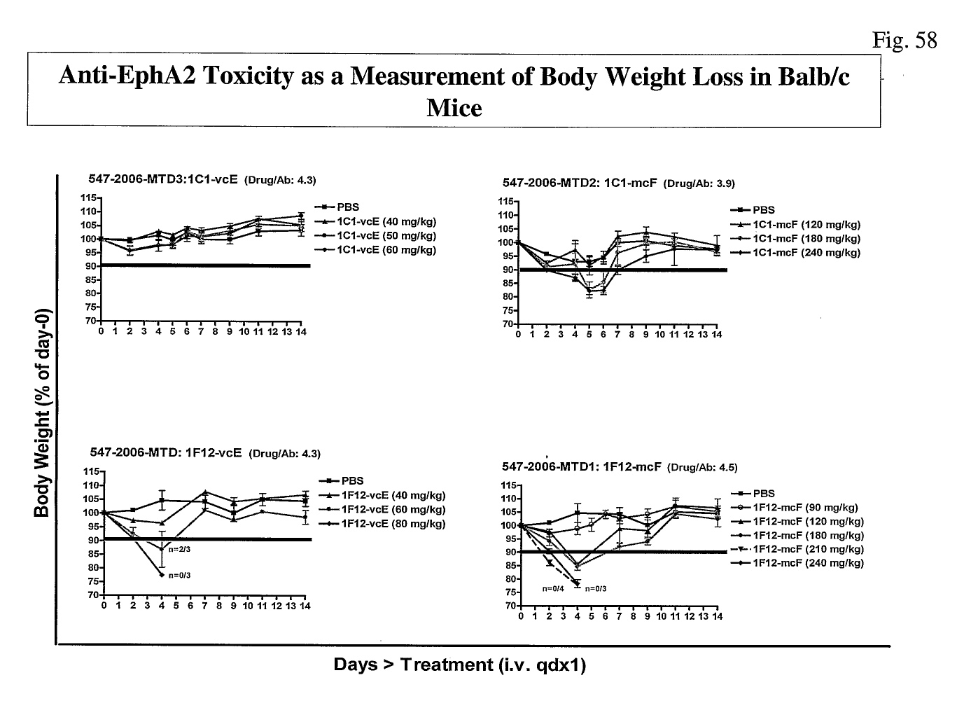

Female Balb/c mice (Harlan, Somerville, N.J.) 4-6 weeks of age were injected via the tail vein (single bolus) with PBS or antibody drug conjugates (1C1 and 1F12 conjugated to MMAE with the vc linker, or conjugated to MMAF with the mc linker) at the following dose levels: the vcMMAE antibodies were at 40 mg/kg, 50 mg/kg, and 60 mg/kg; the 1C1-mcMMAF antibody was at 120 mg/kg, 180 mg/kg, and 240 mg/kg; and the 1F12-mcMMAF antibody was 90 mg/kg, 120 mg/kg, 180 mg/kg, 210 mg/kg, and 240 mg/kg. Daily observations and body weight measurements were recorded for 14 days following drug administration. Each treatment group consisted of 3-4 mice. Any animals demonstrating signs of morbidity (hunched posture, impaired breathing, decreases mobility, greater than 20% weight loss, etc.) were humanely euthanized by CO2 asphyxiation. Results of these in vivo toxicity studies are summarized in FIG. 58 herein and demonstrate the relative tolerability of each antibody drug conjugate as it relates to body weight loss.

Whereas, particular embodiments of the invention have been described above for purposes of description, it will be appreciated by those skilled in the art that numerous variations of the details may be made without departing from the invention as described in the appended claims.

All publications, patents and patent applications mentioned in this specification are herein incorporated by reference into the specification to the same extent as if each individual publication, patent or patent application was specifically and individually indicated to be incorporated herein by reference.

We claim:

1-108. (canceled)

109. An internalizing antibody drug conjugate (ADC) that specifically binds to EphA2, wherein said drug is a toxin.

110. The ADC of claim 109, wherein said ADC comprises a spacer, and a linker.

111. The ADC of claim 110, wherein said linker is a maleimidocaproyl-citrulline linker.

112. The ADC of claim 110, wherein said linker is a valine-citrulline linker.

113. The ADC of claim 109, wherein said toxin is an anti-tubulin agent.

114. The ADC of claim 113, wherein said anti-tubulin agent is an auristatin.

115. The ADC of claim 114, wherein said auristatin is an auristatin E or an auristatin F.

116. The ADC of claim 115, wherein said auristatin is monomethyl auristatin F (MMAF).

117. The ADC of claim 116, wherein said auristatin is monomethyl auristatin E (MMAE).

118. The ADC of claim 109 comprising a variable heavy (VH) domain and a variable light (VL) domain wherein said VH domain comprises an amino acid sequence selected from the group consisting of:
a. 12G3H11 (SEQ ID NO:165);
b. B233 (SEQ ID NO:87);
c. B208 (SEQ ID NO: 95);
d. G5 (SEQ ID NO: 103);
e. 10C12 (SEQ ID NO: 140);
f. 4H5 (SEQ ID NO: 138);
g. 3F2 (SEQ ID NO: 63);
h. 1C1 (SEQ ID NO:3);
i. 1F12 (SEQ ID NO:13);
j. 1H3 (SEQ ID NO:23);
k. 1D3 (SEQ ID NO:33);
l. 2B12 (SEQ ID NO:43); and
m. 5A8 (SEQ ID NO:53).

119. The ADC of claim 109 comprising a variable heavy (VH) domain and a variable light (VL) domain wherein said VL domain comprises an amino acid sequence selected from the group consisting of:
a. 12G3H11 (SEQ ID NO: 166);
b. B233 (SEQ ID NO:88);
c. B208 (SEQ ID NO: 96);
d. G5 (SEQ ID NO:104);
e. 10C12 (SEQ ID NO:142);
f. 4H5 (SEQ ID NO:137);
g. 3F2 (SEQ ID NO: 64);
h. 1C1 (SEQ ID NO:4);
i. 1F12 (SEQ ID NO:14);
j. 1H3 (SEQ ID NO:24);
k. 1D3 (SEQ ID NO:34);
l. 2B12 (SEQ ID NO:44); and
m. 5A8 (SEQ ID NO:54).

120. The ADC of claim 109 comprising six complementary determining regions (CDRs) wherein said CDRs comprise an amino acid sequence selected from the group consisting of:
a. B233 VH CDR1(SEQ ID NO:89), VH CDR2(SEQ ID NO:90), VH CDR3(SEQ ID NO:91), VL CDR1(SEQ ID NO:92), VL CDR2(SEQ ID NO:93), and VL CDR3(SEQ ID NO:94);
b. B208 VH CDR1(SEQ ID NO:97), VH CDR2(SEQ ID NO:98), VH CDR3(SEQ ID NO:99), VL CDR1(SEQ ID NO: 100), VL CDR2(SEQ ID NO:101), and VL CDR3(SEQ ID NO:102);
c. G5 VH CDR1(SEQ ID NO: 105), VH CDR2(SEQ ID NO:106), VH CDR3(SEQ ID NO: 107), VL CDR1(SEQ ID NO: 108), VL CDR2(SEQ ID NO: 109), and VL CDR3(SEQ ID NO: 110);
d. 3F2 VH CDR1(SEQ ID NO:65), VH CDR2(SEQ ID NO:66), VH CDR3(SEQ ID NO:67), VL CDR1(SEQ ID NO:68), VL CDR2(SEQ ID NO:69), and VL CDR3(SEQ ID NO:70);
e. 1C1 VH CDR1(SEQ ID NO:5), VH CDR2(SEQ ID NO:6), VH CDR3(SEQ ID NO:7), VL CDR1(SEQ ID NO:8), VL CDR2(SEQ ID NO:9), and VL CDR3(SEQ ID NO: 10);
f. 1F12 VH CDR1(SEQ ID NO:15), VH CDR2(SEQ ID NO:16), VH CDR3(SEQ ID NO:17), VL CDR1(SEQ ID NO: 18), VL CDR2(SEQ ID NO: 19), and VL CDR3(SEQ ID NO:20);
g. 1H3 VH CDR1(SEQ ID NO:25), VH CDR2(SEQ ID NO:25), VH CDR3(SEQ ID NO:27), VL CDR1(SEQ ID NO:28), VL CDR2(SEQ ID NO:29), and VL CDR3(SEQ ID NO:30);
h. 1D3 VH CDR1(SEQ ID NO:35), VH CDR2(SEQ ID NO:36), VH CDR3(SEQ ID NO:37), VL CDR1(SEQ ID NO:38), VL CDR2(SEQ ID NO:39), and VL CDR3(SEQ ID NO:40);
i. 2B12 VH CDR1(SEQ ID NO:45), VH CDR2(SEQ ID NO:46), VH CDR3(SEQ ID NO:47), VL CDR1(SEQ ID NO:48), VL CDR2(SEQ ID NO:49), and VL CDR3(SEQ ID NO:50); and
j. 5A8 VH CDR1(SEQ ID NO:55), VH CDR2(SEQ ID NO:56), VH CDR3(SEQ ID NO:57), VL CDR1(SEQ ID NO:58), VL CDR2(SEQ ID NO:59), and VL CDR3(SEQ ID NO:60).

121. A vector comprising the nucleic acid that encodes the ADC of any of claims 109-120.

122. A host cell comprising the vector of claim 121.

123. A method of treating cancer in a patient in need thereof, said method comprising administering to said patient a therapeutically effective amount of the ADC of claim 109.

124. The method of claim 123, wherein said administration increases EphA2 phosphorylation in a cancer cell relative to the level of EphA2 phosphorylation in an untreated cancer cell.

125. The method of claim 123, wherein said cancer is of an epithelial cell origin.

126. The method of claim 125 wherein said cancer comprises cells that overexpress EphA2 relative to non-cancer cells having the tissue type of said cancer cells.

127. The method of claim 125, wherein said cancer is a cancer of the skin, lung, colon, breast, prostate, bladder, kidney, or pancreas or is a renal cell carcinoma or melanoma.

128. The method of claim 123, wherein said cancer is a metastatic cancer.

129. The method of claim 123 comprising the administration of an additional anti-cancer therapy that is not an EphA2 antibody.

130. The method of claim 129, wherein said additional cancer therapy is selected from the group consisting of chemotherapy, biological therapy, immunotherapy, radiation therapy, hormonal therapy and surgery.

131. A pharmaceutical composition comprising a therapeutically effective amount of the ADCs of any one of claims 109-120 and a pharmaceutically acceptable carrier.

132. The pharmaceutical composition of claim 131, comprising an anti-cancer agent that is not an EphA2 antibody.

133. The pharmaceutical composition of claim 131, wherein said anti-cancer agent is a chemotherapeutic agent, a radiation therapeutic agent, a hormonal therapeutic agent, a biological therapeutic or an immunotherapeutic agent.
